SEQUENCE LISTING (350 sequences)

SEQ ID NO: 1 - protein, 311 aa, synthetic construct
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - protein, 279 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 3 - protein, 162 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 4 - protein, 21 aa, synthetic construct
ALATGACIVGILCLPLILLLV

SEQ ID NO: 5 - protein, 96 aa, synthetic construct
YKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 6 - protein, 136 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQV

SEQ ID NO: 7 - protein, 431 aa, synthetic construct
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 8 - protein, 430 aa, synthetic construct
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 9 - protein, 406 aa, synthetic construct
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 10 - protein, 409 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 11 - protein, 409 aa, synthetic construct; identical to SEQ ID NO: 10

SEQ ID NO: 12 - protein, 385 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 13 - protein, 219 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 14 - protein, 195 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIG

SEQ ID NO: 15 - protein, 21 aa, synthetic construct
LIAGAIGTGAVIIIFCIALIL

SEQ ID NO: 16 - protein, 169 aa, synthetic construct
GAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 17 - protein, 114 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVT

SEQ ID NO: 18 - protein, 91 aa, synthetic construct
PSAPHCQIQGSQDIGSDVILLCSSEEGIPRPTYLWEKLDNTLKLPPTATQDQVQGTVTIRNISALSSGLYQCVASNAIGTSTCLLDLQVIS

SEQ ID NO: 19 - protein, 414 aa, synthetic construct
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 20 - protein, 393 aa, synthetic construct
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 21 - protein, 242 aa, synthetic construct
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIG

SEQ ID NO: 22 - protein, 21 aa, synthetic construct
VIIGIVLGSLLALGCLAVGIW

SEQ ID NO: 23 - protein, 130 aa, synthetic construct
GLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 24 - protein, 120 aa, synthetic construct
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVT

SEQ ID NO: 25 - protein, 112 aa, synthetic construct
PAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVS

SEQ ID NO: 26 - protein, 14 aa, synthetic construct
SRGEVQTCSERRPI

SEQ ID NO: 27 - protein, 14 aa, synthetic construct
FQDLHLHHGGHQAA

SEQ ID NO: 28 - protein, 14 aa, synthetic construct
CLVVEIRHHHSEHR

SEQ ID NO: 29 - protein, 11 aa, synthetic construct
DKGHDVTFYKT

SEQ ID NO: 30 - protein, 11 aa, synthetic construct
RHHHSEHRVHG

SEQ ID NO: 31 - protein, 102 aa, synthetic construct
DKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHG

SEQ ID NO: 32 - protein, 119 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSS

SEQ ID NO: 33 - protein, 9 aa, synthetic construct
GYSITSDYA

SEQ ID NO: 34 - protein, 7 aa, synthetic construct
ITYSGNI

SEQ ID NO: 35 - protein, 12 aa, synthetic construct
ARSLYYPWYFDV

SEQ ID NO: 36 - protein, 25 aa, synthetic construct
EVKLVESGPGLVKPSQSLSLTCTVT

SEQ ID NO: 37 - protein, 17 aa, synthetic construct
WNWIRQFPGNKLEWMGY

SEQ ID NO: 38 - protein, 38 aa, synthetic construct
SYNPSLRSRISITRDTSKNQFFLQLNSVTPEDTATYSC

SEQ ID NO: 39 - protein, 11 aa, synthetic construct
WGAGTTVTVSS

SEQ ID NO: 40 - protein, 107 aa, synthetic construct
DIVITQTPAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVK

SEQ ID NO: 41 - protein, 5 aa, synthetic construct
SSVGY

SEQ ID NO: 42 - skipped

SEQ ID NO: 43 - protein, 10 aa, synthetic construct
QQWSSYPPIT

SEQ ID NO: 44 - protein, 26 aa, synthetic construct
DIVITQTPAILSTSPGEKVTMTCRAS

SEQ ID NO: 45 - protein, 17 aa, synthetic construct
IHWYQQKPGSSPKPWIY

SEQ ID NO: 46 - protein, 36 aa, synthetic construct
NLASGVPARFSGSGSGTSNSLTITRVEAEDAATYYC

SEQ ID NO: 47 - protein, 10 aa, synthetic construct
FGGGTKLEVK

SEQ ID NO: 48 - protein, 119 aa, synthetic construct
EVMLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSS

SEQ ID NO: 49 - protein, 25 aa, synthetic construct
EVMLVESGPGLVKPSQSLSLTCTVT

SEQ ID NO: 50 - protein, 107 aa, synthetic construct
DIVLTQTTAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVK

SEQ ID NO: 51 - protein, 25 aa, synthetic construct
DIVLTQTTAILSTSPGEKVTMTCRA

SEQ ID NO: 52 - protein, 119 aa, synthetic construct
EVQLLESGGGLVQPGGSLRLSCAASGYTITSDYAMSWVRQAPGKGLEWVSVITYSGNISYADSVKGRFTISRDNSKNTLYLQMNSLRAEDTAVYYCARSLYYPWYFDVWGAGTTVTVSS

SEQ ID NO: 53 - protein, 9 aa, synthetic construct
GYTITSDYA

SEQ ID NO: 54 - protein, 25 aa, synthetic construct
EVQLLESGGGLVQPGGSLRLSCAAS

SEQ ID NO: 55 - protein, 17 aa, synthetic construct
MSWVRQAPGKGLEWVSV

SEQ ID NO: 56 - protein, 38 aa, synthetic construct
SYADSVKGRFTISRDNSKNTLYLQMNSLRAEDTAVYYC

SEQ ID NO: 57 - protein, 107 aa, synthetic construct
EIVITQSPATLSLSPGERATLSCRASSSVGYLAWYQQKPGQAPRPLIYDTSNRATGIPARFSGSGSGTDNTLTISSLEPEDFAVYYCQQWSSYPPITFGGGTKLEVK

SEQ ID NO: 58 - skipped

SEQ ID NO: 59 - protein, 26 aa, synthetic construct
EIVITQSPATLSLSPGERATLSCRAS

SEQ ID NO: 60 - protein, 17 aa, synthetic construct
LAWYQQKPGQAPRPLIY

SEQ ID NO: 61 - protein, 36 aa, synthetic construct
NRATGIPARFSGSGSGTDNTLTISSLEPEDFAVYYC

SEQ ID NO: 62 - protein, 119 aa, synthetic construct
QVQLQESGPGLVKPSDTLSLTCTVSGYSITSDYAWSWIRQPPGKGLEWIGYITYSGNISYNPSLKSRVTISVDTSKNQFSLKLSSVTAADTAVYDCARSLYYPWYFDVWGAGTTVTVSS

SEQ ID NO: 63 - protein, 25 aa, synthetic construct
QVQLQESGPGLVKPSDTLSLTCTVS

SEQ ID NO: 64 - protein, 17 aa, synthetic construct
WSWIRQPPGKGLEWIGY

SEQ ID NO: 65 - protein, 38 aa, synthetic construct
SYNPSLKSRVTISVDTSKNQFSLKLSSVTAADTAVYDC

SEQ ID NO: 66 - protein, 107 aa, synthetic construct
DIQMTQSPSSLSASVGDRVTITCRASSSVGYLNWYQQKPGKAPKPLIYAASSLQSGVPSRFSGSGSGTDNTLTISSLQPEDFATYYCQQWSSYPPITFGGGTKLEVK

SEQ ID NO: 67 - skipped

SEQ ID NO: 68 - protein, 25 aa, synthetic construct
DIQMTQSPSSLSASVGDRVTITCRA

SEQ ID NO: 69 - protein, 17 aa, synthetic construct
LNWYQQKPGKAPKPLIY

SEQ ID NO: 70 - protein, 36 aa, synthetic construct
SLQSGVPSRFSGSGSGTDNTLTISSLQPEDFATYYC

SEQ ID NO: 71 - protein, 121 aa, synthetic construct
EVQLQQSGAELVRPGTSVKTSCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSA

SEQ ID NO: 72 - protein, 8 aa, synthetic construct
GYTFTNYW

SEQ ID NO: 73 - protein, 8 aa, synthetic construct
IFPGGGHT

SEQ ID NO: 74 - protein, 14 aa, synthetic construct
AQIPLYYGHYRSAY

SEQ ID NO: 75 - protein, 25 aa, synthetic construct
EVQLQQSGAELVRPGTSVKTSCKAS

SEQ ID NO: 76 - protein, 17 aa, synthetic construct
LGWVKERAGHGLEWIGE

SEQ ID NO: 77 - protein, 38 aa, synthetic construct
NYKEKFKGKATLTADTSSSTAYMKLSSLTSEDSAVYFC

SEQ ID NO: 78 - protein, 11 aa, synthetic construct
WGQGTLVTVSA

SEQ ID NO: 79 - protein, 107 aa, synthetic construct
DIQMMQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIK

SEQ ID NO: 80 - protein, 6 aa, synthetic construct
ENIYGA

SEQ ID NO: 81 - skipped

SEQ ID NO: 82 - protein, 9 aa, synthetic construct
QNVLSTPYT

SEQ ID NO: 83 - protein, 26 aa, synthetic construct
DIQMMQSPASLSASVGETVAITCGAS

SEQ ID NO: 84 - protein, 17 aa, synthetic construct
LNWYQRKQGKSPQLLIY

SEQ ID NO: 85 - protein, 36 aa, synthetic construct
NLADGMSSRFSGSGSGRQYSLKISSLHPDDVATYYC

SEQ ID NO: 86 - protein, 10 aa, synthetic construct
FGGGTKLEIK

SEQ ID NO: 87 - protein, 123 aa, synthetic construct
QVTLKECGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSS

SEQ ID NO: 88 - protein, 10 aa, synthetic construct
GFSLSTSGMG

SEQ ID NO: 89 - protein, 7 aa, synthetic construct
IYWDDDK

SEQ ID NO: 90 - protein, 15 aa, synthetic construct
ARRLDGYNDPYYFDY

SEQ ID NO: 91 - protein, 25 aa, synthetic construct
QVTLKECGPGILQPSQTLSLTCSFS

SEQ ID NO: 92 - protein, 17 aa, synthetic construct
VSWIRQPSGKGLEWLAH

SEQ ID NO: 93 - protein, 38 aa, synthetic construct
RYNPSLKSRLTISKDSSSNQVFLKITSVDTADTATYYC

SEQ ID NO: 94 - protein, 11 aa, synthetic construct
WGQGTTLTVSS

SEQ ID NO: 95 - protein, 107 aa, synthetic construct
DIVMTQSPSSLSASLGDTVTITCHASQNVNVWLSWYQQKPGNIPKLLIYKASNLHAGVPSRFSGSGSGTGFTLTISSLQPEDIATYYCQQGQSYPLTFGGGTKLEIK

SEQ ID NO: 96 - protein, 6 aa, synthetic construct
QNVNVW

SEQ ID NO: 97 - skipped

SEQ ID NO: 98 - protein, 9 aa, synthetic construct
QQGQSYPLT

SEQ ID NO: 99 - protein, 26 aa, synthetic construct
DIVMTQSPSSLSASLGDTVTITCHAS

SEQ ID NO: 100 - protein, 17 aa, synthetic construct
LSWYQQKPGNIPKLLIY

SEQ ID NO: 101 - protein, 36 aa, synthetic construct
NLHAGVPSRFSGSGSGTGFTLTISSLQPEDIATYYC

SEQ ID NO: 102 - protein, 121 aa, synthetic construct
QVQLQQSGAELVRPGTSVKISCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSA

SEQ ID NO: 103 - protein, 25 aa, synthetic construct
QVQLQQSGAELVRPGTSVKISCKAS

SEQ ID NO: 104 - protein, 107 aa, synthetic construct
DIVMTQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIK

SEQ ID NO: 105 - protein, 26 aa, synthetic construct
DIVMTQSPASLSASVGETVAITCGAS

SEQ ID NO: 106 - protein, 121 aa, synthetic construct
EVQLQESGPGLVKPSQSLSLTCTVTGYSITSDYAWNWIRQFPGNRLEWMGFISYSGFTTYSPSLESRISITRDTSKNQFFLQLISVTTEDTATYYCARNHYGGSYWYFDVWGAGTSVTVSS

SEQ ID NO: 107 - protein, 7 aa, synthetic construct
ISYSGFT

SEQ ID NO: 108 - protein, 14 aa, synthetic construct
ARNHYGGSYWYFDV

SEQ ID NO: 109 - protein, 25 aa, synthetic construct
EVQLQESGPGLVKPSQSLSLTCTVT

SEQ ID NO: 110 - protein, 17 aa, synthetic construct
WNWIRQFPGNRLEWMGF

SEQ ID NO: 111 - protein, 38 aa, synthetic construct
TYSPSLESRISITRDTSKNQFFLQLISVTTEDTATYYC

SEQ ID NO: 112 - protein, 11 aa, synthetic construct
WGAGTSVTVSS

SEQ ID NO: 113 - protein, 111 aa, synthetic construct
DIVMTQSPGSLAVSLGQRATISCRASESVEYYGTSLMQWYLQKPGQPPKLLIYAASNVESGVPDRFRGSGSGTDFSLNIHPVEEDDIAMYFCQQSRKVPWTFGGGTKLEIK

SEQ ID NO: 114 - protein, 10 aa, synthetic construct
ESVEYYGTSL

SEQ ID NO: 115 - protein, 9 aa, synthetic construct
QQSRKVPWT

SEQ ID NO: 116 - protein, 26 aa, synthetic construct
DIVMTQSPGSLAVSLGQRATISCRAS

SEQ ID NO: 117 - protein, 17 aa, synthetic construct
MQWYLQKPGQPPKLLIY

SEQ ID NO: 118 - protein, 36 aa, synthetic construct
NVESGVPDRFRGSGSGTDFSLNIHPVEEDDIAMYFC

SEQ ID NO: 119 - protein, 119 aa, synthetic construct
EVKVEESGGGLVQPGGSMKLSCAASGFTFSDAWMDWVHQSPEKGLEWVAEIRSKANNHATYYVESVEGRFTISRDDSKSSVFLQVNSLRPEDTGIYYCTRRDGYYFAYWGQGTLVTVSA

SEQ ID NO: 120 - protein, 8 aa, synthetic construct
GFTFSDAW

SEQ ID NO: 121 - protein, 10 aa, synthetic construct
IRSKANNHAT

SEQ ID NO: 122 - protein, 10 aa, synthetic construct
TRRDGYYFAY

SEQ ID NO: 123 - protein, 25 aa, synthetic construct
EVKVEESGGGLVQPGGSMKLSCAAS

SEQ ID NO: 124 - protein, 17 aa, synthetic construct
MDWVHQSPEKGLEWVAE

SEQ ID NO: 125 - protein, 38 aa, synthetic construct
YYVESVEGRFTISRDDSKSSVFLQVNSLRPEDTGIYYC

SEQ ID NO: 126 - protein, 105 aa, synthetic construct
DIVLTQSPAIMSASLGEEITLTCSASSSVRDMHWYQQKSGTSPKVLIYNTFNLASGVPSRFSGSGSGTFYSLTISSVEAGDAAAYYCHQWSSYPTFGGGTKLEIK

SEQ ID NO: 127 - protein, 6 aa, synthetic construct
SSSVRD

SEQ ID NO: 128 - skipped

SEQ ID NO: 129 - protein, 8 aa, synthetic construct
HQWSSYPT

SEQ ID NO: 130 - protein, 25 aa, synthetic construct
DIVLTQSPAIMSASLGEEITLTCSA

SEQ ID NO: 131 - protein, 17 aa, synthetic construct
MHWYQQKSGTSPKVLIY

SEQ ID NO: 132 - protein, 36 aa, synthetic construct
NLASGVPSRFSGSGSGTFYSLTISSVEAGDAAAYYC

SEQ ID NO: 133 - protein, 123 aa, synthetic construct
QVTLKVSGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSS

SEQ ID NO: 134 - protein, 25 aa, synthetic construct
QVTLKVSGPGILQPSQTLSLTCSFS

SEQ ID NO: 135 - protein, 11 aa, synthetic construct
WGQGTTLTVSS

SEQ ID NO: 136 - protein, 106 aa, synthetic construct
DVVMTQTPALMSASPGEKVTMTCSASSSVSYMYWYQQKPRSSPKPWIYLTSNLASGVPARFSGSGSGTSYSLTISSMEAEDAATYYCQQWNSNPYTFGGGTKLEIK

SEQ ID NO: 137 - protein, 5 aa, synthetic construct
SSVSY

SEQ ID NO: 138 - skipped

SEQ ID NO: 139 - protein, 9 aa, synthetic construct
QQWNSNPYT

SEQ ID NO: 140 - protein, 26 aa, synthetic construct
DVVMTQTPALMSASPGEKVTMTCSAS

SEQ ID NO: 141 - protein, 17 aa, synthetic construct
MYWYQQKPRSSPKPWIY

SEQ ID NO: 142 - protein, 36 aa, synthetic construct
NLASGVPARFSGSGSGTSYSLTISSMEAEDAATYYC

SEQ ID NO: 143 - protein, 118 aa, synthetic construct
QVQLQQSGAELARPGASVRMSCKASGYTFTSYTMNWVKQRPGQGLEWIGFINPDSDYTTYDQKFKDKATLTADSSSSTAYMQLSSLTYDDSAVYYCTRHSYGNYGDYWGQGTTLTVSS

SEQ ID NO: 144 - protein, 8 aa, synthetic construct
GYTFTSYT

SEQ ID NO: 145 - protein, 8 aa, synthetic construct
INPDSDYT

SEQ ID NO: 146 - protein, 11 aa, synthetic construct
TRHSYGNYGDY

SEQ ID NO: 147 - protein, 25 aa, synthetic construct
QVQLQQSGAELARPGASVRMSCKAS

SEQ ID NO: 148 - protein, 17 aa, synthetic construct
MNWVKQRPGQGLEWIGF

SEQ ID NO: 149 - protein, 38 aa, synthetic construct
TYDQKFKDKATLTADSSSSTAYMQLSSLTYDDSAVYYC

SEQ ID NO: 150 - protein, 107 aa, synthetic construct
DIVLTQSPKFLLVSAGDRVTITCKASQSVTNDVAWYQQKPGQSPKLLIYYASSRYTGVPDRFTGSGFGTDFTFTINTVQAEDLAVYFCQQDYSSPYTFGGGTKLEIK

SEQ ID NO: 151 - protein, 6 aa, synthetic construct
QSVTND

SEQ ID NO: 152 - skipped

SEQ ID NO: 153 - protein, 9 aa, synthetic construct
QQDYSSPYT

SEQ ID NO: 154 - protein, 26 aa, synthetic construct
DIVLTQSPKFLLVSAGDRVTITCKAS

SEQ ID NO: 155 - protein, 17 aa, synthetic construct
VAWYQQKPGQSPKLLIY

SEQ ID NO: 156 - protein, 36 aa, synthetic construct
SRYTGVPDRFTGSGFGTDFTFTINTVQAEDLAVYFC

SEQ ID NO: 157 - protein, 118 aa, synthetic construct
QVQLQQSGADLARPGASVKMSCKASGYTFIDYTVHWVKQRPGQGLEWIGFINPSNDYTSYNQKFKDKASLTADTSSTTAYMQLSSLTSDDSAVYYCARHSYGNYGDYWGQGTTLTVSS

SEQ ID NO: 158 - protein, 8 aa, synthetic construct
GYTFIDYT

SEQ ID NO: 159 - protein, 8 aa, synthetic construct
INPSNDYT

SEQ ID NO: 160 - protein, 11 aa, synthetic construct
ARHSYGNYGDY

SEQ ID NO: 161 - protein, 25 aa, synthetic construct
QVQLQQSGADLARPGASVKMSCKAS

SEQ ID NO: 162 - protein, 17 aa, synthetic construct
VHWVKQRPGQGLEWIGF

SEQ ID NO: 163 - protein, 38 aa, synthetic construct
SYNQKFKDKASLTADTSSTTAYMQLSSLTSDDSAVYYC

SEQ ID NO: 164 - protein, 107 aa, synthetic construct
DIVMTQAPKFLLVSAGDRVTITCKASQSVTNGVAWYQQKPGQSPKLLIYYASNRYTGVPDRFTGSGFGTDFTFTISTVQAEDLAVYFCQQDYSSPYTFGGGTKLEIK

SEQ ID NO: 165 - protein, 6 aa, synthetic construct
QSVTNG

SEQ ID NO: 166 - protein, 26 aa, synthetic construct
DIVMTQAPKFLLVSAGDRVTITCKAS

SEQ ID NO: 167 - protein, 36 aa, synthetic construct
NRYTGVPDRFTGSGFGTDFTFTISTVQAEDLAVYFC

SEQ ID NO: 168 - protein, 119 aa, synthetic construct
QVQLQQSGAELVKPGASVRLSCKASGYTFTSYWMHWVRQRPGLGLEWIGEIDPSDSYTNCNQRFKGKATLTVDKSSSTAYTQLSSLTSEDSAVYYCARWAYGPYAMDYWGQGTSVTVSS

SEQ ID NO: 169 - protein, 8 aa, synthetic construct
GYTFTSYW

SEQ ID NO: 170 - protein, 8 aa, synthetic construct
IDPSDSYT

SEQ ID NO: 171 - protein, 12 aa, synthetic construct
ARWAYGPYAMDY

SEQ ID NO: 172 - protein, 25 aa, synthetic construct
QVQLQQSGAELVKPGASVRLSCKAS

SEQ ID NO: 173 - protein, 17 aa, synthetic construct
MHWVRQRPGLGLEWIGE

SEQ ID NO: 174 - protein, 38 aa, synthetic construct
NCNQRFKGKATLTVDKSSSTAYTQLSSLTSEDSAVYYC

SEQ ID NO: 175 - protein, 11 aa, synthetic construct
WGQGTSVTVSS

SEQ ID NO: 176 - protein, 112 aa, synthetic construct
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 177 - protein, 11 aa, synthetic construct
QSLVHSNGNTY

SEQ ID NO: 178 - skipped

SEQ ID NO: 179 - protein, 9 aa, synthetic construct
SQSTHVPWT

SEQ ID NO: 180 - protein, 26 aa, synthetic construct
DIVMTQTPLSLPVSLGDQASISCRSS

SEQ ID NO: 181 - protein, 17 aa, synthetic construct
LHWYLQKPGQSPKLLIY

SEQ ID NO: 182 - protein, 36 aa, synthetic construct [sequence omitted]

SEQ ID NO: 183 - protein, 122 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARGGYYYGSSWYFDVWGAGTTVTVSS

SEQ ID NO: 184 - protein, 8 aa, synthetic construct
IYPGGGYT

SEQ ID NO: 185 - protein, 15 aa, synthetic construct
ARGGYYYGSSWYFDV

SEQ ID NO: 186 - protein, 17 aa, synthetic construct
LGWVKQRPGHGLEWIGD

SEQ ID NO: 187 - protein, 38 aa, synthetic construct
NYNEKFKGKATLTADTSSSTAYMQLSSLTSEDSAVYFC

SEQ ID NO: 188 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 189 - protein, 11 aa, synthetic construct
QSIVHSNGNTY

SEQ ID NO: 190 - protein, 9 aa, synthetic construct
FQGSHVPPT

SEQ ID NO: 191 - protein, 26 aa, synthetic construct
DVLMTQTPLSLPVSLGDQASISCRSS

SEQ ID NO: 192 - protein, 17 aa, synthetic construct
LEWYLQKPGQSPKLLIY

SEQ ID NO: 193 - protein, 36 aa, synthetic construct
NRFSGVPDRFSGSGSGTDFTLKISRVEAEDLGVYYC

SEQ ID NO: 194 - protein, 122 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCVRSGYYYGSSWYFDVWGAGTTVTVSS

SEQ ID NO: 195 - protein, 15 aa, synthetic construct
VRSGYYYGSSWYFDV

SEQ ID NO: 196 - protein, 112 aa, synthetic construct
DVLMTQTPLSLPVSLGDQASISCRSSQSIVHSSGNTYLEWYLQKPDQSPKLLIYKVSNRFSGVPDRFSGSGSGTDFTLKISRVEAEDLGVYYCFQGSHVPPTFGGGTKLEIK

SEQ ID NO: 197 - protein, 11 aa, synthetic construct
QSIVHSSGNTY

SEQ ID NO: 198 - protein, 17 aa, synthetic construct
LEWYLQKPDQSPKLLIY

SEQ ID NO: 199 - protein, 122 aa, synthetic construct
QVQLQQSGAELVRPGTSVKISCKASGYTFTNYWLGWVKQRPGHGLEWIGDIYPGGGYTNYNEKFKGKATLTADISSSTAYMQLSSLTSEDSADYFCVRGGYYYGSSWYFDVWGAGTTVTVSS

SEQ ID NO: 200 - protein, 15 aa, synthetic construct
VRGGYYYGSSWYFDV

SEQ ID NO: 201 - protein, 38 aa, synthetic construct
NYNEKFKGKATLTADISSSTAYMQLSSLTSEDSADYFC

SEQ ID NO: 202 - protein, 112 aa, synthetic construct
DVLMTQTPLSLPVSLGDQASISCRSSQSTVHSIGNTYLEWYLQKPGQSPKLLIYKVSNRFSGVPDRFSGSESGTDFTLKISRVEAEDLGVYYCFQGSHVPPTFGGGTKLEIK

SEQ ID NO: 203 - protein, 11 aa, synthetic construct
QSTVHSIGNTY

SEQ ID NO: 204 - protein, 36 aa, synthetic construct
NRFSGVPDRFSGSESGTDFTLKISRVEAEDLGVYYC

SEQ ID NO: 205 - protein, 330 aa, synthetic construct [sequence omitted]

SEQ ID NO: 206 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 207 - protein, 12 aa, synthetic construct
EPKSCDKTHTCP

SEQ ID NO: 208 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 209 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 107 aa, synthetic construct [sequence omitted]

SEQ ID NO: 212 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 214 aa, synthetic construct
DIVITQTPAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 449 aa, synthetic construct
EVMLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 214 aa, synthetic construct
DIVLTQTTAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 449 aa, synthetic construct
EVQLLESGGGLVQPGGSLRLSCAASGYTITSDYAMSWVRQAPGKGLEWVSVITYSGNISYADSVKGRFTISRDNSKNTLYLQMNSLRAEDTAVYYCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 214 aa, synthetic construct
EIVITQSPATLSLSPGERATLSCRASSSVGYLAWYQQKPGQAPRPLIYDTSNRATGIPARFSGSGSGTDNTLTISSLEPEDFAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 449 aa, synthetic construct
QVQLQESGPGLVKPSDTLSLTCTVSGYSITSDYAWSWIRQPPGKGLEWIGYITYSGNISYNPSLKSRVTISVDTSKNQFSLKLSSVTAADTAVYDCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 214 aa, synthetic construct
DIQMTQSPSSLSASVGDRVTITCRASSSVGYLNWYQQKPGKAPKPLIYAASSLQSGVPSRFSGSGSGTDNTLTISSLQPEDFATYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 451 aa, synthetic construct
EVQLQQSGAELVRPGTSVKTSCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 214 aa, synthetic construct
DIQMMQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 453 aa, synthetic construct
QVTLKECGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 214 aa, synthetic construct
DIVMTQSPSSLSASLGDTVTITCHASQNVNVWLSWYQQKPGNIPKLLIYKASNLHAGVPSRFSGSGSGTGFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 451 aa, synthetic construct
QVQLQQSGAELVRPGTSVKISCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 214 aa, synthetic construct
DIVMTQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 451 aa, synthetic construct
EVQLQESGPGLVKPSQSLSLTCTVTGYSITSDYAWNWIRQFPGNRLEWMGFISYSGFTTYSPSLESRISITRDTSKNQFFLQLISVTTEDTATYYCARNHYGGSYWYFDVWGAGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 218 aa, synthetic construct
DIVMTQSPGSLAVSLGQRATISCRASESVEYYGTSLMQWYLQKPGQPPKLLIYAASNVESGVPDRFRGSGSGTDFSLNIHPVEEDDIAMYFCQQSRKVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 449 aa, synthetic construct
EVKVEESGGGLVQPGGSMKLSCAASGFTFSDAWMDWVHQSPEKGLEWVAEIRSKANNHATYYVESVEGRFTISRDDSKSSVFLQVNSLRPEDTGIYYCTRRDGYYFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 212 aa, synthetic construct
DIVLTQSPAIMSASLGEEITLTCSASSSVRDMHWYQQKSGTSPKVLIYNTFNLASGVPSRFSGSGSGTFYSLTISSVEAGDAAAYYCHQWSSYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 453 aa, synthetic construct
QVTLKVSGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 213 aa, synthetic construct
DVVMTQTPALMSASPGEKVTMTCSASSSVSYMYWYQQKPRSSPKPWIYLTSNLASGVPARFSGSGSGTSYSLTISSMEAEDAATYYCQQWNSNPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 448 aa, synthetic construct
QVQLQQSGAELARPGASVRMSCKASGYTFTSYTMNWVKQRPGQGLEWIGFINPDSDYTTYDQKFKDKATLTADSSSSTAYMQLSSLTYDDSAVYYCTRHSYGNYGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 214 aa, synthetic construct
DIVLTQSPKFLLVSAGDRVTITCKASQSVTNDVAWYQQKPGQSPKLLIYYASSRYTGVPDRFTGSGFGTDFTFTINTVQAEDLAVYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 448 aa, synthetic construct
QVQLQQSGADLARPGASVKMSCKASGYTFIDYTVHWVKQRPGQGLEWIGFINPSNDYTSYNQKFKDKASLTADTSSTTAYMQLSSLTSDDSAVYYCARHSYGNYGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 214 aa, synthetic construct
DIVMTQAPKFLLVSAGDRVTITCKASQSVTNGVAWYQQKPGQSPKLLIYYASNRYTGVPDRFTGSGFGTDFTFTISTVQAEDLAVYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 449 aa, synthetic construct
QVQLQQSGAELVKPGASVRLSCKASGYTFTSYWMHWVRQRPGLGLEWIGEIDPSDSYTNCNQRFKGKATLTVDKSSSTAYTQLSSLTSEDSAVYYCARWAYGPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 219 aa, synthetic construct
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 452 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARGGYYYGSSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 219 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 452 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCVRSGYYYGSSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 219 aa, synthetic construct
DVLMTQTPLSLPVSLGDQASISCRSSQSIVHSSGNTYLEWYLQKPDQSPKLLIYKVSNRFSGVPDRFSGSGSGTDFTLKISRVEAEDLGVYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 452 aa, synthetic construct
QVQLQQSGAELVRPGTSVKISCKASGYTFTNYWLGWVKQRPGHGLEWIGDIYPGGGYTNYNEKFKGKATLTADISSSTAYMQLSSLTSEDSADYFCVRGGYYYGSSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 219 aa, synthetic construct
DVLMTQTPLSLPVSLGDQASISCRSSQSTVHSIGNTYLEWYLQKPGQSPKLLIYKVSNRFSGVPDRFSGSESGTDFTLKISRVEAEDLGVYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 9 aa, synthetic construct
  VARIANT 3: note = X = S or T
GYXITSDYA

SEQ ID NO: 245 - skipped

SEQ ID NO: 246 - protein, 15 aa, synthetic construct
  VARIANT 1: note = X = V or A
  VARIANT 3: note = X = G or S
XRXGYYYGSSWYFDV

SEQ ID NO: 247 - protein, 11 aa, synthetic construct
  VARIANT 3: note = X = I or T
  VARIANT 7: note = X = N, S or I
QSXVHSXGNTY

SEQ ID NO: 248 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 249 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 250 - protein, 214 aa, synthetic construct
DIVITQTPAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 251 - protein, 97 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 252 - protein, 16 aa, synthetic construct
EPRGPTIKPCPPCKCP

SEQ ID NO: 253 - protein, 110 aa, synthetic construct
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 254 - protein, 110 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPK

SEQ ID NO: 255 - protein, 107 aa, synthetic construct
GSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 256 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 257 - protein, 217 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 258 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYQSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 259 - protein, 110 aa, synthetic construct
APNLLGGPSVFIFPPKIKDVLMISLSPIVTCVVVDVSEDDPDVQISWFVNNVEVHTAQTQTHREDYQSTLRVVSALPIQHQDWMSGKEFKCKVNNKDLPAPIERTISKPK

SEQ ID NO: 260 - protein, 217 aa, synthetic construct
APNLLGGPSVFIFPPKIKDVLMISLSPIVTCVVVDVSEDDPDVQISWFVNNVEVHTAQTQTHREDYQSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 261 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 262 - protein, 97 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKI

SEQ ID NO: 263 - protein, 13 aa, synthetic construct
VPRDCGCKPCICT

SEQ ID NO: 264 - protein, 107 aa, synthetic construct
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTK

SEQ ID NO: 265 - protein, 107 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 266 - protein, 443 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 267 - protein, 449 aa, synthetic construct
QVQLQQSGAELMKPGASVKISCKATGYTFSTHWIEWVKQRPGHGLEWIGEILPGSGSTSYNEKFKGKATFTADTSSNTAYMQLSSLTSEDSAVYYCARWLLYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 219 aa, synthetic construct
DVLMTQTPLSLPVSLGDQASISCRSSQSIVHSNGNTYLEWYLQKPGQSPKLLIYKLSNRFSGVPDRFSGSGSGTDFTLKISRVEAEDLGVYYCFQGSHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 450 aa, synthetic construct
QVQLVQSGAEVKKPGSSVKVSCKASGGTFSSYAISWVRQAPGQGLEWMGGLLPIFGTANYAQKFQGRVTITADESTSTAYMELSSLRSEDTAVYYCARSSYGWSYEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 214 aa, synthetic construct
DIQMTQSPSSLSASVGDRVTITCRASQSIDTRLNWYQQKPGKAPKLLIYSASSLQSGVPSRFSGSGSGTDFTLTISSLQPEDFATYYCQQSAYNPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 10 aa, synthetic construct
PVDKGHDVTF

SEQ ID NO: 272 - protein, 12 aa, synthetic construct
RRPIRNLTFQDL

SEQ ID NO: 273 - protein, 14 aa, synthetic construct
TWYRSSRGEVQTCS

SEQ ID NO: 274 - protein, 17 aa, synthetic construct
EIRHHHSEHRVHGAMEL

SEQ ID NO: 275 - protein, 32 aa, synthetic construct
FKVATPYSLYVCPEGQNVTLTCRLLGPVDKGH

SEQ ID NO: 276 - protein, 119 aa, synthetic construct
QVQLQESGPGLVKPSDTLSLTCTVSGYSITSDYAWNWIRQTPGKGLEWIGYITYSGYISYNPSLRSRVTISRDTSKNQFSLKLSSVTAADTAVYSCARALYYPWYFDVWGTGTTVTVSS

SEQ ID NO: 277 - protein, 7 aa, synthetic construct
ITYSGYI

SEQ ID NO: 278 - protein, 12 aa, synthetic construct
ARALYYPWYFDV

SEQ ID NO: 279 - protein, 17 aa, synthetic construct
WNWIRQTPGKGLEWIGY

SEQ ID NO: 280 - protein, 38 aa, synthetic construct
SYNPSLRSRVTISRDTSKNQFSLKLSSVTAADTAVYSC

SEQ ID NO: 281 - protein, 11 aa, synthetic construct
WGTGTTVTVSS

SEQ ID NO: 282 - protein, 107 aa, synthetic construct
EIVITQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVK

SEQ ID NO: 283 - protein, 17 aa, synthetic construct
IHWYQQKPGQAPRPLIY

SEQ ID NO: 284 - protein, 36 aa, synthetic construct
NRATGIPARFSGSGSGTDNTLTISSLEPEDSAVYYC

SEQ ID NO: 285 - protein, 119 aa, synthetic construct
QVQLQESGPGLVKPSDTLSLTCTVSGYSITSDYAWNWIRQTPGKGLEWIGYITYSGYVSYNPSLRSRVTISRDTSKNQFSLKLSSVTAADTAVYSCARALYYPWYFDVWGTGTTVTVSS

SEQ ID NO: 286 - protein, 7 aa, synthetic construct
ITYSGYV

SEQ ID NO: 287 - protein, 107 aa, synthetic construct
EIVLTQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVK

SEQ ID NO: 288 - protein, 26 aa, synthetic construct [sequence omitted]

SEQ ID NO: 289 - protein, 119 aa, synthetic construct
QVQLQESGPGLVKPSDTLSLTCTVSGYSITSDYTWNWIRQTPGKGLEWIGHITYSGSVSYNPSLRSRVTISRDTSKNQFSLKLSSVTAADTATYSCARALYYPWYFDVWGTGTTVTVSS

SEQ ID NO: 290 - protein, 9 aa, synthetic construct
GYSITSDYT

SEQ ID NO: 291 - protein, 7 aa, synthetic construct
ITYSGSV

SEQ ID NO: 292 - protein, 17 aa, synthetic construct
WNWIRQTPGKGLEWIGH

SEQ ID NO: 293 - protein, 38 aa, synthetic construct
SYNPSLRSRVTISRDTSKNQFSLKLSSVTAADTATYSC

SEQ ID NO: 294 - protein, 107 aa, synthetic construct
EIVLTQSPATLSLSPGERATLSCRASSSVGYIHWYQQKPGQAPRPLIYTTSYRATGIPARFSGSGSGTDNTLTISSLEPEDSAVYYCQQWSSYPPITFGGGTKLEVK

SEQ ID NO: 295 - skipped

SEQ ID NO: 296 - protein, 36 aa, synthetic construct
YRATGIPARFSGSGSGTDNTLTISSLEPEDSAVYYC

SEQ ID NO: 297 - protein, 107 aa, synthetic construct
EIVLTQSPATLSLSPGERATLSCRASSSVGYIHWYQQKPGQAPRPIIYTTSNRATGIPARFSGSGSGTDNTLTISSLEPEDSAVYYCQQWSSYPPITFGGGTKLEVK

SEQ ID NO: 298 - protein, 17 aa, synthetic construct
IHWYQQKPGQAPRPIIY

SEQ ID NO: 299 - protein, 107 aa, synthetic construct
EIVLTQSPATLSLSPGERATLSCRASSSVGYIHWYQQKPGQAPRPLIYATYNRATGIPARFSGSGSGTDNTLTISSLEPEDSAVYYCQQWSSYPPITFGGGTKLEVK

SEQ ID NO: 300 - skipped

SEQ ID NO: 301 - protein, 107 aa, synthetic construct
EIVLTQSPATLSLSPGERATLSCRASSSVGYIHWYQQKPGQAPRPLIYATSYRATGIPARFSGSGSGTDNTLTISSLEPEDSAVYYCQQWSSYPPITFGGGTKLEVK

SEQ ID NO: 302 - protein, 107 aa, synthetic construct
EIVLTQSPATLSLSPGERATLSCRASSSVGYIHWYQQKPGQAPRPLIYATYYRATGIPARFSGSGSGTDNTLTISSLEPEDSAVYYCQQWSSYPPITFGGGTKLEVK

SEQ ID NO: 303 - protein, 107 aa, synthetic construct
EIVLTQSPATLSLSPGERATLSCRASSSVGYIHWYQQKPGQAPRPLIYATYYRTTGIPARFSGSGSGTDNTLTISSLEPEDSAVYYCQQWSSYPPITFGGGTKLEVK

SEQ ID NO: 304 - protein, 36 aa, synthetic construct
YRTTGIPARFSGSGSGTDNTLTISSLEPEDSAVYYC

SEQ ID NO: 305 - protein, 9 aa, synthetic construct
  VARIANT 3: note = X = S or T
  VARIANT 9: note = X = T or A
GYXITSDYX

SEQ ID NO: 306 - protein, 7 aa, synthetic construct
  VARIANT 6: note = X = S, N or Y
  VARIANT 7: note = X = V or I
ITYSGXX

SEQ ID NO: 307 - protein, 12 aa, synthetic construct
  VARIANT 3: note = X = A or S
ARXLYYPWYFDV

SEQ ID NO: 308-309 - skipped (2 entries)

SEQ ID NO: 310 - protein, 109 aa, synthetic construct
  VARIANT 49: note = X = T or A
  VARIANT 51: note = X = S or Y
  VARIANT 52: note = X = N or Y
EIVLTQSPATLSLSPGERATLSCRASSSVGYIHWYQQKPGQAPRPJIYXTXXYNRATGIPARFSGSGSGTDNTLTISSLEPEDSAVYYCQQWSSYPPITFGGGTKLEVK

SEQ ID NO: 311 - protein, 449 aa, synthetic construct
QVQLQESGPGLVKPSDTLSLTCTVSGYSITSDYAWNWIRQTPGKGLEWIGYITYSGYISYNPSLRSRVTISRDTSKNQFSLKLSSVTAADTAVYSCARALYYPWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 214 aa, synthetic construct
EIVITQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 313 - protein, 449 aa, synthetic construct
QVQLQESGPGLVKPSDTLSLTCTVSGYSITSDYAWNWIRQTPGKGLEWIGYITYSGYVSYNPSLRSRVTISRDTSKNQFSLKLSSVTAADTAVYSCARALYYPWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 214 aa, synthetic construct
EIVLTQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 315 - protein, 449 aa, synthetic construct
QVQLQESGPGLVKPSDTLSLTCTVSGYSITSDYTWNWIRQTPGKGLEWIGHITYSGSVSYNPSLRSRVTISRDTSKNQFSLKLSSVTAADTATYSCARALYYPWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 214 aa, synthetic construct
EIVLTQSPATLSLSPGERATLSCRASSSVGYIHWYQQKPGQAPRPLIYTTSYRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 317 - protein, 214 aa, synthetic construct
EIVLTQSPATLSLSPGERATLSCRASSSVGYIHWYQQKPGQAPRPIIYTTSNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - protein, 214 aa, synthetic construct
EIVLTQSPATLSLSPGERATLSCRASSSVGYIHWYQQKPGQAPRPLIYATYNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 214 aa, synthetic construct
EIVLTQSPATLSLSPGERATLSCRASSSVGYIHWYQQKPGQAPRPLIYATSYRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 214 aa, synthetic construct
EIVLTQSPATLSLSPGERATLSCRASSSVGYIHWYQQKPGQAPRPLIYATYYRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 321 - protein, 214 aa, synthetic construct
EIVLTQSPATLSLSPGERATLSCRASSSVGYIHWYQQKPGQAPRPLIYATYYRTTGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 6 aa, synthetic construct
SRGEVQ

SEQ ID NO: 323 - protein, 412 aa, synthetic construct
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 324 - protein, 428 aa, synthetic construct
MAVGASGLEGDKMAGAMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 325 - protein, 395 aa, synthetic construct
LQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 326 - protein, 303 aa, synthetic construct
LQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQC

SEQ ID NO: 327 - protein, 21 aa, synthetic construct
LLAILILALVATIFFVCTVVL

SEQ ID NO: 328 - protein, 71 aa, synthetic construct
AVRLSRKGHMYPVRNYSPTEMVCISSLLPDGGEGPSATANGGLSKAKSPGLTPEPREDREGDDLTLHSFLP

SEQ ID NO: 329 - protein, 140 aa, synthetic construct
QTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEA

SEQ ID NO: 330 - protein, 446 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 331 - protein, 446 aa, synthetic construct
QVQLQESGPGLVKPSDTLSLTCTVSGYSITSDYTWNWIRQTPGKGLEWIGHITYSGSVSYNPSLRSRVTISRDTSKNQFSLKLSSVTAADTATYSCARALYYPWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 332 - protein, 1093 aa, synthetic construct
MARPVRGGLGAPRRSPCLLLLWLLLLRLEPVTAAAGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFTIAVVSSIVLTSLVWVCIIYQTRKKSEEYSVTNTDETVVPPDVPSYLSSQGTLSDRQETVVRTEGGPQANGHIESNGVCPRDASHFPEPDTHSVACRQPKLCAGSAYHKEPWKAMEKAEGTPGPHKMEHGGRVVCSDCNTEVDCYSRGQAFHPQPVSRDSAQPSAPNGPEPGGSDQEHSPHHQCSRTAAGSCPECQGSLYPSNHDRMLTAVKKKPMASLDGKGDSSWTLARLYHPDSTELQPASSLTSGSPERAEAQYLLVSNGHLPKACDASPESTPLTGQLPGKQRVPLLLAPKS

SEQ ID NO: 333 - protein, 1059 aa, synthetic construct
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFTIAVVSSIVLTSLVWVCIIYQTRKKSEEYSVTNTDETVVPPDVPSYLSSQGTLSDRQETVVRTEGGPQANGHIESNGVCPRDASHFPEPDTHSVACRQPKLCAGSAYHKEPWKAMEKAEGTPGPHKMEHGGRVVCSDCNTEVDCYSRGQAFHPQPVSRDSAQPSAPNGPEPGGSDQEHSPHHQCSRTAAGSCPECQGSLYPSNHDRMLTAVKKKPMASLDGKGDSSWTLARLYHPDSTELQPASSLTSGSPERAEAQYLLVSNGHLPKACDASPESTPLTGQLPGKQRVPLLLAPKS

SEQ ID NO: 334 - protein, 1070 aa, synthetic construct
MARPVRGGLGAPRRSPCLLLLWLLLLRLEPVTAAAGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLLLLEPSQSAGCSSPSQPHMSAGGRTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAAQTPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFTIAVVSSIVLTSLVWVCIIYQTRKKSEEYSVTNTDETVVPPDVPSYLSSQGTLSDRQETVVRTEGGPQANGHIESNGVCPRDASHFPEPDTHSVACRQPKLCAGSAYHKEPWKAMEKAEGTPGPHKMEHGGRVVCSDCNTEVDCYSRGQAFHPQPVSRDSAQPSAPNGPEPGGSDQEHSPHHQCSRTAAGSCPECQGSLYPSNHDRMLTAVKKKPMASLDGKGDSSWTLARLYHPDSTELQPASSLTSGSPERAEAQYLLVSNGHLPKACDASPESTPLTGQLPGKQRVPLLLAPKS

SEQ ID NO: 335 - protein, 1036 aa, synthetic construct
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLLLLEPSQSAGCSSPSQPHMSAGGRTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAAQTPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFTIAVVSSIVLTSLVWVCIIYQTRKKSEEYSVTNTDETVVPPDVPSYLSSQGTLSDRQETVVRTEGGPQANGHIESNGVCPRDASHFPEPDTHSVACRQPKLCAGSAYHKEPWKAMEKAEGTPGPHKMEHGGRVVCSDCNTEVDCYSRGQAFHPQPVSRDSAQPSAPNGPEPGGSDQEHSPHHQCSRTAAGSCPECQGSLYPSNHDRMLTAVKKKPMASLDGKGDSSWTLARLYHPDSTELQPASSLTSGSPERAEAQYLLVSNGHLPKACDASPESTPLTGQLPGKQRVPLLLAPKS

SEQ ID NO: 336 - protein, 760 aa, synthetic construct
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVG

SEQ ID NO: 337 - protein, 737 aa, synthetic construct
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLLLLEPSQSAGCSSPSQPHMSAGGRTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAAQTPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVG

SEQ ID NO: 338 - protein, 21 aa, synthetic construct
IFTIAVVSSIVLTSLVWVCII

SEQ ID NO: 339 - protein, 278 aa, synthetic construct
YQTRKKSEEYSVTNTDETVVPPDVPSYLSSQGTLSDRQETVVRTEGGPQANGHIESNGVCPRDASHFPEPDTHSVACRQPKLCAGSAYHKEPWKAMEKAEGTPGPHKMEHGGRVVCSDCNTEVDCYSRGQAFHPQPVSRDSAQPSAPNGPEPGGSDQEHSPHHQCSRTAAGSCPECQGSLYPSNHDRMLTAVKKKPMASLDGKGDSSWTLARLYHPDSTELQPASSLTSGSPERAEAQYLLVSNGHLPKACDASPESTPLTGQLPGKQRVPLLLAPKS

SEQ ID NO: 340 - protein, 29 aa, synthetic construct
YKTWYRSSRGEVQTCSERRPIRNLTFQDL

SEQ ID NO: 341 - protein, 13 aa, synthetic construct
ERRPIRNLTFQDL

SEQ ID NO: 342 - protein, 17 aa, synthetic construct
YKTWYRSSRGEVQTCSE

SEQ ID NO: 343 - protein, 25 aa, synthetic construct
FYKTWYRSSRGEVQTCSERRPIRNL

SEQ ID NO: 344 - protein, 38 aa, synthetic construct
TFYKTWYRSSRGEVQTCSERRPIRNLTFQDLHLHHGGH

SEQ ID NO: 345 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 346 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 347 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 348 - protein, 311 aa, synthetic construct
MGVPTAPEAGCWRWGSLLFALFLAASLGPVAAFKVATLYSLYVCPEGQNVTLTCRVFGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSSCVAYPSSSQESENITAAALATGACIVGILCLPLILLLVYKQRQAASNRRAQELVRMDSNTQGIENPGFEASSPAQGILEAKVRHPLSYVAQRQPSESGRHLLSEPGTPLSPPGPGDVFFPSLDPVPDSPNFEVI

SEQ ID NO: 349 - protein, 309 aa, synthetic construct
MGVPTVPEASSLRWGTLLLTIFLAASRGLVAAIKVATPYSLYVCPEGQNVTLTCRILDSVSKGHDANFLKTWFLSSRGEVQVCKEHRPIRNFISHHQHHRSHPAVNASHDQPQKHGLEIAYDNHGNFSITLHNVTLSDSGLYCCLVIEVKHHHPERRLYGYMELQVQTGKGSASTCTAYPPNEQDSDSITAAALATGACIVGILCLPLILLLVYKQRQAASHRRAQELVRMDSNTQGIENPGYETTPPFQGMPEAKTRPPLSYVAQRQPSESGRHLLSDPSTPLSPPGPGDVFFPSLDPVPDSPNSEAI

SEQ ID NO: 350 - protein, 308 aa, synthetic construct
MGVPAVPEASSPRWGTLLLAIFLAASRGLVAAFKVATPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIEVKHHHPEQRFYGSMELQVQAGKGSGSTCMASNEQDSDSITAAALATGACIVGILCLPLILLLVYKQRQVASHRRAQELVRMDSNTQGIENPGFETTPPFQGMPEAKTRPPLSYVAQRQPSESGRYLLSDPSTPLSPPGPGDVFFPSLDPVPDSPNSEAI

(19) United States
(12) Patent Application Publication
(10) Pub. No.: US 2026/0098093 A1
(43) Pub. Date: Apr. 9, 2026

(54) TREATMENT AND PREVENTION OF CANCER USING VISTA ANTIGEN-BINDING MOLECULES

(71) Applicant: Hummingbird Bioscience Pte. Ltd., Singapore (SG)

(72) Inventors: Bhushan Dharmadhikari, Singapore (SG); Olga Zharkova, Singapore (SG); Debleena Ray, Singapore (SG); Roberto Tirado-Magallanes, Singapore (SG); Dipti Thakkar, Singapore (SG); Konrad Paszkiewicz, Singapore (SG); Jerome Boyd-Kirkup, Singapore (SG); Piers Ingram, Singapore (SG)

(21) Appl. No.: 19/114,367

(22) PCT Filed: Sep. 21, 2023
(86) PCT No.: PCT/EP2023/076152

§ 371 Date: Mar. 21, 2025

Related U.S. Application Data
(60) Provisional application No. 63/409,003, filed on Sep. 22, 2022.

(51) Int. Cl. C07K 16/28; A61K 39/00; A61P 35/00; A61P 37/04

(52) U.S. Cl. CPC C07K 16/2827; A61P 35/00; A61P 37/04; A61K 2039/505; C07K 2317/565; C07K 2317/567; C07K 2317/76; C07K 2317/92

(57) ABSTRACT

VISTA antigen-binding molecules are disclosed. Also disclosed are nucleic acids and expression vectors encoding, composition comprising, and methods using, the VISTA antigen-binding molecules.

This application claims priority from U.S. 63/409,003 filed 22 Sep. 2022, the contents and elements of which are herein incorporated by reference for all purposes.

### TECHNICAL FIELD

The present disclosure relates to the fields of molecular biology, more specifically antibody technology and methods of medical treatment and prophylaxis.

### BACKGROUND

Myeloid Derived Suppressor Cell (MDSC)-mediated suppression of immune response has been identified in multiple solid tumors and lymphomas. MDSCs are elevated in advanced colorectal cancer (Toor et al, Front Immunol. 2016; 7:560). MDSCs are also observed in breast cancer, and the percentage of MDSCs in the peripheral blood is increased in patients with later stage breast cancer (Markowitz et al, Breast Cancer Res Treat. 2013 July; 140(1):13-21). MDSC abundance is also correlated with poor prognosis in solid tumors (Charoentong et al, Cell Rep. 2017 Jan. 3; 18(1):248-262).

MDSCs exert suppression over T cells through multiple mechanisms, including the production of reactive oxygen species, nitric oxide, and arginase. These ultimately lead to suppression of DC, NK and T cell activity and increased tumor burden (Umansky et al., Vaccines (Basel) (2016) 4(4):36). MDSCs also contribute to the tumor development and metastasis through the production of soluble factors such as matrix metalloproteinases, VEGF, bFGF, TGF-β and S100A8/A9 which promote neovascularisation, invasion, proliferation and metastasis.

Targeting V-type immunoglobulin domain-containing suppressor of T-cell activation (VISTA), an immune checkpoint molecule expressed primarily on MDSCs, is an attractive therapeutic strategy for removing MDSC-mediated suppression of effector immune cell function.

WO 2017/137830 A1 discloses anti-VISTA antibody VSTB174, which is disclosed at e.g. paragraph [00221] to comprise the variable regions of anti-VISTA antibody VSTB112. Paragraph [00362] discloses that VSTB123 comprises the variable regions of VSTB174. Example 25 of WO 2017/137830 A1 at paragraph [0417] and FIG. 42A disclose that mIgG2a antibody VSTB123 was able to inhibit tumor growth in a MB49 tumor model. Paragraph [0418] and FIG. 42A disclose that by contrast VSTB124—which is the same antibody provided in IgG2a LALA format; see paragraph [0408]—did not inhibit tumor growth. Based on these results Example 25 concludes at paragraph [0419] that efficacy with anti-VISTA antibody treatment might require active Fc. Accordingly, the proposed mechanism of action for the anti-VISTA antibody represented schematically at FIG. 47 (see the legend to FIG. 47 at paragraph [0053]) involves Fc-mediated engagement of FcγRIII expressed by NK cells.

Hamster monoclonal anti-VISTA antibody mAb13F3 is disclosed in Le Mercier et al. Cancer Res. (2014) 74(7):1933-44 to inhibit tumor growth in B160VA and B16-BL6 melanoma models. Page 1942, paragraph spanning left and right columns teaches that immunogenicity and the FcR binding activity of the VISTA mAb might be critical limiting factors for achieving optimal target neutralization and therapeutic efficacy. VISTA-binding antibodies are also disclosed e.g. in WO 2019/185879 A1.

### SUMMARY

In a first aspect, the present disclosure provides an antigen-binding molecule that binds to VISTA for use in a method of treating or preventing a cancer in a subject, wherein the treatment or prophylaxis comprises:
(i) increasing the number and/or proportion of antigen-specific CD8+ T cells;
(ii) increasing CD8+ T cell activity;
(iii) reducing the level of T cell exhaustion;
(iv) reducing the number and/or proportion of tumour-associated macrophages (TAMs);
(v) increasing the number and/or proportion of M1-type macrophages; and/or
(vi) increasing M1-type macrophage activity.

The present disclosure also provides, use of an antigen-binding molecule that binds to VISTA in the manufacture of a medicament for treating or preventing a cancer in a subject, wherein the treatment or prophylaxis comprises:
(i) increasing the number and/or proportion of antigen-specific CD8+ T cells;
(ii) increasing CD8+ T cell activity;
(iii) reducing the level of T cell exhaustion;
(iv) reducing the number and/or proportion of tumour-associated macrophages (TAMs);
(v) increasing the number and/or proportion of M1-type macrophages; and/or
(vi) increasing M1-type macrophage activity.

The present disclosure also provides, a method of treating or preventing a cancer in a subject, wherein the method comprises administering to a subject a therapeutically or prophylactically effective amount of an antigen-binding molecule that binds to VISTA, wherein the treatment or prophylaxis comprises:
(i) increasing the number and/or proportion of antigen-specific CD8+ T cells;
(ii) increasing CD8+ T cell activity;
(iii) reducing the level of T cell exhaustion;
(iv) reducing the number and/or proportion of tumour-associated macrophages (TAMs);
(v) increasing the number and/or proportion of M1-type macrophages; and/or
(vi) increasing M1-type macrophage activity.

In some embodiments, the cancer comprises a tumor comprising cells expressing VISTA.

The present disclosure also provides, a method of selecting a subject for treatment with an antigen-binding molecule that binds to VISTA, comprising:
(a) analysing a subject's cancer in order to determine whether the cancer is characterised by:
   (i) low number and/or proportion of antigen-specific CD8+ T cells;
   (ii) low CD8+ T cell activity;
   (iii) presence and/or high levels of exhausted T cells;
   (iv) presence and/or high number and/or proportion of TAMs;
   (v) low number and/or proportion of M1-type macrophages; and/or
   (vi) low M1-type macrophage activity; and
(b) selecting a subject for treatment with an antigen-binding molecule that binds to VISTA where the subject's cancer is determined in step (a) to have one or more of (i) to (vi).

The present disclosure also provides, a method of determining the response in a patient to treatment with an antigen-binding molecule that binds to VISTA, comprising:
(a) analysing a subject's cancer at a first timepoint in order to determine the:
   (i) number and/or proportion of antigen-specific CD8+ T cells;
   (ii) CD8+ T cell activity;
   (iii) level of T cell exhaustion;
   (iv) number and/or proportion of tumour-associated macrophages (TAMs);
   (v) number and/or proportion of M1-type macrophages; and/or
   (vi) M1-type macrophage activity;
(b) analysing a subject's cancer at a subsequent timepoint in order to determine one or more of (i) to (vi); and
(c) determining the difference between (a) and (b), wherein a(n):
   (i) increased number and/or proportion of antigen-specific CD8+ T cells;
   (ii) increased CD8+ T cells activity;
   (iii) reduced level of T cell exhaustion;
   (iv) reduced number and/or proportion of tumour-associated macrophages (TAMs);
   (v) increased number and/or proportion of M1-type macrophages; and/or
   (vi) increased M1-type macrophage activity, in (b) relative to (a) signifies a positive response to treatment with an antigen-binding molecule that binds to VISTA.

In some embodiments, the antigen-binding molecule comprises:
(i) a heavy chain variable (VH) region incorporating the following CDRs: HC-CDR1 having the amino acid sequence of SEQ ID NO:305 HC-CDR2 having the amino acid sequence of SEQ ID NO:306 HC-CDR3 having the amino acid sequence of SEQ ID NO:307; and
(ii) a light chain variable (VL) region incorporating the following CDRs: LC-CDR1 having the amino acid sequence of SEQ ID NO:41 LC-CDR2 having the amino acid sequence of SEQ ID NO:308 LC-CDR3 having the amino acid sequence of SEQ ID NO:43.

In some embodiments, the antigen-binding molecule comprises:
(i) a VH region incorporating the following CDRs: HC-CDR1 having the amino acid sequence of SEQ ID NO:290 HC-CDR2 having the amino acid sequence of SEQ ID NO:291 HC-CDR3 having the amino acid sequence of SEQ ID NO:278; and
(ii) a VL region incorporating the following CDRs: LC-CDR1 having the amino acid sequence of SEQ ID NO:41 LC-CDR2 having the amino acid sequence of SEQ ID NO:295 LC-CDR3 having the amino acid sequence of SEQ ID NO:43.

In some embodiments, the antigen-binding molecule comprises:
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:297.

In some embodiments, the antigen-binding molecule comprises:
a VH region incorporating the following framework regions (FRs):
HC-FR1 having the amino acid sequence of SEQ ID NO:63
HC-FR2 having the amino acid sequence of SEQ ID NO:292
HC-FR3 having the amino acid sequence of SEQ ID NO:293
HC-FR4 having the amino acid sequence of SEQ ID NO:281.

In some embodiments, the antigen-binding molecule comprises:
a VL region incorporating the following framework regions (FRs):
LC-FR1 having the amino acid sequence of SEQ ID NO:288
LC-FR2 having the amino acid sequence of SEQ ID NO:298
LC-FR3 having the amino acid sequence of SEQ ID NO:284
LC-FR4 having the amino acid sequence of SEQ ID NO:47.

In some embodiments, the antigen-binding molecule comprises a heavy chain comprising the amino acid sequence of SEQ ID NO:331.

In some embodiments, the antigen-binding molecule comprises a light chain comprising the amino acid sequence of SEQ ID NO:317.

In some embodiments, the cancer is selected from: a hematological cancer, leukemia, acute myeloid leukemia, lymphoma, B cell lymphoma, T cell lymphoma, multiple myeloma, mesothelioma, epithelioid mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

In some embodiments, the cancer is selected from: colorectal cancer, pancreatic cancer, breast cancer, triple-negative breast cancer, liver cancer, prostate cancer, ovarian cancer, head and neck cancer, leukemia, lymphoma, melanoma, thymoma, lung cancer, non-small cell lung cancer (NSCLC) and a solid tumor.

In some embodiments, the cancer is epithelioid mesothelioma.

### DESCRIPTION

The present disclosure relates to VISTA-binding molecules that modify the tumour microenvironment.

Aspects and embodiments of the present disclosure are concerned in particular with antigen-binding molecules that bind to VISTA, and that affect remodelling of the tumour microenvironment. Such antigen-binding molecules are useful for the treatment/prevention of cancers.

### VISTA, Interaction Partners and VISTA-Mediated Signalling

V-type immunoglobulin domain-containing suppressor of T-cell activation (VISTA; also known e.g. as B7-H5, SISP1, PD-1H) is the protein identified by UniProt Q9H7M9, having the amino acid sequence shown in SEQ ID NO:1 (Q9H7M9-1, v3). The structure and function of VISTA is described e.g. in Lines et al., Cancer Res. (2014) 74(7): 1924-1932, which is hereby incorporated by reference in its entirety. VISTA is a ˜50 kDa single-pass type I transmembrane that functions as an immune checkpoint and is encoded by the C10orf54 gene. The extracellular domain of VISTA is homologous to PD-L1.

The N-terminal 32 amino acids of SEQ ID NO:1 constitutes a signal peptide, and so the mature form of VISTA (i.e. after processing to remove the signal peptide) has the amino acid sequence shown in SEQ ID NO:2. Positions 33 to 194 of SEQ ID NO:1 form the extracellular domain (SEQ ID NO:3), positions 195 to 215 form a transmembrane domain (SEQ ID NO:4), and positions 216 to 311 form the cytoplasmic domain (SEQ ID NO:5). The extracellular domain comprises an Ig-like V-type domain (positions 33 to 168 of SEQ ID NO:1, shown in SEQ ID NO:6).

In this specification “VISTA” refers to VISTA from any species and includes VISTA isoforms, fragments, variants (including mutants) or homologues from any species.

As used herein, a “fragment”, “variant” or “homologue” of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments fragments, variants, isoforms and homologues of a reference protein may be characterised by ability to perform a function performed by the reference protein.

A “fragment” generally refers to a fraction of the reference protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein. Homologues include orthologues.

A “fragment” may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein. A fragment of VISTA may have a minimum length of one of 10, 20, 30, 40, 50, 100, 150, 200, 250 or 300 amino acids, and may have a maximum length of one of 20, 30, 40, 50, 100, 150, 200, 250 or 300 amino acids.

In some embodiments, the VISTA is VISTA from a mammal (e.g. a primate (rhesus, cynomolgous, non-human primate or human) and/or a rodent (e.g. rat or murine) VISTA). Isoforms, fragments, variants or homologues of VISTA may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VISTA isoform from a given species, e.g. human.

Isoforms, fragments, variants or homologues may optionally be functional isoforms, fragments, variants or homologues, e.g. having a functional property/activity of the reference VISTA, as determined by analysis by a suitable assay for the functional property/activity. For example, an isoform, fragment, variant or homologue of VISTA may e.g. display association with LRIG1, VSIG3, PSGL-1 and/or VSIG8.

In some embodiments, the VISTA comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1 or 2. In some embodiments, a fragment of VISTA comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:2, 3 or 6.

VISTA is a member of the B7 family of proteins, and is primarily expressed by leukocytes, and in particular CD14+ monocytes (including monocyte-derived suppressor cells (MDSCs)) and CD33+ myeloid cells. VISTA is also expressed by CD56+NK cells, dendritic cells, and to a lesser extent on CD4+ and CD8+ T cells. VISTA is highly expressed on MDSCs, in particular tumor-infiltrating MDSCs, and also on tumor-infiltrating myeloid DCs (Le Mercier et al, Cancer Res. (2014) 74(7):1933-44), as well as on tumor-associated macrophages (TAMs) and neutrophils.

There is evidence that VISTA can act as both a ligand and a receptor on T cells to inhibit T cell effector function and maintain peripheral tolerance; tumors engineered to overexpress VISTA evade immune control and grow faster than tumors which do not overexpress VISTA (Wang et al., Journal of Experimental Medicine. (2011) 208 (3): 577-92; Lines et al., Cancer Res. (2014) 74(7): 1924-1932). VISTA has been shown to be a co-inhibitory receptor on CD4+ T cells or a co-inhibitory ligand for T cells. VISTA^{-/-} CD4+ T cells have been reported to display stronger antigen-specific proliferation and cytokine production than wildtype CD4+ T cells, suggesting that VISTA functions as an inhibitory receptor on CD4+ T cells. Blocking VISTA function using monoclonal anti-VISTA antibody has been shown to enhance infiltration, proliferation and effector function of tumor-reactive T cells within the tumor microenvironment (Le Mercier et al, Cancer Res. (2014) 74(7):1933-4).

VISTA has been proposed to interact with VSIG3 (IGSF11)—see e.g. Wang et al., J Immunol (2017), 198 (1 Supplement) 154.1, which is hereby incorporated by reference in its entirety. Engagement of VSIG3 through VISTA on activated T cells inhibits T cell proliferation, and reduces production of cytokines and chemokines such as IFN-γ, IL-2, IL-17, CCL5/RANTES, CCL3/MIP-1a, and CXCL11/I-TAC.

VSIG3 is the protein identified by UniProt Q5DX21. Alternative splicing of mRNA encoded by the human IGSF11 gene yields three different isoforms: isoform 1 (UniProt: Q5DX21-1, v3; SEQ ID NO:7); isoform 2 (UniProt: Q5DX21-2; SEQ ID NO:8), which comprises a different sequence to SEQ ID NO:7 at positions 1 to 17; and isoform 3 (UniProt: Q5DX21-3; SEQ ID NO:9), which comprises a different sequence to SEQ ID NO:7 at positions 1 to 17, and which also comprises a different sequence to SEQ ID NO:7 at positions 211-235.

The N-terminal 22 amino acids of SEQ ID NOs:7, 8 and 9 constitute a signal peptide, and so the mature form of VSIG3 isoforms 1, 2 and 3 (i.e. after processing to remove the signal peptide) have the amino acid sequences shown in SEQ ID NOs:10, 11 and 12, respectively. Positions 23 to 241 of SEQ ID NOs:7, and 8 form the extracellular domain of VSIG3 isoforms 1 and 2 (SEQ ID NO:13), and positions 23 to 216 of SEQ ID NO:9 form the extracellular domain of VSIG3 isoform 3 (SEQ ID NO:14). The transmembrane domain of VSIG3 is shown in SEQ ID NO:15, and the cytoplasmic domain is shown in SEQ ID NO:16.

The extracellular domain comprises an Ig-like V-type domain (shown in SEQ ID NO:17), and the extracellular domains of VSIG3 isoforms 1 and 2 additionally comprise an Ig-like C2-type domain (shown in SEQ ID NO:18).

In this specification “VSIG3” refers to VSIG3 from any species and includes VSIG3 isoforms, fragments, variants (including mutants) or homologues from any species.

A fragment of VSIG3 may have a minimum length of one of 10, 20, 30, 40, 50, 100, 150, 200, 250, 300, 350 or 400 amino acids, and may have a maximum length of one of 20, 30, 40, 50, 100, 150, 200, 250, 300, 350 or 400 amino acids.

In some embodiments, the VSIG3 is VSIG3 from a mammal (e.g. a primate (rhesus, cynomolgous, non-human primate or human) and/or a rodent (e.g. rat or murine) VSIG3). Isoforms, fragments, variants or homologues of VSIG3 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VSIG3 isoform from a given species, e.g. human.

Isoforms, fragments, variants or homologues may optionally be functional isoforms, fragments, variants or homologues, e.g. having a functional property/activity of the reference VSIG3, as determined by analysis by a suitable assay for the functional property/activity. For example, an isoform, fragment, variant or homologue of VSIG3 may e.g. display association with VISTA.

In some embodiments, the VSIG3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:7 to 12. In some embodiments, a fragment of VSIG3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:10 to 14, 17 or 18.

VISTA has also been proposed to interact with VSIG8—see e.g. WO/2016/090347 A1. VSIG8 is the protein identified by UniProt PODPA2 (SEQ ID NO:19). The N-terminal 21 amino acids of SEQ ID NO:19 constitutes a signal peptide, and so the mature form of VSIG8 (i.e. after processing to remove the signal peptide) has the amino acid sequence shown in SEQ ID NO:20. Positions 22 to 263 of SEQ ID NO:19 form the extracellular domain of VSIG8 (SEQ ID NO:21). The transmembrane domain of VSIG8 is shown in SEQ ID NO:22, and the cytoplasmic domain is shown in SEQ ID NO:23. The extracellular domain comprises an Ig-like V-type domain 1 (shown in SEQ ID NO:24), and an Ig-like V-type domain 2 (shown in SEQ ID NO:25).

In this specification “VSIG8” refers to VSIG8 from any species and includes VSIG8 isoforms, fragments, variants (including mutants) or homologues from any species.

A fragment of VSIG8 may have a minimum length of one of 10, 20, 30, 40, 50, 100, 150, 200, 250, 300, 350 or 400 amino acids, and may have a maximum length of one of 20, 30, 40, 50, 100, 150, 200, 250, 300, 350 or 400 amino acids.

In some embodiments, the VSIG8 is VSIG8 from a mammal (e.g. a primate (rhesus, cynomolgous, non-human primate or human) and/or a rodent (e.g. rat or murine) VSIG8). Isoforms, fragments, variants or homologues of VSIG8 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VSIG8 isoform from a given species, e.g. human.

Isoforms, fragments, variants or homologues may optionally be functional isoforms, fragments, variants or homologues, e.g. having a functional property/activity of the reference VSIG8, as determined by analysis by a suitable assay for the functional property/activity. For example, an isoform, fragment, variant or homologue of VSIG8 may e.g. display association with VISTA.

In some embodiments, the VSIG8 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:19 or 20. In some embodiments, a fragment of VSIG8 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:20, 21, 24 or 25.

VISTA has also been proposed to interact with PSGL-1—see e.g. WO 2018/132476 A1. Johnston et al., Nature (2019) 574: 565-570 discloses that PSGL-1 associates with VISTA via interaction involving positions Y46, Y48, Y51, E56 and T57 of PSGL-1, and positions H98, H100, H153, H154 and H155 of VISTA.

PSGL-1 isoform 1 is the protein identified by UniProt Q14242-1 (SEQ ID NO:323). PSGL-1 isoform 2 is the protein identified by UniProt Q14242-2 (SEQ ID NO:324), and differs from PSGL-1 isoform 1 in that it comprises an additional 16 amino acids after position 1 of SEQ ID NO:323.

The N-terminal 17 amino acids of SEQ ID NO:323 constitutes a signal peptide, and so the mature form of PSGL-1 (i.e. after processing to remove the signal peptide) has the amino acid sequence shown in SEQ ID NO:325. Positions 18 to 320 of SEQ ID NO:323 form the extracellular domain of PSGL-1 (SEQ ID NO:326). The transmembrane domain of PSGL-1 is shown in SEQ ID NO:327, and the cytoplasmic domain is shown in SEQ ID NO:328. The extracellular domain comprises 12, 10 amino acid tandem repeats; the repeat region is shown in SEQ ID NO:329.

In this specification “PSGL-1” refers to PSGL-1 from any species and includes PSGL-1 isoforms, fragments, variants (including mutants) or homologues from any species.

A fragment of PSGL-1 may have a minimum length of one of 10, 20, 30, 40, 50, 100, 150, 200, 250, 300, 350 or 400 amino acids, and may have a maximum length of one of 20, 30, 40, 50, 100, 150, 200, 250, 300, 350 or 400 amino acids.

In some embodiments, the PSGL-1 is PSGL-1 from a mammal (e.g. a primate (rhesus, cynomolgous, non-human primate or human) and/or a rodent (e.g. rat or murine) PSGL-1). Isoforms, fragments, variants or homologues of PSGL-1 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature PSGL-1 isoform from a given species, e.g. human.

Isoforms, fragments, variants or homologues may optionally be functional isoforms, fragments, variants or homologues, e.g. having a functional property/activity of the reference PSGL-1, as determined by analysis by a suitable assay for the functional property/activity. For example, an isoform, fragment, variant or homologue of PSGL-1 may e.g. display association with VISTA.

In some embodiments, the PSGL-1 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:323 or 324. In some embodiments, a fragment of PSGL-1 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:325, 326 or 329.

VISTA has also been proposed to interact with LRIG1—see e.g. WO/2019/165233 A1. WO/2019/165233 A1 discloses that LRIG1 associates with VISTA via interaction involving positions 245 to 260 of LRIG1, and positions 68 to 92 of VISTA.

LRIG1 isoform 1 is the protein identified by UniProt Q96JA1-1 (SEQ ID NO:332). LRIG1 isoform 2 is the protein identified by UniProt Q96JA1-2 (SEQ ID NO:334), and differs from LRIG1 isoform 1 in that it comprises an additional 14 amino acids after position 387 of SEQ ID NO:332, and in that positions 644 to 691 of SEQ ID NO:332 are instead Q.

The N-terminal 34 amino acids of SEQ ID NO:332 constitutes a signal peptide, and so the mature form of LRIG1 isoforms 1 and 2 (i.e. after processing to remove the signal peptide) have the amino acid sequences shown in SEQ ID NOs:333 and 335, respectively. The extracellular domain of LRIG1 isoform 1 is shown in SEQ ID NO:336, and the extracellular domain of LRIG1 isoform 2 is shown in SEQ ID NO:337. The transmembrane domain of LRIG1 is shown in SEQ ID NO:338, and the cytoplasmic domain is shown in SEQ ID NO:339. The extracellular domain comprises 15, leucine-rich repeats, followed by three Ig-like domains proximal to the transmembrane domain (see e.g. Xu et al. J Mol Biol. (2015) 427(10): 1934-1948).

In this specification “LRIG1” refers to LRIG1 from any species and includes LRIG1 isoforms, fragments, variants (including mutants) or homologues from any species.

A fragment of LRIG1 may have a minimum length of one of 10, 20, 30, 40, 50, 100, 150, 200, 250, 300, 350, 400, 500, 600 or 700 amino acids, and may have a maximum length of one of 20, 30, 40, 50, 100, 150, 200, 250, 300, 350, 400, 500, 600 or 700 amino acids.

In some embodiments, the LRIG1 is LRIG1 from a mammal (e.g. a primate (rhesus, cynomolgus, non-human primate or human) and/or a rodent (e.g. rat or murine) LRIG1). Isoforms, fragments, variants or homologues of LRIG1 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature LRIG1 isoform from a given species, e.g. human.

Isoforms, fragments, variants or homologues may optionally be functional isoforms, fragments, variants or homologues, e.g. having a functional property/activity of the reference LRIG1, as determined by analysis by a suitable assay for the functional property/activity. For example, an isoform, fragment, variant or homologue of LRIG1 may e.g. display association with VISTA.

In some embodiments, the LRIG1 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:332, 333, 334 or 335. In some embodiments, a fragment of LRIG1 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:336 or 337.

As explained in the experimental examples of the present disclosure, VSIG3 and LRIG1 are both thought to bind to VISTA via interaction with the C-C′ loop region of VISTA, the amino acid sequence of which is shown in SEQ ID NO:344.

### Regions of Particular Interest on the Target Molecule

The antigen-binding molecules of the present disclosure were specifically designed to target regions of VISTA of particular interest. In a two-step approach, VISTA regions to be targeted were selected following analysis for predicted antigenicity, function and safety. Antibodies specific for the target regions of VISTA were then prepared using peptides corresponding to the target regions as immunogens to raise specific monoclonal antibodies, and subsequent screening to identify antibodies capable of binding to VISTA in the native state. This approach provides exquisite control over the antibody epitope.

The antigen-binding molecules of the present disclosure may be defined by reference to the region of VISTA which they bind to. The antigen-binding molecules of the present disclosure may bind to a particular region of interest of VISTA. In some embodiments, the antigen-binding molecule may bind to a linear epitope of VISTA, consisting of a contiguous sequence of amino acids (i.e. an amino acid primary sequence). In some embodiments, the antigen-binding molecule may bind to a conformational epitope of VISTA, consisting of a discontinuous sequence of amino acids of the amino acid sequence.

In some embodiments, the antigen-binding molecule of the present disclosure binds to VISTA. In some embodiments, the antigen-binding molecule binds to the extracellular region of VISTA (e.g. the region shown in SEQ ID NO:3). In some embodiments, the antigen-binding molecule binds to the Ig-like V-type domain of VISTA (e.g. the region shown in SEQ ID NO:6). In some embodiments, the antigen-binding molecule binds to VISTA in the region corresponding to positions 61 to 162 of SEQ ID NO:1 (shown in SEQ ID NO:31).

In some embodiments, the antigen-binding molecule binds to the region of VISTA shown in SEQ ID NO:322. In some embodiments, the antigen-binding molecule binds to the region of VISTA shown in SEQ ID NO:26. In some embodiments, the antigen-binding molecule binds to the region of VISTA shown in SEQ ID NO:27. In some embodiments, the antigen-binding molecule binds to the region of VISTA shown in SEQ ID NO:28. In some embodiments, the antigen-binding molecule binds to the region of VISTA shown in SEQ ID NO:29. In some embodiments, the antigen-binding molecule binds to the region of VISTA shown in SEQ ID NO:30.

In some embodiments, the antigen-binding molecule does not bind to the region of VISTA shown in SEQ ID NO:271. In some embodiments, the antigen-binding molecule does not bind to the region of VISTA shown in SEQ ID NO:272. In some embodiments, the antigen-binding molecule does not bind to the region of VISTA shown in SEQ ID NO:273. In some embodiments, the antigen-binding molecule does not bind to the region of VISTA shown in SEQ ID NO:274. In some embodiments, the antigen-binding molecule does not bind to the region of VISTA shown in SEQ ID NO:275.

The region of a peptide/polypeptide to which an antibody binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In preferred embodiments, the region of a peptide/polypeptide to which an antigen-binding molecule according the present disclosure binds is evaluated by hydrogen-deuterium exchange mass spectrometry (HDXMS) analysis, e.g. as described in the experimental examples of the present disclosure.

In some embodiments, the antigen-binding molecule binds to the C-C′ region of VISTA. In some embodiments, the antigen-binding molecule binds to the region of VISTA shown in SEQ ID NO:344. In some embodiments, the antigen-binding molecule binds to a polypeptide comprising or consisting of the amino acid sequence shown in SEQ ID NO:344. In some embodiments, the antigen-binding molecule contacts the region of VISTA shown in SEQ ID NO:344. In some embodiments, the antigen-binding molecule binds to VISTA via contact with one or more amino acids of the region shown in SEQ ID NO:344. In some embodiments, the epitope of the antigen-binding molecule comprises or consists of the amino acid sequence shown in SEQ ID NO:344.

In some embodiments, the antigen-binding molecule binds to the region of VISTA shown in SEQ ID NO:340. In some embodiments, the antigen-binding molecule binds to a polypeptide comprising or consisting of the amino acid sequence shown in SEQ ID NO:340. In some embodiments, the antigen-binding molecule contacts the region of VISTA shown in SEQ ID NO:340. In some embodiments, the antigen-binding molecule binds to VISTA via contact with one or more amino acids of the region shown in SEQ ID NO:340. In some embodiments, the epitope of the antigen-binding molecule comprises or consists of the amino acid sequence shown in SEQ ID NO:340.

In some embodiments, the antigen-binding molecule binds to the region of VISTA shown in SEQ ID NO:341. In some embodiments, the antigen-binding molecule binds to a polypeptide comprising or consisting of the amino acid sequence shown in SEQ ID NO:341. In some embodiments, the antigen-binding molecule contacts the region of VISTA shown in SEQ ID NO:341. In some embodiments, the antigen-binding molecule binds to VISTA via contact with one or more amino acids of the region shown in SEQ ID NO:341. In some embodiments, the epitope of the antigen-binding molecule comprises or consists of the amino acid sequence shown in SEQ ID NO:341.

In some embodiments, the antigen-binding molecule binds to the region of VISTA shown in SEQ ID NO:342. In some embodiments, the antigen-binding molecule binds to a polypeptide comprising or consisting of the amino acid sequence shown in SEQ ID NO:342. In some embodiments, the antigen-binding molecule contacts the region of VISTA shown in SEQ ID NO:342. In some embodiments, the antigen-binding molecule binds to VISTA via contact with one or more amino acids of the region shown in SEQ ID NO:342. In some embodiments, the epitope of the antigen-binding molecule comprises or consists of the amino acid sequence shown in SEQ ID NO:342.

In some embodiments, the antigen-binding molecule binds to the region of VISTA shown in SEQ ID NO:341, and/or binds to the region of VISTA shown in SEQ ID NO:342. In some embodiments, the antigen-binding molecule binds to a polypeptide comprising or consisting of the amino acid sequence shown in SEQ ID NO:341 and/or binds to a polypeptide comprising or consisting of the amino acid sequence shown in SEQ ID NO:342. In some embodiments, the antigen-binding molecule contacts the region of VISTA shown in SEQ ID NO:341 and/or contacts the region of VISTA shown in SEQ ID NO:342.

In some embodiments, the antigen-binding molecule binds to VISTA via contact with one or more amino acids of the region shown in SEQ ID NO:341 and/or binds to VISTA via contact with one or more amino acids of the region shown in SEQ ID NO:342. In some embodiments, the epitope of the antigen-binding molecule comprises or consists of the amino acid sequence shown in SEQ ID NO:341, and/or the amino acid sequence shown in SEQ ID NO:342.

In some embodiments, the antigen-binding molecule binds to the region of VISTA bound by an interaction partner for VISTA that binds to C-C′ region of VISTA (e.g. LRIG1 or VSIG3). In some embodiments, the antigen-binding molecule binds to the region of VISTA bound by LRIG1. In some embodiments, the antigen-binding molecule binds to the region of VISTA bound by VSIG3.

In some embodiments, the antigen-binding molecule binds to the region of VISTA shown in SEQ ID NO:343. In some embodiments, the antigen-binding molecule binds to a polypeptide comprising or consisting of the amino acid sequence shown in SEQ ID NO:343. In some embodiments, the antigen-binding molecule contacts the region of VISTA shown in SEQ ID NO:343. In some embodiments, the antigen-binding molecule binds to VISTA via contact with one or more amino acids of the region shown in SEQ ID NO:343. In some embodiments, the epitope of the antigen-binding molecule comprises or consists of the amino acid sequence shown in SEQ ID NO:343.

In some embodiments, the antigen-binding molecule is capable of binding the same region of VISTA, or an overlapping region of VISTA, to the region of VISTA which is bound by an antibody comprising the VH and VL sequences of one of antibody clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2-D2, 13D5p, 13D5-1, 13D5-13, 5M1-A11 or 9M2-C12 described herein. In some embodiments, the antigen-binding molecule is capable of binding the same region of VISTA, or an overlapping region of VISTA, to the region of VISTA which is bound by an antibody comprising the VH and VL sequences of one of antibody clones 4M2-C12, V4H1, V4H2, V4-C1, V4-C9, V4-C24, V4-C26, V4-C27, V4-C28, V4-C30, V4-C31. In some embodiments, the antigen-binding molecule is capable of binding the same region of VISTA, or an overlapping region of VISTA, to the region of VISTA which is bound by an antibody comprising the VH and VL sequences of V4-C26.

As used herein, a “peptide” refers to a chain of two or more amino acid monomers linked by peptide bonds. A peptide typically has a length in the region of about 2 to 50 amino acids. A “polypeptide” is a polymer chain of two or more peptides. Polypeptides typically have a length greater than about 50 amino acids.

In some embodiments, the antigen-binding molecule of the present disclosure is capable of binding to a polypeptide comprising, or consisting of, the amino acid sequence of one of SEQ ID NOs:1, 2, 3, 6 or 31.

In some embodiments, the antigen-binding molecule is capable of binding to a peptide/polypeptide comprising, or consisting of, the amino acid sequence of SEQ ID NO:322. In some embodiments, the antigen-binding molecule is capable of binding to a peptide/polypeptide comprising, or consisting of, the amino acid sequence of SEQ ID NO:26. In some embodiments, the antigen-binding molecule is capable of binding to a peptide/polypeptide comprising, or consisting of, the amino acid sequence of SEQ ID NO:27. In some embodiments, the antigen-binding molecule is capable of binding to a peptide/polypeptide comprising, or consisting of, the amino acid sequence of SEQ ID NO:28. In some embodiments, the antigen-binding molecule is capable of binding to a peptide/polypeptide comprising, or consisting of, the amino acid sequence of SEQ ID NO:29. In some embodiments, the antigen-binding molecule is capable of binding to a peptide/polypeptide comprising, or consisting of, the amino acid sequence of SEQ ID NO:30.

In some embodiments, the antigen-binding molecule is not capable of binding to a peptide consisting of the amino acid sequence of SEQ ID NO:271. In some embodiments, the antigen-binding molecule is not capable of binding to a peptide consisting of the amino acid sequence of SEQ ID NO:272. In some embodiments, the antigen-binding molecule is not capable of binding to a peptide consisting of the amino acid sequence of SEQ ID NO:273. In some embodiments, the antigen-binding molecule is not capable of binding to a peptide consisting of the amino acid sequence of SEQ ID NO:274. In some embodiments, the antigen-binding molecule is not capable of binding to a peptide consisting of the amino acid sequence of SEQ ID NO:275.

The ability of an antigen-binding molecule to bind to a given peptide/polypeptide can be analysed by methods well known to the skilled person, including analysis by ELISA, immunoblot (e.g. western blot), immunoprecipitation, Surface Plasmon Resonance (SPR; see e.g. Hearty et al., Methods Mol Biol (2012) 907:411-442) or Bio-Layer Interferometry (see e.g. Lad et al., (2015) J Biomol Screen 20(4): 498-507).

In embodiments where the antigen binding molecule is capable of binding to a peptide/polypeptide comprising a reference amino acid sequence, the peptide/polypeptide may comprise one or more additional amino acids at one or both ends of the reference amino acid sequence. In some embodiments, the peptide/polypeptide comprises e.g. 1-5, 1-10, 1-20, 1-30, 1-40, 1-50, 5-10, 5-20, 5-30, 5-40, 5-50, 10-20, 10-30, 10-40, 10-50, 20-30, 20-40 or 20-50 additional amino acids at one or both ends of the reference amino acid sequence.

In some embodiments, the additional amino acid(s) provided at one or both ends (i.e. the N-terminal and C-terminal ends) of the reference sequence correspond to the positions at the ends of the reference sequence in the context of the amino acid sequence of VISTA. By way of example, where the antigen-binding molecule is capable of binding to a peptide/polypeptide comprising the sequence of SEQ ID NO:26, and an additional two amino acids at the C-terminal end of SEQ ID NO:26, the additional two amino acids may be arginine and asparagine, corresponding to positions 90 and 91 of SEQ ID NO:1.

In some embodiments, the antigen-binding molecule is capable of binding to a peptide/polypeptide which is bound by an antibody comprising the VH and VL sequences of one of antibody clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2-D2, 13D5p, 13D5-1, 13D5-13, 5M1-A11 or 9M2-C12 described herein.

### Myeloid-Derived Suppressor Cells (MDSCs)

Myeloid-Derived Suppressor Cells (MDSCs) are a heterogeneous group of immune cells of the myeloid lineage of cells, characterised by an immunosuppressive phenotype. MDSC biology is reviewed in Kumar et al., Trends Immunol. (2016); 37(3): 208-220, which is hereby incorporated by reference in its entirety.

MDSC are characterised by a number of biochemical and genomic features that distinguish these cells from mature myeloid cells (i.e. macrophages, dendritic cells and neutrophils) such as: increased expression of NADPH oxidase (Nox2), increased production of reactive oxygen species (ROS) (such as superoxide anion (O²⁻), hydrogen peroxide (H₂O₂), and peroxynitrite (PNT; ONOO⁻)); increased expression of arginase 1 and nitric oxide synthase 2 (nos2), and increased production of nitric oxide (NO); increased expression of c/EBPP and STAT3; decreased expression of IRF8; and increased production of S100A8/9 proteins.

There are two different types of MDSC; polymorphonuclear MDSCs (PMN-MDSCs), which are morphologically and phenotypically similar to neutrophils, and monocytic MDSCs (M-MDSCs) which are more similar to monocytes. The morphologic and phenotypic characteristics of MDSCs are described e.g. in Marvel and Gabrilovich J Clin Invest. 2015 Sep. 1; 125(9): 3356-3364, which is hereby incorporated by reference in its entirety. In mice, MDSCs are broadly identified as CD11 b+Gr1⁺ cells. Gr-1^{hi} cells are mostly PMN-MDSCs, and Gr-1^{lo} cells are mostly M-MDSCs. These subsets can be more accurately identified based on Ly6C and Ly6G markers; M-MDSCs are CD11 b⁺Ly6C^{hi}Ly6G⁻, and PMN-MDSCs are CD11 b⁺Ly6C^{lo}Ly6G⁺). In humans, MDSCs are identified in the mononuclear fraction. PMN-MDSCs are CD14⁻CD11 b⁺CD33⁺CD15⁺ or CD66b⁺ cells, and M-MDSCs are CD14⁺HLA-DR−/¹ cells. Populations of Lin-HLA-DR-CD33⁺ MDSCs represent a mixed group of cells enriched for myeloid progenitors.

Factors implicated in MDSC-mediated immune suppression include expression of arginase (ARG1), inducible NOS (iNOS), TGF-β, IL-10, and COX2, sequestration of cysteine, decreased expression of I-selectin by T cells, and induction of Tregs. M-MDSCs and PMN-MDSCs employ different mechanisms of immune suppression. M-MDSCs suppress both antigen-specific and non-specific T cell responses through production of NO and cytokines, and are more strongly immunosuppressive than PMN-MDSCs. PMN-MDSCs suppress immune responses in an antigen-specific manner through production of ROS. MDSCs are pathologically implicated in the development and progression of cancer and infectious disease. The role of MDSCs in human disease is reviewed e.g. in Kumar et al., Trends Immunol. (2016); 37(3): 208-220 (incorporated by reference herein) and Greten et al., Int Immunopharmacol. (2011) 11(7):802-807, which is hereby incorporated by reference in its entirety.

MDSCs are abundant in tumor tissues, and contribute to the development and progression of cancer through multiple mechanisms, reviewed e.g. in Umansky et al., Vaccines (Basel) (2016) 4(4):36. MDSCs are recruited to the tumor site through chemokine expression, and proinflammatory factors in the tumor microenvironment result in significant upregulation of immunosuppressive function by MDSCs. MDSCs contribute to tumor development, neovascularization and metastasis through suppression of effector immune cell function (e.g. effector T cell and NK cell function), promotion of regulatory T cell production/activity, production of growth factors such as VEGF and bFGF, and production of ECM-modifying factors such as matrix metalloproteinases.

MDSCs may be characterised by reference to expression of VISTA. In embodiments of the various aspects of the present disclosure, the MDSCs may be “VISTA-expressing MDSCs” or “VISTA+ MDSCs”. The MDSCs may express VISTA at the cell surface (i.e. VISTA may be expressed in or at the cell membrane).

### Antigen-Binding Molecules

The present disclosure relates to the therapeutic and prophylactic use of antigen-binding molecules which bind to VISTA.

An “antigen-binding molecule” refers to a molecule which is capable of binding to a target antigen. Antigen-binding molecules include e.g. monoclonal antibodies, polyclonal antibodies, monospecific and multispecific antibodies (e.g., bispecific antibodies), and antibody fragments (e.g. Fv, scFv, Fab, scFab, F(ab′)₂, Fab₂, diabodies, triabodies, scFv-Fc, minibodies, single domain antibodies (e.g. VhH), etc.), as long as they display binding to the relevant target molecule(s).

Antigen-binding molecules according to the present disclosure also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody. For example, antigen-binding molecules according to the present disclosure include antibody-drug conjugates (ADCs) comprising a (cytotoxic) drug moiety (e.g. as described hereinbelow). Antigen-binding molecules according to the present disclosure also include multispecific antigen-binding molecules such as immune cell engager molecules comprising a domain for recruiting (effector) immune cells (reviewed e.g. in Goebeler and Bargou, Nat. Rev. Clin. Oncol. (2020) 17: 418-434 and Ellerman, Methods (2019) 154:102-117, both of which are hereby incorporated by reference in their entirety), including BiTEs, BiKEs and TriKEs. Antigen-binding molecules according to the present disclosure also include chimeric antigen receptors (CARs), which are recombinant receptors providing both antigen-binding and T cell activating functions (CAR structure, function and engineering is reviewed e.g. in Dotti et al., Immunol Rev (2014) 257(1), which is hereby incorporated by reference in its entirety).

The antigen-binding molecule of the present disclosure comprises a moiety capable of binding to a target antigen(s). In some embodiments, the moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3):181-202). In some embodiments, the moiety capable of binding to a target antigen comprises or consists of a antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)) affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

The antigen-binding molecules of the present disclosure generally comprise an antigen-binding domain comprising a VH and a VL of an antibody capable of specific binding to the target antigen. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present disclosure may be designed and prepared using the sequences of monoclonal antibodies (mAbs) capable of binding to VISTA. Antigen-binding regions of antibodies, such as single chain variable fragment (scFv), Fab and F(ab′)₂ fragments may also be used/provided. An “antigen-binding region” is any fragment of an antibody which is capable of binding to the target for which the given antibody is specific.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody which binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77.

In some embodiments, the antigen-binding molecule comprises the CDRs of an antigen-binding molecule which is capable of binding to VISTA. In some embodiments, the antigen-binding molecule comprises the FRs of an antigen-binding molecule which is capable of binding to VISTA. In some embodiments, the antigen-binding molecule comprises the CDRs and the FRs of an antigen-binding molecule which is capable of binding to VISTA. That is, in some embodiments, the antigen-binding molecule comprises the VH region and the VL region of an antigen-binding molecule which is capable of binding to VISTA.

In some embodiments, the antigen-binding molecule comprises a VH region and a VL region which is, or which is derived from, the VH/VL region of a VISTA-binding antibody clone described herein (i.e. anti-VISTA antibody clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2-D2, 13D5p, 13D5-1, 13D5-13, 5M1-A11 or 9M2-C12).

In some embodiments, the antigen-binding molecule comprises a VH region according to one of (1) to (18) below:
(1) (4M2-C12 derived consensus) a VH region incorporating the following CDRs:
   HC-CDR1 having the amino acid sequence of SEQ ID NO:305
   HC-CDR2 having the amino acid sequence of SEQ ID NO:306
   HC-CDR3 having the amino acid sequence of SEQ ID NO:307,
or a variant thereof in which one or two or three amino acids in one or more of HC-CDR1, HC-CDR2, or HC-CDR3 are substituted with another amino acid.
(2) (V4-C24, V4-C26, V4-C27, V4-C28, V4-C30, V4-C31) a VH region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278,
or a variant thereof in which one or two or three amino acids in one or more of HC-CDR1, HC-CDR2, or HC-CDR3 are substituted with another amino acid.
(3) (V4-C1) a VH region incorporating the following CDRs:
   HC-CDR1 having the amino acid sequence of SEQ ID NO:33
   HC-CDR2 having the amino acid sequence of SEQ ID NO:277
   HC-CDR3 having the amino acid sequence of SEQ ID NO:278,
or a variant thereof in which one or two or three amino acids in one or more of HC-CDR1, HC-CDR2, or HC-CDR3 are substituted with another amino acid.
(4) (V4-C9) a VH region incorporating the following CDRs:
   HC-CDR1 having the amino acid sequence of SEQ ID NO:33
   HC-CDR2 having the amino acid sequence of SEQ ID NO:286
   HC-CDR3 having the amino acid sequence of SEQ ID NO:278,
   or a variant thereof in which one or two or three amino acids in one or more of HC-CDR1, HC-CDR2, or HC-CDR3 are substituted with another amino acid.
(5) (4M2-C12/V4H1/V4H2 consensus) a VH region incorporating the following CDRs:
   HC-CDR1 having the amino acid sequence of SEQ ID NO:244
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid.
(6) (4M2-C12, 4M2-B4, V4H2) a VH region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid.
(7) (V4H1) a VH region incorporating the following CDRs:
   HC-CDR1 having the amino acid sequence of SEQ ID NO:53
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid.
(8) (2M1-B12, 2M1-D2) a VH region incorporating the following CDRs:
   HC-CDR1 having the amino acid sequence of SEQ ID NO:72
   HC-CDR2 having the amino acid sequence of SEQ ID NO:73
   HC-CDR3 having the amino acid sequence of SEQ ID NO:74,
   or a variant thereof in which one or two or three amino acids in one or more of HC-CDR1, HC-CDR2, or HC-CDR3 are substituted with another amino acid.
(9) (4M2-C9, 5M1-A11) a VH region incorporating the following CDRs:
   HC-CDR1 having the amino acid sequence of SEQ ID NO:88
   HC-CDR2 having the amino acid sequence of SEQ ID NO:89
   HC-CDR3 having the amino acid sequence of SEQ ID NO:90,
   or a variant thereof in which one or two or three amino acids in one or more of HC-CDR1, HC-CDR2, or HC-CDR3 are substituted with another amino acid.
(10) (4M2-D9) a VH region incorporating the following CDRs:
   HC-CDR1 having the amino acid sequence of SEQ ID NO:33
   HC-CDR2 having the amino acid sequence of SEQ ID NO:107
   HC-CDR3 having the amino acid sequence of SEQ ID NO:108,
   or a variant thereof in which one or two or three amino acids in one or more of HC-CDR1, HC-CDR2, or HC-CDR3 are substituted with another amino acid.
(11) (1M2-D2) a VH region incorporating the following CDRs:
   HC-CDR1 having the amino acid sequence of SEQ ID NO:120
   HC-CDR2 having the amino acid sequence of SEQ ID NO:121
   HC-CDR3 having the amino acid sequence of SEQ ID NO:122,
   or a variant thereof in which one or two or three amino acids in one or more of HC-CDR1, HC-CDR2, or HC-CDR3 are substituted with another amino acid.
(12) (4M2-D5) a VH region incorporating the following CDRs:
   HC-CDR1 having the amino acid sequence of SEQ ID NO:144
   HC-CDR2 having the amino acid sequence of SEQ ID NO:145
   HC-CDR3 having the amino acid sequence of SEQ ID NO:146,
   or a variant thereof in which one or two or three amino acids in one or more of HC-CDR1, HC-CDR2, or HC-CDR3 are substituted with another amino acid.
(13) (4M2-A8) a VH region incorporating the following CDRs:
   HC-CDR1 having the amino acid sequence of SEQ ID NO:158
   HC-CDR2 having the amino acid sequence of SEQ ID NO:159
   HC-CDR3 having the amino acid sequence of SEQ ID NO:160,
   or a variant thereof in which one or two or three amino acids in one or more of HC-CDR1, HC-CDR2, or HC-CDR3 are substituted with another amino acid.
(14) (9M2-C12) a VH region incorporating the following CDRs:
   HC-CDR1 having the amino acid sequence of SEQ ID NO:169
   HC-CDR2 having the amino acid sequence of SEQ ID NO:170
   HC-CDR3 having the amino acid sequence of SEQ ID NO:171,
   or a variant thereof in which one or two or three amino acids in one or more of HC-CDR1, HC-CDR2, or HC-CDR3 are substituted with another amino acid.
(15) (13D5 derived) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:246,
   or a variant thereof in which one or two or three amino acids in one or more of HC-CDR1, HC-CDR2, or HC-CDR3 are substituted with another amino acid.
(16) (13D5p) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:185,
   or a variant thereof in which one or two or three amino acids in one or more of HC-CDR1, HC-CDR2, or HC-CDR3 are substituted with another amino acid.
(17) (13D5-1) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:195,
   or a variant thereof in which one or two or three amino acids in one or more of HC-CDR1, HC-CDR2, or HC-CDR3 are substituted with another amino acid.
(18) (13D5-13) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:200,
   or a variant thereof in which one or two or three amino acids in one or more of HC-CDR1, HC-CDR2, or HC-CDR3 are substituted with another amino acid.

In some embodiments, the antigen-binding molecule comprises a VH region according to one of (19) to (35) below:
(19) (V4-C24, V4-C26, V4-C27, V4-C28, V4-C30, V4-C31) a VH region incorporating the following FRs:
   HC-FR1 having the amino acid sequence of SEQ ID NO:63
   HC-FR2 having the amino acid sequence of SEQ ID NO:292
   HC-FR3 having the amino acid sequence of SEQ ID NO:293
   HC-FR4 having the amino acid sequence of SEQ ID NO:281,
   or a variant thereof in which one or two or three amino acids in one or more of HC-FR1, HC-FR2, HC-FR3, or HC-FR4 are substituted with another amino acid.
(20) (V4-C1, V4-C9) a VH region incorporating the following FRs:
   HC-FR1 having the amino acid sequence of SEQ ID NO:63
   HC-FR2 having the amino acid sequence of SEQ ID NO:279
   HC-FR3 having the amino acid sequence of SEQ ID NO:280
   HC-FR4 having the amino acid sequence of SEQ ID NO:281,
   or a variant thereof in which one or two or three amino acids in one or more of HC-FR1, HC-FR2, HC-FR3, or HC-FR4 are substituted with another amino acid.
(21) (4M2-C12) a VH region incorporating the following FRs:
   HC-FR1 having the amino acid sequence of SEQ ID NO:36
   HC-FR2 having the amino acid sequence of SEQ ID NO:37
   HC-FR3 having the amino acid sequence of SEQ ID NO:38
   HC-FR4 having the amino acid sequence of SEQ ID NO:39,
   or a variant thereof in which one or two or three amino acids in one or more of HC-FR1, HC-FR2,
   HC-FR3, or HC-FR4 are substituted with another amino acid.
(22) (4M2-B4) a VH region incorporating the following FRs:
   HC-FR1 having the amino acid sequence of SEQ ID NO:49
   HC-FR2 having the amino acid sequence of SEQ ID NO:37
   HC-FR3 having the amino acid sequence of SEQ ID NO:38
   HC-FR4 having the amino acid sequence of SEQ ID NO:39,
   or a variant thereof in which one or two or three amino acids in one or more of HC-FR1, HC-FR2, HC-FR3, or HC-FR4 are substituted with another amino acid.
(23) (V4H1) a VH region incorporating the following FRs:
   HC-FR1 having the amino acid sequence of SEQ ID NO:54
   HC-FR2 having the amino acid sequence of SEQ ID NO:55
   HC-FR3 having the amino acid sequence of SEQ ID NO:56
   HC-FR4 having the amino acid sequence of SEQ ID NO:39,
   or a variant thereof in which one or two or three amino acids in one or more of HC-FR1, HC-FR2, HC-FR3, or HC-FR4 are substituted with another amino acid.
(24) (V4H2) a VH region incorporating the following FRs:
   HC-FR1 having the amino acid sequence of SEQ ID NO:63
   HC-FR2 having the amino acid sequence of SEQ ID NO:64
   HC-FR3 having the amino acid sequence of SEQ ID NO:65
   HC-FR4 having the amino acid sequence of SEQ ID NO:39,
   or a variant thereof in which one or two or three amino acids in one or more of HC-FR1, HC-FR2, HC-FR3, or HC-FR4 are substituted with another amino acid.
(25) (2M1-B12) a VH region incorporating the following FRs:
   HC-FR1 having the amino acid sequence of SEQ ID NO:75
   HC-FR2 having the amino acid sequence of SEQ ID NO:76
   HC-FR3 having the amino acid sequence of SEQ ID NO:77
   HC-FR4 having the amino acid sequence of SEQ ID NO:78,
   or a variant thereof in which one or two or three amino acids in one or more of HC-FR1, HC-FR2, HC-FR3, or HC-FR4 are substituted with another amino acid.
(26) (4M2-C9) a VH region incorporating the following FRs:
   HC-FR1 having the amino acid sequence of SEQ ID NO:91
   HC-FR2 having the amino acid sequence of SEQ ID NO:92
   HC-FR3 having the amino acid sequence of SEQ ID NO:93
   HC-FR4 having the amino acid sequence of SEQ ID NO:94,
   or a variant thereof in which one or two or three amino acids in one or more of HC-FR1, HC-FR2, HC-FR3, or HC-FR4 are substituted with another amino acid.
(27) (2M1-D2) a VH region incorporating the following FRs:
   HC-FR1 having the amino acid sequence of SEQ ID NO: 103
   HC-FR2 having the amino acid sequence of SEQ ID NO:76
   HC-FR3 having the amino acid sequence of SEQ ID NO:77
   HC-FR4 having the amino acid sequence of SEQ ID NO:78,
   or a variant thereof in which one or two or three amino acids in one or more of HC-FR1, HC-FR2, HC-FR3, or HC-FR4 are substituted with another amino acid.
(28) (4M2-D9) a VH region incorporating the following FRs:
   HC-FR1 having the amino acid sequence of SEQ ID NO: 109
   HC-FR2 having the amino acid sequence of SEQ ID NO: 110
   HC-FR3 having the amino acid sequence of SEQ ID NO: 111
   HC-FR4 having the amino acid sequence of SEQ ID NO: 112,
   or a variant thereof in which one or two or three amino acids in one or more of HC-FR1, HC-FR2, HC-FR3, or HC-FR4 are substituted with another amino acid.
(29) (1M2-D2) a VH region incorporating the following FRs:
   HC-FR1 having the amino acid sequence of SEQ ID NO:123
   HC-FR2 having the amino acid sequence of SEQ ID NO:124
   HC-FR3 having the amino acid sequence of SEQ ID NO:125
   HC-FR4 having the amino acid sequence of SEQ ID NO:78,
   or a variant thereof in which one or two or three amino acids in one or more of HC-FR1, HC-FR2, HC-FR3, or HC-FR4 are substituted with another amino acid.
(30) (5M1-A11) a VH region incorporating the following FRs:
   HC-FR1 having the amino acid sequence of SEQ ID NO:134
   HC-FR2 having the amino acid sequence of SEQ ID NO:92
   HC-FR3 having the amino acid sequence of SEQ ID NO:93
   HC-FR4 having the amino acid sequence of SEQ ID NO:135,
   or a variant thereof in which one or two or three amino acids in one or more of HC-FR1, HC-FR2, HC-FR3, or HC-FR4 are substituted with another amino acid.
(31) (4M2-D5) a VH region incorporating the following FRs:
   HC-FR1 having the amino acid sequence of SEQ ID NO:147
   HC-FR2 having the amino acid sequence of SEQ ID NO:148
   HC-FR3 having the amino acid sequence of SEQ ID NO:149
   HC-FR4 having the amino acid sequence of SEQ ID NO:135,
   or a variant thereof in which one or two or three amino acids in one or more of HC-FR1, HC-FR2, HC-FR3, or HC-FR4 are substituted with another amino acid.
(32) (4M2-A8) a VH region incorporating the following FRs:
   HC-FR1 having the amino acid sequence of SEQ ID NO:161
   HC-FR2 having the amino acid sequence of SEQ ID NO:162
   HC-FR3 having the amino acid sequence of SEQ ID NO:163
   HC-FR4 having the amino acid sequence of SEQ ID NO:135,
   or a variant thereof in which one or two or three amino acids in one or more of HC-FR1, HC-FR2, HC-FR3, or HC-FR4 are substituted with another amino acid.
(33) (9M2-C12) a VH region incorporating the following FRs:
   HC-FR1 having the amino acid sequence of SEQ ID NO:172
   HC-FR2 having the amino acid sequence of SEQ ID NO: 173
   HC-FR3 having the amino acid sequence of SEQ ID NO: 174
   HC-FR4 having the amino acid sequence of SEQ ID NO: 175,
   or a variant thereof in which one or two or three amino acids in one or more of HC-FR1, HC-FR2, HC-FR3, or HC-FR4 are substituted with another amino acid.
(34) (13D5p, 13D5-1) a VH region incorporating the following FRs:
   HC-FR1 having the amino acid sequence of SEQ ID NO:103
   HC-FR2 having the amino acid sequence of SEQ ID NO:186
   HC-FR3 having the amino acid sequence of SEQ ID NO:187
   HC-FR4 having the amino acid sequence of SEQ ID NO:86,
   or a variant thereof in which one or two or three amino acids in one or more of HC-FR1, HC-FR2, HC-FR3, or HC-FR4 are substituted with another amino acid.
(35) (13D5-13) a VH region incorporating the following FRs:
   HC-FR1 having the amino acid sequence of SEQ ID NO:103
   HC-FR2 having the amino acid sequence of SEQ ID NO:186
   HC-FR3 having the amino acid sequence of SEQ ID NO:201
   HC-FR4 having the amino acid sequence of SEQ ID NO:86,
   or a variant thereof in which one or two or three amino acids in one or more of HC-FR1, HC-FR2, HC-FR3, or HC-FR4 are substituted with another amino acid.

In some embodiments, the antigen-binding molecule comprises a VH region comprising the CDRs according to one of (1) to (18) above, and the FRs according to one of (19) to (35) above.

In some embodiments, the antigen-binding molecule comprises a VH region according to one of (36) to (57) below:
(36) a VH region comprising the CDRs according to (1) and the FRs according to (19), (20), (21), (22), (23) or (24).
(37) a VH region comprising the CDRs according to (2) and the FRs according to (19).
(38) a VH region comprising the CDRs according to (3) and the FRs according to (20).
(39) a VH region comprising the CDRs according to (4) and the FRs according to (20).
(40) a VH region comprising the CDRs according to (5) and the FRs according to (21), (22), (23) or (24).
(41) a VH region comprising the CDRs according to (6) and the FRs according to (21).
(42) a VH region comprising the CDRs according to (6) and the FRs according to (22).
(43) a VH region comprising the CDRs according to (6) and the FRs according to (24).
(44) a VH region comprising the CDRs according to (7) and the FRs according to (23).
(45) a VH region comprising the CDRs according to (8) and the FRs according to (25).
(46) a VH region comprising the CDRs according to (8) and the FRs according to (27).
(47) a VH region comprising the CDRs according to (9) and the FRs according to (26).
(48) a VH region comprising the CDRs according to (9) and the FRs according to (30).
(49) a VH region comprising the CDRs according to (10) and the FRs according to (28).
(50) a VH region comprising the CDRs according to (11) and the FRs according to (29).
(51) a VH region comprising the CDRs according to (12) and the FRs according to (31).
(52) a VH region comprising the CDRs according to (13) and the FRs according to (32).
(53) a VH region comprising the CDRs according to (14) and the FRs according to (33).
(54) a VH region comprising the CDRs according to (15) and the FRs according to (34) or (35).
(55) a VH region comprising the CDRs according to (16) and the FRs according to (34).
(56) a VH region comprising the CDRs according to (17) and the FRs according to (34).
(57) a VH region comprising the CDRs according to (18) and the FRs according to (35).

In some embodiments, the antigen-binding molecule comprises a VH region according to one of (58) to (76) below:
(58) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:276.
(59) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:285.
(60) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289.
(61) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32.
(62) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:48.
(63) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.
(64) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:62.
(65) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:71.
(66) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:87.
(67) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:102.
(68) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:106.
(69) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:119.
(70) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:133.
(71) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:143.
(72) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:157.
(73) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:168.
(74) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:183.
(75) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:194.
(76) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:199.

In some embodiments, the antigen-binding molecule comprises a VL region according to one of (77) to (96) below:
(77) (4M2-C12 derived consensus) a VL region incorporating the following CDRs:
   LC-CDR1 having the amino acid sequence of SEQ ID NO:41
   LC-CDR2 having the amino acid sequence of SEQ ID NO:308
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(78) (C24/C26/C27 consensus) a VL region incorporating the following CDRs:
   LC-CDR1 having the amino acid sequence of SEQ ID NO:41
   LC-CDR2 having the amino acid sequence of SEQ ID NO:309
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(79) (V4-C24, V4-C26) a VL region incorporating the following CDRs:
   LC-CDR1 having the amino acid sequence of SEQ ID NO:41
   LC-CDR2 having the amino acid sequence of SEQ ID NO:295
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(80) (V4-C27, V4-C30, V4-C31) a VL region incorporating the following CDRs:
   LC-CDR1 having the amino acid sequence of SEQ ID NO:41
   LC-CDR2 having the amino acid sequence of SEQ ID NO:300
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(81) (4M2-C12/V4H1/V4H2 consensus) a VL region incorporating the following CDRs:
   LC-CDR1 having the amino acid sequence of SEQ ID NO:41
   LC-CDR2 having the amino acid sequence of SEQ ID NO:245
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(82) (4M2-C12, 4M2-B4, V4-C1, V4-C9, V4-C28) a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(83) (V4H1) a VL region incorporating the following CDRs:
   LC-CDR1 having the amino acid sequence of SEQ ID NO:41
   LC-CDR2 having the amino acid sequence of SEQ ID NO:58
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(84) (V4H2) a VL region incorporating the following CDRs:
   LC-CDR1 having the amino acid sequence of SEQ ID NO:41
   LC-CDR2 having the amino acid sequence of SEQ ID NO:67
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(85) (2M1-B12, 2M1-D2) a VL region incorporating the following CDRs:
   LC-CDR1 having the amino acid sequence of SEQ ID NO:80
   LC-CDR2 having the amino acid sequence of SEQ ID NO:81
   LC-CDR3 having the amino acid sequence of SEQ ID NO:82;
   or a variant thereof in which one or two or three amino acids in one or more of LC-CDR1, LC-CDR2 or LC-CDR3 are substituted with another amino acid.
(86) (4M2-C9) a VL region incorporating the following CDRs:
   LC-CDR1 having the amino acid sequence of SEQ ID NO:96
   LC-CDR2 having the amino acid sequence of SEQ ID NO:97
   LC-CDR3 having the amino acid sequence of SEQ ID NO:98;
   or a variant thereof in which one or two or three amino acids in one or more of LC-CDR1, LC-CDR2 or LC-CDR3 are substituted with another amino acid.
(87) (4M2-D9) a VH region incorporating the following CDRs:
   LC-CDR1 having the amino acid sequence of SEQ ID NO:114
   LC-CDR2 having the amino acid sequence of SEQ ID NO:67
   LC-CDR3 having the amino acid sequence of SEQ ID NO:115,
   or a variant thereof in which one or two or three amino acids in one or more of LC-CDR1, LC-CDR2, or LC-CDR3 are substituted with another amino acid.
(88) (1M2-D2) a VL region incorporating the following CDRs:
   LC-CDR1 having the amino acid sequence of SEQ ID NO:127
   LC-CDR2 having the amino acid sequence of SEQ ID NO:128
   LC-CDR3 having the amino acid sequence of SEQ ID NO:129;
   or a variant thereof in which one or two or three amino acids in one or more of LC-CDR1, LC-CDR2 or LC-CDR3 are substituted with another amino acid.
(89) (5M1-A11) a VL region incorporating the following CDRs:
   LC-CDR1 having the amino acid sequence of SEQ ID NO:137
   LC-CDR2 having the amino acid sequence of SEQ ID NO:138
   LC-CDR3 having the amino acid sequence of SEQ ID NO:139;
   or a variant thereof in which one or two or three amino acids in one or more of LC-CDR1, LC-CDR2 or LC-CDR3 are substituted with another amino acid.
(90) (4M2-D5) a VL region incorporating the following CDRs:
   LC-CDR1 having the amino acid sequence of SEQ ID NO:151
   LC-CDR2 having the amino acid sequence of SEQ ID NO:152
   LC-CDR3 having the amino acid sequence of SEQ ID NO:153;
   or a variant thereof in which one or two or three amino acids in one or more of LC-CDR1, LC-CDR2 or LC-CDR3 are substituted with another amino acid.
(91) (4M2-A8) a VL region incorporating the following CDRs:
   LC-CDR1 having the amino acid sequence of SEQ ID NO:165
   LC-CDR2 having the amino acid sequence of SEQ ID NO:152
   LC-CDR3 having the amino acid sequence of SEQ ID NO:153;
   or a variant thereof in which one or two or three amino acids in one or more of LC-CDR1, LC-CDR2 or LC-CDR3 are substituted with another amino acid.
(92) (9M2-C12) a VL region incorporating the following CDRs:
   LC-CDR1 having the amino acid sequence of SEQ ID NO:177
   LC-CDR2 having the amino acid sequence of SEQ ID NO:178
   LC-CDR3 having the amino acid sequence of SEQ ID NO:179;
   or a variant thereof in which one or two or three amino acids in one or more of LC-CDR1, LC-CDR2 or LC-CDR3 are substituted with another amino acid.
(93) (13D5p derived) a VL region incorporating the following CDRs:
   LC-CDR1 having the amino acid sequence of SEQ ID NO:247
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.
(94) (13D5p) a VL region incorporating the following CDRs:
   LC-CDR1 having the amino acid sequence of SEQ ID NO:189
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.
(95) (13D5-1) a VL region incorporating the following CDRs:
   LC-CDR1 having the amino acid sequence of SEQ ID NO:197
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.
(96) (13D5-13) a VL region incorporating the following CDRs:
   LC-CDR1 having the amino acid sequence of SEQ ID NO:203
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.

In some embodiments, the antigen-binding molecule comprises a VL region according to one of (97) to (120) below:
(97) (V4-C1) a VL region incorporating the following FRs:
   LC-FR1 having the amino acid sequence of SEQ ID NO:59
   LC-FR2 having the amino acid sequence of SEQ ID NO:283
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(98) (V4-C9) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(99) (V4-C24) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:296
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(100) (V4-C26) a VL region incorporating the following FRs:
   LC-FR1 having the amino acid sequence of SEQ ID NO:288
   LC-FR2 having the amino acid sequence of SEQ ID NO:298
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(101) (V4-C27) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(102) (V4-C28) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:296
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(103) (V4-C30) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:296
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(104) (V4-C31) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:304
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(105) (4M2-C12) a VL region incorporating the following FRs:
   LC-FR1 having the amino acid sequence of SEQ ID NO:44
   LC-FR2 having the amino acid sequence of SEQ ID NO:45
   LC-FR3 having the amino acid sequence of SEQ ID NO:46
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(106) (4M2-B4) a VL region incorporating the following FRs:
   LC-FR1 having the amino acid sequence of SEQ ID NO:51
   LC-FR2 having the amino acid sequence of SEQ ID NO:45
   LC-FR3 having the amino acid sequence of SEQ ID NO:46
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(107) (V4H1) a VL region incorporating the following FRs:
   LC-FR1 having the amino acid sequence of SEQ ID NO:59
   LC-FR2 having the amino acid sequence of SEQ ID NO:60
   LC-FR3 having the amino acid sequence of SEQ ID NO:61
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(108) (V4H2) a VL region incorporating the following FRs:
   LC-FR1 having the amino acid sequence of SEQ ID NO:68
   LC-FR2 having the amino acid sequence of SEQ ID NO:69
   LC-FR3 having the amino acid sequence of SEQ ID NO:70
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(109) (2M1-B12) a VL region incorporating the following FRs:
   LC-FR1 having the amino acid sequence of SEQ ID NO:83
   LC-FR2 having the amino acid sequence of SEQ ID NO:84
   LC-FR3 having the amino acid sequence of SEQ ID NO:85
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(110) (4M2-C9) a VL region incorporating the following FRs:
   LC-FR1 having the amino acid sequence of SEQ ID NO:99
   LC-FR2 having the amino acid sequence of SEQ ID NO:100
   LC-FR3 having the amino acid sequence of SEQ ID NO:101
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(111) (2M1-D2) a VL region incorporating the following FRs:
   LC-FR1 having the amino acid sequence of SEQ ID NO:105
   LC-FR2 having the amino acid sequence of SEQ ID NO:84
   LC-FR3 having the amino acid sequence of SEQ ID NO:85
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(112) (4M2-D9) a VL region incorporating the following FRs:
   LC-FR1 having the amino acid sequence of SEQ ID NO:116
   LC-FR2 having the amino acid sequence of SEQ ID NO:117
   LC-FR3 having the amino acid sequence of SEQ ID NO:118
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(113) (1M2-D2) a VL region incorporating the following FRs:
   LC-FR1 having the amino acid sequence of SEQ ID NO:130
   LC-FR2 having the amino acid sequence of SEQ ID NO:131
   LC-FR3 having the amino acid sequence of SEQ ID NO:132
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(114) (5M1-A11) a VL region incorporating the following FRs:
   LC-FR1 having the amino acid sequence of SEQ ID NO:140
   LC-FR2 having the amino acid sequence of SEQ ID NO:141
   LC-FR3 having the amino acid sequence of SEQ ID NO:142
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(115) (4M2-D5) a VL region incorporating the following FRs:
   LC-FR1 having the amino acid sequence of SEQ ID NO:154
   LC-FR2 having the amino acid sequence of SEQ ID NO:155
   LC-FR3 having the amino acid sequence of SEQ ID NO:156
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(116) (4M2-A8) a VL region incorporating the following FRs:
   LC-FR1 having the amino acid sequence of SEQ ID NO:166
   LC-FR2 having the amino acid sequence of SEQ ID NO:155
   LC-FR3 having the amino acid sequence of SEQ ID NO:167
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(117) (9M2-C12) a VL region incorporating the following FRs:
   LC-FR1 having the amino acid sequence of SEQ ID NO:180
   LC-FR2 having the amino acid sequence of SEQ ID NO:181
   LC-FR3 having the amino acid sequence of SEQ ID NO:182
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(118) (13D5p) a VL region incorporating the following FRs:
   LC-FR1 having the amino acid sequence of SEQ ID NO:191
   LC-FR2 having the amino acid sequence of SEQ ID NO:192
   LC-FR3 having the amino acid sequence of SEQ ID NO:193
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(119) (13D5-1) a VL region incorporating the following FRs:
   LC-FR1 having the amino acid sequence of SEQ ID NO:191
   LC-FR2 having the amino acid sequence of SEQ ID NO:198
   LC-FR3 having the amino acid sequence of SEQ ID NO:193
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(120) (13D5-13) a VL region incorporating the following FRs:
   LC-FR1 having the amino acid sequence of SEQ ID NO:191
   LC-FR2 having the amino acid sequence of SEQ ID NO:192
   LC-FR3 having the amino acid sequence of SEQ ID NO:204
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.

In some embodiments, the antigen-binding molecule comprises a VL region comprising the CDRs according to one of (77) to (96) above, and the FRs according to one of (97) to (120) above.

In some embodiments, the antigen-binding molecule comprises a VL region according to one of (121) to (148) below:
(121) a VL region comprising the CDRs according to (77) and the FRs according to (97), (98), (99), (100), (101), (102), (103), (104), (105), (106), (107) or (108).
(122) a VL region comprising the CDRs according to (78) and the FRs according to (99), (100) or (101).
(123) a VL region comprising the CDRs according to (79) and the FRs according to (99).
(124) a VL region comprising the CDRs according to (79) and the FRs according to (100).
(125) a VL region comprising the CDRs according to (80) and the FRs according to (101).
(126) a VL region comprising the CDRs according to (82) and the FRs according to (97).
(127) a VL region comprising the CDRs according to (82) and the FRs according to (98).
(128) a VL region comprising the CDRs according to (82) and the FRs according to (102).
(129) a VL region comprising the CDRs according to (80) and the FRs according to (103).
(130) a VL region comprising the CDRs according to (80) and the FRs according to (104).
(131) a VL region comprising the CDRs according to (81) and the FRs according to (105), (106), (107) or (108).
(132) a VL region comprising the CDRs according to (82) and the FRs according to (105).
(133) a VL region comprising the CDRs according to (82) and the FRs according to (106).
(134) a VL region comprising the CDRs according to (83) and the FRs according to (107).
(135) a VL region comprising the CDRs according to (84) and the FRs according to (108).
(136) a VL region comprising the CDRs according to (85) and the FRs according to (109).
(137) a VL region comprising the CDRs according to (85) and the FRs according to (111).
(138) a VL region comprising the CDRs according to (86) and the FRs according to (110).
(139) a VL region comprising the CDRs according to (87) and the FRs according to (112).
(140) a VL region comprising the CDRs according to (88) and the FRs according to (113).
(141) a VL region comprising the CDRs according to (89) and the FRs according to (114).
(142) a VL region comprising the CDRs according to (90) and the FRs according to (115).
(143) a VL region comprising the CDRs according to (91) and the FRs according to (116).
(144) a VL region comprising the CDRs according to (92) and the FRs according to (117).
(145) a VL region comprising the CDRs according to (93) and the FRs according to (118), (119) or (120).
(146) a VL region comprising the CDRs according to (94) and the FRs according to (118).
(147) a VL region comprising the CDRs according to (95) and the FRs according to (119).
(148) a VL region comprising the CDRs according to (96) and the FRs according to (120).

In some embodiments, the antigen-binding molecule comprises a VL region according to one of (149) to (173) below:
(149) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:310.
(150) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:282.
(151) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:287.
(152) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:294.
(153) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:297.
(154) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:299.
(155) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:301.
(156) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:302.
(157) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:303.
(158) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:40.
(159) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:50.
(160) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:57.
(161) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:66.
(162) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:79.
(163) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:95.
(164) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:104.
(165) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:113.
(166) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:126.
(167) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:136.
(168) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:150.
(169) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:164.
(170) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:176.
(171) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:188.
(172) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:196.
(173) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:202.

In some embodiments, the antigen-binding molecule comprises a VH region according to any one of (1) to (76) above, and a VL region according to any one of (77) to (173) above.

In some embodiments, the antigen-binding molecule comprises the CDRs of, or comprises the VH and VL of, a VISTA-binding antibody clone selected from: 4M2-C12, V4H1, V4H2, V4-C1, V4-C9, V4-C24, V4-C26, V4-C27, V4-C28, V4-C30 or V4-C31. In some embodiments, the antigen-binding molecule comprises the CDRs of, or comprises the VH and VL of, V4-C26.

In some embodiments, the antigen-binding molecule comprises:
(A) a VH region incorporating the following CDRs:
   HC-CDR1 having the amino acid sequence of SEQ ID NO:305
   HC-CDR2 having the amino acid sequence of SEQ ID NO:306
   HC-CDR3 having the amino acid sequence of SEQ ID NO:307,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:41
   LC-CDR2 having the amino acid sequence of SEQ ID NO:308
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(B) a VH region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278,
   or a variant thereof in which one or two or three amino acids in one or more of HC-CDR1, HC-CDR2, or HC-CDR3 are substituted with another amino acid; and a VL region incorporating the following CDRs:
   LC-CDR1 having the amino acid sequence of SEQ ID NO:41
   LC-CDR2 having the amino acid sequence of SEQ ID NO:295
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(C) a VH region incorporating the following CDRs:
   HC-CDR1 having the amino acid sequence of SEQ ID NO:53
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid; and
   a VL region incorporating the following CDRs:
   LC-CDR1 having the amino acid sequence of SEQ ID NO:41
   LC-CDR2 having the amino acid sequence of SEQ ID NO:58
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(D) a VH region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid; and a VL region incorporating the following CDRs:
   LC-CDR1 having the amino acid sequence of SEQ ID NO:41
   LC-CDR2 having the amino acid sequence of SEQ ID NO:67
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(E) a VH region incorporating the following CDRs:
   HC-CDR1 having the amino acid sequence of SEQ ID NO:33
   HC-CDR2 having the amino acid sequence of SEQ ID NO:277
   HC-CDR3 having the amino acid sequence of SEQ ID NO:278,
   or a variant thereof in which one or two or three amino acids in one or more of HC-CDR1, HC-CDR2, or HC-CDR3 are substituted with another amino acid; and a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(F) a VH region incorporating the following CDRs:
   HC-CDR1 having the amino acid sequence of SEQ ID NO:33
   HC-CDR2 having the amino acid sequence of SEQ ID NO:286
   HC-CDR3 having the amino acid sequence of SEQ ID NO:278,
   or a variant thereof in which one or two or three amino acids in one or more of HC-CDR1, HC-CDR2, or HC-CDR3 are substituted with another amino acid; and a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(G) a VH region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278,
   or a variant thereof in which one or two or three amino acids in one or more of HC-CDR1, HC-CDR2, or HC-CDR3 are substituted with another amino acid; and a VL region incorporating the following CDRs:
   LC-CDR1 having the amino acid sequence of SEQ ID NO:41
   LC-CDR2 having the amino acid sequence of SEQ ID NO:300
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(H) a VH region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(I) a VH region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.

In some embodiments, the antigen-binding molecule comprises:
(J) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:297.
(K) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:57.
(L) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:62; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:66.
(M) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:276; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:282.
(N) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:285; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:287.
(O) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:294.
(P) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:299.
(Q) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:301.
(R) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:302.
(S) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32; and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:40.

In some embodiments, the antigen-binding molecule comprises, or consists of:
(i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:331; and
(ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:317.

In embodiments in accordance with the present disclosure in which one or more amino acids are substituted with another amino acid, the substitutions may be conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) may be functionally conservative. That is, in some embodiments, the substitution may not affect (or may not substantially affect) one or more functional properties (e.g. target binding) of the antigen-binding molecule comprising the substitution as compared to the equivalent unsubstituted molecule.

The VH and VL region of an antigen-binding region of an antibody together constitute the Fv region. In some embodiments, the antigen-binding molecule according to the present disclosure comprises, or consists of, an Fv region which binds to VISTA. In some embodiments, the VH and VL regions of the Fv are provided as single polypeptide joined by a linker region, i.e. a single chain Fv (scFv).

In some embodiments, the antigen-binding molecule of the present disclosure comprises one or more regions of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM. In some embodiments, the immunoglobulin heavy chain constant sequence is, or is derived from, the heavy chain constant sequence of IgG4.

In some embodiments, the immunoglobulin heavy chain constant sequence is human immunoglobulin G 1 constant (IGHG1; UniProt: P01857-1, v1; SEQ ID NO:205). Positions 1 to 98 of SEQ ID NO:205 form the CH1 region (SEQ ID NO:206). Positions 99 to 110 of SEQ ID NO:205 form a hinge region between CH1 and CH2 regions (SEQ ID NO:207). Positions 111 to 223 of SEQ ID NO:205 form the CH2 region (SEQ ID NO:208). Positions 224 to 330 of SEQ ID NO:205 form the CH3 region (SEQ ID NO:209).

The exemplified antigen-binding molecules may be prepared using pFUSE-CHIg-hG1, which comprises the substitutions D356E, L358M (positions numbered according to EU numbering) in the CH3 region. The amino acid sequence of the CH3 region encoded by pFUSE-CHIg-hG1 is shown in SEQ ID NO:210. It will be appreciated that CH3 regions may be provided with further substitutions in accordance with modification to an Fc region of the antigen-binding molecule as described herein.

In some embodiments a CH1 region comprises or consists of the sequence of SEQ ID NO:206, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:206. In some embodiments a CH1-CH2 hinge region comprises or consists of the sequence of SEQ ID NO:207, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:207. In some embodiments a CH2 region comprises or consists of the sequence of SEQ ID NO:208, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:208. In some embodiments a CH3 region comprises or consists of the sequence of SEQ ID NO:209 or 210, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:209 or 210.

In some embodiments, the antigen-binding molecule of the present disclosure comprises the sequence of SEQ ID NO:345, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:345. In some embodiments, the antigen-binding molecule of the present disclosure comprises the sequence of SEQ ID NO:346, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:346.

In some embodiments, the antigen-binding molecule of the present disclosure comprises one or more regions of an immunoglobulin light chain constant sequence. In some embodiments, the immunoglobulin light chain constant sequence is human immunoglobulin kappa constant (IGKC; CK; UniProt: P01834-1, v2; SEQ ID NO:211). In some embodiments, the immunoglobulin light chain constant sequence is a human immunoglobulin lambda constant (IGLC; CA), e.g. IGLC1, IGLC2, IGLC3, IGLC6 or IGLC7. In some embodiments a CL region comprises or consists of the sequence of SEQ ID NO:211, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:211.

The VL and light chain constant (CL) region, and the VH region and heavy chain constant 1 (CH1) region of an antigen-binding region of an antibody together constitute the Fab region. In some embodiments, the antigen-binding molecule comprises a Fab region comprising a VH, a CH1, a VL and a CL (e.g. CK or CA). In some embodiments, the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments, the Fab region comprises a polypeptide comprising a VH and a CL (e.g. a VH-CL fusion polypeptide) and a polypeptide comprising a VL and a CH (e.g. a VL-CH1 fusion polypeptide); that is, in some embodiments, the Fab region is a CrossFab region. In some embodiments, the VH, CH1, VL and CL regions of the Fab or CrossFab are provided as single polypeptide joined by linker regions, i.e. as a single chain Fab (scFab) or a single chain CrossFab (scCrossFab).

In some embodiments, the antigen-binding molecule of the present disclosure comprises, or consists of, a Fab region which binds to VISTA.

In some embodiments, the antigen-binding molecule described herein comprises, or consists of, a whole antibody which binds to VISTA. As used herein, “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (A).

In some embodiments, the antigen-binding molecule described herein comprises, or consists of, an IgG (e.g. IgG1, IgG2, IgG3, IgG4), IgA (e.g. IgA1, IgA2), IgD, IgE, or IgM which binds to VISTA. In preferred embodiments, the antigen-binding molecule is an IgG4.

In some embodiments, the antigen-binding molecule of the present disclosure is at least monovalent binding for VISTA. Binding valency refers to the number of binding sites in an antigen-binding molecule for a given antigenic determinant. Accordingly, in some embodiments, the antigen-binding molecule comprises at least one binding site for VISTA.

In some embodiments, the antigen-binding molecule comprises more than one binding site for VISTA, e.g. 2, 3 or 4 binding sites. The binding sites may be the same or different. In some embodiments, the antigen-binding molecule is e.g. bivalent, trivalent or tetravalent for VISTA.

Aspects of the present disclosure relate to multispecific antigen-binding molecules. By “multispecific” it is meant that the antigen-binding molecule displays specific binding to more than one target. In some embodiments, the antigen-binding molecule is a bispecific antigen-binding molecule. In some embodiments, the antigen-binding molecule comprises at least two different antigen-binding domains (i.e. at least two antigen-binding domains, e.g. comprising non-identical VHs and VLs).

In some embodiments, the antigen-binding molecule binds to VISTA and another target (e.g. an antigen other than VISTA), and so is at least bispecific. The term “bispecific” means that the antigen-binding molecule is able to bind specifically to at least two distinct antigenic determinants.

It will be appreciated that an antigen-binding molecule according to the present disclosure (e.g. a multispecific antigen-binding molecule) may comprise antigen-binding molecules capable of binding to the targets for which the antigen-binding molecule is specific. For example, an antigen-binding molecule which is capable of binding to VISTA and an antigen other than VISTA may comprise: (i) an antigen-binding molecule which is capable of binding to VISTA, and (ii) an antigen-binding molecule which is capable of binding to an antigen other than VISTA.

It will also be appreciated that an antigen-binding molecule according to the present disclosure (e.g. a multispecific antigen-binding molecule) may comprise antigen-binding polypeptides or antigen-binding polypeptide complexes capable of binding to the targets for which the antigen-binding molecule is specific. For example, an antigen-binding molecule according to the present disclosure may comprise e.g. (i) an antigen-binding polypeptide complex capable of binding to VISTA, comprising a light chain polypeptide (comprising the structure VL-CL) and a heavy chain polypeptide (comprising the structure VH-CH1-CH2-CH3), and (ii) an antigen-binding polypeptide complex capable of binding to an antigen other than VISTA, comprising a light chain polypeptide (comprising the structure VL-CL) and a heavy chain polypeptide (comprising the structure VH-CH1-CH2-CH3).

In some embodiments, a component antigen-binding molecule of a larger antigen-binding molecule (e.g. a multispecific antigen-biding molecule) may be referred to e.g. as an “antigen-binding domain” or “antigen-binding region” of the larger antigen-binding molecule.

In some embodiments, the antigen-binding molecule comprises an antigen-binding molecule capable of binding to VISTA, and an antigen-binding molecule capable of binding to an antigen other than VISTA. In some embodiments, the antigen other than VISTA is an immune cell surface molecule. In some embodiments, the antigen other than VISTA is a cancer cell antigen. In some embodiments, the antigen other than VISTA is a receptor molecule, e.g. a cell surface receptor. In some embodiments, the antigen other than VISTA is a cell signalling molecule, e.g. a cytokine, chemokine, interferon, interleukin or lymphokine. In some embodiments, the antigen other than VISTA is a growth factor or a hormone.

A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments, the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

An immune cell surface molecule may be any peptide/polypeptide, glycoprotein, lipoprotein, glycan, glycolipid, lipid, or fragment thereof expressed at or on the cell surface of an immune cell. In some embodiments, the part of the immune cell surface molecule which is bound by the antigen-binding molecule of the present disclosure is on the external surface of the immune cell (i.e. is extracellular). The immune cell surface molecule may be expressed at the cell surface of any immune cell. In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell). In some embodiments, the immune cell surface molecule may be a costimulatory molecule (e.g. CD28, OX40, 4-1 BB, ICOS or CD27) or a ligand thereof. In some embodiments, the immune cell surface molecule may be a checkpoint molecule (e.g. PD-1, CTLA-4, LAG-3, TIM-3, TIGIT or BTLA) or a ligand thereof.

Multispecific antigen-binding molecules according to the present disclosure may be provided in any suitable format, such as those formats described in described in Brinkmann and Kontermann MAbs (2017) 9(2): 182-212, which is hereby incorporated by reference in its entirety. Suitable formats include those shown in FIG. 2 of Brinkmann and Kontermann MAbs (2017) 9(2): 182-212: antibody conjugates, e.g. IgG₂, F(ab′)₂ or CovX-Body; IgG or IgG-like molecules, e.g. IgG, chimeric IgG, κλ-body common HC; CH1/CL fusion proteins, e.g. scFv2-CH1/CL, VHH2-CH1/CL; ‘variable domain only’ bispecific antigen-binding molecules, e.g. tandem scFv (taFV), triplebodies, diabodies (Db), dsDb, Db(kih), DART, scDB, dsFv-dsFv, tandAbs, triple heads, tandem dAb/VHH, tertravalent dAb.VHH; Non-Ig fusion proteins, e.g. scFv₂-albumin, scDb-albumin, taFv-albumin, taFv-toxin, miniantibody, DNL-Fab₂, DNL-Fab₂-scFv, DNL-Fab₂-IgG-cytokine2, ImmTAC (TCR-scFv); modified Fc and CH3 fusion proteins, e.g. scFv-Fc(kih), scFv-Fc(CH3 charge pairs), scFv-Fc (EW-RVT), scFv-fc (HA-TF), scFv-Fc (SEEDbody), taFv-Fc(kih), scFv-Fc(kih)-Fv, Fab-Fc(kih)-scFv, Fab-scFv-Fc(kih), Fab-scFv-Fc(BEAT), Fab-scFv-Fc (SEEDbody), DART-Fc, scFv-CH3(kih), TriFabs; Fc fusions, e.g. Di-diabody, scDb-Fc, taFv-Fc, scFv-Fc-scFv, HCAb-VHH, Fab-scFv-Fc, scFv₄-Ig, scFv₂-Fcab; CH3 fusions, e.g. Dia-diabody, scDb-CH3; IgE/IgM CH2 fusions, e.g. scFv-EHD2-scFv, scFvMHD2-scFv; Fab fusion proteins, e.g. Fab-scFv (bibody), Fab-scFv₂ (tribody), Fab-Fv, Fab-dsFv, Fab-VHH, orthogonal Fab-Fab; non-Ig fusion proteins, e.g. DNL-Fab₃, DNL-Fab₂-scFv, DNL-Fab₂-IgG-cytokine₂; asymmetric IgG or IgG-like molecules, e.g. IgG(kih), IgG(kih) common LC, ZW1 IgG common LC, Biclonics common LC, CrossMab, CrossMab(kih), scFab-IgG(kih), Fab-scFab-IgG(kih), orthogonal Fab IgG(kih), DuetMab, CH3 charge pairs+CH1/CL charge pairs, hinge/CH3 charge pairs, SEED-body, Duobody, four-in-one-CrossMab(kih), LUZ-Y common LC; LUZ-Y scFab-IgG, FcFc*; appended and Fc-modified IgGs, e.g. IgG(kih)-Fv, IgG HA-TF-Fv, IgG(kih)scFab, scFab-Fc(kih)-scFv₂, scFab-Fc(kih)-scFv, half DVD-Ig, DVI-Ig (four-in-one), CrossMab-Fab; modified Fc and CH3 fusion proteins, e.g. Fab-Fc(kih)-scFv, Fab-scFv-Fc(kih), Fab-scFv-Fc(BEAT), Fab-scFv-Fc-SEEDbody, TriFab; appended IgGs-HC fusions, e.g. IgG-HC, scFv, IgG-dAb, IgG-taFV, IgG-CrossFab, IgG-orthogonal Fab, IgG-(CaCp) Fab, scFv-HC-IgG, tandem Fab-IgG (orthogonal Fab) Fab-IgG(CaCp Fab), Fab-IgG(CR3), Fab-hinge-IgG(CR3); appended IgGs-LC fusions, e.g. IgG-scFv(LC), scFv(LC)-IgG, dAb-IgG; appended IgGs-HC and LC fusions, e.g. DVD-Ig, TVD-Ig, CODV-Ig, scFv₄-IgG, Zybody; Fc fusions, e.g. Fab-scFv-Fc, scFv₄-Ig; F(ab′)₂ fusions, e.g. F(ab′)₂-scFv₂; CH1/CL fusion proteins e.g. scFv₂-CH1-hinge/CL; modified IgGs, e.g. DAF (two-in one-IgG), DutaMab, Mab²; and non-Ig fusions, e.g. DNL-Fab₄-IgG.

The skilled person is able to design and prepare bispecific antigen-binding molecules. Methods for producing bispecific antigen-binding molecules include chemically crosslinking of antigen-binding molecules or antibody fragments, e.g. with reducible disulphide or non-reducible thioether bonds, for example as described in Segal and Bast, 2001. Production of Bispecific Antigen-binding molecules. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH-groups, to create disulfide-linked bispecific F(ab)2 heterodimers.

Other methods for producing bispecific antigen-binding molecules include fusing antibody-producing hybridomas e.g. with polyethylene glycol, to produce a quadroma cell capable of secreting bispecific antibody, for example as described in D. M. and Bast, B. J. 2001. Production of Bispecific Antigen-binding molecules. Current Protocols in Immunology. 14:IV:2.13:2.13.1-2.13.16.

Bispecific antigen-binding molecules according to the present disclosure can also be produced recombinantly, by expression from e.g. a nucleic acid construct encoding polypeptides for the antigen-binding molecules, for example as described in Antibody Engineering: Methods and Protocols, Second Edition (Humana Press, 2012), at Chapter 40: Production of Bispecific Antigen-binding molecules: Diabodies and Tandem scFv (Hornig and FArber-Schwarz), or French, How to make bispecific antigen-binding molecules, Methods Mol. Med. 2000; 40:333-339, the entire contents of both of which are hereby incorporated by reference. For example, a DNA construct encoding the light and heavy chain variable domains for the two antigen-binding fragments (i.e. the light and heavy chain variable domains for the antigen-binding fragment capable of binding VISTA, and the light and heavy chain variable domains for the antigen-binding fragment capable of binding to another target protein), and including sequences encoding a suitable linker or dimerization domain between the antigen-binding fragments can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

### Fc Regions

In some embodiments, the antigen-binding molecules of the present disclosure comprise an Fc region.

In IgG IgA and IgD isotype Fc regions are composed of CH2 and CH3 regions from one polypeptide, and CH2 and CH3 regions from another polypeptide. The CH2 and CH3 regions from the two polypeptides together form the Fc region. In IgM and IgE isotypes the Fc regions contain three constant domains (CH2, CH3 and CH4), and CH2 to CH4 from the two polypeptides together form the Fc region.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, NK cells and T cells) through interaction between the Fc region and Fc receptors expressed by the immune cells, recruitment of complement pathway components through binding of the Fc region to complement protein C1q, and consequent activation of the complement cascade.

Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Modifications to antibody Fc regions that influence Fc-mediated functions are known in the art, such as those described e.g. in Wang et al., Protein Cell (2018) 9(1):63-73, which is hereby incorporated by reference in its entirety. In particular, exemplary Fc region modifications known to influence antibody effector function are summarised in Table 1 of Wang et al., Protein Cell (2018) 9(1):63-73. Modifications to Fc regions which influence antibody effector activity are described hereinbelow.

Where an Fc region/CH2/CH3 is described as comprising modification(s) “corresponding to” reference substitution(s), equivalent substitution(s) in the homologous Fc/CH2/CH3 are contemplated. By way of illustration, L234A/L235A substitutions in human IgG1 (numbered according to the EU numbering system as described in Kabat et al., Sequences of Proteins of Immunological Interest, 5th Ed. Public Health Service, National Institutes of Health, Bethesda, MD, 1991) correspond to L to A substitutions at positions 117 and 118 of the mouse Ig gamma-2A chain C region, A allele, numbered according to SEQ ID NO:256.

Where an Fc region is described as comprising a modification, the modification may be present in one or both of the polypeptide chains which together form the Fc region.

In some embodiments, the antigen-binding molecule of the present disclosure comprises an Fc region comprising modification. In some embodiments, the antigen-binding molecule of the present disclosure comprises an Fc region comprising modification in one or more of the CH2 and/or CH3 regions.

In some embodiments, the Fc region comprises modification to increase an Fc-mediated function. In some embodiments, the Fc region comprises modification to increase ADCC. In some embodiments, the Fc region comprises modification to increase ADCP. In some embodiments, the Fc region comprises modification to increase CDC. An antigen-binding molecule comprising an Fc region comprising modification to increase an Fc-mediated function (e.g. ADCC, ADCP, CDC) induces an increased level of the relevant effector function as compared to an antigen-binding molecule comprising the corresponding unmodified Fc region.

In some embodiments, the Fc region comprises modification to increase binding to an Fc receptor. In some embodiments, the Fc region comprises modification to increase binding to an Fcγ receptor. In some embodiments, the Fc region comprises modification to increase binding to one or more of FcγRI, FcγRIIa, FcγRIIb, FcγRIIc, FcγRIIIa and FcγRIIIb. In some embodiments, the Fc region comprises modification to increase binding to FcγRIIIa. In some embodiments, the Fc region comprises modification to increase binding to FcγRIIa. In some embodiments, the Fc region comprises modification to increase binding to FcγRIIb. In some embodiments, the Fc region comprises modification to increase binding to FcRn. In some embodiments, the Fc region comprises modification to increase binding to a complement protein. In some embodiments, the Fc region comprises modification to increase binding to C1q. In some embodiments, the Fc region comprises modification to promote hexamerisation of the antigen-binding molecule. In some embodiments, the Fc region comprises modification to increase antigen-binding molecule half-life. In some embodiments, the Fc region comprises modification to increase co-engagement.

In some embodiments, the Fc region comprises modification corresponding to the combination of substitutions F243L/R292P/Y300L/V3051/P396L as described in Stavenhagen et al. Cancer Res. (2007) 67:8882-8890. In some embodiments, the Fc region comprises modification corresponding to the combination of substitutions S239D/1332E or S239D/1332E/A330L as described in Lazar et al., Proc Natl Acad Sci USA. (2006)103:4005-4010. In some embodiments, the Fc region comprises modification corresponding to the combination of substitutions S298A/E333A/K334A as described in Shields et al., J Biol Chem. (2001) 276:6591-6604. In some embodiments, the Fc region comprises modification to one of heavy chain polypeptides corresponding to the combination of substitutions L234Y/L235Q/G236W/S239M/H268D/D270E/S298A, and modification to the other heavy chain polypeptide corresponding to the combination of substitutions D270E/K326D/A330M/K334E, as described in Mimoto et al., MAbs. (2013): 5:229-236. In some embodiments, the Fc region comprises modification corresponding to the combination of substitutions G236A/S239D/1332E as described in Richards et al., Mol Cancer Ther. (2008) 7:2517-2527.

In some embodiments, the Fc region comprises modification corresponding to the combination of substitutions K326W/E333S as described in Idusogie et al. J Immunol. (2001) 166(4):2571-5. In some embodiments, the Fc region comprises modification corresponding to the combination of substitutions S267E/H268F/S324T as described in Moore et al. MAbs. (2010) 2(2):181-9. In some embodiments, the Fc region comprises modification corresponding to the combination of substitutions described in Natsume et al., Cancer Res. (2008) 68(10):3863-72. In some embodiments, the Fc region comprises modification corresponding to the combination of substitutions E345R/E430G/S440Y as described in Diebolder et al. Science (2014) 343(6176):1260-3.

In some embodiments, the Fc region comprises modification corresponding to the combination of substitutions M252Y/S254T/T256E as described in Dall'Acqua et al. J Immunol. (2002) 169:5171-5180. In some embodiments, the Fc region comprises modification corresponding to the combination of substitutions M428L/N434S as described in Zalevsky et al. Nat Biotechnol. (2010) 28:157-159.

In some embodiments, the Fc region comprises modification corresponding to the combination of substitutions S267E/L328F as described in Chu et al., Mol Immunol. (2008) 45:3926-3933. In some embodiments, the Fc region comprises modification corresponding to the combination of substitutions N325S/L328F as described in Shang et al. Biol Chem. (2014) 289:15309-15318.

In some embodiments, the Fc region comprises modification to reduce/prevent an Fc-mediated function. In some embodiments, the Fc region comprises modification to reduce/prevent ADCC. In some embodiments, the Fc region comprises modification to reduce/prevent ADCP. In some embodiments, the Fc region comprises modification to reduce/prevent CDC. An antigen-binding molecule comprising an Fc region comprising modification to reduce/prevent an Fc-mediated function (e.g. ADCC, ADCP, CDC) induces an reduced level of the relevant effector function as compared to an antigen-binding molecule comprising the corresponding unmodified Fc region.

In some embodiments, the Fc region comprises modification to reduce/prevent binding to an Fc receptor. In some embodiments, the Fc region comprises modification to reduce/prevent binding to an Fcγ receptor. In some embodiments, the Fc region comprises modification to reduce/prevent binding to one or more of FcγRI, FcγRIIa, FcγRIIb, FcγRIIc, FcγRIIIa and FcγRIIIb. In some embodiments, the Fc region comprises modification to reduce/prevent binding to FcγRIIIa. In some embodiments, the Fc region comprises modification to reduce/prevent binding to FcγRIIa. In some embodiments, the Fc region comprises modification to reduce/prevent binding to FcγRIIb. In some embodiments, the Fc region comprises modification to reduce/prevent binding to a complement protein. In some embodiments, the Fc region comprises modification to reduce/prevent binding to C1q. In some embodiments, the Fc region comprises modification to reduce/prevent glycosylation of the amino acid residue corresponding to N297. In some embodiments, the Fc region is not able to induce one or more Fc-mediated functions (i.e. lacks the ability to elicit the relevant Fc-mediated function(s)). Accordingly, antigen-binding molecules comprising such Fc regions also lack the ability to induce the relevant function(s). Such antigen-binding molecules may be described as being devoid of the relevant function(s).

In some embodiments, the Fc region is not able to induce ADCC. In some embodiments, the Fc region is not able to induce ADCP. In some embodiments, the Fc region is not able to induce CDC. In some embodiments, the Fc region is not able to induce ADCC and/or is not able to induce ADCP and/or is not able to induce CDC.

In some embodiments, the Fc region is not able to bind to an Fc receptor. In some embodiments, the Fc region is not able to bind to an Fcγ receptor. In some embodiments, the Fc region is not able to bind to one or more of FcγRI, FcγRIIa, FcγRIIb, FcγRIIc, FcγRIIIa and FcγRIIIb. In some embodiments, the Fc region is not able to bind to FcγRIIIa. In some embodiments, the Fc region is not able to bind to FcγRIIa. In some embodiments, the Fc region is not able to bind to FcγRIIb. In some embodiments, the Fc region is not able to bind to FcRn. In some embodiments, the Fc region is not able to bind to a complement protein. In some embodiments, the Fc region is not able to bind to C1q. In some embodiments, the Fc region is not glycosylated at the amino acid residue corresponding to N297.

In some embodiments, the Fc region comprises modification corresponding to N297A or N297Q or N297G as described in Leabman et al., MAbs. (2013) 5:896-903. In some embodiments, the Fc region comprises modification corresponding to L235E as described in Alegre et al., J Immunol. (1992) 148:3461-3468. In some embodiments, the Fc region comprises modification corresponding to the combination of substitutions L234A/L235A or F234A/L235A as described in Xu et al., Cell Immunol. (2000) 200:16-26. In some embodiments, the Fc region comprises modification corresponding to P329A or γ329G as described in Schlothauer et al., Protein Engineering, Design and Selection (2016), 29(10):457-466. In some embodiments, the Fc region comprises modification corresponding to the combination of substitutions L234A/L235A/P329G as described in Lo et al. J. Biol. Chem (2017) 292(9):3900-3908. In some embodiments, the Fc region comprises modification corresponding to the combination of substitutions described in Rother et al., Nat Biotechnol. (2007) 25:1256-1264. In some embodiments, the Fc region comprises modification corresponding to the combination of substitutions S228P/L235E as described in Newman et al., Clin. Immunol. (2001) 98:164-174. In some embodiments, the Fc region comprises modification corresponding to the combination of substitutions H268Q/V309L/A330S/P331 S as described in An et al., MAbs. (2009) 1:572-579. In some embodiments, the Fc region comprises modification corresponding to the combination of substitutions V234A/G237A/P238S/H268A/V309L/A330S/P331 S as described in Vafa et al., Methods. (2014) 65:114-126. In some embodiments, the Fc region comprises modification corresponding to the combination of substitutions L234A/L235E/G237A/A330S/P331 S as described in US 2015/0044231 A1.

The combination of substitutions “L234A/L235A” and corresponding substitutions (such as e.g. F234A/L235A in human IgG4) are known to disrupt binding of Fc to Fcγ receptors and inhibit ADCC, ADCP, and also to reduce C1q binding and thus CDC (Schlothauer et al., Protein Engineering, Design and Selection (2016), 29(10):457-466, hereby incorporated by reference in entirety). The substitutions “P329G” and “P329A” reduce C1q binding (and thereby CDC). Substitution of “N297” with “A”, “G” or “Q” is known to eliminate glycosylation, and thereby reduce Fc binding to C1q and Fcγ receptors, and thus CDC and ADCC. Lo et al. J. Biol. Chem (2017) 292(9):3900-3908 (hereby incorporated by reference in its entirety) reports that the combination of substitutions L234A/L235A/P329G eliminated complement binding and fixation as well as Fc γ receptor dependent, antibody-dependent, cell-mediated cytotoxicity in both murine IgG2a and human IgG1.

The combination of substitutions L234A/L235E/G237A/A330S/P331 S in IgG1 Fc is disclosed in US 2015/0044231 A1 to abolish induction of phagocytosis, ADCC and CDC.

In some embodiments, the Fc region comprises modification corresponding to the substitution S228P as described in Silva et al., J Biol Chem. (2015) 290(9):5462-5469. The substitution S228P in IgG4 Fc reduces Fab-arm exchange (Fab arm exchange can be undesirable).

In some embodiments, the Fc region comprises modification corresponding to corresponding to the combination of substitutions L234A/L235A. In some embodiments, the Fc region comprises modification corresponding to corresponding to the substitution P329G. In some embodiments, the Fc region comprises modification corresponding to corresponding to the substitution N297Q.

In some embodiments, the Fc region comprises modification corresponding to corresponding to the combination of substitutions L234A/L235A/P329G.

In some embodiments, the Fc region comprises modification corresponding to corresponding to the combination of substitutions L234A/L235A/P329G/N297Q.

In some embodiments, the Fc region comprises modification corresponding to corresponding to the combination of substitutions L234A/L235E/G237A/A330S/P331 S.

In some embodiments, the Fc region comprises modification corresponding to corresponding to the substitution S228P, e.g. in IgG4.

In some embodiments, the antigen-binding molecule of the present disclosure comprises an Fc region comprising modification in one or more of the CH2 and CH3 regions promoting association of the Fc region. Recombinant co-expression of constituent polypeptides of an antigen-binding molecule and subsequent association leads to several possible combinations. To improve the yield of the desired combinations of polypeptides in antigen-binding molecules in recombinant production, it is advantageous to introduce in the Fc regions modification(s) promoting association of the desired combination of heavy chain polypeptides. Modifications may promote e.g. hydrophobic and/or electrostatic interaction between CH2 and/or CH3 regions of different polypeptide chains. Suitable modifications are described e.g. in Ha et al., Front. Immnol (2016) 7:394, which is hereby incorporated by reference in its entirety. In some embodiments, the antigen antigen-binding molecule of the present disclosure comprises an Fc region comprising paired substitutions in the CH3 regions of the Fc region according to one of the following formats, as shown in Table 1 of Ha et al., Front. Immnol (2016) 7:394: KiH, KiHs-s, HA-TF, ZW1, 7.8.60, DD-KK, EW-RVT, EW-RVTs-s, SEED or A107.

In some embodiments, the Fc region comprises the “knob-into-hole” or “KiH” modification, e.g. as described e.g. in U.S. Pat. No. 7,695,936 and Carter, J Immunol Meth 248, 7-15 (2001). In such embodiments, one of the CH3 regions of the Fc region comprises a “knob” modification, and the other CH3 region comprises a “hole” modification. The “knob” and “hole” modifications are positioned within the respective CH3 regions so that the “knob” can be positioned in the “hole” in order to promote heterodimerisation (and inhibit homodimerisation) of the polypeptides and/or stabilise heterodimers. Knobs are constructed by substituting amino acids having small chains with those having larger side chains (e.g. tyrosine or tryptophan). Holes are created by substituting amino acids having large side chains with those having smaller side chains (e.g. alanine or threonine).

In some embodiments, one of the CH3 regions of the Fc region of the antigen-binding molecule of the present disclosure comprises the substitution (numbering of positions/substitutions in the Fc, CH2 and CH3 regions herein is according to the EU numbering system as described in Kabat et al., Sequences of Proteins of Immunological Interest, 5th Ed. Public Health Service, National Institutes of Health, Bethesda, MD, 1991) T366W, and the other CH3 region of the Fc region comprises the substitution Y407V. In some embodiments, one of the CH3 regions of the Fc region of the antigen-binding molecule comprises the substitution T366W, and the other CH3 region of the Fc region comprises the substitutions T366S and L368A. In some embodiments, one of the CH3 regions of the Fc region of the antigen-binding molecule comprises the substitution T366W, and the other CH3 region of the Fc region comprises the substitutions Y407V, T366S and L368A.

In some embodiments, the Fc region comprises the “DD-KK” modification as described e.g. in WO 2014/131694 A1. In some embodiments, one of the CH3 regions comprises the substitutions K392D and K409D, and the other CH3 region of the Fc region comprises the substitutions E356K and D399K. The modifications promote electrostatic interaction between the CH3 regions.

In some embodiments, the antigen-binding molecule of the present disclosure comprises an Fc region modified as described in Labrijn et al., Proc Natl Acad Sci USA. (2013) 110(13):5145-50, referred to as ‘Duobody’ format. In some embodiments one of the CH3 regions comprises the substitution K409R, and the other CH3 region of the Fc region comprises the substitution K405L.

In some embodiments, the antigen-binding molecule of the present disclosure comprises an Fc region comprising the “EEE-RRR” modification as described in Strop et al., J Mol Biol. (2012) 420(3):204-19. In some embodiments one of the CH3 regions comprises the substitutions D221E, P228E and L368E, and the other CH3 region of the Fc region comprises the substitutions D221R, P228R and K409R. In some embodiments, the antigen-binding molecule comprises an Fc region comprising the “EW-RVT” modification described in Choi et al., Mol Cancer Ther (2013) 12(12):2748-59. In some embodiments one of the CH3 regions comprises the substitutions K360E and K409W, and the other CH3 region of the Fc region comprises the substitutions Q347R, D399V and F405T.

In some embodiments, one of the CH3 regions comprises the substitution S354C, and the other CH3 region of the Fc region comprises the substitution Y349C. Introduction of these cysteine residues results in formation of a disulphide bridge between the two CH3 regions of the Fc region, further stabilizing the heterodimer (Carter (2001), J Immunol Methods 248, 7-15).

In some embodiments, the Fc region comprises the “KiHs-s” modification. In some embodiments one of the CH3 regions comprises the substitutions T366W and S354C, and the other CH3 region of the Fc region comprises the substitutions T366S, L368A, Y407V and Y349C.

In some embodiments, the antigen-binding molecule of the present disclosure comprises an Fc region comprising the “SEED” modification as described in Davis et al., Protein Eng Des Sel (2010) 23(4):195-202, in which β-strand segments of human IgG1 CH3 and IgA CH3 are exchanged.

In some embodiments, one of the CH3 regions comprises the substitutions S364H and F405A, and the other CH3 region of the Fc region comprises the substitutions Y349T and T394F (see e.g. Moore et al., MAbs (2011) 3(6):546-57).

In some embodiments, one of the CH3 regions comprises the substitutions T350V, L351Y, F405A and Y407V, and the other CH3 region of the Fc region comprises the substitutions T350V, T366L, K392L and T394W (see e.g. Von Kreudenstein et al., MAbs (2013) 5(5):646-54).

In some embodiments, one of the CH3 regions comprises the substitutions K360D, D399M and Y407A, and the other CH3 region of the Fc region comprises the substitutions E345R, Q347R, T366V and K409V (see e.g. Leaver-Fay et al., Structure (2016) 24(4):641-51).

In some embodiments, one of the CH3 regions comprises the substitutions K370E and K409W, and the other CH3 region of the Fc region comprises the substitutions E357N, D399V and F405T (see e.g. Choi et al., PLoS One (2015) 10(12):e0145349).

In some embodiments, the antigen-binding molecule of the present disclosure comprises an Fc region which does not bind to an Fc γ receptor. In some embodiments, the antigen-binding molecule comprises an Fc region which does not bind to one or more of FcγRI, FcγRIIa, FcγRIIb, FcγRIIc, FcγRIIIa and FcγRIIIb. In some embodiments, the antigen-binding molecule comprises an Fc region which does not bind to one or more of FcγRIIa, FcγRIIb and FcγRIIIa. In some embodiments, the antigen-binding molecule comprises an Fc region which does not bind to one or both of FcγRIIa and FcγRIIb.

The ability of an Fc region, or an antigen-binding molecule comprising an Fc region, to bind to a reference protein (e.g. an Fc receptor) can be analysed according to methods well known in the art, such as ELISA, immunoblot, immunoprecipitation, Surface Plasmon Resonance (SPR; see e.g. Hearty et al., Methods Mol Biol (2012) 907:411-442) or Bio-Layer Interferometry (BLI; see e.g. Lad et al., (2015) J Biomol Screen 20(4): 498-507).

As used herein, an Fc region “which does not bind to” a reference protein may display substantially no binding to the reference protein, e.g. as determined by ELISA, immunoblot (e.g. western blot), immunoprecipitation, SPR or BLI). “Substantially no binding” may be a level of interaction that is not significantly greater than the level of interaction determined for proteins that do not bind to one another in a given assay. “Substantially no binding” may be a level of interaction which is ≤5 times, e.g. ≤4 times, ≤3 times, s 2.5 times, ≤2 times or ≤1.5 times the level of interaction determined for proteins that do not bind to one another, in a given assay.

In some embodiments, the antigen-binding molecule comprises an Fc region which binds to FcRn.

In some embodiments, the antigen-binding molecule comprises an Fc region which binds to FcRn, and which does not bind to one or more of FcγRIIa, FcγRIIb and FcγRIIIa. In some embodiments, the antigen-binding molecule comprises an Fc region which binds to FcRn, and which does not bind to one or both of FcγRIIa and FcγRIIb.

In some embodiments, the antigen-binding molecule of the present disclosure comprises an Fc region which does not induce ADCC. In some embodiments, the antigen-binding molecule of the present disclosure comprises an Fc region which does not induce ADCP. In some embodiments, the antigen-binding molecule of the present disclosure comprises an Fc region which does not induce CDC. In some embodiments, the antigen-binding molecule of the present disclosure comprises an Fc region which does not induce ADCC, ADCP or CDC.

As used herein, an Fc region/antigen-binding molecule which does not induce (i.e. is not able to induce) ADCC/ADCP/CDC elicits substantially no ADCC/ADCP/CDC activity, e.g. as determined by analysis in an appropriate assay for the relevant activity. “Substantially no ADCC/ADCP/CDC activity” refers to a level of ADCC/ADCP/CDC that is not significantly greater than ADCC/ADCP/CDC determined for an appropriate negative control molecule in a given assay (e.g. an antigen-binding molecule lacking an Fc region, or an antigen-binding molecule comprising a ‘silent’ Fc region (e.g. as described in Schlothauer et al., Protein Engineering, Design and Selection (2016), 29(10):457-466, which is incorporated by reference hereinabove)). “Substantially no activity” may be a level of the relevant activity which is ≤5 times, e.g. ≤4 times, ≤3 times, ≤2.5 times, ≤2 times or ≤1.5 times the level of activity determined for an appropriate negative control molecule in a given assay.

The ability of an Fc region, or an antigen-binding molecule comprising an Fc region, to induce ADCC can be analysed e.g. according to the method described in Yamashita et al., Scientific Reports (2016) 6:19772 (hereby incorporated by reference in its entirety), or by ⁵¹Cr release assay as described e.g. in Jedema et al., Blood (2004) 103: 2677-82 (hereby incorporated by reference in its entirety). The ability of an Fc region, or an antigen-binding molecule comprising an Fc region, to induce ADCP can be analysed e.g. according to the method described in Kamen et al., J Immunol (2017) 198 (1 Supplement) 157.17 (hereby incorporated by reference in its entirety). The ability of an Fc region, or an antigen-binding molecule comprising an Fc region, to induce CDC can be analysed e.g. using a C1q binding assay, e.g. as described in Schlothauer et al., Protein Engineering, Design and Selection (2016), 29(10):457-466 (incorporated by reference hereinabove).

In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:254. In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:257. In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:259. In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:260.

In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:347.

In some embodiments, the antigen-binding molecules of the present disclosure lack an Fc region.

### Fc Receptors

Fc receptors are polypeptides which bind to the Fc region of immunoglobulins. Fc receptor structure and function is reviewed e.g. in Masuda et al., Inflamm Allergy Drug Targets (2009) 8(1): 80-86, and Bruhns, Blood (2012) 119:5640-5649, both of which are hereby incorporated by reference in their entirety.

Fc receptors are expressed at surface of hematopoietic cells including macrophages, neutrophils, dendritic cells, eosinophils, basophils, mast cells, and NK cells. They include the IgG-binding Fc γ receptors, the high-affinity receptor for IgE (FcεRI), the IgA receptor, and the polymeric Ig receptor for IgA and IgM. The neonatal Fc receptor (FcRn) is a further Fc receptor for IgG, and is involved in IgG transport across epithelial barriers (transcytosis), protecting IgG from degradation, and antigen presentation. Humans have six different classes of Fc γ receptor (mouse orthologues are shown in brackets): FcγRI (mFcγRI), FcγRIIa (mFcγRIII), FcγRIIb (mFcγRIIb), FcγRIIc, FcγRIIIa (mFcγRIV) and FcγRIIIb. FcγRI, FcγRIIa, FcγRIIc and FcγRIIIa comprise immunoreceptor tyrosine-based activation motifs (ITAMs) in their intracellular domains, and ligation by Fc leads to activation of cells expressing the receptors. FcγRIIb comprises immunoreceptor tyrosine-based inhibitory motifs (ITIMs) in its intracellular domain, and negatively regulates cell activation and degranulation, cell proliferation, endocytosis, and phagocytosis upon ligation by Fc.

In this specification an “Fcγ receptor” may be from any species, and includes isoforms, fragments, variants (including mutants) or homologues from any species. Similarly, “FcγRI”, “FcγRIIa”, “FcγRIIb”, “FcγRIIc”, “FcγRIIIa” and “FcγRIIIb” refer respectively to FcγRI/FcγRIIa/FcγRIIb/FcγRIIc/FcγRIIIa/FcγRIIIb from any species, and include isoforms, fragments, variants (including mutants) or homologues from any species.

In some embodiments, the Fc γ receptor (e.g. FcγRI/FcγRIIa/FcγRIIb/FcγRIIc/FcγRIIIa/FcγRIIIb) is from a mammal (e.g. a primate (rhesus, cynomolgous, non-human primate or human) and/or a rodent (e.g. rat or mouse). Isoforms, fragments, variants or homologues may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature isoform of an Fc γ receptor (e.g. FcγRI/FcγRIIa/FcγRIIb/FcγRIIc/FcγRIIIa/FcγRIIIb) from a given species, e.g. human.

Isoforms, fragments, variants or homologues may optionally be functional isoforms, fragments, variants or homologues, e.g. having a functional property/activity of the reference Fc γ receptor, as determined by analysis by a suitable assay for the functional property/activity. For example, an isoform, fragment, variant or homologue of FcγRI may e.g. display association with human IgG1 Fc.

In this specification an “FcRn receptor” may be from any species, and includes isoforms, fragments, variants (including mutants) or homologues from any species.

In some embodiments, the FcRn receptor is from a mammal (e.g. a primate (rhesus, cynomolgous, non-human primate or human) and/or a rodent (e.g. rat or mouse). Isoforms, fragments, variants or homologues may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature isoform of an FcRn receptor from a given species, e.g. human.

Isoforms, fragments, variants or homologues may optionally be functional isoforms, fragments, variants or homologues, e.g. having a functional property/activity of the reference FcRn, as determined by analysis by a suitable assay for the functional property/activity. For example, an isoform, fragment, variant or homologue of FcRn may e.g. display association with human IgG1 Fc.

### Polypeptides

The present disclosure also provides polypeptide constituents of antigen-binding molecules. The polypeptides may be provided in isolated or substantially purified form.

The antigen-binding molecule of the present disclosure may be, or may comprise, a complex of polypeptides.

In the present specification where a polypeptide comprises more than one domain or region, it will be appreciated that the plural domains/regions are preferably present in the same polypeptide chain. That is, the polypeptide comprises more than one domain or region is a fusion polypeptide comprising the domains/regions.

In some embodiments a polypeptide according to the present disclosure comprises, or consists of, a VH as described herein. In some embodiments a polypeptide according to the present disclosure comprises, or consists of, a VL as described herein.

In some embodiments, the polypeptide additionally comprises one or more antibody heavy chain constant regions (CH). In some embodiments, the polypeptide additionally comprises one or more antibody light chain constant regions (CL). In some embodiments, the polypeptide comprises a CH1, CH2 region and/or a CH3 region of an immunoglobulin (Ig).

In some embodiments, the polypeptide comprises one or more regions of an immunoglobulin heavy chain constant sequence. In some embodiments, the polypeptide comprises a CH1 region as described herein.

In some embodiments, the polypeptide comprises a CH1-CH2 hinge region as described herein. In some embodiments, the polypeptide comprises a CH2 region as described herein. In some embodiments, the polypeptide comprises a CH3 region as described herein.

In some embodiments, the polypeptide comprises a CH2 and/or CH3 region comprising any one of the following amino acid substitutions/combinations of amino acid substitutions: F243L/R292P/Y300L/V3051/P396L; S239D/1332E; S239D/1332E/A330L; S298A/E333A/K334A; L234Y/L235Q/G236W/S239M/H268D/D270E/S298A; D270E/K326D/A330M/K334E; G236A/S239D/1332E; K326W/E333S; S267E/H268F/S324T; E345R/E430G/S440Y; M252Y/S254T/T256E; M428L/N434S; S267E/L328F; N325S/L328F; N297A; N297Q; N297G; L235E; L234A/L235A; F234A/L235A; P329A; P329G; L234A/L235A/P329G; H268Q/V309L/A330S/P331S; and V234A/G237A/P238S/H268A/V309L/A330S/P331S.

In some embodiments, the polypeptide comprises a CH3 region comprising any one of the following 35 amino acid substitutions/combinations of amino acid substitutions (shown e.g. in Table 1 of Ha et al., Front. Immnol (2016) 7:394, incorporated by reference hereinabove): T366W; T366S, L368A and Y407V; T366W and S354C; T366S, L368A, Y407V and Y349C; S364H and F405A; Y349T and T394F; T350V, L351Y, F405A and Y407V; T350V, T366L, K392L and T394W; K360D, D399M and Y407A; E345R, Q347R, T366V and K409V; K409D and K392D; D399K and E356K; K360E and K409W; Q347R, D399V and F405T; K360E, K409W and Y349C; Q347R, D399V, F405T and S354C; K370E and K409W; and E357N, D399V and F405T.

In some embodiments, the CH2 and/or CH3 regions of the polypeptide comprise one or more amino acid substitutions for promoting association of the polypeptide with another polypeptide comprising a CH2 and/or CH3 region.

In some embodiments, the polypeptide comprises one or more regions of an immunoglobulin light chain constant sequence. In some embodiments, the polypeptide comprises a CL region as described herein.

In some embodiments, the polypeptide lacks one or more regions of an immunoglobulin heavy chain constant sequence. In some embodiments, the polypeptide lacks a CH2 region. In some embodiments, the polypeptide lacks a CH3 region. In some embodiments, the polypeptide lacks a CH2 region and also lacks a CH3 region.

In some embodiments, the polypeptide according to the present disclosure comprises a structure from N- to C-terminus according to one of the following:

(i) VH
(ii) VL
(iii) VH-CH1
(iv) VL-CL
(v) VL-CH1
(vi) VH-CL
(vii) VH-CH1-CH2-CH3
(viii) VL-CL-CH2-CH3
(ix) VL-CH1-CH2-CH3
(x) VH-CL-CH2-CH3

Also provided by the present disclosure are antigen-binding molecules composed of the polypeptides of the present disclosure. In some embodiments, the antigen-binding molecule of the present disclosure comprises one of the following combinations of polypeptides:

(A) VH + VL
(B) VH-CH1 + VL-CL
(C) VL-CH1 + VH-CL
(D) VH-CH1-CH2-CH3 + VL-CL
(E) VH-CL-CH2-CH3 + VL-CH1
(F) VL-CH1-CH2-CH3 + VH-CL
(G) VL-CL-CH2-CH3 + VH-CH1
(H) VH-CH1-CH2-CH3 + VL-CL-CH2-CH3
(I) VH-CL-CH2-CH3 + VL-CH1-CH2-CH3

In some embodiments, the antigen-binding molecule comprises more than one of a polypeptide of the combinations shown in (A) to (I) above. By way of example, with reference to (D) above, in some embodiments, the antigen-binding molecule comprises two polypeptides comprising the structure VH-CH1-CH2-CH3, and two polypeptides comprising the structure VL-CL.

In some embodiments, the antigen-binding molecule of the present disclosure comprises one of the following combinations of polypeptides:

(J) VH (anti-VISTA) + VL (anti-VISTA)
(K) VH (anti-VISTA)-CH1 + VL (anti-VISTA)-CL
(L) VL (anti-VISTA)-CH1 + VH (anti-VISTA)-CL
(M) VH (anti-VISTA)-CH1-CH2-CH3 + VL (anti-VISTA)-CL
(N) VH (anti-VISTA)-CL-CH2-CH3 + VL (anti-VISTA)-CH1
(O) VL (anti-VISTA)-CH1-CH2-CH3 + VH (anti-VISTA)-CL
(P) VL (anti-VISTA)-CL-CH2-CH3 + VH (anti-VISTA)-CH1
(Q) VH (anti-VISTA)-CH1-CH2-CH3 + VL (anti-VISTA)-CL-CH2-CH3
(R) VH (anti-VISTA)-CL-CH2-CH3 + VL (anti-VISTA)-CH1-CH2-CH3

Wherein: “VH (anti-VISTA)” refers to the VH of an antigen-binding molecule capable of binding to VISTA as described herein, e.g. as defined in one of (1) to (76); “VL (anti-VISTA)” refers to the VL of an antigen-binding molecule capable of binding to VISTA as described herein, e.g. as defined in one of (77) to (173).

In some embodiments, the polypeptide comprises or consists of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of one of SEQ ID NOs:212 to 243, 248 to 250, 258, 266 or 311 to 321.

### Linkers and Additional Sequences

In some embodiments, the antigen-binding molecules and polypeptides of the present disclosure comprise a hinge region. In some embodiments a hinge region is provided between a CH1 region and a CH2 region. In some embodiments a hinge region is provided between a CL region and a CH2 region. In some embodiments, the hinge region comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:207.

In some embodiments, the antigen-binding molecules and polypeptides of the present disclosure comprise one or more linker sequences between amino acid sequences. A linker sequence may be provided at one or both ends of one or more of a VH, VL, CH1-CH2 hinge region, CH2 region and a CH3 region of the antigen-binding molecule/polypeptide.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments, the linker sequence consists of glycine and serine residues. In some embodiments, the linker sequence has a length of 1-2, 1-3, 1-4, 1-5 or 1-10 amino acids.

The antigen-binding molecules and polypeptides of the present disclosure may additionally comprise further amino acids or sequences of amino acids. For example, the antigen-binding molecules and polypeptides may comprise amino acid sequence(s) to facilitate expression, folding, trafficking, processing, purification or detection of the antigen-binding molecule/polypeptide. For example, the antigen-binding molecule/polypeptide may comprise a sequence encoding a His, (e.g. 6×His), Myc, GST, MBP, FLAG, HA, E, or Biotin tag, optionally at the N- or C-terminus of the antigen-binding molecule/polypeptide. In some embodiments, the antigen-binding molecule/polypeptide comprises a detectable moiety, e.g. a fluorescent, lunminescent, immuno-detectable, radio, chemical, nucleic acid or enzymatic label.

The antigen-binding molecules and polypeptides of the present disclosure may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the antigen-binding molecule/polypeptide, and may be present in the newly synthesised antigen-binding molecule/polypeptide. The signal peptide provides for efficient trafficking and secretion of the antigen-binding molecule/polypeptide. Signal peptides are often removed by cleavage, and thus are not comprised in the mature antigen-binding molecule/polypeptide secreted from the cell expressing the antigen-binding molecule/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

### Labels and Conjugates

In some embodiments, the antigen-binding molecules of the present disclosure additionally comprise a detectable moiety.

In some embodiments, the antigen-binding molecule comprises a detectable moiety, e.g. a fluorescent label, phosphorescent label, luminescent label, immuno-detectable label (e.g. an epitope tag), radiolabel, chemical, nucleic acid or enzymatic label. The antigen-binding molecule may be covalently or non-covalently labelled with the detectable moiety.

Fluorescent labels include e.g. fluorescein, rhodamine, allophycocyanin, eosine and NDB, green fluorescent protein (GFP) chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as lodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thuliuml¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase.

In some embodiments, the antigen-binding molecules of the present disclosure are conjugated to a chemical moiety. The chemical moiety may be a moiety for providing a therapeutic effect. Antibody-drug conjugates are reviewed e.g. in Parslow et al., Biomedicines. 2016 September; 4(3):14. In some embodiments, the chemical moiety may be a drug moiety (e.g. a cytotoxic agent). In some embodiments, the drug moiety may be a chemotherapeutic agent. In some embodiments, the drug moiety is selected from calicheamicin, DM1, DM4, monomethylauristatin E (MMAE), monomethylauristatin F (MMAF), SN-38, doxorubicin, duocarmycin, D6.5 and PBD.

### Particular Exemplary Embodiments of the Antigen-Binding Molecules

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:212; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:213.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:214; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:215.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:216; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:217.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:218; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:219.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:220; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:221.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:222; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:223.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:224; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:225.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:226; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:227.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:228; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:229.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:230; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:231.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:232; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:233.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:234; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:235.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:236; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:237.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:238; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:239.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:240; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:241.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:242; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:243.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:248; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:249; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:258; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:266; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments, the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:330; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:213.

### Functional Properties of the Antigen-Binding Molecules

The antigen-binding molecules described herein may be characterised by reference to certain functional properties. In some embodiments, the antigen-binding molecule described herein may possess one or more of the following properties:
binds to VISTA (e.g. human, murine and/or cynomolgus macaque VISTA);
does not bind to PD-L1 and/or HER3;
does not bind to an Fcγ receptor;
does not bind to C1q;
does not induce ADCC;
does not induce ADCP;
does not induce CDC;
binds to an FcRn receptor;
binds to VISTA with similar affinity at pH from 5.5 to pH 7.5;
binds to VISTA-expressing cells;
inhibits interaction between VISTA and an interaction partner for VISTA (e.g. LRIG1, PSGL-1, VSIG3 or VSIG8);
inhibits VISTA-mediated signalling;
inhibits VISTA-mediated signalling independently of Fc-mediated function;
increases killing of VISTA-expressing cells;
does not induce/increase killing of VISTA-expressing cells;
reduces the number/proportion of VISTA-expressing cells;
does not reduce the number/proportion of VISTA-expressing cells;
increases effector immune cell number/activity;
reduces suppressor immune cell number/activity;
reduces suppressor immune cell proliferation;
decreases immune suppression mediated by VISTA-expressing cells;
increases antigen presentation by antigen-presenting cells;
increases production of IL-6 by immune cells;
increases production of IFN-γ, IL-2 and/or IL-17 in a mixed lymphocyte reaction (MLR) assay;
increases T cell proliferation, IFN-γ production, TNFa production and/or T cell-mediated lysis of cancer cells;
inhibits the development and/or progression of cancer in vivo;
does not induce cytokine release syndrome in vivo;
increases the number and/or proportion of antigen-specific CD8+ T cells;
increases CD8+ T cell activity;
upregulates one or more cytotoxicity-associated markers (e.g. Granzyme B, CX3CR1, ICOS, CD27);
upregulates one or more genes associated with pro-inflammatory macrophage activation;
upregulates one or more genes associated with cytotoxic activity of T cells
increases production of Granzyme B;
reduces the level of T cell exhaustion;
reduces the number and/or proportion of tumour-associated macrophages (TAMs);
reduces tumour-associated macrophage activity;
increases the number and/or proportion of M1-type macrophages;
increases M1-type macrophage activity.

The term “proportion” of a cell type/subtype as used herein may be the proportion of said cell type/subtype within a population of cells, e.g. CD45+ cells, e.g. CD45+ cells obtained from a tumour.

It will be appreciated that a given antigen-binding molecule may display more than one of the properties recited in the preceding paragraph. A given antigen-binding molecule may be evaluated for the properties recited in the preceding paragraph using suitable assays. The assays may be e.g. in vitro assays, which may be cell-free or cell-based assays. Alternatively, the assays may be e.g. in vivo assays, i.e. performed in non-human animals.

Where assays are cell-based assays, they may comprise contacting cells with a given antigen-binding molecule in order to determine whether the antigen-binding molecule displays one or more of the recited properties. Assays may employ species labelled with detectable entities in order to facilitate their detection. Assays may comprise evaluating the recited properties following treatment of cells separately with a range of quantities/concentrations of antigen-binding molecule (e.g. a dilution series). It will be appreciated that the cells are preferably cells that express VISTA, e.g. MDSCs.

Analysis of the results of such assays may comprise determining the concentration at which 50% of the maximal level of the relevant activity is attained. The concentration of antigen-binding molecule at which 50% of the maximal level of the relevant activity is attained may be referred to as the ‘half-maximal effective concentration’ of the antigen-binding molecule in relation to the relevant activity, which may also be referred to as the ‘EC₅₀’. By way of illustration, the EC₅₀ of a given antigen-binding molecule for binding to VISTA may be the concentration at which 50% of the maximal level of binding to the relevant species is achieved.

Depending on the property, the EC₅₀ may also be referred to as the ‘half-maximal inhibitory concentration’ or ‘IC₅₀’, this being the concentration of antigen-binding molecule at which 50% of the maximal level of inhibition of a given property is observed. By way of illustration, the IC₅₀ of a given antigen-binding molecule for inhibiting interaction between VISTA and an interaction partner for VISTA (e.g. LRIG1, PSGL-1, VSIG3 or VSIG8) may be the concentration at which 50% of the maximal level of inhibition is achieved.

The antigen-binding molecules described herein bind to VISTA. In preferred embodiments, the antigen-binding molecules display specific binding to VISTA. As used herein, “specific binding” refers to binding which is selective for the antigen, and which can be discriminated from non-specific binding to non-target antigen. An antigen-binding molecule that specifically binds to VISTA preferably binds to VISTA with greater affinity, and/or with greater duration than it binds to other, non-target molecules.

The ability of a given polypeptide to bind specifically to a given molecule can be determined by analysis according to methods known in the art, such as by ELISA, Surface Plasmon Resonance (SPR; see e.g. 40 Hearty et al., Methods Mol Biol (2012) 907:411-442), Bio-Layer Interferometry (see e.g. Lad et al., (2015) J Biomol Screen 20(4): 498-507), flow cytometry, or by a radiolabeled antigen-binding assay (RIA) enzyme-linked immunosorbent assay. Through such analysis binding to a given molecule can be measured and quantified. In some embodiments, the binding may be the response detected in a given assay.

In some embodiments, the extent of binding of the antigen-binding molecule to a non-target molecule is less than about 10% of the binding of the antibody to the target molecule as measured, e.g. by ELISA, SPR, Bio-Layer Interferometry or by RIA. Alternatively, binding specificity may be reflected in terms of binding affinity where the antigen-binding molecule binds with a dissociation constant (K_{D}) that is at least 0.1 order of magnitude (i.e. 0.1×10ⁿ, where n is an integer representing the order of magnitude) greater than the K_{D} of the antigen-binding molecule towards a non-target molecule. This may optionally be one of at least 0.2, 0.3, 0.4, 0.5, 0.6, 0.7, 0.8, 0.9, 1.0, 1.5, or 2.0.

In some embodiments, an antigen-binding molecule according to the present disclosure binds to VISTA with an affinity in the micromolar range, i.e. K_{D}=9.9×10⁻⁴ to 1×10⁻⁶ M. In some embodiments, an antigen-binding molecule binds to VISTA with sub-micromolar affinity, i.e. K_{D}<1×10⁻⁶ M. In some embodiments, an antigen-binding molecule binds to VISTA with an affinity in the nanomolar range, i.e. K_{D}=9.9×10⁻⁷ to 1×10⁻⁹ M. In some embodiments, an antigen-binding molecule binds to VISTA with sub-nanomolar affinity, i.e. K_{D}<1×10⁻⁹ M. In some embodiments, an antigen-binding molecule binds to VISTA with an affinity in the picomolar range, i.e. K_{D}=9.9×10⁻¹⁰ to 1×10⁻¹² M. In some embodiments, an antigen-binding molecule binds to VISTA with sub-picomolar affinity, i.e. K_{D}<1×10⁻¹² M.

In some embodiments, an antigen-binding molecule according to the present disclosure binds to VISTA with a K_{D} of 10 pM or less, preferably one of ≤5 pM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM, ≤3 nM, ≤2 nM, ≤1 nM, ≤500 pM, ≤400 pM, ≤300 pM, ≤200 pM, ≤100 pM, ≤50 pM, ≤40 pM, ≤30 pM, ≤20 pM, ≤10 pM or ≤1 pM. In some embodiments, an antigen-binding molecule according to the present disclosure binds to VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM, ≤400 pM, ≤300 pM).

In some embodiments, an antigen-binding molecule according to the present disclosure binds to human VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM). In some embodiments, an antigen-binding molecule according to the present disclosure binds to cynomolgus macaque VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM, ≤400 pM). In some embodiments, an antigen-binding molecule according to the present disclosure binds to rat VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM, ≤400 pM). In some embodiments, an antigen-binding molecule according to the present disclosure binds to mouse VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM).

In some embodiments, an antigen-binding molecule according to the present disclosure binds to VISTA with an EC₅₀ (e.g. as determined by ELISA, e.g. an ELISA as described in the Examples of the present disclosure) of ≤1 μM or less, e.g. one of ≤500 nM, ≤100 nM, ≤50 nM, ≤40 nM, ≤30 nM, ≤20 nM, ≤10 nM, ≤5 nM, ≤4 nM, ≤3 nM, ≤2 nM, ≤1 nM, ≤500 pM, ≤400 pM, ≤300 pM, ≤200 pM, ≤100 pM, ≤50 pM, ≤40 pM, ≤30 pM, ≤20 pM, ≤15 pM, ≤10 pM, ≤5 pM or 51 pM.

In some embodiments, the antigen-binding molecule displays binding to human VISTA, murine (e.g. mouse) VISTA, rat VISTA, and/or cynomolgus macaque (Macaca fascicularis) VISTA. In some embodiments, the antigen-binding molecule binds to human VISTA and mouse VISTA and rat VISTA and cynomolgus macaque VISTA. In some embodiments, the antigen-binding molecule is cross-reactive for human VISTA and mouse VISTA and rat VISTA and cynomolgus macaque VISTA. In some embodiments, the antigen-binding molecule of the present disclosure displays cross-reactivity with VISTA of a non-human primate. Cross-reactivity to VISTA in model species allows in vivo exploration of efficacy in syngeneic models without relying on surrogate molecules.

In some embodiments, an antigen-binding molecule according to the present disclosure binds to human VISTA with an EC₅₀ (e.g. as determined by ELISA, e.g. an ELISA as described in the Examples of the present disclosure) of ≤20 pM (e.g. one of ≤15 pM, ≤12.5 pM, ≤10 pM, ≤7.5 pM). In some embodiments, an antigen-binding molecule according to the present disclosure binds to cynomolgus macaque VISTA with an EC₅₀ (e.g. as determined by ELISA, e.g. an ELISA as described in the Examples of the present disclosure) of ≤50 pM (e.g. one of ≤25 pM, ≤20 pM, ≤15 pM). In some embodiments, an antigen-binding molecule according to the present disclosure binds to rat VISTA with an EC₅₀ (e.g. as determined by ELISA, e.g. an ELISA as described in the Examples of the present disclosure) of 520 pM (e.g. one of ≤15 pM, ≤12.5 pM, ≤10 pM, ≤7.5 pM). In some embodiments, an antigen-binding molecule according to the present disclosure binds to mouse VISTA with an EC₅₀ (e.g. as determined by ELISA, e.g. an ELISA as described in the Examples of the present disclosure) of 520 pM (e.g. one of:15 pM, ≤12.5 pM, ≤10 pM, ≤7.5 pM, ≤5 pM).

In some embodiments, an antigen-binding molecule according to the present disclosure binds to VISTA (e.g. human VISTA) with similar affinity at pH from 5.5 to pH 7.5. For example, in some embodiments, the antigen-binding molecule displays similar affinity for VISTA at pH 5.5 as the affinity for VISTA at pH 7.5.

Herein, a binding affinity which is ‘similar’ to a reference binding affinity means a binding affinity which is within 50%, e.g. within one of 40%, 45%, 30%, 25%, 20% 19%, 18%, 17%, 16%, 15%, 14%, 13%, 12%, 11%, 10%, 9%, 8%, 7%, 6%, 5%, 4%, 3%, 2% or 1% of the reference binding affinity, as determined under comparable conditions.

The K_{D} for binding to VISTA (e.g. human VISTA) may be similar at pH from 5.5 to pH 7.5. The EC₅₀ for binding to VISTA (e.g. human VISTA) may be similar at pH from 5.5 to pH 7.5.

Herein, a ‘similar’ K_{D} or EC₅₀ value to a reference value may be ≥0.5 times and ≥2 times, e.g. one of ≥0.7 times and ≤1.5 times, ≥0.75 times and ≤1.25 times, ≥0.8 times and ≤1.2 times, ≥0.85 times and ≤1.15 times, ≥0.9 times and ≤1.1 times, ≥0.91 times and ≤1.09 times, ≥0.92 times and ≤1.08 times, ≥0.93 times and ≤1.07 times, ≥0.94 times and ≤1.06 times, ≥0.95 times and ≤1.05 times, ≥0.96 times and ≤1.04 times, ≥0.97 times and ≤1.03 times, ≤0.98 times and ≥1.02 times, or ≥0.99 times and ≤1.01 times the reference value.

In some embodiments, the antigen-binding molecule does not display specific binding to PD-L1 (e.g. human PD-L1). In some embodiments, the antigen-binding molecule does not display specific binding to HER3 (e.g. human HER3). In some embodiments, the antigen-binding molecule does not display specific binding to (i.e. does not cross-react with) another member of the B7 family of proteins. In some embodiments, the antigen-binding molecule does not display specific binding to PD-L1, PD-L2 CD80, CD86, ICOSLG, CD276, VTCN1, NCR3LG1, HHLA2 and/or CTLA4.

In some embodiments, the antigen-binding molecule does not display specific binding to PD-1, PD-L1, B7H3, VTCN1 (B7H4), NCR3LG1 (B7H6), HHLA2 (B7H7) and/or CTLA4.

In some embodiments, the antigen-binding molecule is not able to induce one or more Fc-mediated functions (i.e. lacks the ability to elicit the relevant Fc-mediated function(s)). Such antigen-binding molecules may be described as being devoid of the relevant function(s).

As explained hereinabove, an Fc region/antigen-binding molecule which does not induce (i.e. is not able to induce) ADCC/ADCP/CDC elicits substantially no ADCC/ADCP/CDC activity, e.g. as determined by analysis in an appropriate assay for the relevant activity. Similarly, an antigen-binding molecule “which does not bind to” a reference protein (e.g. a given Fc receptor or complement protein) may display substantially no binding to the reference protein in an appropriate assay.

In some embodiments, the antigen-binding molecule does not induce ADCC. In some embodiments, the antigen-binding molecule does not induce ADCP. In some embodiments, the antigen-binding molecule does not induce CDC. In some embodiments, the antigen-binding molecule does not induce ADCC and/or does not induce ADCP and/or does not induce CDC.

In some embodiments, the antigen-binding molecule does not bind to an Fc receptor. In some embodiments, the antigen-binding molecule does not bind to an Fcγ receptor. In some embodiments, the antigen-binding molecule does not bind to one or more of FcγRI, FcγRIIa, FcγRIIb, FcγRIIc, FcγRIIIa and FcγRIIIb. In some embodiments, the antigen-binding molecule does not bind to FcγRIII (e.g. FcγRIIIa and/or FcγRIIIb). In some embodiments, the antigen-binding molecule does not bind to FcγRIIIa. In some embodiments, the antigen-binding molecule does not bind to FcγRIIa. In some embodiments, the antigen-binding molecule does not bind to FcγRIIb. In some embodiments, the antigen-binding molecule binds to FcRn. In some embodiments, the antigen-binding molecule does not bind to a complement protein. In some embodiments, the antigen-binding molecule does not bind to C1q. In some embodiments, the antigen-binding molecule is not glycosylated at the amino acid residue corresponding to N297.

In some embodiments, the antigen-binding molecule binds to human VISTA, murine VISTA and/or cynomolgus macaque VISTA; and does not bind to PD-L1, PD-1, B7H3, VTCN1 (B7H4), NCR3LG1 (B7H6), HHLA2 (B7H7) and/or CTLA4 (e.g. human PD-L1/PD-1/B7H3/VTCN1/NCR3LG1/HHLA2/CTLA4).

In some embodiments, the antigen-binding molecule described herein binds to VISTA (e.g. human VISTA, mouse VISTA) with a K_{D} of 10 pM or less, preferably one of ≤5 pM, ≤2 pM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM, ≤3 nM, ≤2 nM, ≤1 nM or ≤500 pM. In some embodiments, the antigen-binding molecule binds to VISTA (e.g. human VISTA, mouse VISTA) with an affinity of K_{D}=510 nM, ≤9 nM, ≤8 nM, ≤7 nM or ≤6 nM, ≤5 nM, ≤4 nM, ≤3 nM, ≤2 nM or ≤1 nM. In some embodiments, the antigen-binding molecule binds to VISTA (e.g. human VISTA, mouse VISTA) with an affinity of K_{D}=5500 pM, ≤100 pM, ≤90 pM, ≤80 pM, ≤70 pM or ≤60 pM, ≤50 pM, ≤40 pM, ≤30 pM, ≤20 pM, ≤10 pM, ≤9 pM, ≤8 pM, ≤7 pM or 56 pM, ≤5 pM, ≤4 pM, ≤3 pM, ≤2 pM or ≤1 pM.

The antigen-binding molecules of the present disclosure may bind to a particular region of interest of VISTA. The antigen-binding region of an antigen-binding molecule according to the present domain may bind to a linear epitope of VISTA, consisting of a contiguous sequence of amino acids (i.e. an amino acid primary sequence). In some embodiments, the antigen-binding region molecule may bind to a conformational epitope of VISTA, consisting of a discontinuous sequence of amino acids of the amino acid sequence.

In some embodiments, the antigen-binding molecule of the present disclosure is capable of binding to VISTA. In some embodiments, the antigen-binding molecule is capable of binding to VISTA in an extracellular region of VISTA. In some embodiments, the antigen-binding molecule is capable of binding to VISTA in the Ig-like V-type domain (e.g. the region shown in SEQ ID NO:6). In some embodiments, the antigen-binding molecule is capable of binding to VISTA in the region shown in SEQ ID NO:3i.

In some embodiments, the antigen-binding molecule is capable of binding to a polypeptide comprising or consisting of the amino acid sequence shown in SEQ ID NO:6. In some embodiments, the antigen-binding molecule is capable of binding to a polypeptide comprising or consisting of the amino acid sequence shown in SEQ ID NO:31. In some embodiments, the antigen-binding molecule is capable of binding to a peptide or polypeptide comprising or consisting of the amino acid sequence shown in SEQ ID NO:322. In some embodiments, the antigen-binding molecule is capable of binding to a peptide or polypeptide comprising or consisting of the amino acid sequence shown in SEQ ID NO:26. In some embodiments, the antigen-binding molecule is capable of binding to a peptide or polypeptide comprising or consisting of the amino acid sequence shown in SEQ ID NO:27. In some embodiments, the antigen-binding molecule is capable of binding to a peptide or polypeptide comprising or consisting of the amino acid sequence shown in SEQ ID NO:28. In some embodiments, the antigen-binding molecule is capable of binding to a peptide or polypeptide comprising or consisting of the amino acid sequence shown in SEQ ID NO:29. In some embodiments, the antigen-binding molecule is capable of binding to a peptide or polypeptide comprising or consisting of the amino acid sequence shown in SEQ ID NO:30.

In some embodiments, the antigen-binding molecule does not bind to the region of VISTA bound by IGN175A (described e.g. in WO 2014/197849 A2). In some embodiments, the antigen-binding molecule does not bind to the region of VISTA bound by an antigen-binding molecule comprised of a polypeptide consisting of the sequence of SEQ ID NO:267 and a polypeptide consisting of the sequence of SEQ ID NO:268.

In some embodiments, the antigen-binding molecule does not compete with IGN175A (described e.g. in WO 2014/197849 A2) for binding to VISTA. In some embodiments, the antigen-binding molecule does not compete with an antigen-binding molecule comprised of a polypeptide consisting of the sequence of SEQ ID NO:267 and a polypeptide consisting of the sequence of SEQ ID NO:268 for binding to VISTA.

The ability of a given antigen-binding molecule to compete with IGN175A or the antigen-binding molecule comprised of a polypeptide consisting of the sequence of SEQ ID NO:267 and a polypeptide consisting of the sequence of SEQ ID NO:268 for binding to VISTA can be analysed e.g. by competition ELISA, or by epitope binning as described in Abdiche et al., J Immunol Methods (2012) 382(-2):101-116 (hereby incorporated by reference in its entirety). Epitope binning can be performed e.g. by BLI analysis, e.g. as described in Example 8 of the present application.

In some embodiments, the antigen-binding molecule is not capable of binding to a peptide consisting of the amino acid sequence shown in SEQ ID NO:275.

As used herein, a “peptide” refers to a chain of two or more amino acid monomers linked by peptide bonds. A peptide typically has a length in the region of about 2 to 50 amino acids. A “polypeptide” is a polymer chain of two or more peptides. Polypeptides typically have a length greater than about 50 amino acids.

The ability of an antigen-binding molecule to bind to a given peptide/polypeptide can be analysed by methods well known to the skilled person, including analysis by ELISA, immunoblot (e.g. western blot), immunoprecipitation, surface plasmon resonance and biolayer interferometry.

In some embodiments, the antigen-binding molecule is capable of binding the same region of VISTA, or an overlapping region of VISTA, to the region of VISTA which is bound by an antibody comprising the VH and VL sequences of one of clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2- D2, 13D5p, 13D5-1, 13D5-13, 5M1-A11 or 9M2-C12.

In some embodiments, the antigen-binding molecule is capable of binding to a region of VISTA which is different to the region of VISTA bound by IGN175A (described e.g. in WO 2014/197849 A2). In some embodiments, the antigen-binding molecule is capable of binding to a region of VISTA which is different to the region of VISTA bound by an antigen-binding molecule comprised of a polypeptide consisting of the sequence of SEQ ID NO:267 and a polypeptide consisting of the sequence of SEQ ID NO:268.

In some embodiments, the antigen-binding molecule is capable of binding to a region of VISTA which does not overlap the region of VISTA bound by IGN175A (described e.g. in WO 2014/197849 A2). In some embodiments, the antigen-binding molecule is capable of binding to a region of VISTA which does not overlap with the region of VISTA bound by an antigen-binding molecule comprised of a polypeptide consisting of the sequence of SEQ ID NO:267 and a polypeptide consisting of the sequence of SEQ ID NO:268.

In some embodiments, the antigen-binding molecule binds to VISTA through contact with residues of VISTA which are non-identical to the residues of VISTA which are contacted by VSTB112 (described e.g. in WO 2015/097536 A2). In some embodiments, the antigen-binding molecule binds to VISTA through contact with residues of VISTA which are non-identical to the residues of VISTA which are contacted by an antigen-binding molecule comprised of a polypeptide consisting of the sequence of SEQ ID NO:269 and a polypeptide consisting of the sequence of SEQ ID NO:270.

In some embodiments, the epitope for the antigen-binding molecule is non-identical to the epitope for VSTB112. In some embodiments, the epitope for the antigen-binding molecule is non-identical to the epitope for an antigen-binding molecule comprised of a polypeptide consisting of the sequence of SEQ ID NO:269 and a polypeptide consisting of the sequence of SEQ ID NO:270.

The region of a peptide/polypeptide to which an antibody binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments, the antigen-binding molecule of the present disclosure binds to VISTA in a region which is accessible to an antigen-binding molecule (i.e., an extracellular antigen-binding molecule) when VISTA is expressed at the cell surface (i.e. in or at the cell membrane). In some embodiments, the antigen-binding molecule is capable of binding to VISTA expressed at the cell surface of a cell expressing VISTA. In some embodiments, the antigen-binding molecule is capable of binding to VISTA-expressing cells (e.g. CD14+ monocytes (such as monocyte-derived suppressor cells (MDSCs)) and/or CD33+ myeloid cells, tumor associated macrophages (TAMs), and neutrophils).

The ability of an antigen-binding molecule to bind to a given cell type can be analysed by contacting cells with the antigen-binding molecule, and detecting antigen-binding molecule bound to the cells, e.g. after a washing step to remove unbound antigen-binding molecule. The ability of an antigen-binding molecule to bind to immune cell surface molecule-expressing cells and/or cancer cell antigen-expressing cells can be analysed by methods such as flow cytometry and immunofluorescence microscopy.

The antigen-binding molecule of the present disclosure may be an antagonist of VISTA. In some embodiments, the antigen-binding molecule is capable of inhibiting a function or process (e.g. interaction, signalling or other activity) mediated by VISTA and/or an interaction partner for VISTA (e.g. LRIG1, VSIG3, PSGL-1, VSIG8). Herein, ‘inhibition’ refers to a reduction, decrease or lessening relative to a control condition. An antigen-binding molecule which inhibits a given interaction/activity/process may be referred to as inhibitor or antagonist of the interaction/activity/process, and may be said to ‘block’ or ‘neutralise’ the interaction/activity/process.

VISTA-binding antigen-binding molecules described herein are able to inhibit VISTA-mediated functions/processes by a mechanism not requiring Fc-mediated functions such as ADCC, ADCP and CDC. That is, VISTA-binding antigen-binding molecules described herein are able to inhibit the immunosuppressive activity of VISTA-expressing cells without the need to elicit ADCC, ADCP and/or CDC.

In particular, VISTA-binding antigen-binding molecules described herein are able to inhibit VISTA via a mechanism not requiring binding to Fcγ receptors and/or binding to C1q.

In some embodiments, the antigen-binding molecule of the present disclosure is capable of inhibiting interaction between VISTA and an interaction partner for VISTA (e.g. LRIG1, VSIG3, PSGL-1, VSIG8).

In some embodiments, the antigen-binding molecule is capable of inhibiting interaction between VISTA and an interaction partner for VISTA that binds to the C-C′ region of VISTA.

In some embodiments, an interaction partner for VISTA that binds to the C-C′ region of VISTA binds to the region of VISTA shown in SEQ ID NO:344. In some embodiments, an interaction partner for VISTA that binds to the C-C′ region of VISTA binds to a polypeptide comprising or consisting of the amino acid sequence shown in SEQ ID NO:344. In some embodiments, an interaction partner for VISTA that binds to the C-C′ region of VISTA contacts the region of VISTA shown in SEQ ID NO:344. In some embodiments, an interaction partner for VISTA that binds to the C-C′ region of VISTA binds to VISTA via contact with one or more amino acids of the region shown in SEQ ID NO:344.

In some embodiments, an interaction partner for VISTA that binds to the C-C′ region of VISTA is selected from LRIG1 and VSIG3. In some embodiments, an interaction partner for VISTA is LRIG1. In some embodiments, an interaction partner for VISTA is VSIG3.

In some embodiments, the antigen-binding molecule of the present disclosure is capable of inhibiting interaction between VISTA and LRIG1. In some embodiments, the antigen-binding molecule of the present disclosure is capable of inhibiting interaction between VISTA and PSGL-1. In some embodiments, the antigen-binding molecule of the present disclosure is capable of inhibiting interaction between VISTA and VSIG3.

The ability of an antigen-binding molecule to inhibit interaction between two factors can be determined for example by analysis of interaction in the presence of, or following incubation of one or both of the interaction partners with, the antibody/fragment. Assays for determining whether a given antigen-binding molecule is capable of inhibiting interaction between two interaction partners include competition ELISA assays and analysis by SPR.

An antigen-binding molecule which is capable of inhibiting a given interaction (e.g. between VISTA and an interaction partner for VISTA) is identified by the observation of a reduction/decrease in the level of interaction between the interaction partners in the presence of—or following incubation of one or both of the interaction partners with—the antigen-binding molecule, as compared to the level of interaction in the absence of the antigen-binding molecule (or in the presence of an appropriate control antigen-binding molecule). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the antigen-binding molecule may be labelled or used in conjunction with a detectable entity for the purposes of detecting and/or measuring the level of interaction.

The ability of an antigen-binding molecule to inhibit interaction between two binding partners can also be determined by analysis of the downstream functional consequences of such interaction. For example, downstream functional consequences of interaction between VISTA and an interaction partner for VISTA may include VISTA-mediated signalling. For example, the ability of an antigen-binding molecule to inhibit interaction of VISTA and an interaction partner for VISTA may be determined by analysis of production of IL-2, IFN-γ and/or IL-17 in an MLR assay.

In some embodiments, the antigen-binding molecule of the present disclosure is capable of inhibiting interaction between VISTA and an interaction partner for VISTA (e.g. LRIG1, VSIG3, PSGL-1, VSIG8) to less than less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction between VISTA and the binding partner for VISTA in the absence of the antigen-binding molecule (or in the presence of an appropriate control antigen-binding molecule).

In some embodiments, the antigen-binding molecule inhibits VISTA-mediated signalling. In some embodiments, VISTA-mediated signalling may be signalling mediated by a polypeptide complex comprising VISTA. In some embodiments, VISTA-mediated signalling may be signalling mediated by a polypeptide complex comprising VISTA and an interaction partner for VISTA (e.g. LRIG1, VSIG3, PSGL-1, VSIG8). In some embodiments, VISTA-mediated signalling may be signalling mediated by a polypeptide complex comprising VISTA and an interaction partner for VISTA that binds to the C-C′ region of VISTA (e.g. LRIG1 or VSIG3). In some embodiments, VISTA-mediated signalling may be signalling mediated by a polypeptide complex comprising VISTA and LRIG1. In some embodiments, VISTA-mediated signalling may be signalling mediated by a polypeptide complex comprising VISTA and VSIG3.

VISTA-mediated signalling can be analysed e.g. using an assay of effector immune cell number/activity, such as an MLR assay as described in the experimental examples herein. Inhibition of VISTA-mediated signalling can be identified by detection of an increase in the number and/or activity of effector immune cells, as determined e.g. by an increase in production of IL-2, IFN-γ and/or IL-17.

In some embodiments, the antigen-binding molecule is able to inhibit VISTA-mediated signalling by a mechanism not requiring or involving Fc-mediated function. In some embodiments, the antigen-binding molecule is able to inhibit VISTA-mediated signalling independently of Fc-mediated function. That is, in some embodiments, the antigen-binding molecule is able to inhibit VISTA-mediated signalling in an Fc region-independent manner.

The ability of an antigen-binding molecule to inhibit VISTA-mediated signalling by a mechanism not requiring/involving Fc-mediated function can be evaluated e.g. by analysing the ability of the antigen-binding molecule provided in a format lacking a functional Fc region to inhibit VISTA-mediated signalling.

For example, the effect on VISTA-mediated signalling can be investigated using an antigen-binding molecule comprising a ‘silent’ Fc region (e.g. comprising LALA PG substitutions), or using an antigen-binding molecule provided in a format lacking an Fc region (e.g. scFv, Fab etc.).

In some embodiments, the antigen-binding molecule is able to inhibit VISTA-mediated signalling by a mechanism not involving ADCC. In some embodiments, the antigen-binding molecule is able to inhibit VISTA-mediated signalling by a mechanism not involving ADCP. In some embodiments, the antigen-binding molecule is able to inhibit VISTA-mediated signalling by a mechanism not involving CDC.

In some embodiments, the antigen-binding molecule is able to inhibit VISTA-mediated signalling by a mechanism not requiring binding of the antigen-binding molecule to an Fc receptor. In some embodiments, the antigen-binding molecule is able to inhibit VISTA-mediated signalling by a mechanism not requiring binding of the antigen-binding molecule to an Fcγ receptor. In some embodiments, the antigen-binding molecule is able to inhibit VISTA-mediated signalling by a mechanism not requiring binding of the antigen-binding molecule to one or more of FcγRI, FcγRIIa, FcγRIIb, FcγRIIc, FcγRIIIa and FcγRIIIb. In some embodiments, the antigen-binding molecule is able to inhibit VISTA-mediated signalling by a mechanism not requiring binding to FcγRIIIa. In some embodiments, the antigen-binding molecule is able to inhibit VISTA-mediated signalling by a mechanism not requiring binding to FcγRIIa. In some embodiments, the antigen-binding molecule is able to inhibit VISTA-mediated signalling by a mechanism not requiring binding to FcγRIIb. In some embodiments, the antigen-binding molecule is able to inhibit VISTA-mediated signalling by a mechanism not requiring binding to a complement protein. In some embodiments, the antigen-binding molecule is able to inhibit VISTA-mediated signalling by a mechanism not requiring binding to C1 q. In some embodiments, the antigen-binding molecule is able to inhibit VISTA-mediated signalling by a mechanism not requiring N297 glycosylation.

In some embodiments, the antigen-binding molecule of the present disclosure is capable of increasing killing of VISTA-expressing cells. Killing of VISTA-expressing cells may be increased through an effector function of the antigen-binding molecule. In embodiments wherein antigen-binding molecule comprises an Fc region the antigen-binding molecule may increase killing of VISTA-expressing cells through one or more of complement dependent cytotoxicity (CDC), antibody-dependent cell-mediated cytotoxicity (ADCC) and antibody-dependent cellular phagocytosis (ADCP).

An antigen-binding molecule which is capable of increasing killing of VISTA-expressing cells can be identified by observation of an increased level of killing of VISTA-expressing cells in the presence of—or following incubation of the VISTA-expressing cells with—the antigen-binding molecule, as compared to the level of cell killing detected in the absence of the antigen-binding molecule (or in the presence of an appropriate control antigen-binding molecule), in an appropriate assay. Assays of CDC, ADCC and ADCP are well known the skilled person. The level of killing of VISTA-expressing cells can also be determined by measuring the number/proportion of viable and/or non-viable VISTA-expressing cells following exposure to different treatment conditions.

In some embodiments, the antigen-binding molecule of the present disclosure is capable of increasing killing of VISTA-expressing cells (e.g. VISTA-expressing MDSCs) to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of killing observed in the absence of the antigen-binding molecule (or in the presence of an appropriate control antigen-binding molecule).

In some embodiments, the antigen-binding molecule of the present disclosure is capable of reducing the number of VISTA-expressing cells (e.g. VISTA-expressing MDSCs) to less than less than 1 times, e.g. 23 0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number of VISTA-expressing cells (e.g. VISTA-expressing MDSCs, TAMs, neutrophils) detected following incubation in the absence of the antigen-binding molecule (or following incubation in the presence of an appropriate control antigen-binding molecule), in a comparable assay.

In some embodiments, the antigen-binding molecule is a non-depleting antigen-binding molecule. That is, in some embodiments, the antigen-binding molecule does not cause substantial depletion of VISTA-expressing cells. In some embodiments, the antigen-binding molecule does not elicit/increase ADCC, ADCP and/or CDC against VISTA-expressing cells.

In some embodiments, the antigen-binding molecule of the present disclosure does not induce/increase killing of VISTA-expressing cells, e.g. in embodiments wherein the antigen-binding molecule lacks an Fc region, or embodiments wherein the antigen-binding molecule comprises an Fc region which is not able to induce an Fc-mediated antibody effector function. In some embodiments, the antigen-binding molecule of the present disclosure does not reduce the number/proportion of VISTA-expressing cells.

In some embodiments, the antigen-binding molecule of the present disclosure (i) inhibits VISTA-mediated signalling, and (ii) does not induce/increase killing of VISTA-expressing cells. In some embodiments, the antigen-binding molecule of the present disclosure (i) inhibits VISTA-mediated signalling, and (ii) does not reduce the number/proportion of VISTA-expressing cells.

This can be particularly advantageous, because VISTA is expressed by cells that it is not desirable to deplete. For example, VISTA is expressed at low levels by immune cells (e.g. certain types of T cells and dendritic cells) that it is not desirable to kill or reduce the number/proportion of.

In some embodiments, the antigen-binding molecule of the present disclosure is capable of increasing the number and/or activity of effector immune cells relative to a negative control condition, e.g. in an appropriate in vitro assay, or in vivo. By way of explanation, the antigen-binding molecules of the present disclosure may be capable of releasing effector immune cells from MDSC-mediated suppression of effector immune cell proliferation and function. In some embodiments, the effector immune cells may be e.g. CD8+ T cells, CD8+ cytotoxic T lymphocytes (CD8+CTLs), CD4+ T cells, CD4+T helper cells, NK cells, IFNγ-producing cells, memory T cells, central memory T cells, antigen-experienced T cells or CD45RO+ T cells.

In some embodiments, the antigen-binding molecule of the present disclosure is capable of increasing the number of an effector immune cell type to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the number observed in the absence of the antigen-binding molecule (or in the presence of an appropriate control antigen-binding molecule). In some embodiments, the antigen-binding molecule of the present disclosure is capable of increasing the level of a correlate of effector immune cell activity to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

Cell numbers and proportions can be determined e.g. by flow cytometry analysis using antibodies allowing detection of cell types. Cell division can be analysed, for example, by in vitro analysis of incorporation of ³H-thymidine or by CFSE dilution assay, e.g. as described in Fulcher and Wong, Immunol Cell Biol (1999) 77(6): 559-564, hereby incorporated by reference in entirety. Effector immune cell activity can be analysed by measuring a correlate of such activity. In some embodiments effector immune cell activity can be determined e.g. by analysis of production of IL-2, IFN-γ and/or IL-17.

In some embodiments, the antigen-binding molecule of the present disclosure is capable of decreasing the level of immune suppression mediated by VISTA-expressing cells. A change in the level of immune suppression may be determined using methods to measure the expression of arginase 1 and/or the production of reactive oxygen species (ROS) by VISTA-expressing cells, for example as described in Ochoa et al., Ann Surg. 2001 March; 233(3): 393-399 and Dikalov and Harrison Antioxid Redox Signal. 2014 Jan. 10; 20(2): 372-382.

In some embodiments, the antigen-binding molecule of the present disclosure is capable of increasing antigen presentation by antigen-presenting cells, e.g. as determined using a suitable assay of antigen presentation. In some embodiments, the antigen-binding molecule of the present disclosure is capable of increasing phagocytosis by phagocytic cells (e.g. neutrophils, monocytes, macrophages, mast cells, and/or dendritic cells), e.g. as determined using a suitable assay of the level of phagocytosis.

In some embodiments, the antigen-binding molecule of the present disclosure is capable of increasing the number and/or activity of antigen-presenting cells (e.g. CD11 b+MHCII+ cells) relative to a negative control condition, e.g. in an appropriate in vitro assay, or in vivo (e.g. in a tumor). In some embodiments, the antigen-binding molecule is capable of increasing the number and/or activity of macrophages (e.g. CD11 b+F4/80+ cells) relative to a negative control condition, e.g. in an appropriate in vitro assay, or in vivo (e.g. in a tumor). In some embodiments, the antigen-binding molecule is capable of increasing the number and/or activity of dendritic cells (e.g. CD11c+ cells) relative to a negative control condition, e.g. in an appropriate in vitro assay, or in vivo (e.g. in a tumor).

In some embodiments, the antigen-binding molecule of the present disclosure is capable of increasing the number of a cell type recited in the preceding paragraph to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the number observed in the absence of the antigen-binding molecule (or in the presence of an appropriate control antigen-binding molecule). In some embodiments, the antigen-binding molecule of the present disclosure is capable of increasing the level of a correlate of activity of a cell type recited in the preceding paragraph to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing production of IL-6 by immune cells. The immune cells may be e.g. PBMCs, lymphocytes, T cells, B cells, NK cells, or monocytes. In some embodiments, the immune cells are monocytes. In some embodiments, the antigen-binding molecule is capable of increasing production of IL-6 by immune cells following stimulation, e.g. with LPS. The ability of an antigen-binding molecule to increase production of IL-6 by immune cells can be analysed in an in vitro assay e.g. as described in Example 10 herein. Such methods may comprise stimulating monocytes (e.g. THP1 cells) with LPS, and incubating the stimulated cells with the antigen-binding molecule.

In some embodiments, the antigen-binding molecule of the present disclosure is capable of increasing IL-6 production by immune cells (e.g. LPS-stimulated THP1 cells) to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing the number and/or activity of Th1/Th17 cells. In some embodiments, the antigen-binding molecule is capable of upregulating the Th1/Th17 response. In some embodiments, the antigen-binding molecule favours the Th1/Th17 response over the Th2 response. In some embodiments, the antigen-binding molecule of the present disclosure is capable of increasing T cell proliferation, IL-2 production, IFN-γ production, TNFα production and/or IL-17A production in a Mixed Lymphocyte Reaction (MLR) assay. MLR assays may be performed as described in Bromelow et al J. Immunol Methods, 2001 January 1; 247(1-2):1-8, (hereby incorporated by reference in its entirety), or as described in the experimental examples herein. IL-2, IFNγ and/or IL-17 production may be analysed e.g. by antibody-based methods well known to the skilled person, such as western blot, immunohistochemistry, immunocytochemistry, flow cytometry, ELISA, ELISPOT, or by reporter-based methods.

In some embodiments, the antigen-binding molecule of the present disclosure is capable of increasing T cell (e.g. Th1/Th17 cell) proliferation, IL-2 production, IFN-γ production and/or IL-17 production in an MLR assay to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing T cell (e.g. Th1/Th17 cell) proliferation, IFN-γ production and/or TNFa production, e.g. in the presence of VISTA/VISTA expressing cells. Antigen-binding molecules may be evaluated for such properties e.g. in in vitro assays as described in the experimental examples herein.

In some embodiments, the antigen-binding molecule of the present disclosure is capable of increasing T cell (e.g. Th1/Th17 cell) proliferation, IFN-γ production and/or TNFa production (e.g. in the presence of VISTA/VISTA expressing cells) to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing T cell (e.g. CD4+ T cell and/or CD8+ T cell, e.g. Th1/Th17 cell) proliferation to a greater extent than a VISTA-binding antibody disclosed in the prior art (e.g. VSTB112, described e.g. in WO 2015/097536 A2). T cell proliferation may be evaluated in an in vitro assay e.g. as described in Example 9 herein, and may involve stimulating T cell proliferation by culture in the presence of agonist anti-CD3 antibody. In some embodiments, the antigen-binding molecule of the present disclosure is capable of increasing T cell proliferation in such an assay to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level proliferation induced by the prior art VISTA-binding antibody (e.g. VSTB112).

In some embodiments, the antigen-binding molecule of the present disclosure is capable of increasing T cell-mediated lysis of cancer cells to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing T cell-mediated lysis of cancer cells, e.g. in the presence of VISTA/VISTA expressing cells. Antigen-binding molecules may be evaluated for such properties e.g. in in vitro assays as described in the experimental examples herein.

In some embodiments, the antigen-binding molecule of the present disclosure is capable of increasing T cell-mediated lysis (e.g. in the presence of VISTA/VISTA expressing cells) to more than 1 times, e.g. 21.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, 26 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing IL-6 production by THP1 cells to a greater extent than a VISTA-binding antibody disclosed in the prior art (e.g. VSTB112, described e.g. in WO 2015/097536 A2). IL-6 production by THP1 cells may be evaluated in an in vitro assay e.g. as described in Example 10 herein, and may involve stimulating THP1 cells with LPS. In some embodiments, the antigen-binding molecule of the present disclosure is capable of increasing IL-6 production in such an assay to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level induced by the prior art VISTA-binding antibody (e.g. VSTB112).

In some embodiments, the antigen-binding molecule of the present disclosure is capable of: reducing the number and/or activity of suppressor immune cells, inhibiting proliferation of suppressor immune cells, and/or reducing the proportion of suppressor immune cells within a population of cells (e.g. CD45+ cells, e.g. CD45+ cells obtained from a tumor) relative to control condition, e.g. as determined in an appropriate in vitro assay, or in vivo.

The suppressor immune cells may be e.g. VISTA-expressing cells, Arg1-expressing cells, MDSCs, granulocytic MDSCs (g-MDSCs) or monocytic MDSCs (m-MDSCs). In some embodiments, the suppressor immune cells are CD11 b+GR1+MHCII− cells.

In some embodiments, the reduction in the number/activity/proliferation/proportion is to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, 50.15 times, ≤0.1 times, ≤0.05 times, or ≤0.01 times the number/activity/proliferation/proportion observed in the absence of the antigen-binding molecule (or in the presence of an appropriate control antigen-binding molecule).

In some embodiments, the antigen-binding molecule is able to reduce the number/activity/proliferation/proportion of suppressor immune cells by a mechanism not involving Fc-mediated function. In some embodiments, the antigen-binding molecule is able to reduce the number/activity/proliferation/proportion of suppressor immune cells independently of Fc-mediated function (i.e. in an Fc region-independent manner). In some embodiments, the antigen-binding molecule is able to reduce the number/activity/proliferation/proportion of suppressor immune cells by a mechanism not involving ADCC, ADCP and/or CDC. In some embodiments, the antigen-binding molecule is able to reduce the number/activity/proliferation/proportion of suppressor immune cells by a mechanism not involving depletion of VISTA-expressing cells.

In some embodiments, the antigen-binding molecule of the present disclosure inhibits the development and/or progression of cancer in vivo.

In some embodiments, the antigen-binding molecule causes an increase in the killing of cancer cells, e.g. by effector immune cells. In some embodiments, the antigen-binding molecule causes a reduction in the number of cancer cells in vivo, e.g. as compared to an appropriate control condition. In some embodiments, the antigen-binding molecule inhibits tumor growth, e.g. as determined by measuring tumor size/volume over time.

In some embodiments, the antigen-binding molecule of the present disclosure is capable of increasing serum levels of IFN-γ and/or IL-23 in mice treated with the antigen-binding molecule. Serum levels of IFN-γ and/or IL-23 can be analysed e.g. by ELISA of serum derived from blood samples obtained from the mice. In some embodiments, administration of the antigen-binding molecule of the present disclosure increases serum level of IFN-γ and/or IL-23 to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of administration of the antigen-binding molecule (or the level observed following administration of an appropriate control antigen-binding molecule).

The antigen-binding molecule of the present disclosure may be analysed for the ability to inhibit development and/or progression of cancer in an appropriate in vivo model, e.g. cell line-derived xenograft model such as CT26 cell-derived model, a 4T-1 cell-derived model, an LL2 cell-derived model, a B116 cell-derived model, or an EL4 cell-derived model. The cancer may be a cancer in which VISTA-expressing cells and/or MDSCs (e.g. VISTA-expressing MDSCs, TAMs, neutrophils) are pathologically implicated.

Cancers in which MDSCs are ‘pathologically implicated’ include cancers in which MDSCs, or an increased number/proportion of MDSCs, is positively associated with onset, development or progression of the cancer, and/or severity of one or more symptoms of the cancer, or a cancer for which MDSCs, or an increased number/proportion of MDSCs, is a risk factor for the onset, development or progression of the cancer. The cancer may comprise MDSCs in an organ/tissue which is affected by the disease (e.g. an organ/tissue in which the symptoms of the disease/condition manifest) or in a tumor.

In some embodiments, administration of an antigen-binding molecule according to the present disclosure may cause one or more of: inhibition of the development/progression of the cancer, a delay to/prevention of onset of the cancer, a reduction in/delay to/prevention of tumor growth, a reduction in/delay to/prevention of metastasis, a reduction in the severity of the symptoms of the cancer, a reduction in the number of cancer cells, a reduction in tumour size/volume, and/or an increase in survival (e.g. progression free survival), e.g. as determined in an CT26 cell, 4T-1 cell, an LL2 cell, a B116 cell, or an EL4 cell-derived xenograft model.

In some embodiments, administration of the antigen-binding molecule of the present disclosure is capable of inhibiting greater than 5%, e.g. ≥10%, ≥15%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90% or ≥95% of the tumor growth observed in the absence of administration of the antigen-binding molecule (or following administration of an appropriate control antigen-binding molecule).

In some embodiments, administration of the antigen-binding molecule at the dose and periodicity described in the experiments in the CT26 cell-derived model of the experimental examples of the present disclosure inhibits greater than 5%, e.g. ≥10%, ≥15%, ≥20%, ≥25%, ≥30%, ≥35%, ≥40%, ≥45%, ≥50%, ≥55%, ≥60%, ≥65%, ≥70%, ≥75% or ≥80% of the tumor growth observed in the absence of administration of the antigen-binding molecule (or following administration of an appropriate control antigen-binding molecule). In some embodiments, administration of the antigen-binding molecule at the dose and periodicity described in the experiments in the 4T-1 cell-derived model of the experimental examples of the present disclosure inhibits greater than 5%, e.g. ≥10%, ≥15%, ≥20%, ≥25%, ≥30%, ≥35%, ≥40%, 245%, 250% of the tumor growth observed in the absence of administration of the antigen-binding molecule (or following administration of an appropriate control antigen-binding molecule).

In some embodiments, administration of an antigen-binding molecule according to the present disclosure is not associated with cytokine release syndrome. In some embodiments, administration of an antigen-binding molecule is not associated with the systemic activation of leukocytes (e.g. B cells, T cells, NK cells, macrophages, dendritic cells and/or monocytes). In some embodiments, administration of an antigen-binding molecule is not associated with systemic upregulation of expression of inflammatory cytokines and/or chemokines (e.g. IL-6, IFN-γ, IL-8, IL-10, GM-CSF, MIP-1a/p, MCP-1, CXCL9 and/or CXCL10) by leukocytes.

Aspects and embodiments of the present disclosure are concerned in particular with antigen-binding molecules capable of inhibiting interaction between VISTA and an interaction partner for VISTA that binds to the C-C′ region of VISTA (e.g. LRIG1 or VSIG3).

In some embodiments, an antigen-binding molecule according to the present disclosure binds to VISTA in the region which is bound by an interaction partner for VISTA that binds to the C-C′ region of VISTA (e.g. LRIG1 or VSIG3). In some embodiments, the antigen-binding molecule is a competitive inhibitor of binding of an interaction partner for VISTA that binds to the C-C′ region of VISTA (e.g. LRIG1 or VSIG3) to VISTA. In some embodiments, the antigen-binding molecule is an allosteric inhibitor of binding of an interaction partner for VISTA that binds to the C-C′ region of VISTA (e.g. LRIG1 or VSIG3) to VISTA. In some embodiments, the antigen-binding molecule displaces an interaction partner for VISTA that binds to the C-C′ region of VISTA (e.g. LRIG1 or VSIG3) from a complex comprising VISTA and an interaction partner for VISTA that binds to the C-C′ region of VISTA (e.g. LRIG1 or VSIG3). In some embodiments, the antigen-binding molecule does not bind to a complex comprising VISTA and an interaction partner for VISTA that binds to the C-C′ region of VISTA (e.g. LRIG1 or VSIG3).

The ability of an antigen-binding molecule to inhibit interaction between VISTA and an interaction partner for VISTA that binds to the C-C′ region of VISTA (e.g. LRIG1 or VSIG3) can be determined for example by analysis of interaction in the presence of, or following incubation of one or both of the interaction partners with, the antigen-binding molecule. Assays for determining whether a given antigen-binding molecule is capable of inhibiting interaction between VISTA and an interaction partner for VISTA that binds to the C-C′ region of VISTA (e.g. LRIG1 or VSIG3) include competition ELISA assays and analysis by SPR.

An antigen-binding molecule which is capable of inhibiting interaction between VISTA and an interaction partner for VISTA that binds to the C-C′ region of VISTA (e.g. LRIG1 or VSIG3) may identified by the observation of a reduction/decrease in the level of interaction between the interaction partners in the presence of—or following incubation of one or both of the interaction partners with—the antigen-binding molecule, as compared to the level of interaction in the absence of the antigen-binding molecule (or in the presence of an appropriate control antigen-binding molecule known not to inhibit such interaction). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the antigen-binding molecule may be labelled or used in conjunction with a detectable entity for the purposes of detecting and/or measuring the level of interaction.

For example, Example 2 of the present disclosure describes an ELISA assay in which VISTA-binding antigen-binding molecules were analysed for their ability to inhibit interaction between VISTA and LRIG1. Briefly, wells of plates were coated with Fc-tagged human VISTA proteins, and after blocking and incubation with VISTA-binding antigen-binding molecules, HIS-tagged LRIG1 was added to the wells. VISTA-LRIG1 complexes were detected by detection of captured HIS-tagged LRIG1, using an HRP-conjugated anti-HIS antibody, and subsequent development with 3,3′,5,5′-tetramethylbenzidine. Inhibition of interaction between VISTA and LRIG1 by a given VISTA-binding antigen-binding molecule is inferred in this assay based on detection of a level of HRP activity which is less than the level of HRP activity observed in a control condition in which an isotype-matched antigen-binding molecule which does not bind VISTA.

In some embodiments, an antigen-binding molecule according to the present disclosure inhibits interaction between VISTA and an interaction partner for VISTA that binds to the C-C′ region of VISTA (e.g. LRIG1 or VSIG3) to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction observed in the absence of the antigen-binding molecule, or in the presence of the same quantity of an appropriate control antigen-binding molecule known not to inhibit interaction between VISTA and the interaction partner for VISTA, in a given assay.

In some embodiments, an antigen-binding molecule according to the present disclosure inhibits interaction between VISTA and an interaction partner for VISTA that binds to the C-C′ region of VISTA (e.g. LRIG1 or VSIG3) with an IC₅₀ (e.g. as determined by ELISA, e.g. an ELISA as described in the Examples of the present disclosure) of ≤1 μM or less, e.g. one of 5500 nM, ≤100 nM, ≤50 nM, ≤40 nM, ≤30 nM, ≤20 nM, ≤10 nM, ≤5 nM, ≤4 nM.

Aspects and embodiments of the present disclosure are concerned in particular with antigen-binding molecules capable of inhibiting interaction between VISTA and LRIG1.

In some embodiments, an antigen-binding molecule according to the present disclosure binds to VISTA in the region which is bound by LRIG1. In some embodiments, the antigen-binding molecule is a competitive inhibitor of binding of LRIG1 to VISTA. In some embodiments, the antigen-binding molecule is an allosteric inhibitor of binding of LRIG1 to VISTA. In some embodiments, the antigen-binding molecule displaces LRIG1 from a complex comprising VISTA and LRIG1. In some embodiments, the antigen-binding molecule does not bind to a complex comprising VISTA and LRIG1.

In some embodiments, an antigen-binding molecule according to the present disclosure inhibits interaction between VISTA and LRIG1 to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction observed in the absence of the antigen-binding molecule, or in the presence of the same quantity of an appropriate control antigen-binding molecule known not to inhibit interaction between VISTA and LRIG1, in a given assay.

In some embodiments, an antigen-binding molecule according to the present disclosure inhibits interaction between VISTA and LRIG1 with an IC₅₀ (e.g. as determined by ELISA, e.g. an ELISA as described in the Examples of the present disclosure) of ≤1 μM or less, e.g. one of 5500 nM, ≤100 nM, ≤50 nM, ≤40 nM, ≤30 nM, ≤20 nM, ≤10 nM, ≤5 nM, ≤4 nM.

Aspects and embodiments of the present disclosure are concerned in particular with antigen-binding molecules capable of inhibiting interaction between VISTA and VSIG3.

In some embodiments, an antigen-binding molecule according to the present disclosure binds to VISTA in the region which is bound by VSIG3. In some embodiments, the antigen-binding molecule is a competitive inhibitor of binding of VSIG3 to VISTA. In some embodiments, the antigen-binding molecule is an allosteric inhibitor of binding of VSIG3 to VISTA. In some embodiments, the antigen-binding molecule displaces VSIG3 from a complex comprising VISTA and VSIG3. In some embodiments, the antigen-binding molecule does not bind to a complex comprising VISTA and VSIG3.

In some embodiments, an antigen-binding molecule according to the present disclosure inhibits interaction between VISTA and VSIG3 to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction observed in the absence of the antigen-binding molecule, or in the presence of the same quantity of an appropriate control antigen-binding molecule known not to inhibit interaction between VISTA and VSIG3, in a given assay.

In some embodiments, an antigen-binding molecule according to the present disclosure inhibits interaction between VISTA and VSIG3 with an IC₅₀ (e.g. as determined by ELISA, e.g. an ELISA as described in the Examples of the present disclosure) of ≤1 μM or less, e.g. one of 5500 nM, ≤100 nM, ≤50 nM, ≤40 nM, ≤30 nM, ≤20 nM, ≤10 nM, ≤5 nM, ≤4 nM, ≤3 nM, ≤2 nM, ≤1 nM, ≤900 pM, ≤800 pM, ≤700 pM.

In some embodiments, the antigen-binding molecule of the present disclosure is capable of increasing the number and/or proportion of antigen-specific CD8+ T cells (e.g. gp70+CD8+ T cells) relative to a negative control condition, e.g. in an appropriate in vitro assay, or in vivo. The ability of an antigen-binding molecule to increase the number and/or proportion of antigen-specific CD8+ T cells can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule.

In some embodiments, the antigen-binding molecule of the present disclosure is capable of increasing the number of antigen-specific CD8+ T cells to more than a number observed in the absence of the antigen-binding molecule (or in the presence of an appropriate control antigen-binding molecule).

In some embodiments, the antigen-binding molecule of the present disclosure is capable of increasing the number or proportion of antigen-specific CD8+ T cells to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, ≥10 times, ≥20 times, ≥30 times, ≥40 times, or ≥50 times the number/proportion of antigen-specific CD8+ T cells observed in the absence of the antigen-binding molecule (or in the presence of an appropriate control antigen-binding molecule). In some embodiments, the antigen-binding molecule of the present disclosure is capable of increasing the number or proportion of antigen-specific CD8+ T cells by at least 1%, e.g. at least 2%, 3%, 4%, 5%, 10%, 15%, 20%, 25%, 30%, 35%, 40%, 45%, 50%, 55%, 60%, 65%, 70%, 75%, 80%, 85%, 90%, 95% or 100% the number/proportion of antigen-specific CD8+ T cells observed in the absence of the antigen-binding molecule (or in the presence of an appropriate control antigen-binding molecule).

Cell numbers and proportions can be determined e.g. by flow cytometry analysis using antibodies allowing detection of cell types. Antigen-specific CD8+ T cells can be identified by the presence and/or absence of one or more cell markers, e.g. gp70+, CD8+. By way of example, Example 8 illustrates the determination of the proportion of tumour antigen-specific CD8+ T cells by flow cytometric analysis.

Antigen-specific CD8+ T cells may be identified by use of peptide-MHC multimers (pMHC multimers) comprising an antigen of interest, e.g. an antigen expected to be found in a cancer/tumour.

In some embodiments, the antigen-binding molecule of the present disclosure is capable of increasing CD8+ T cell activity (e.g. cytotoxic CD8+ T cell activity) relative to a negative control condition, e.g. in an appropriate in vitro assay, or in vivo. The ability of an antigen-binding molecule to increase CD8+ T cell activity can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule.

In some embodiments, the antigen-binding molecule of the present disclosure is capable of increasing CD8+ T cell activity to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the activity of CD8+ T cells observed in the absence of the antigen-binding molecule (or in the presence of an appropriate control antigen-binding molecule). In some embodiments, the antigen-binding molecule of the present disclosure is capable of increasing the level of a correlate of CD8+ T cell activity to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, 25 times, ≥6 times, ≥7 times, ≥8 times, ≥9 times or ≥10 times the level observed in the absence of the antigen-binding molecule (or in the presence of an appropriate control antigen-binding molecule).

In some embodiments CD8+ T cell activity can be analysed by measuring a correlate of such activity. In some embodiments CD8+ T cell activity can be determined, e.g. by analysis of production of Granzyme B, CX3CR1, ICOS, CD27. By way of example, Example 8 illustrates the determination of CD8+ T cell activity by measuring the proportion of cells expressing Granzyme B, CX3CR1, ICOS and/or CD27 by flow cytometric analysis.

In some embodiments, the antigen-binding molecule of the present disclosure is capable of upregulating one or more cytotoxicity-associated markers relative to a negative control condition, e.g. in an appropriate in vitro assay, or in vivo. The ability of an antigen-binding molecule upregulate one or more cytotoxicity-associated markers can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule or an appropriate control antigen-binding molecule.

In some embodiments, the antigen-binding molecule of the present disclosure is capable of upregulating one or more cytotoxicity-associated markers to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule). In some embodiments, the antigen-binding molecule of the present disclosure is capable of increasing the level of one or more cytotoxicity-associated markers to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

Cytotoxicity-associated markers are well known to the skilled person, including Granzyme B (i.e. GZMB), CX3CR1, ICOS, CD27, TNFa, INFy, IL-2, CXCR3, TBX21, IL-4, CCR4, GATA3, IL-9, IL-10, IRF4, CCR6, KLRB1, IL-17, IRF4, RORc.

In some embodiments, the antigen-binding molecule of the present disclosure is capable of upregulating one or more genes associated with pro-inflammatory macrophage activation, e.g. in an appropriate in vitro assay, or in vivo. The ability of an antigen-binding molecule to upregulate one or more genes associated with pro-inflammatory macrophage activation can be analysed in an assay, e.g. as described in Example 9 herein. Such methods may comprise analysing the transcriptome of a tumour cell-line or tumour following administration of the antigen-binding molecule of an appropriate control antigen-binding molecule.

In some embodiments, the antigen-binding molecule of the present disclosure is capable of upregulating one or more genes associated with pro-inflammatory macrophage activation to more than 1 times, e.g. 21.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, 26 times, ≥7 times, ≥8 times, ≥9 times or ≥10 times the level observed in the absence of the antigen-binding molecule (or in the presence of an appropriate control antigen-binding molecule).

In some embodiments, the one or more genes associated with pro-inflammatory macrophage activation may be selected from those shown in FIG. 18. Gene expression can be determined by means well known to the skilled person. The level of RNA encoding one or more genes associated with pro-inflammatory macrophage activation can be determined e.g. by techniques such as RT-qPCR, northern blot, etc.

In some embodiments, the antigen-binding molecule of the present disclosure is capable of upregulating one or more genes associated with cytotoxic activity of T cells, e.g. in an appropriate in vitro assay, or in vivo. The ability of an antigen-binding molecule to upregulate one or more genes associated with cytotoxic activity of T cells can be analysed in an assay, e.g. as described in Example 9 herein. Such methods may comprise analysing the transcriptome of a tumour cell-line or tumour following administration of the antigen-binding molecule of an appropriate control antigen-binding molecule.

In some embodiments, the antigen-binding molecule of the present disclosure is capable of upregulating one or more genes associated with cytotoxic activity of T cells to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the one or more genes associated cytotoxic activity of T cells may be selected from those shown in FIG. 18. Gene expression can be determined by means well known to the skilled person. The level of RNA encoding one or more genes associated with cytotoxic activity of T cells can be determined e.g. by techniques such as RT-qPCR, northern blot, etc.

In some embodiments, the antigen-binding molecule of the present disclosure is capable of increasing production of Granzyme B by immune cells relative to a negative control condition, e.g. in an appropriate in vitro assay, or in vivo. The ability of an antigen-binding molecule to increase production of Granzyme B by immune cells can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule or an appropriate control antigen-binding molecule.

In some embodiments, the antigen-binding molecule of the present disclosure is capable of increasing production of Granzyme B by immune cells to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the production of Granzyme B by immune cells observed in the absence of the antigen-binding molecule (or in the presence of an appropriate control antigen-binding molecule). In some embodiments, the antigen-binding molecule of the present disclosure is capable of increasing the level of Granzyme B to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, 22 times, ≥3 times, ≥4 times, ≥5 times, ≥6 times, ≥7 times, ≥8 times, ≥9 times or ≥10 times the level of Granzyme B observed in the absence of the antigen-binding molecule (or in the presence of an appropriate control antigen-binding molecule).

In some embodiments, the antigen-binding molecule of the present disclosure is capable of increasing Granzyme B production in a Mixed Lymphocyte Reaction (MLR) assay. MLR assays may be performed as described in Bromelow et al J. Immunol Methods, 2001 Jan 1,247(1-2):1-8, (hereby incorporated by reference in its entirety). Granzyme B production may be analysed e.g. by antibody-based methods well known to the skilled person, such as western blot, immunohistochemistry, immunocytochemistry, flow cytometry, ELISA, ELISPOT, or by reporter-based methods.

In some embodiments, the antigen-binding molecule of the present disclosure is capable of reducing the level of T cell exhaustion relative to a negative control condition, e.g. in an appropriate in vitro assay, or in vivo. The ability of an antigen-binding molecule to reduce the level of T cell exhaustion can be analysed in an assay, e.g. as described in Example 10 herein. Such methods may comprise immunoprofiling of a tumour from a non-human animal model (e.g. a cell-line derived mouse model) of a cancer, following administration of the antigen-binding molecule or an appropriate control antigen-binding molecule.

In some embodiments, the antigen-binding molecule of the present disclosure is capable of reducing the level of T cell exhaustion to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of T cell exhaustion observed in the absence of the antigen-binding molecule (or in the presence of an appropriate control antigen-binding molecule). In some embodiments, the antigen-binding molecule of the present disclosure is capable of reducing the level of a correlate of T cell exhaustion to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of a correlate of T cell exhaustion in the absence of the antigen-binding molecule (or in the presence of an appropriate control antigen-binding molecule).

In some embodiments, T cell exhaustion can be analysed by determining the number and/or proportion of exhausted T cells. Cell numbers and proportions can be determined e.g. by flow cytometry analysis using antibodies allowing detection of cell types. Exhausted T cells can be identified by the presence and/or absence of one or more cell markers, e.g. gp70+, CD8+, PD-1+, IL-7Ra−. By way of example, Example 8 illustrates the determination of the proportion of exhausted T cells using flow cytometric analysis to detect gp70+CD8+PD-1+IL-7Ra− cells.

In some embodiments T cell exhaustion can be analysed by measuring a correlate of T cell exhaustion. In some embodiments T cell exhaustion can be determined e.g. by analysis of the presence/absence of cell markers of T cell exhaustion, e.g. PD-1+, LAG-3+, TIM-3+, IL-7Ra−.

In some embodiments, the antigen-binding molecule of the present disclosure is capable of reducing the number and/or proportion of tumour-associated macrophages (TAMs) relative to a negative control condition, e.g. in an appropriate in vitro assay, or in vivo. The ability of an antigen-binding molecule to reduce number and/or proportion of tumour-associated macrophages can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule or an appropriate control antigen-binding molecule.

In some embodiments, the antigen-binding molecule of the present disclosure is capable of reducing the number of tumour-associated macrophages to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number of tumour-associated macrophages observed in the absence of the antigen-binding molecule (or in the presence of an appropriate control antigen-binding molecule).

In some embodiments, the antigen-binding molecule of the present disclosure is capable of reducing the proportion of tumour-associated macrophages to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the proportion of tumour-associated macrophages observed in the absence of the antigen-binding molecule (or in the presence of an appropriate control antigen-binding molecule).

Cell numbers and proportions can be determined e.g. by flow cytometry analysis using antibodies allowing detection of cell types tumour-associated macrophages can be identified by the presence and/or absence of one or more cell markers, e.g CD45+, F4/80+, MHCII−. TAMs may exhibit as M2-type phenotype-acquired macrophages, e.g. exhibit the characteristics of M2-type macrophages (also known as alternative-activated macrophages). TAMs may secrete anti-inflammatory cytokines (e.g. such as IL-10, IL-13, and IL-4), express arginase-1, express mannose receptor (MR, CD206), and/or express scavenger receptors, e.g. MARCO. Tumour-associated macrophages are described in Lin et al., Journal of Hematology & Oncology (2019) 12:76 (which is hereby incorporated by reference in its entirety). By way of example, Example 8 illustrates the determination of the proportion of tumour-associated macrophages using flow cytometry to detect CD45+F4/80+MHCII− cells.

In some embodiments, the antigen-binding molecule of the present disclosure is capable of reducing tumour-associated macrophage activity. In some embodiments, antigen-binding molecule of the present disclosure is capable of reducing tumour-associated macrophage activity relative to a negative control condition, e.g. in an appropriate in vitro assay, or in vivo.

In some embodiments, the antigen-binding molecule of the present disclosure is capable of reducing tumour-associated macrophage activity to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the tumour-associated macrophage activity observed in the absence of the antigen-binding molecule (or in the presence of an appropriate control antigen-binding molecule). In some embodiments, the antigen-binding molecule of the present disclosure is capable of reducing the level of a correlate of tumour-associated macrophage activity to less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of a correlate of tumour-associated macrophage activity in the absence of the antigen-binding molecule (or in the presence of an appropriate control antigen-binding molecule).

In some embodiments tumour-associated macrophage activity can be analysed by measuring a correlate of tumour-associated macrophage activity. In some embodiments tumour-associated macrophage activity can be determined e.g. by analysis of production of IL-10, IL-13, IL-4.

In some embodiments, the antigen-binding molecule of the present disclosure is capable of increasing the number and/or proportion of M1-type macrophages (i.e. M1 macrophages) relative to a negative control condition, e.g. in an appropriate in vitro assay, or in vivo. The ability of an antigen-binding molecule to increase the number and/or proportion of M1-type macrophages can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule or an appropriate control antigen-binding molecule.

In some embodiments, the antigen-binding molecule of the present disclosure is capable of increasing the number of M1-type macrophages to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the number of M1-type macrophages observed in the absence of the antigen-binding molecule (or in the presence of an appropriate control antigen-binding molecule).

In some embodiments, the antigen-binding molecule of the present disclosure is capable of increasing the proportion of M1-type macrophages to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, ≥10 times, ≥20 times, ≥30 times, ≥40 times, or ≥50 times the proportion of M1-type macrophages observed in the absence of the antigen-binding molecule (or in the presence of an appropriate control antigen-binding molecule). In some embodiments, the antigen-binding molecule of the present disclosure is capable of increasing the proportion of M1-type macrophages by at least 1%, e.g. at least 2%, 3%, 4%, 5%, 10%, 15%, 20%, 25%, 30%, 35%, 40%, 45%, 50%, 55%, 60%, 65%, 70%, 75%, 80%, 85%, 90%, 95% or 100% the proportion of M1-type macrophages observed in the absence of the antigen-binding molecule (or in the presence of an appropriate control antigen-binding molecule).

Cell numbers and proportions can be determined e.g. by flow cytometry analysis using antibodies allowing detection of cell types. M1-type macrophages can be identified by the presence and/or absence of one or more cell markers, e.g F4/80+, MHCII+, CD206−, TNFa+, MMP2/9−, B7-H4−, STAT-3−, iNOS+, HLA-DR+, CD68+, CD14+, CD163−, CD204. M1 macrophages may be characterized as cells that produce cytokines such as IL-12, IL-1, IL-6, tumor necrosis factor (TNF)-α, reactive oxygen species (ROS), and/or nitric oxide (NO) and/or exhibit increased expression of the MHC II class. M1-type macrophages are described in Lin et al., Journal of Hematology & Oncology (2019) 12:76 (which is hereby incorporated by reference in its entirety). By way of example, Example 8 illustrates the determination of the proportion of M1-type macrophages using flow cytometry to detect F4/80+MHCII+CD206− TNFa+ cells.

In some embodiments, the antigen-binding molecule of the present disclosure is capable of increasing M1-type macrophage activity relative to a negative control condition, e.g. in an appropriate in vitro assay, or in vivo. The ability of an antigen-binding molecule to increase M1-type macrophage activity can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule or an appropriate control antigen-binding molecule.

In some embodiments, the antigen-binding molecule of the present disclosure is capable of increasing M1-type macrophage activity to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥110 times the M1-type macrophage activity observed in the absence of the antigen-binding molecule (or in the presence of an appropriate control antigen-binding molecule). In some embodiments, the antigen-binding molecule of the present disclosure is capable of increasing the level of a correlate of M1-type macrophage activity to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments M1-type macrophage activity can be analysed by measuring a correlate of such activity. In some embodiments M1-type macrophage activity can be determined e.g. by analysis of production of TNFa, IL-12, CXCL-10, IFNγ, NOS. By way of example, Example 8 illustrates the determination of M1-type macrophage activity by measuring TNFa by flow cytometry.

In some embodiments, the antigen-binding molecule of the present disclosure is capable of increasing production of TNFa by immune cells relative to a negative control condition, e.g. in an appropriate in vitro assay, or in vivo. The ability of an antigen-binding molecule to increase production of TNFa by immune cells can be analysed in an assay, e.g. as described in Example 8 herein. Such methods may comprise immunoprofiling of a tumour from a non-human animal model (e.g. a cell-line derived mouse model) of a cancer, following administration of the antigen-binding molecule or an appropriate control antigen-binding molecule.

### Chimeric Antigen Receptors (CARs)

The present disclosure also provides Chimeric Antigen Receptors (CARs) comprising the antigen-binding molecules or polypeptides of the present disclosure.

CARs are recombinant receptors that provide both antigen-binding and T cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

The CAR of the present disclosure comprises an antigen-binding region which comprises or consists of the antigen-binding molecule of the present disclosure, or which comprises or consists of a polypeptide according to the present disclosure.

The cell membrane anchor region is provided between the antigen-binding region and the signalling region of the CAR and provides for anchoring the CAR to the cell membrane of a cell expressing a CAR, with the antigen-binding region in the extracellular space, and signalling region inside the cell. In some embodiments, the CAR comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins such as FcγRI have also been employed in CARs (Haynes et al., 2001 J Immunol 166(1):182-187). Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1 BB, ICOS and CD27. In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1 BB-mediated signalling is through TNF receptor associated factor (TRAF) adaptor proteins. Signalling regions of CARs therefore sometimes contain co-stimulatory sequences derived from signalling regions of more than one co-stimulatory molecule. In some embodiments, the CAR of the present disclosure comprises one or more co-stimulatory sequences comprising or consisting of an amino acid sequence which comprises, consists of, or is derived from, the amino acid sequence of the intracellular domain of one or more of CD28, OX40, 4-1 BB, ICOS and CD27.

An optional hinge region may provide separation between the antigen-binding domain and the transmembrane domain, and may act as a flexible linker. Hinge regions may be derived from IgG1. In some embodiments, the CAR of the present disclosure comprises a hinge region comprising or consisting of an amino acid sequence which comprises, consists of, or is derived from, the amino acid sequence of the hinge region of IgG1.

Also provided is a cell comprising a CAR according to the present disclosure. The CAR according to the present disclosure may be used to generate CAR-expressing immune cells, e.g. CAR-T or CAR-NK cells. Engineering of CARs into immune cells may be performed during culture, in vitro.

The antigen-binding region of the CAR of the present disclosure may be provided with any suitable format, e.g. scFv, scFab, etc.

### Nucleic Acids and Vectors

The present disclosure provides a nucleic acid, or a plurality of nucleic acids, encoding an antigen-binding molecule, polypeptide or CAR according to the present disclosure.

In some embodiments, the nucleic acid is purified or isolated, e.g. from other nucleic acid, or naturally-occurring biological material. In some embodiments, the nucleic acid(s) comprise or consist of DNA and/or RNA.

The present disclosure also provides a vector, or plurality of vectors, comprising the nucleic acid or plurality of nucleic acids according to the present disclosure.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of an antigen-binding molecule according to the present disclosure may be encoded by different nucleic acids of the plurality of nucleic acids, or by different vectors of the plurality of vectors.

### Cells Comprising/Expressing the Antigen-Binding Molecules and Polypeptides

The present disclosure also provides a cell comprising or expressing an antigen-binding molecule, polypeptide or CAR according to the present disclosure. Also provided is a cell comprising or expressing a nucleic acid, a plurality of nucleic acids, a vector or a plurality of vectors according to the present disclosure.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

The present disclosure also provides a method for producing a cell comprising a nucleic acid(s) or vector(s) according to the present disclosure, comprising introducing a nucleic acid, a plurality of nucleic acids, a vector or a plurality of vectors according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising an antigen-binding molecule, polypeptide or CAR according to the present disclosure, comprising introducing a nucleic acid, a plurality of nucleic acids, a vector or a plurality of vectors according to the present disclosure in a cell. In some embodiments, the methods additionally comprise culturing the cell under conditions suitable for expression of the nucleic acid(s) or vector(s) by the cell. In some embodiments, the methods are performed in vitro.

The present disclosure also provides cells obtained or obtainable by the methods according to the present disclosure.

### Producing the Antigen-Binding Molecules and Polypeptides

Antigen-binding molecules and polypeptides according to the present disclosure may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18: 4373-4388, which is hereby incorporated by reference in its entirety.

Alternatively, antigen-binding molecules and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146 both of which are hereby incorporated by reference in their entirety. Methods for the recombinant production of antigen-binding molecules are also described in Frenzel et al., Front Immunol. (2013); 4: 217 and Kunert and Reinhart, Appl Microbiol Biotechnol. (2016) 100: 3451-3461, both of which are hereby incorporated by reference in their entirety.

In some cases the antigen-binding molecule of the present disclosure are comprised of more than one polypeptide chain. In such cases, production of the antigen-binding molecules may comprise transcription and translation of more than one polypeptide, and subsequent association of the polypeptide chains to form the antigen-binding molecule.

For recombinant production according to the present disclosure, any cell suitable for the expression of polypeptides may be used. The cell may be a prokaryote or eukaryote. In some embodiments, the cell is a prokaryotic cell, such as a cell of archaea or bacteria. In some embodiments, the bacteria may be Gram-negative bacteria such as bacteria of the family Enterobacteriaceae, for example Escherichia coli.

In some embodiments, the cell is a eukaryotic cell such as a yeast cell, a plant cell, insect cell or a mammalian cell, e.g. CHO, HEK (e.g. HEK293), HeLa or COS cells. In some embodiments, the cell is a CHO cell that transiently or stably expresses the polypeptides.

In some cases the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according using a system described in Zemella et al. Chembiochem (2015) 16(17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the antigen-binding molecule/polypeptide(s), the polypeptide(s) of interest may be isolated. Any suitable method for separating proteins from cells known in the art may be used. In order to isolate the polypeptide it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

### Compositions

The present disclosure also provides compositions comprising the antigen-binding molecules, polypeptides, CARs, nucleic acids, expression vectors and cells described herein.

The antigen-binding molecules, polypeptides, CARs, nucleic acids, expression vectors and cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the composition is formulated for injection or infusion, e.g. into a blood vessel or tumor.

In accordance with the present disclosure methods are also provided for the production of pharmaceutically useful compositions, such methods of production may comprise one or more steps selected from: producing an antigen-binding molecule, polypeptide, CAR, nucleic acid (or plurality thereof), expression vector (or plurality thereof) or cell described herein; isolating an antigen-binding molecule, polypeptide, CAR, nucleic acid (or plurality thereof), expression vector (or plurality thereof) or cell described herein; and/or mixing an antigen-binding molecule, polypeptide, CAR, nucleic acid (or plurality thereof), expression vector (or plurality thereof) or cell described herein with a pharmaceutically acceptable carrier, adjuvant, excipient or diluent.

For example, a further aspect the present disclosure relates to a method of formulating or producing a medicament or pharmaceutical composition for use in the treatment of a disease/condition (e.g. a cancer), the method comprising formulating a pharmaceutical composition or medicament by mixing an antigen-binding molecule, polypeptide, CAR, nucleic acid (or plurality thereof), expression vector (or plurality thereof) or cell described herein with a pharmaceutically acceptable carrier, adjuvant, excipient or diluent.

### Therapeutic and Prophylactic Applications

The antigen-binding molecules, polypeptides, CARs, nucleic acids, expression vectors, cells and compositions described herein find use in therapeutic and prophylactic methods.

The present disclosure provides an antigen-binding molecule, polypeptide, CAR, nucleic acid (or plurality thereof), expression vector (or plurality thereof), cell or composition described herein for use in a method of medical treatment or prophylaxis. Also provided is the use of an antigen-binding molecule, polypeptide, CAR, nucleic acid (or plurality thereof), expression vector (or plurality thereof), cell or composition described herein in the manufacture of a medicament for treating or preventing a disease or condition. Also provided is a method of treating or preventing a disease or condition, comprising administering to a subject a therapeutically or prophylactically effective amount of an antigen-binding molecule, polypeptide, CAR, nucleic acid (or plurality thereof), expression vector (or plurality thereof), cell or composition described herein.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments, the methods may lead to an improvement in the disease/condition, e.g. a reduction in the symptoms of the disease/condition or reduction in some other correlate of the severity/activity of the disease/condition. In some embodiments, the methods may prevent development of the disease/condition to a later stage (e.g. a chronic stage or metastasis).

It will be appreciated that the articles of the present disclosure may be used for the treatment/prevention of any disease/condition that would derive therapeutic or prophylactic benefit from a reduction in the number and/or activity of cells expressing VISTA (e.g. MDSCs). It will also be clear that the therapeutic and prophylactic utility of the present disclosure extends to essentially any disease/condition which would benefit from a reduction in the number or activity of MDSCs and/or other cells expressing VISTA, e.g. tumor-associated macrophages (TAMs) and neutrophils. Antagonism of VISTA effectively releases effector immune cells from suppression by MDSCs and/or other cells expressing VISTA.

For example, the disease/condition may be a disease/condition in which cells expressing VISTA (e.g. MDSCs) are pathologically implicated, e.g. a disease/condition in which an increased number/proportion of cells expressing VISTA (e.g. MDSCs) is positively associated with the onset, development or progression of the disease/condition, and/or severity of one or more symptoms of the disease/condition, or for which an increased number/proportion of cells expressing VISTA (e.g. MDSCs), is a risk factor for the onset, development or progression of the disease/condition.

In some embodiments, the disease/condition to be treated/prevented in accordance with the present disclosure is a disease/condition characterised by an increase in the number/proportion/activity of cells expressing VISTA (e.g. MDSCs), e.g. as compared to the number/proportion/activity of cells expressing VISTA (e.g. MDSCs) in the absence of the disease/condition.

In some embodiments, a subject may be selected for treatment described herein based on the detection of an increase in the number/proportion/activity of cells expressing VISTA (e.g. MDSCs), e.g. in the periphery, or in an organ/tissue which is affected by the disease/condition (e.g. an organ/tissue in which the symptoms of the disease/condition manifest), or by the presence of cells expressing VISTA (e.g. MDSCs or tumor-associated macrophages) in a tumor. The disease/condition may affect any tissue or organ or organ system. In some embodiments, the disease/condition may affect several tissues/organs/organ systems.

In some embodiments a subject may be selected for therapy/prophylaxis in accordance with the present disclosure based on determination that the subject has an increase in the number/proportion/activity of cells expressing VISTA (e.g. MDSCs) in the periphery or in an organ/tissue relative to the number/proportion/activity of such cells in a healthy subject, or based on determination that the subject has a tumor comprising cells expressing VISTA (e.g. MDSCs).

It will also be appreciated that the articles of the present disclosure may be used for the treatment/prevention of any disease/condition that would derive therapeutic or prophylactic benefit from:
(i) an increase in the number and/or proportion of antigen-specific CD8+ T cells;
(ii) an increase in CD8+ T cell activity;
(iii) a reduction in the level of T cell exhaustion;
(iv) a reduction in the number and/or proportion of tumour-associated macrophages (TAMs);
(v) an increase in the number and/or proportion of M1-type macrophages; and/or
(vi) an increase in M1-type macrophage activity.

In some embodiments, the disease/condition is a cancer/tumour and the therapeutic or prophylactic benefit would be derived from one or more of (i) to (vi) above, within a tumour microenvironment.

In connection with the preceding paragraph, it will be appreciated that the “increase” or “reduction” in the number/proportion of the relevant cell type/subtype, or the level of the relevant activity, is relative to the number/proportion/level observed in the absence of such therapeutic/prophylactic intervention (i.e., the number/proportion/level observed in the untreated state).

For example, the disease/condition may be a disease/condition in which:
(i) a reduced or low number/proportion of antigen-specific CD8+ T cells;
(ii) reduced or low CD8+ T cell activity;
(iii) the presence, or an increased or high number/proportion, of exhausted T cells;
(iv) the presence, or an increased or high number/proportion, of TAMs;
(v) a reduced or low number/proportion of M1-type macrophages; and/or
(vi) reduced or low M1-type macrophage activity;
   are pathologically implicated, e.g. a disease/condition in which one or more of (i) to (vi) above is positively associated with the onset, development or progression of the disease/condition, and/or severity of one or more symptoms of the disease/condition, or for which one or more of (i) to (vi) above is a risk factor for the onset, development or progression of the disease/condition. In some embodiments, the one or more of (i) to (vi) above is in a tumour, e.g. within a tumour microenvironment.

A “reduced” or “low” number/proportion of a given cell type/subtype, or an “reduced” or “low” level of a given activity in accordance with the present disclosure refers to a number/proportion of the given cell type/subtype, or a level of the relevant activity, which is less than a reference value for the number/proportion of the given cell type/subtype, or less than a reference value for the level of the relevant activity. In some embodiments, a “reduced” or “low” number/proportion of a given cell type/subtype, or a “reduced” or “low” level of a given activity may be less than 1 times, e.g. 50.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the reference value. Conversely, an “increased” or “high” number/proportion of a given cell type/subtype, or an “increased” or “high” level of a given activity in accordance with the present disclosure refers to a number/proportion of the given cell type/subtype, or a level of the relevant activity, which is greater than a reference value for the number/proportion of the given cell type/subtype, or greater than a reference value for the level of the relevant activity. In some embodiments, an “increased” or “high” number/proportion of a given cell type/subtype, or an “increased” or “high” level of a given activity may be more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the reference value.

The reference value in accordance with the preceding paragraph may be the average (e.g. the mean) value for that given cell type/subtype, in the context of the relevant disease/condition. In some embodiments, the reference value may be the average (e.g. the mean) value for that given cell type/subtype in a tissue/organ affected by the disease/condition (e.g. a tissue/organ in which one or more symptoms of the disease/condition manifest). In some embodiments, the reference value may be the average (e.g. the mean) value for that given cell type/subtype in cancers (i.e. in general), a representative subset of cancers, a given type of cancer, or a given type of tumour.

The presence or absence of a given cell type/subtype as referred to hereinabove may be as determined by analysis by an appropriate method, e.g. by flow cytometry using antibodies providing for the detection of the given cell type/subtype.

In some embodiments, the disease/condition to be treated/prevented is a cancer.

It will be appreciated that the antigen-binding molecules are useful for the treatment of cancers in general, because antigen-binding molecules of the present disclosure are useful to release effector immune cells from MDSC-mediated suppression or suppression by cells expressing VISTA, and thereby enhance the anticancer immune response.

The cancer may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma.

Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

MDSCs are elevated in advanced colorectal cancer (Toor et al, Front Immunol. 2016; 7:560). MDSCs are also observed in breast cancer, and the percentage of MDSCs in the peripheral blood is increased in patients with later stage breast cancer (Markowitz et al, Breast Cancer Res Treat. 2013 July; 140(1):13-21). MDSC abundance is also correlated with poor prognosis in solid tumors (Charoentong et al, Cell Rep. 2017 Jan. 3; 18(1):248-262), and MDSCs are enriched in liver cancer models (Connolly et al., J Leukoc Biol. (2010) 87(4):713-25). Prostate and breast carcinomas, melanomas, colorectal cancer and Lewis lung carcinoma have been reported to produce chemokines which attract MDSCs and contribute to immune suppression (Umansky et al., Vaccines (Basel) (2016) 4(4):36)), and MDSCs in pancreatic cancer patients have been positively correlated with tumor burden (Xu et al., Hepatobiliary Pancreat Dis Int. (2016) 15(1):99-105). VISTA has also been reported to be a target for the treatment of ovarian cancer (see e.g. U.S. Pat. No. 9,631,018 B2) and lymphoma (see e.g. WO 2017/023749 A1).

Blando et al. Proc Natl Acad Sci USA. (2019) 116(5):1692-1697 recently reported significant infiltration of VISTA-expressing myeloid cells in pancreatic cancer, and expansion of VISTA-expressing myeloid cells has been observed following treatment with CTLA4 antagonist in prostate cancer, and both pre- and post-treatment with PD-L1 antagonist in melanoma.

In some embodiments, a cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

In some embodiments, the cancer is colorectal cancer (e.g. colon carcinoma, colon adenocarcinoma), pancreatic cancer, breast cancer (e.g. triple-negative breast cancer), liver cancer, prostate cancer, ovarian cancer, head and neck cancer, leukemia (e.g. T cell leukemia), lymphoma, melanoma, thymoma, lung cancer, non-small cell lung cancer (NSCLC) and/or a solid tumor.

In some embodiments, the cancer is mesothelioma. In some embodiments, the cancer is epithelioid mesothelioma.

The treatment/prevention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

The articles of the present disclosure (i.e. antigen-binding molecules, compositions, etc.) are particularly useful for the treatment/prevention of diseases/conditions (e.g. cancers/tumours) characterised by:
(i) a reduced or low number/proportion of antigen-specific CD8+ T cells;
(ii) reduced or low CD8+ T cell activity;
(iii) the presence, or an increased or high number/proportion, of exhausted T cells;
(iv) the presence, or an increased or high number/proportion, of TAMs;
(v) a reduced or low number/proportion of M1-type macrophages; and/or
(vi) reduced or low M1-type macrophage activity.

The articles of the present disclosure (i.e. antigen-binding molecules, compositions, etc.) are particularly useful for the treatment/prevention of diseases/conditions (e.g. cancers) characterised by the presence of cells expressing VISTA and/or diseases/conditions (e.g. cancers) characterised by signalling mediated by a complex comprising VISTA.

A cancer characterised by the presence of cells expressing VISTA may comprise cancerous cells expressing VISTA. That is, cells of the cancer may express VISTA.

Alternatively, or additionally, a cancer characterised by ‘the presence of’ cells expressing VISTA may be characterised by the presence of cells expressing VISTA in the vicinity of (i.e. proximal to) cells of the cancer. The cancer may comprise infiltration of cells expressing VISTA. The cancer may comprise a tumor displaying infiltration of cells expressing VISTA. In connection with such embodiments, the cells expressing VISTA are not necessarily cells of the cancer, and may e.g. be non-cancerous cells. In some embodiments, the cells are immune cells. The immune cells expressing VISTA may be or comprise cells of hematopoietic origin, e.g. neutrophils, eosinophils, basophils, dendritic cells, lymphocytes, or monocytes. In some embodiments, the immune cells expressing VISTA may be or comprise lymphocytes, e.g. a T cells, B cells, NK cells, NKT cells, innate lymphoid cells (ILCs), or precursors thereof. In some embodiments, the immune cells expressing VISTA are effector immune cells (e.g. CD8+ T cells, CD8+ cytotoxic T lymphocytes (CD8+CTLs), CD4+ T cells, CD4+T helper cells, NK cells, IFNγ-producing cells, memory T cells, central memory T cells, antigen-experienced T cells and/or CD45RO+ T cells). In some embodiments, the immune cells expressing VISTA are antigen-presenting cells (APCs), macrophages, dendritic cells, T cells (e.g. CD8+ T cells) and/or MDSCs. In embodiments wherein the cancer comprises infiltration of immune cells expressing VISTA, or wherein the cancer comprises a tumor displaying infiltration of such immune cells, the immune cells may be referred to as tumor-infiltrating immune cells.

For example, a cancer characterised by the presence of cells expressing VISTA may comprise a tumor comprising cells (e.g. non-cancerous cells, e.g. tumor-infiltrating immune cells) expressing VISTA.

It will be appreciated that cells that are said to be ‘in the vicinity of’ or ‘proximal to’ cells of a cancer are provided in close physical proximity to cells of a cancer, e.g. in vivo in a subject having such a cancer. In some embodiments, cells which are in the vicinity of/proximal to cells of a cancer are comprised in the same organ/tissue as cells of the cancer. In some embodiments, cells which are in the vicinity of/proximal to cells of a cancer are comprised in a tumor of the cancer. In some embodiments, cells which are in the vicinity of/proximal to cells of a cancer are in contact with cells of the cancer.

In some embodiments, the cancer to be treated/prevented comprises cells expressing VISTA. In some embodiments, the cancer to be treated/prevented comprises cancerous cells expressing VISTA. In some embodiments, the cells expressing VISTA are MDSCs (e.g. g-MDSCs and/or m-MDSCs). In some embodiments, the cancer comprises a tumor comprising cells expressing VISTA (e.g. MDSCs). In some embodiments, the cancer to be treated/prevented comprises a tumor comprising MDSCs. In some embodiments, the cancer to be treated/prevented comprises infiltration of cells expressing VISTA (e.g. MDSCs). In some embodiments, the cancer to be treated/prevented comprises a tumor displaying infiltration of cells expressing VISTA (e.g. MDSCs).

In some embodiments, the cancer to be treated/prevented comprises a tumor comprising a population of CD45+ cells comprising greater than 1%, e.g. ≥2%, ≥5%, ≥10%, ≥15%, ≥20%, ≥25% or ≥30% MDSCs (e.g. as determined by immunoprofiling of the tumor).

VISTA-binding antigen-binding molecules described herein are demonstrated to inhibit interaction between VISTA and interaction partners for VISTA (e.g. interaction partners for VISTA that bind to the C-C′ region of VISTA, e.g. LRIG1 and VSIG3). Inhibition of interaction between VISTA and its interaction partners using the antigen-binding molecules described herein is moreover demonstrated to release effector immune cells from MDSC-mediated suppression of their activity.

Accordingly, the articles of the present disclosure (i.e. antigen-binding molecules, compositions, etc.) are particularly useful for the treatment/prevention of diseases/conditions (e.g. cancers) characterised by the presence of cells expressing an interaction partner for VISTA (e.g. an interaction partner that binds to the C-C′ region of VISTA, e.g. LRIG1 or VSIG3), and/or diseases/conditions (e.g. cancers) characterised by signalling mediated by a complex comprising VISTA and an interaction partner for VISTA (e.g. an interaction partner that binds to the C-C′ region of VISTA, e.g. LRIG1 or VSIG3).

A cancer characterised by the presence of cells expressing an interaction partner for VISTA (e.g. an interaction partner that binds to the C-C′ region of VISTA, e.g. LRIG1 or VSIG3) may comprise cancerous cells expressing the interaction partner for VISTA. That is, cells of the cancer may express the interaction partner for VISTA.

Alternatively, or additionally, a cancer characterised by ‘the presence of’ cells expressing an interaction partner for VISTA (e.g. an interaction partner that binds to the C-C′ region of VISTA, e.g. LRIG1 or VSIG3) may be characterised by the presence of cells expressing the interaction partner for VISTA in the vicinity of (i.e. proximal to) cells of the cancer. The cancer may comprise infiltration of cells expressing an interaction partner for VISTA (e.g. an interaction partner that binds to the C-C′ region of VISTA, e.g. LRIG1 or VSIG3). The cancer may comprise a tumor displaying infiltration of cells expressing an interaction partner for VISTA (e.g. an interaction partner that binds to the C-C′ region of VISTA, e.g. LRIG1 or VSIG3). In connection with such embodiments, the cells expressing the interaction partner for VISTA are not necessarily cells of the cancer, and may e.g. be non-cancerous cells. In some embodiments, the cells are immune cells. The immune cells expressing an interaction partner for VISTA (e.g. an interaction partner that binds to the C-C′ region of VISTA, e.g. LRIG1 or VSIG3) may be or comprise cells of hematopoietic origin, e.g. neutrophils, eosinophils, basophils, dendritic cells, lymphocytes, or monocytes. In some embodiments, the immune cells expressing an interaction partner for VISTA (e.g. an interaction partner that binds to the C-C′ region of VISTA, e.g. LRIG1 or VSIG3) may be or comprise lymphocytes, e.g. a T cells, B cells, NK cells, NKT cells, innate lymphoid cells (ILCs), or precursors thereof. In some embodiments, the immune cells expressing an interaction partner for VISTA (e.g. an interaction partner that binds to the C-C′ region of VISTA, e.g. LRIG1 or VSIG3) are effector immune cells (e.g. CD8+ T cells, CD8+ cytotoxic T lymphocytes (CD8+CTLs), CD4+ T cells, CD4+T helper cells, NK cells, IFNγ-producing cells, memory T cells, central memory T cells, antigen-experienced T cells and/or CD45RO+ T cells). In some embodiments, the immune cells expressing an interaction partner for VISTA (e.g. an interaction partner that binds to the C-C′ region of VISTA, e.g. LRIG1 or VSIG3) are antigen-presenting cells (APCs), macrophages, dendritic cells, T cells (e.g. CD8+ T cells) and/or MDSCs. In embodiments wherein the cancer comprises infiltration of immune cells expressing an interaction partner for VISTA (e.g. an interaction partner that binds to the C-C′ region of VISTA, e.g. LRIG1 or VSIG3), or wherein the cancer comprises a tumor displaying infiltration of such immune cells, the immune cells may be referred to as tumor-infiltrating immune cells.

For example, a cancer characterised by the presence of cells expressing an interaction partner for VISTA (e.g. an interaction partner that binds to the C-C′ region of VISTA, e.g. LRIG1 or VSIG3) may comprise a tumor comprising cells (e.g. non-cancerous cells, e.g. tumor-infiltrating immune cells) expressing the interaction partner for VISTA.

In some embodiments, the cancer to be treated/prevented comprises cells expressing an interaction partner for VISTA (e.g. an interaction partner that binds to the C-C′ region of VISTA, e.g. LRIG1 or VSIG3). In some embodiments, the cancer to be treated/prevented comprises cells expressing an interaction partner for VISTA that binds to the C-C′ region of VISTA. In some embodiments, the cancer to be treated/prevented comprises cells expressing LRIG1. In some embodiments, the cancer to be treated/prevented comprises cells expressing VSIG3.

It will be appreciated that in embodiments herein, cancers comprising cells having specified characteristics may be or comprise tumors comprising cells having those characteristics.

In some embodiments, the cancer to be treated/prevented comprises a tumor characterised by the presence of cells (which may e.g. be non-cancerous cells, e.g. tumor-infiltrating immune cells) expressing an interaction partner for VISTA (e.g. an interaction partner that binds to the C-C′ region of VISTA, e.g. LRIG1 or VSIG3). In some embodiments, the cancer to be treated/prevented comprises a tumor characterised by the presence of cells expressing an interaction partner for VISTA that binds to the C-C′ region of VISTA. In some embodiments, the cancer to be treated/prevented comprises a tumor characterised by the presence of cells expressing LRIG1. In some embodiments, the cancer to be treated/prevented comprises a tumor characterised by the presence of cells expressing VSIG3.

In some embodiments, the cancer to be treated/prevented comprises a tumor comprising cells expressing an interaction partner for VISTA (e.g. an interaction partner that binds to the C-C′ region of VISTA, e.g. LRIG1 or VSIG3). In some embodiments, the cancer to be treated/prevented comprises infiltration of cells expressing an interaction partner for VISTA (e.g. an interaction partner that binds to the C-C′ region of VISTA, e.g. LRIG1 or VSIG3). In some embodiments, the cancer to be treated/prevented comprises a tumor displaying infiltration of cells expressing an interaction partner for VISTA (e.g. an interaction partner that binds to the C-C′ region of VISTA, e.g. LRIG1 or VSIG3).

In some embodiments, cells expressing an interaction partner for VISTA (e.g. an interaction partner that binds to the C-C′ region of VISTA, e.g. LRIG1 or VSIG3) may display increased expression of the interaction partner, relative to a reference level of expression, e.g. by cells of the same type (e.g. from the same organ/issue) under non-pathological conditions.

In some embodiments, cells expressing an interaction partner for VISTA (e.g. an interaction partner that binds to the C-C′ region of VISTA, e.g. LRIG1 or VSIG3) may overexpress the interaction partner for VISTA. Such cells may express the relevant molecule at a level which is greater than the level of expression by equivalent non-cancerous cells/non-tumor tissue.

Cells may display increased expression or overexpression of an interaction partner for VISTA (e.g. an interaction partner that binds to the C-C′ region of VISTA, e.g. LRIG1 or VSIG3) e.g. as a result of genetic variation resulting in increased expression of the interaction partner. Accordingly, in some embodiments, the cancer comprises cells—or comprises a tumor comprising cells—harbouring a genetic variant (e.g. a mutation) which causes increased (gene and/or protein) expression of an interaction partner for VISTA (e.g. an interaction partner that binds to the C-C′ region of VISTA, e.g. LRIG1 or VSIG3), relative to the level of expression by comparable cells harbouring a reference allele not comprising the genetic variant (e.g. a non-mutated, or ‘wildtype’ allele). The genetic variant may be or comprise insertion, deletion, substitution to, or larger-scale translocation/rearrangement of, the nucleotide sequence relative to the reference allele.

A genetic variant ‘resulting in’ increased expression of an interaction partner for VISTA (e.g. an interaction partner that binds to the C-C′ region of VISTA, e.g. LRIG1 or VSIG3) may be known or predicted to cause, or may be associated with, increased gene/protein expression of the relevant molecule. In some embodiments, a genetic variant resulting in increased expression of the interaction partner for VISTA (e.g. an interaction partner that binds to the C-C′ region of VISTA, e.g. LRIG1 or VSIG3) may cause an increase in the level of the interaction partner for VISTA on or at the cell surface of a cell comprising the genetic variant, relative to an equivalent cell not comprising the genetic variant. In some embodiments, a genetic variant resulting in increased expression of the interaction partner for VISTA (e.g. an interaction partner that binds to the C-C′ region of VISTA, e.g. LRIG1 or VSIG3) may cause an increase in the level of secretion of the interaction partner for VISTA from a cell comprising the genetic variant, relative to an equivalent cell not comprising the genetic variant.

In some embodiments, the cancer to be treated/prevented is characterised by signalling mediated by a complex comprising VISTA and an interaction partner for VISTA (e.g. an interaction partner that binds to the C-C′ region of VISTA, e.g. LRIG1 or VSIG3). In some embodiments, the cancer to be treated/prevented is characterised by VISTA/LRIG1-mediated signalling. In some embodiments, the cancer to be treated/prevented is characterised by VISTA/VSIG3-mediated signalling.

In some embodiments, the cancer to be treated/prevented is characterised by an elevated level of signalling mediated by a complex comprising VISTA and an interaction partner for VISTA (e.g. an interaction partner that binds to the C-C′ region of VISTA, e.g. LRIG1 or VSIG3), i.e. as compared to the level of signalling by the relevant complex in the absence of the cancer.

In some embodiments, the cancer to be treated/prevented is characterised by the presence of (i) cells expressing VISTA, and (ii) cells expressing an interaction partner for VISTA (e.g. an interaction partner that binds to the C-C′ region of VISTA, e.g. LRIG1 or VSIG3). In some embodiments, the cancer to be treated/prevented is characterised by the presence of (i) cells expressing VISTA, and (ii) cells expressing LRIG1 and/or VSIG3.

In some embodiments, the cancer to be treated/prevented comprises (i) cells expressing VISTA, and (ii) cells expressing an interaction partner for VISTA (e.g. an interaction partner that binds to the C-C′ region of VISTA, e.g. LRIG1 or VSIG3). In some embodiments, the cancer to be treated/prevented comprises (i) cells expressing VISTA, and (ii) cells expressing LRIG1 and/or VSIG3.

In some embodiments, the cancer to be treated/prevented comprises a tumor characterised by the presence of (i) cells expressing VISTA, and (ii) cells expressing an interaction partner for VISTA (e.g. an interaction partner that binds to the C-C′ region of VISTA, e.g. LRIG1 or VSIG3). In some embodiments, the cancer to be treated/prevented comprises a tumor characterised by the presence of (i) cells expressing VISTA, and (ii) cells expressing LRIG1 and/or VSIG3.

In some embodiments, the cancer to be treated/prevented comprises a tumor comprising (i) cells expressing VISTA, and (ii) cells expressing an interaction partner for VISTA (e.g. an interaction partner that binds to the C-C′ region of VISTA, e.g. LRIG1 or VSIG3). In some embodiments, the cancer to be treated/prevented comprises a tumor comprising (i) cells expressing VISTA, and (ii) cells expressing LRIG1 and/or VSIG3.

In some embodiments, the cancer to be treated/prevented comprises infiltration of (i) cells expressing VISTA, and (ii) cells expressing an interaction partner for VISTA (e.g. an interaction partner that binds to the C-C′ region of VISTA, e.g. LRIG1 or VSIG3). In some embodiments, the cancer to be treated/prevented comprises infiltration of (i) cells expressing VISTA, and (ii) cells expressing LRIG1 and/or VSIG3.

In some embodiments, the cancer to be treated/prevented comprises a tumor displaying infiltration of (i) cells expressing VISTA, and (ii) cells expressing an interaction partner for VISTA (e.g. an interaction partner that binds to the C-C′ region of VISTA, e.g. LRIG1 or VSIG3). In some embodiments, the cancer to be treated/prevented comprises a tumor displaying infiltration of (i) cells expressing VISTA, and (ii) cells expressing LRIG1 and/or VSIG3.

In some embodiments, the cancer to be treated/prevented is a cancer characterised by signalling mediated by a complex comprising VISTA and an interaction partner for VISTA (e.g. an interaction partner that binds to the C-C′ region of VISTA, e.g. LRIG1 or VSIG3). In some embodiments, the cancer to be treated/prevented is characterised by VISTA/LRIG1-mediated signalling. In some embodiments, the cancer to be treated/prevented is characterised by VISTA/VSIG3-mediated signalling.

In some embodiments, the cancer to be treated/prevented is a cancer characterised by an elevated level of signalling mediated by a complex comprising VISTA and an interaction partner for VISTA (e.g. an interaction partner that binds to the C-C′ region of VISTA, e.g. LRIG1 or VSIG3), e.g. as compared to the level of signalling by the complex in the relevant cell type/tissue/organ in the absence of cancer. In some embodiments, the cancer to be treated/prevented is characterised by an elevated level of VISTA/LRIG1-mediated signalling compared to the level of VISTA/LRIG1-mediated signalling in the relevant cell type/tissue/organ in the absence of cancer. In some embodiments, the cancer to be treated/prevented is characterised by an elevated level of VISTA/VSIG3-mediated signalling compared to the level of VISTA/VSIG3-mediated signalling in the relevant cell type/tissue/organ in the absence of cancer.

In some embodiments, the disease/condition in which the VISTA-expressing cells are pathologically implicated is an infectious disease, e.g. bacterial, viral, fungal, or parasitic infection. In some embodiments it may be particularly desirable to treat chronic/persistent infections, e.g. where such infections are associated with T cell dysfunction or T cell exhaustion. It is well established that T cell exhaustion is a state of T cell dysfunction that arises during many chronic infections (including viral, bacterial and parasitic), as well as in cancer (Wherry Nature Immunology Vol. 12, No. 6, p 492-499, June 2011).

Examples of bacterial infections that may be treated include infection by Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa. For example, the bacterial infection may be sepsis or tuberculosis. Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV). Examples of fungal infections that may be treated include infection by Alternaria sp, Aspergillus sp, Candida sp and Histoplasma sp. The fungal infection may be fungal sepsis or histoplasmosis. Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

In some embodiments, the disease/condition to be treated/prevented in accordance with the present disclosure is a cancer characterised by a reduced or low number and/or proportion of antigen-specific CD8+ T cells.

In some embodiments, the disease/condition to be treated/prevented in accordance with the present disclosure is a cancer characterised by a reduced or low level of CD8+ T cell activity.

In some embodiments, the disease/condition to be treated/prevented in accordance with the present disclosure is a cancer characterised by an increased or high level of T cell exhaustion.

In some embodiments, the disease/condition to be treated/prevented in accordance with the present disclosure is a cancer characterised by an increased or high number and/or proportion of tumour-associated macrophages (TAMs).

In some embodiments, the disease/condition to be treated/prevented in accordance with the present disclosure is a cancer characterised by a reduced or low number and/or proportion of M1-type macrophages.

In some embodiments, the disease/condition to be treated/prevented in accordance with the present disclosure is a cancer characterised by a reduced or low level of M1-type macrophage activity.

In some embodiments, the antigen-binding molecule exerts its therapeutic/prophylactic effect via a molecular mechanism which does not involve an Fc region-mediated effector function (e.g. ADCC, ADCP, CDC). In some embodiments, the molecular mechanism does not involve binding of the antigen-binding molecule to an Fcγ receptor (e.g. one or more of FcγRI, FcγRIIa, FcγRIIb, FcγRIIc, FcγRIIIa and FcγRIIIb). In some embodiments, the molecular mechanism does not involve binding of the antigen-binding molecule to a complement protein (e.g. C1q).

In some embodiments (e.g. embodiments wherein the antigen-binding molecule lacks an Fc region, or embodiments wherein the antigen-binding molecule comprises an Fc region which is not able to induce an Fc-mediated antibody effector function), the treatment does not induce/increase killing of VISTA-expressing cells. In some embodiments, the treatment does not reduce the number/proportion of VISTA-expressing cells.

In some embodiments, the treatment (i) inhibits VISTA-mediated signalling, and (ii) does not induce/increase killing of VISTA-expressing cells. In some embodiments, the treatment (i) inhibits VISTA-mediated signalling, and (ii) does not reduce the number/proportion of VISTA-expressing cells.

In some embodiments, a subject may be selected for treatment described herein based on the detection of a cancer comprising cells expressing VISTA (e.g. MDSCs), or detection of a tumor comprising cells expressing VISTA (e.g. MDSCs), e.g. in a sample obtained from the subject. In some embodiments, a subject may be selected for treatment described herein based on the detection of a cancer comprising cells expressing an interaction partner for VISTA (e.g. an interaction partner that binds to the C-C′ region of VISTA, e.g. LRIG1 or VSIG3), or detection of a tumor comprising cells expressing an interaction partner for VISTA (e.g. an interaction partner that binds to the C-C′ region of VISTA, e.g. LRIG1 or VSIG3), e.g. in a sample obtained from the subject.

In some embodiments, a subject may be selected for treatment described herein based on the detection of:
(i) a reduced or low number/proportion of antigen-specific CD8+ T cells;
(ii) reduced or low CD8+ T cell activity;
(iii) the presence, or an increased or high number/proportion, of exhausted T cells;
(iv) the presence, or an increased or high number/proportion, of TAMs;
(v) a reduced or low number/proportion of M1-type macrophages; and/or
(vi) reduced or low M1-type macrophage activity;
   e.g. in the periphery, or in an organ/tissue which is affected by the disease/condition (e.g. an organ/tissue in which the symptoms of the disease/condition manifest), or in a tumour (e.g. in the tumour microenvironment). The disease/condition may affect any tissue or organ or organ system. In some embodiments, the disease/condition may affect several tissues/organs/organ systems.

In some embodiments a subject may be selected for therapy/prophylaxis in accordance with the present disclosure based on determination that the subject has one or more of (i) to (vi) above in the periphery or in an organ/tissue or in a tumour (e.g. in the tumour microenvironment) relative to a reference level, e.g. the corresponding number/proportion/activity in a subject having a cancer of the same type.

Administration of the articles of the present disclosure is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments, either simultaneously or sequentially dependent upon the condition to be treated. The antigen-binding molecule or composition described herein and a therapeutic agent may be administered simultaneously or sequentially.

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments, the therapeutic or prophylactic intervention comprises a stem cell transplant.

In some embodiments, the antigen-binding molecule is administered in combination with an agent capable of inhibiting signalling mediated by an immune checkpoint molecule other than VISTA. In some embodiments, the immune checkpoint molecule is e.g. PD-1, CTLA-4, LAG-3, TIM-3, TIGIT or BTLA. In some embodiments, the antigen-binding molecule is administered in combination with an agent capable of promoting signalling mediated by a costimulatory receptor. In some embodiments, the costimulatory receptor is e.g. CD28, CD80, CD40L, CD86, OX40, 4-1 BB, CD27 or ICOS.

Accordingly, the present disclosure provides compositions comprising an article according to the present disclosure (e.g. an antigen-binding molecule according to the present disclosure) and an agent capable of inhibiting signalling mediated by an immune checkpoint molecule other than VISTA. Also provided are compositions comprising the articles of the present disclosure and an agent capable of promoting signalling mediated by a costimulatory receptor. Also provided is the use of such compositions in methods of medical treatment and prophylaxis of diseases/conditions described herein.

Also provided are methods for treating/preventing diseases/conditions described herein comprising administering an article according to the present disclosure (e.g. an antigen-binding molecule according to the present disclosure) and an agent capable of inhibiting signalling mediated by an immune checkpoint molecule other than VISTA. Also provided are methods for treating/preventing diseases/conditions described herein comprising administering an article according to the present disclosure (e.g. an antigen-binding molecule according to the present disclosure) and an agent capable of promoting signalling mediated by a costimulatory receptor.

Agents capable of inhibiting signalling mediated by immune checkpoint molecules are known in the art, and include e.g. antibodies capable of binding to immune checkpoint molecules or their ligands, and inhibiting signalling mediated by the immune checkpoint molecule. Other agents capable of inhibiting signalling mediated by an immune checkpoint molecule include agents capable of reducing gene/protein expression of the immune checkpoint molecule or a ligand for the immune checkpoint molecule (e.g. through inhibiting transcription of the gene(s) encoding the immune checkpoint molecule/ligand, inhibiting post-transcriptional processing of RNA encoding the immune checkpoint molecule/ligand, reducing stability of RNA encoding the immune checkpoint molecule/ligand, promoting degradation of RNA encoding the immune checkpoint molecule/ligand, inhibiting post-translational processing of the immune checkpoint molecule/ligand, reducing stability the immune checkpoint molecule/ligand, or promoting degradation of the immune checkpoint molecule/ligand), and small molecule inhibitors.

Agents capable of promoting signalling mediated by costimulatory receptors are known in the art, and include e.g. agonist antibodies capable of binding to costimulatory receptors and triggering or increasing signalling mediated by the costimulatory receptor. Other agents capable of promoting signalling mediated by costimulatory receptors include agents capable of increasing gene/protein expression of the costimulatory receptor or a ligand for the costimulatory receptor (e.g. through promoting transcription of the gene(s) encoding the costimulatory receptor/ligand, promoting post-transcriptional processing of RNA encoding the costimulatory receptor/ligand, increasing stability of RNA encoding the costimulatory receptor/ligand, inhibiting degradation of RNA encoding the costimulatory receptor/ligand, promoting post-translational processing of the costimulatory receptor/ligand, increasing stability the costimulatory receptor/ligand, or inhibiting degradation of the costimulatory receptor/ligand), and small molecule agonists.

Immune suppression by VISTA-expressing MDSCs has been implicated in the failure of, and development of resistance to, treatment with agents capable of inhibiting signalling mediated by an immune checkpoint molecules. Gao et al., Nature Medicine (2017) 23: 551-555 recently suggested that VISTA may be a compensatory inhibitory pathway in prostate tumors after ipilimumab (i.e. anti-CTLA-4 antibody) therapy.

In particular embodiments the antigen-binding molecule of the present disclosure is administered in combination with an agent capable of inhibiting signalling mediated by PD-1. The agent capable of inhibiting signalling mediated by PD-1 may be a PD-1- or PD-L1-targeted agent. The agent capable of inhibiting signalling mediated by PD-1 may e.g. be an antibody capable of binding to PD-1 or PD-L1 and inhibiting PD-1-mediated signalling. In some embodiments, the agent is an antagonist anti-PD-1 antibody. In some embodiments, the agent is an antagonist anti-PD-L1 antibody.

In some embodiments, the antigen-binding molecule of the present disclosure is administered in combination with an agent capable of inhibiting signalling mediated by CTLA-4. The agent capable of inhibiting signalling mediated by CTLA-4 may be a CTLA-4-targeted agent, or an agent targeted against a ligand for CTLA-4 such as CD80 or CD86. In some embodiments, the agent capable of inhibiting signalling mediated by CTLA-4 may e.g. be an antibody capable of binding to CTLA-4, CD80 or CD86 and inhibiting CTLA-4-mediated signalling.

In some embodiments, the antigen-binding molecule of the present disclosure is administered in combination with an agent capable of inhibiting signalling mediated by LAG-3. The agent capable of inhibiting signalling mediated by LAG-3 may be a LAG-3-targeted agent, or an agent targeted against a ligand for LAG-3 such as MHC class II. In some embodiments, the agent capable of inhibiting signalling mediated by LAG-3 may e.g. be an antibody capable of binding to LAG-3 or MHC class II and inhibiting LAG-3-mediated signalling.

In some embodiments, the antigen-binding molecule of the present disclosure is administered in combination with an agent capable of inhibiting signalling mediated by TIM-3. The agent capable of inhibiting signalling mediated by TIM-3 may be a TIM-3-targeted agent, or an agent targeted against a ligand for TIM-3 such as Galectin 9. In some embodiments, the agent capable of inhibiting signalling mediated by TIM-3 may e.g. be an antibody capable of binding to TIM-3 or Galectin 9 and inhibiting TIM-3-mediated signalling.

In some embodiments, the antigen-binding molecule of the present disclosure is administered in combination with an agent capable of inhibiting signalling mediated by TIGIT. The agent capable of inhibiting signalling mediated by TIGIT may be a TIGIT-targeted agent, or an agent targeted against a ligand for TIGIT such as CD113, CD112 or CD155. In some embodiments, the agent capable of inhibiting signalling mediated by TIGIT may e.g. be an antibody capable of binding to TIGIT, CD113, CD112 or CD155 and inhibiting TIGIT-mediated signalling.

In some embodiments, the antigen-binding molecule of the present disclosure is administered in combination with an agent capable of inhibiting signalling mediated by BTLA. The agent capable of inhibiting signalling mediated by BTLA may be a BTLA-targeted agent, or an agent targeted against a ligand for BTLA such as HVEM. In some embodiments, the agent capable of inhibiting signalling mediated by BTLA may e.g. be an antibody capable of binding to BTLA or HVEM and inhibiting BTLA-mediated signalling.

In some embodiments methods employing a combination of an antigen-binding molecule of the present disclosure and an agent capable of inhibiting signalling mediated by an immune checkpoint molecule (e.g. PD-1 and/or PD-L1) provide an improved treatment effect as compared to the effect observed when either agent is used as a monotherapy. In some embodiments, the combination of an antigen-binding molecule of the present disclosure and an agent capable of inhibiting signalling mediated by an immune checkpoint molecule (e.g. PD-1 and/or PD-L1) provide a synergistic (i.e. super-additive) treatment effect.

In some embodiments, treatment with a combination comprising (i) an antigen-binding molecule of the present disclosure and (ii) an agent capable of inhibiting signalling mediated by an immune checkpoint molecule (e.g. PD-1 and/or PD-L1) may be associated with one or more of:
an improved treatment effect as compared to the treatment effect observed with either component of the combination used alone;
a treatment effect which is synergistic (i.e. super-additive) as compared to the treatment effect observed with either component of the combination used alone;
increased inhibition of tumor growth as compared to inhibition of tumor growth by either component of the combination used alone;
of tumor growth which is synergistic (i.e. super-additive) as compared to inhibition of tumor growth by either component of the combination used alone;
greater reduction in the number/activity of suppressor immune cells as compared to reduction of the number/activity of suppressor immune cells by either component of the combination used alone;
in the number/activity of suppressor immune cells which is synergistic (i.e. super-additive) as compared to reduction of the number/activity of suppressor immune cells by either component of the combination used alone;
greater reduction of proliferation of suppressor immune cells as compared to reduction proliferation of suppressor immune cells by either component of the combination used alone
reduction of proliferation of suppressor immune cells which is synergistic (i.e. super-additive) as compared to reduction proliferation of suppressor immune cells by either component of the combination used alone;
greater reduction in the proportion of suppressor immune cells within a population of cells (e.g. CD45+ cells, e.g. CD45+ cells obtained from a tumor) as compared to the reduction of the proportion of suppressor immune cells by either component of the combination used alone; and
reduction in the proportion of suppressor immune cells within a population of cells (e.g. CD45+ cells, e.g. CD45+ cells obtained from a tumor) which is synergistic (i.e. super-additive) as compared to the reduction of the proportion of suppressor immune cells by either component of the combination used alone.
greater increase in the number and/or proportion and/or activity of antigen-specific CD8+ T cells as compared to the increase in the number and/or proportion and/or activity of antigen-specific CD8+ T cells by either component of the combination used alone;
increase in the number and/or proportion and/or activity of antigen-specific CD8+ T cells which is synergistic (i.e. super-additive) as compared to the increase in the number and/or proportion and/or activity of antigen-specific CD8+ T cells by either component of the combination used alone;
greater reduction in the level of T cell exhaustion as compared to reduction in the level of T cell exhaustion by either component of the combination used alone;
reduction in the level of T cell exhaustion which is synergistic (i.e. super-additive) as compared to reduction in the level of T cell exhaustion by either component of the combination used alone.

Simultaneous administration refers to administration of the antigen-binding molecule, polypeptide, CAR, nucleic acid (or plurality thereof), expression vector (or plurality thereof), cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament.

The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), [No Entries], Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

Multiple doses of the antigen-binding molecule, polypeptide, CAR, nucleic acid (or plurality thereof), expression vector (or plurality thereof), cell or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

The present disclosure also provides methods for determining a subject's response therapeutic/prophylactic intervention as described herein, e.g. a subject's response to intervention comprising administering an antigen-binding molecule according to the present disclosure to the subject.

The methods may be performed for the purpose of determining is a subject is responding to the intervention (e.g. a positive response). The methods may be performed for the purpose of determining if a subject is not responding to the intervention.

In some aspects and embodiments, the methods comprise analysing a subject's cancer/tumour in order to determine the:
(i) number and/or proportion of antigen-specific CD8+ T cells;
(ii) CD8+ T cell activity;
(iii) level of T cell exhaustion;
(iv) number and/or proportion of tumour-associated macrophages (TAMs);
(v) number and/or proportion of M1-type macrophages; and/or
(vi) M1-type macrophage activity.

In some embodiments, the methods comprise undertaking analysis of one or more of (i) to (vi) above at a first timepoint, and also at a subsequent timepoint. In some embodiments, the methods comprise analysing a subject's cancer/tumour at a plurality of timepoints to determine one or more of (i) to (vi) above.

In some embodiments, the first timepoint in accordance with the preceding paragraph is prior to administration of an antigen-binding molecule according to the present disclosure. In some embodiments, the first timepoint is at or during the course of administration of an antigen-binding molecule according to the present disclosure. In some embodiments, the subsequent timepoint is after administration of an antigen-binding molecule according to the present disclosure (e.g. after administration of one or more doses of the antigen-binding molecule). In some embodiments, the subsequent timepoint is post-treatment with an antigen-binding molecule according to the present disclosure (e.g. after administration of a final dose of the antigen-binding molecule). In some embodiments, one or more doses of an antigen-binding molecule according to the present disclosure (e.g. an antigen-binding molecule that binds to VISTA), are administered to the subject between the first timepoint and the subsequent timepoint.

In some embodiments the length of time between timepoints may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or31 days, or 1, 2, 3, 4, 5, or 6 months.

In some embodiments, the methods further comprise determining the difference between one or more of (i) to (vi) above as determined at the first and subsequent timepoints.

In some embodiments, a(n):
(vii) increased number and/or proportion of antigen-specific CD8+ T cells;
(viii) increased CD8+ T cells activity;
(ix) reduced level of T cell exhaustion;
(x) reduced number and/or proportion of tumour-associated macrophages (TAMs);
(xi) increased number and/or proportion of M1-type macrophages; and/or
(xii) increased M1-type macrophage activity,
   as determined at the subsequent timepoint relative to the first timepoint is indicative of a positive response to intervention with an antigen-binding molecule according to the present disclosure.

In some embodiments, no difference (e.g. no significant difference) between one or more of (i) to (vi) as determined at the subsequent timepoint relative to the first timepoint is indicative of no response to treatment with an antigen-binding molecule according to the present disclosure.

In some embodiments, a(n):
(xiii) reduced number and/or proportion of antigen-specific CD8+ T cells;
(xiv) reduced CD8+ T cells activity;
(xv) increased level of T cell exhaustion;
(xvi) increased number and/or proportion of tumour-associated macrophages (TAMs);
(xvii) reduced number and/or proportion of M1-type macrophages; and/or
(xviii) reduced M1-type macrophage activity,
   as determined at the subsequent timepoint relative to the first timepoint is indicative of no response to treatment with an antigen-binding molecule according to the present disclosure.

### Methods of Detection

The present disclosure also provides the articles of the present disclosure for use in methods for detecting, localizing or imaging VISTA, or cells expressing VISTA (e.g. MDSCs). The antigen-binding molecules described herein may be used in methods that involve the antigen-binding molecule to VISTA. Such methods may involve detection of the bound complex of the antigen-binding molecule and VISTA.

In particular, detection of VISTA may be useful in methods of diagnosing/prognosing a disease/condition in which cells expressing VISTA (e.g. MDSCs) are pathologically implicated, identifying subjects at risk of developing such diseases/conditions, and/or may be useful in methods of predicting a subject's response to a therapeutic intervention.

As such, a method is provided, comprising contacting a sample containing, or suspected to contain, VISTA with an antigen-binding molecule as described herein, and detecting the formation of a complex of the antigen-binding molecule and VISTA. Also provided is a method comprising contacting a sample containing, or suspected to contain, a cell expressing VISTA with an antigen-binding molecule as described herein and detecting the formation of a complex of the antigen-binding molecule and a cell expressing VISTA.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition). In some embodiments, the sample may be obtained or derived from a cancer, tumor, or cells thereof.

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods of this kind may provide the basis of methods for the diagnostic and/or prognostic evaluation of a disease or condition, e.g. a cancer. Such methods may be performed in vitro on a patient sample, or following processing of a patient sample. Once the sample is collected, the patient is not required to be present for the in vitro method to be performed, and therefore the method may be one which is not practised on the human or animal body. In some embodiments, the method is performed in vivo.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

The present disclosure also provides methods for selecting/stratifying a subject for treatment with a VISTA-targeted agent. In some embodiments a subject is selected for treatment/prevention in accordance with the present disclosure, or is identified as a subject which would benefit from such treatment/prevention, based on detection/quantification of VISTA, or cells expressing VISTA, e.g. in a sample obtained from the subject.

Such methods may involve detecting or quantifying VISTA and/or cells expressing VISTA (e.g. MDSCs), e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Where an increased level of VISTA is detected, or where the presence of—or an increased number/proportion of—cells expressing VISTA (e.g. MDSCs) is detected in a sample obtained from a subject, the subject may be diagnosed as having a disease/condition in which MDSCs are pathologically implicated, or being at risk of developing such a disease/condition. In such methods, an “increased” level of expression or number/proportion of cells refers to a level/number/proportion which is greater than the level/number/proportion determined for an appropriate control condition, such as the level/number/proportion detected in a comparable sample (e.g. a sample of the same kind, e.g. obtained from the same fluid, tissue, organ etc.), e.g. obtained from a healthy subject.

Where an increased level of VISTA is detected, or where the presence of—or an increased number/proportion of—cells expressing VISTA (e.g. MDSCs) is detected in a sample obtained from a subject, the subject may be determined to have a poorer prognosis as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.).

The antigen-binding molecules of the present disclosure are also useful in methods for predicting response to immunotherapy. “Immunotherapy” generally refers to therapeutic intervention aimed at harnessing the immune system to treat a disease/condition. Immunotherapy includes therapeutic intervention to increase the number/proportion/activity of effector immune cells (e.g. effector T cells (e.g. antigen-specific T cells, CAR-T cells), NK cells) in a subject. Immunotherapy to increase the number/proportion/activity of effector immune cells includes intervention to promote proliferation and/or survival of effector immune cells, inhibit signalling mediated by immune checkpoint molecules, promote signalling mediated by costimulatory receptors, enhance antigen presentation by antigen-presenting cells, etc. Immunotherapy to increase the number/proportion/activity of effector immune cells also encompasses intervention to increase the frequency of effector immune cells having a desired specificity or activity in a subject e.g. through adoptive cell transfer (ACT). ACT generally involves obtaining immune cells from a subject, typically by drawing a blood sample from which immune cells are isolated. The cells are then typically treated or altered in some way, and then administered either to the same subject or to a different subject. ACT is typically aimed at providing an immune cell population with certain desired characteristics to a subject, or increasing the frequency immune cells with such characteristics in that subject. In some embodiments ACT may e.g. be of cells comprising a chimeric antigen receptor (CAR) specific for a target antigen or cell type of interest. Immunotherapy also includes therapeutic intervention to decrease the number/proportion/activity of suppressor immune cells (e.g. regulatory T cells, MDSCs) in a subject. Immunotherapy to decrease the number/proportion/activity of suppressor immune cells includes intervention to cause or potentiate cell killing of suppressor immune cells, and inhibit signalling mediated by immune checkpoint molecules.

Where an increased level of VISTA is detected, or where the presence of—or an increased number/proportion of—cells expressing VISTA (e.g. MDSCs) is detected in a sample obtained from a subject, the subject may be predicted to have a poorer response to immunotherapy to increase the number/proportion/activity of effector immune cells in the subject as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.). Where an increased level of VISTA is detected, or where the presence of—or an increased number/proportion of—cells expressing VISTA (e.g. MDSCs) is detected in a sample obtained from a subject, the subject may be predicted to have an improved response to immunotherapy aimed at reducing the number/proportion/activity of suppressor immune cells in the subject as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.).

In some embodiments, the methods comprise determining the relative size/activity of suppressor immune cell compartment and the effector immune cell compartment. For example, in some embodiments, the methods employ the antigen-binding molecules described herein in methods for determining the ratio of VISTA-expressing cells (e.g. MDSCs, TAMs, neutrophils) to effector immune cells. A subject having an increased ratio may be predicted to have an improved response to immunotherapy aimed at reducing the number/proportion/activity of suppressor immune cells, and/or may be predicted to have a poorer response to immunotherapy to increase the number/proportion/activity of effector immune cells as compared to a subject determined to have a lower ratio.

The diagnostic and prognostic methods of the present disclosure may be performed on samples obtained from a subject at multiple time points throughout the course of the disease and/or treatment, and may be used monitor development of the disease/condition over time, e.g. in response to treatment administered to the subject. The results of characterisation in accordance with the methods may be used to inform clinical decisions as to when and what kind of therapy to administer to a subject.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body.

### Diagnosis and Patient Selection

The present disclosure also provides diagnostic, prognostic and predictive methods in connection with the treatment and prevention of cancers described herein.

The methods may be performed for the purpose of diagnosing a cancer (e.g. a cancer described herein). The methods may be performed for the purpose of identifying/selecting a subject for therapeutic/prophylactic intervention as described herein.

In some aspects and embodiments, the methods comprise analysing a subject's cancer in order to determine whether the cancer is a cancer described herein, e.g. a cancer characterised by the presence of cells expressing VISTA, and/or characterised by the presence of cells expressing an interaction partner for VISTA (e.g. an interaction partner that binds to the C-C′ region of VISTA, e.g. LRIG1 or VSIG3).

In some embodiments, the methods comprise evaluating a cancer in order to determine whether it comprises cells expressing VISTA and/or cells expressing an interaction partner for VISTA (e.g. an interaction partner that binds to the C-C′ region of VISTA, e.g. LRIG1 or VSIG3). In some embodiments, the methods comprise evaluating a subject or a sample obtained from a subject to determine whether the subject comprises cells expressing VISTA and/or cells expressing an interaction partner for VISTA (e.g. an interaction partner that binds to the C-C′ region of VISTA, e.g. LRIG1 or VSIG3) in the vicinity of or proximal to cells of a cancer.

A cancer which is identified following such analysis to be a cancer characterised by the presence of cells expressing VISTA and/or the presence of cells expressing an interaction partner for VISTA (e.g. an interaction partner that binds to the C-C′ region of VISTA, e.g. LRIG1 or VSIG3), may be identified as a cancer suitable for intervention with an antigen-binding molecule according to the present disclosure (e.g. an antigen-binding molecule that inhibits interaction between VISTA and the interaction partner for VISTA).

In some aspects and embodiments, the methods comprise analysing a subject's cancer (e.g. a subject's tumour) in order to determine whether the cancer is a cancer described herein, e.g. a cancer characterised by:
(i) a reduced or low number/proportion of antigen-specific CD8+ T cells;
(ii) reduced or low CD8+ T cell activity;
(iii) the presence or high number/proportion of exhausted T cells;
(iv) the presence or high number/proportion of TAMs;
(v) a reduced or low number/proportion of M1-type macrophages; and/or
(vi) reduced or low M1-type macrophage activity.

In some embodiments, the methods comprise evaluating a subject's cancer (e.g. a subject's tumour) in order to determine whether it comprises/is characterised by one or more of (i) to (vi) above. A cancer which is identified by such analysis to be a cancer characterised by one or more of (i) to (vi) above may be identified as a cancer suitable for intervention with an antigen-binding molecule according to the present disclosure.

Aspects and embodiments of the present disclosure also comprise selecting a subject for therapeutic or prophylactic intervention in accordance with the present disclosure.

In some embodiments, a subject is selected/not selected for therapeutic intervention in accordance with the present disclosure based on the result of analysis of their cancer to determine whether the cancer is characterised by the presence of cells expressing VISTA and/or the presence of cells expressing an interaction partner for VISTA (e.g. an interaction partner that binds to the C-C′ region of VISTA, e.g. LRIG1 or VSIG3).

A subject having a cancer which is identified following such analysis to be a cancer characterised by the presence of cells expressing VISTA and/or the presence of cells expressing an interaction partner for VISTA (e.g. an interaction partner that binds to the C-C′ region of VISTA, e.g. LRIG1 or VSIG3), may be selected for treatment using an antigen-binding molecule according to the present disclosure (e.g. an antigen-binding molecule that inhibits interaction between VISTA and the interaction partner for VISTA).

In some embodiments, a method comprises selecting a subject for treatment with an antigen-binding molecule which binds to VISTA according to the present disclosure based on determination that the subject has a cancer characterised by the presence of cells expressing VISTA and/or the presence of cells expressing an interaction partner for VISTA (e.g. an interaction partner that binds to the C-C′ region of VISTA, e.g. LRIG1 or VSIG3).

In some aspects and embodiments, the methods comprise:
(a) analysing a subject's cancer in order to determine whether the cancer is characterised by the presence of cells expressing VISTA and/or the presence of cells expressing an interaction partner for VISTA (e.g. an interaction partner that binds to the C-C′ region of VISTA, e.g. LRIG1 or VSIG3); and
(b) selecting a subject for treatment with an antigen-binding molecule according to the present disclosure (e.g. an antigen-binding molecule that inhibits interaction between VISTA and the interaction partner for VISTA) where the subject's cancer is determined in step (a) to be a cancer characterised by the presence of cells expressing VISTA and/or the presence of cells expressing an interaction partner for VISTA (e.g. an interaction partner that binds to the C-C′ region of VISTA, e.g. LRIG1 or VSIG3).

In some embodiments, the methods further comprise:
(c) administering an antigen-binding molecule according to the present disclosure (e.g. an antigen-binding molecule that inhibits interaction between VISTA and the interaction partner for VISTA) to a subject selected for treatment in step (b).

Analysis of a subject or cancer may comprise analysing a sample obtained from a subject in order to determine whether the sample comprises cells expressing VISTA and/or cells expressing an interaction partner for VISTA (e.g. an interaction partner that binds to the C-C′ region of VISTA, e.g. LRIG1 or VSIG3). Analysis may comprise analysing a sample to determine whether it comprises cells expressing VISTA in or at the cell surface and/or cells expressing an interaction partner for VISTA (e.g. an interaction partner that binds to the C-C′ region of VISTA, e.g. LRIG1 or VSIG3) in or at the cell surface.

It will be appreciated that the sample is a cell containing sample. The sample may be of a tissue/organ of a subject, e.g. an organ affected by a cancer (e.g. a tissue/organ in which symptoms of a cancer manifest). The sample may be of a tumor. The sample may be a biopsy. Such analysis is preferably performed in vitro, but may alternatively be performed in vivo on the human or animal body.

Suitable methods for analysing expression of a given protein are well known to the skilled person, and include analysis by antibody-based methods, for example by western blot, immunohistochemistry, immunocytochemistry and flow cytometry. In accordance with methods comprising analysis of a sample in connection with the diagnostic methods and methods of patient selection described herein, analysis of a sample may employ immunohistochemical, immunocytochemical or flow cytometric methods for the detection of cells expressing VISTA and/or cells expressing an interaction partner for VISTA (e.g. an interaction partner that binds to the C-C′ region of VISTA, e.g. LRIG1 or VSIG3).

In some embodiments, a subject is selected/not selected for therapeutic intervention in accordance with the present disclosure based on the result of analysis of their cancer to determine whether the cancer is characterised by one or more of:
(i) a reduced or low number/proportion of antigen-specific CD8+ T cells;
(ii) reduced or low CD8+ T cell activity;
(iii) the presence or high number/proportion of exhausted T cells;
(iv) the presence or high number/proportion of TAMs;
(v) a reduced or low number/proportion of M1-type macrophages; and/or
(vi) reduced or low M1-type macrophage activity.

A subject having a cancer which is identified following such analysis to be a cancer characterised by one or more of (i) to (vi) above, may be selected for treatment using an antigen-binding molecule according to the present disclosure.

In some embodiments, a method comprises selecting a subject selecting a subject for treatment with an antigen-binding molecule which binds to VISTA according to the present disclosure based on determination that the subject has a cancer characterised by one or more of (i) to (vi) above.

In some aspects and embodiments, the methods comprise:
(a) analysing a subject's cancer in order to determine whether the cancer is characterised by one or more of (i) to (vi) above; and
(b) selecting a subject for treatment with an antigen-binding molecule according to the present disclosure where the subject's cancer is determined in step (a) to be a cancer characterised by one or more of (i) to (vi) above.

In some embodiments, the methods further comprise:
(c) administering an antigen-binding molecule according to the present disclosure (e.g. an antigen-binding molecule that binds to VISTA) to a subject selected for treatment in step (b).

Analysis of a subject or cancer may comprise analysing a sample obtained from a subject (e.g. a sample of a subject's cancer/tumour) in order to determine whether the sample comprises one or more of (i) to (vi) above. Analysis may comprise immunoprofiling of a sample e.g. by flow cytometry analysis using antibodies allowing detection of cell types.

It will be appreciated that the sample is a cell-containing sample. The sample may be of a tissue/organ of a subject, e.g. an organ affected by a cancer (e.g. a tissue/organ in which symptoms of a cancer manifest). The sample may be of a tumor. The sample may be a biopsy. Such analysis is preferably performed in vitro, but may alternatively be performed in vivo on the human or animal body.

Suitable methods for analysing include e.g. immunoprofiling of the sample.

### Subjects

The subject in accordance with aspects the present disclosure may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer), may be suspected of having such a disease/condition, or may be at risk of developing/contracting such a disease/condition.

In embodiments according to the present disclosure the subject is preferably a human subject. In some embodiments, the subject to be treated according to a therapeutic or prophylactic method of the present disclosure herein is a subject having, or at risk of developing, a cancer. In embodiments according to the present disclosure, a subject may be selected for treatment according to the methods based on characterisation for certain markers of such disease/condition. The subject may have (e.g. may have been determined to have) a cancer described herein.

### Kits

In some aspects of the present disclosure a kit of parts is provided. In some embodiments, the kit may have at least one container having a predetermined quantity of an antigen-binding molecule, polypeptide, CAR, nucleic acid (or plurality thereof), expression vector (or plurality thereof), cell or composition described herein.

In some embodiments, the kit may comprise materials for producing an antigen-binding molecule, polypeptide, CAR, nucleic acid (or plurality thereof), expression vector (or plurality thereof), cell or composition described herein.

The kit may provide the antigen-binding molecule, polypeptide, CAR, nucleic acid (or plurality thereof), expression vector (or plurality thereof), cell or composition together with instructions for administration to a patient in order to treat a specified disease/condition.

In some embodiments, the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tumor or to the blood.

### Sequence Identity

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

### Sequences

| SEQ | | |
|---|---|---|
| ID | | |
| NO: | DESCRIPTION | SEQUENCE |
| 1 | Human VISTA | MGVPTALEAGSWRWGSLLFALFLAASLGPVAAFKVATPYSLYVCPEGQNVTLTCRLLGPVDKGH |
| | (Q9H7M9-1, v3) | DVTFYKTWYRSSRGEVQTCSERRPIRNLTFQDLHLHHGGHQAANTSHDLAQRHGLESASDHHG |
| | | NFSITMRNLTLLDSGLYCCLVVEIRHHHSEHRVHGAMELQVQTGKDAPSNCVVYPSSSQDSENIT |
| | | AAALATGACIVGILCLPLILLLVYKQRQAASNRRAQELVRMDSNIQGIENPGFEASPPAQGIPEAKV |
| | | RHPLSYVAQRQPSESGRHLLSEPSTPLSPPGPGDVFFPSLDPVPDSPNFEVI |
| 2 | Mature human | FKVATPYSLYVCPEGQNVTLTCRLLGPVDKGHDVTFYKTWYRSSRGEVQTCSERRPIRNLTFQDL |
| | VISTA (Q9H7M9-1, | HLHHGGHQAANTSHDLAQRHGLESASDHHGNFSITMRNLTLLDSGLYCCLVVEIRHHHSEHRVH |
| | v3 positions 33 to | GAMELQVQTGKDAPSNCVVYPSSSQDSENITAAALATGACIVGILCLPLILLLVYKQRQAASNRRA |
| | 311) | QELVRMDSNIQGIENPGFEASPPAQGIPEAKVRHPLSYVAQRQPSESGRHLLSEPSTPLSPPGPG |
| | | DVFFPSLDPVPDSPNFEVI |
| 3 | Extracellular | FKVATPYSLYVCPEGQNVTLTCRLLGPVDKGHDVTFYKTWYRSSRGEVQTCSERRPIRNLTFQDL |
| | domain human | HLHHGGHQAANTSHDLAQRHGLESASDHHGNFSITMRNLTLLDSGLYCCLVVEIRHHHSEHRVH |
| | VISTA (Q9H7M9-1, | GAMELQVQTGKDAPSNCVVYPSSSQDSENITAA |
| | v3 positions 33 to | |
| | 194) | |
| 4 | Transmembrane | ALATGACIVGILCLPLILLLV |
| | domain human | |
| | VISTA (Q9H7M9-1, | |
| | v3 positions 195 to | |
| | 215) | |
| 5 | Cytoplasmic domain | YKQRQAASNRRAQELVRMDSNIQGIENPGFEASPPAQGIPEAKVRHPLSYVAQRQPSESGRHLL |
| | human VISTA | SEPSTPLSPPGPGDVFFPSLDPVPDSPNFEVI |
| | (Q9H7M9-1, v3 | |
| | positions 216 to | |
| | 311) | |
| 6 | Ig-like V-type | FKVATPYSLYVCPEGQNVTLTCRLLGPVDKGHDVTFYKTWYRSSRGEVQTCSERRPIRNLTFQDL |
| | domain human | HLHHGGHQAANTSHDLAQRHGLESASDHHGNFSITMRNLTLLDSGLYCCLVVEIRHHHSEHRVH |
| | VISTA (Q9H7M9-1, | GAMELQV |
| | v3 positions 33 to | |
| | 168) | |
| 7 | Human VSIG3 | MTSQRSPLAPLLLLSLHGVAASLEVSESPGSIQVARGQPAVLPCTFTTSAALINLNVIWMVTPLSNA |
| | isoform 1 | NQPEQVILYQGGQMFDGAPRFHGRVGFTGTMPATNVSIFINNTQLSDTGTYQCLVNNLPDIGGRN |
| | (Q5DX21-1, v3) | IGVTGLTVLVPPSAPHCQIQGSQDIGSDVILLCSSEEGIPRPTYLWEKLDNTLKLPPTATQDQVQGT |
| | | VTIRNISALSSGLYQCVASNAIGTSTCLLDLQVISPQPRNIGLIAGAIGTGAVIIIFCIALILGAFF |
| | | YWRSKNKEEEEEEIPNEIREDDLPPKCSSAKAFHTEISSSDNNTLTSSNAYNSRYWSNNPKVHRNTE |
| | | SVSHFSDLGQSFSFHSGNANIPSIYANGTHLVPGQHKTLVVTANRGSSPQVMSRSNGSVSRKPRPPH |
| | | THSYTISHATLERIGAVPVMVPAQSRAGSLV |
| 8 | Human VSIG3 | MSLVELLLWWNCFSRTGVAASLEVSESPGSIQVARGQPAVLPCTFTTSAALINLNVIWMVTPLSNA |
| | isoform 2 | NQPEQVILYQGGQMFDGAPRFHGRVGFTGTMPATNVSIFINNTQLSDTGTYQCLVNNLPDIGGRN |
| | (Q5DX21-2) | IGVTGLTVLVPPSAPHCQIQGSQDIGSDVILLCSSEEGIPRPTYLWEKLDNTLKLPPTATQDQVQGT |
| | | VTIRNISALSSGLYQCVASNAIGTSTCLLDLQVISPQPRNIGLIAGAIGTGAVIIIFCIALILGAFF |
| | | YWRSKNKEEEEEEIPNEIREDDLPPKCSSAKAFHTEISSSDNNTLTSSNAYNSRYWSNNPKVHRNTE |
| | | SVSHFSDLGQSFSFHSGNANIPSIYANGTHLVPGQHKTLVVTANRGSSPQVMSRSNGSVSRKPRPPH |
| | | THSYTISHATLERIGAVPVMVPAQSRAGSLV |
| 9 | Human VSIG3 | MSLVELLLWWNCFSRTGVAASLEVSESPGSIQVARGQPAVLPCTFTTSAALINLNVIWMVTPLSNA |
| | isoform 3 | NQPEQVILYQGGQMFDGAPRFHGRVGFTGTMPATNVSIFINNTQLSDTGTYQCLVNNLPDIGGRN |
| | (Q5DX21-3) | IGVTGLTVLVPPSAPHCQIQGSQDIGSDVILLCSSEEGIPRPTYLWEKLDNTLKLPPTATQDQVQGT |
| | | VTIRNISALSSAQPRNIGLIAGAIGTGAVIIIFCIALILGAFFYWRSKNKEEEEEEIPNEIREDDLP |
| | | PKCSSAKAFHTEISSSDNNTLTSSNAYNSRYWSNNPKVHRNTESVSHFSDLGQSFSFHSGNANIPSI |
| | | YANGTHLVPGQHKTLVVTANRGSSPQVMSRSNGSVSRKPRPPHTHSYTISHATLERIGAVPVMVPA |
| | | QSRAGSLV |
| 10 | Mature human | LEVSESPGSIQVARGQPAVLPCTFTTSAALINLNVIWMVTPLSNANQPEQVILYQGGQMFDGAPRF |
| | VSIG3 isoform 1 | HGRVGFTGTMPATNVSIFINNTQLSDTGTYQCLVNNLPDIGGRNIGVTGLTVLVPPSAPHCQIQGS |
| | (Q5DX21-1, v3 | QDIGSDVILLCSSEEGIPRPTYLWEKLDNTLKLPPTATQDQVQGTVTIRNISALSSGLYQCVASNAI |
| | positions 23 | GTSTCLLDLQVISPQPRNIGLIAGAIGTGAVIIIFCIALILGAFFYWRSKNKEEEEEEIPNEIREDD |
| | to 431) | LPPKCSSAKAFHTEISSSDNNTLTSSNAYNSRYWSNNPKVHRNTESVSHFSDLGQSFSFHSGNANIP |
| | | SIYANGTHLVPGQHKTLVVTANRGSSPQVMSRSNGSVSRKPRPPHTHSYTISHATLERIGAVPVMV |
| | | PAQSRAGSLV |
| 11 | Mature human | LEVSESPGSIQVARGQPAVLPCTFTTSAALINLNVIWMVTPLSNANQPEQVILYQGGQMFDGAPRF |
| | VSIG3 isoform 2 | HGRVGFTGTMPATNVSIFINNTQLSDTGTYQCLVNNLPDIGGRNIGVTGLTVLVPPSAPHCQIQGS |
| | (Q5DX21-2 | QDIGSDVILLCSSEEGIPRPTYLWEKLDNTLKLPPTATQDQVQGTVTIRNISALSSGLYQCVASNAI |
| | positions 23 | GTSTCLLDLQVISPQPRNIGLIAGAIGTGAVIIIFCIALILGAFFYWRSKNKEEEEEEIPNEIREDD |
| | to 431) | LPPKCSSAKAFHTEISSSDNNTLTSSNAYNSRYWSNNPKVHRNTESVSHFSDLGQSFSFHSGNANIP |
| | | SIYANGTHLVPGQHKTLVVTANRGSSPQVMSRSNGSVSRKPRPPHTHSYTISHATLERIGAVPVMV |
| | | PAQSRAGSLV |
| 12 | Mature human | LEVSESPGSIQVARGQPAVLPCTFTTSAALINLNVIWMVTPLSNANQPEQVILYQGGQMFDGAPRF |
| | VSIG3 isoform 3 | HGRVGFTGTMPATNVSIFINNTQLSDTGTYQCLVNNLPDIGGRNIGVTGLTVLVPPSAPHCQIQGS |
| | (Q5DX21-3 | QDIGSDVILLCSSEEGIPRPTYLWEKLDNTLKLPPTATQDQVQGTVTIRNISALSSAQPRNIGLIA |
| | positions 23 | GAIGTGAVIIIFCIALILGAFFYWRSKNKEEEEEEIPNEIREDDLPPKCSSAKAFHTEISSSDNNT |
| | to 407) | LTSSNAYNSRYWSNNPKVHRNTESVSHFSDLGQSFSFHSGNANIPSIYANGTHLVPGQHKTLVVTA |
| | | NRGSSPQVMSRSNGSVSRKPRPPHTHSYTISHATLERIGAVPVMVPAQSRAGSLV |
| 13 | Extracellular | LEVSESPGSIQVARGQPAVLPCTFTTSAALINLNVIWMVTPLSNANQPEQVILYQGGQMFDGAPRF |
| | domain human | HGRVGFTGTMPATNVSIFINNTQLSDTGTYQCLVNNLPDIGGRNIGVTGLTVLVPPSAPHCQIQGS |
| | VSIG3 isoforms 1 | QDIGSDVILLCSSEEGIPRPTYLWEKLDNTLKLPPTATQDQVQGTVTIRNISALSSGLYQCVASNAI |
| | and 2 | GTSTCLLDLQVISPQPRNIG |
| 14 | Extracellular | LEVSESPGSIQVARGQPAVLPCTFTTSAALINLNVIWMVTPLSNANQPEQVILYQGGQMFDGAPRF |
| | domain human | HGRVGFTGTMPATNVSIFINNTQLSDTGTYQCLVNNLPDIGGRNIGVTGLTVLVPPSAPHCQIQGS |
| | VSIG3 isoform 3 | QDIGSDVILLCSSEEGIPRPTYLWEKLDNTLKLPPTATQDQVQGTVTIRNISALSSAQPRNIG |
| 15 | Transmembrane | LIAGAIGTGAVIIIFCIALIL |
| | domain human | |
| | VSIG3 | |
| 16 | Cytoplasmic domain | GAFFYWRSKNKEEEEEEIPNEIREDDLPPKCSSAKAFHTEISSSDNNTLTSSNAYNSRYWSNNPK |
| | human VSIG3 | VHRNTESVSHFSDLGQSFSFHSGNANIPSIYANGTHLVPGQHKTLVVTANRGSSPQVMSRSNGS |
| | | VSRKPRPPHTHSYTISHATLERIGAVPVMVPAQSRAGSLV |
| 17 | Ig-like V-type | LEVSESPGSIQVARGQPAVLPCTFTTSAALINLNVIWMVTPLSNANQPEQVILYQGGQMFDGAPRF |
| | domain human | HGRVGFTGTMPATNVSIFINNTQLSDTGTYQCLVNNLPDIGGRNIGVT |
| | VSIG3 | |
| 18 | Ig-like C2-type | PSAPHCQIQGSQDIGSDVILLCSSEEGIPRPTYLWEKLDNTLKLPPTATQDQVQGTVTIRNISALSS |
| | domain human | GLYQCVASNAIGTSTCLLDLQVIS |
| | VSIG3 | |
| 19 | Human VSIG8 | MRVGGAFHLLLVCLSPALLSAVRINGDGQEVLYLAEGDNVRLGCPYVLDPEDYGPNGLDIEWMQ |
| | (PODPA2-1, v1) | VNSDPAHHRENVFLSYQDKRINHGSLPHLQQRVRFAASDPSQYDASINLMNLQVSDTATYECRV |
| | | KKTTMATRKVIVTVQARPAVPMCWTEGHMTYGNDVVLKCYASGGSQPLSYKWAKISGHHYPYR |
| | | AGSYTSQHSYHSELSYQESFHSSINQGLNNGDLVLKDISRADDGLYQCTVANNVGYSVCVVEVKV |
| | | SDSRRIGVIIGIVLGSLLALGCLAVGIWGLVCCCCGGSGAGGARGAFGYGNGGGVGGGACGDLA |
| | | SEIREDAVAPGCKASGRGSRVTHLLGYPTQNVSRSLRRKYAPPPCGGPEDVALAPCTAAAACEA |
| | | GPSPVYVKVKSAEPADCAEGPVQCKNGLLV |
| 20 | Mature human | VRINGDGQEVLYLAEGDNVRLGCPYVLDPEDYGPNGLDIEWMQVNSDPAHHRENVFLSYQDKRI |
| | VSIG8 (PODPA2-1, | NHGSLPHLQQRVRFAASDPSQYDASINLMNLQVSDTATYECRVKKTTMATRKVIVTVQARPAVPM |
| | v1 positions 22 to | CWTEGHMTYGNDVVLKCYASGGSQPLSYKWAKISGHHYPYRAGSYTSQHSYHSELSYQESFHS |
| | 414) | SINQGLNNGDLVLKDISRADDGLYQCTVANNVGYSVCVVEVKVSDSRRIGVIIGIVLGSLLALGCLA |
| | | VGIWGLVCCCCGGSGAGGARGAFGYGNGGGVGGGACGDLASEIREDAVAPGCKASGRGSRVT |
| | | HLLGYPTQNVSRSLRRKYAPPPCGGPEDVALAPCTAAAACEAGPSPVYVKVKSAEPADCAEGPV |
| | | QCKNGLLV |
| 21 | Extracellular | VRINGDGQEVLYLAEGDNVRLGCPYVLDPEDYGPNGLDIEWMQVNSDPAHHRENVFLSYQDKRI |
| | domain human | NHGSLPHLQQRVRFAASDPSQYDASINLMNLQVSDTATYECRVKKTTMATRKVIVTVQARPAVPM |
| | VSIG8 (PODPA2-1, | CWTEGHMTYGNDVVLKCYASGGSQPLSYKWAKISGHHYPYRAGSYTSQHSYHSELSYQESFHS |
| | v1 positions 22 to | SINQGLNNGDLVLKDISRADDGLYQCTVANNVGYSVCVVEVKVSDSRRIG |
| | 263) | |
| 22 | Transmembrane | VIIGIVLGSLLALGCLAVGIW |
| | domain human | |
| | VSIG8 (PODPA2-1, | |
| | v1 positions 264 to | |
| | 284) | |
| 23 | Cytoplasmic domain | GLVCCCCGGSGAGGARGAFGYGNGGGVGGGACGDLASEIREDAVAPGCKASGRGSRVTHLLG |
| | human VSIG8 | YPTQNVSRSLRRKYAPPPCGGPEDVALAPCTAAAACEAGPSPVYVKVKSAEPADCAEGPVQCKN |
| | (PODPA2-1, v1 | GLLV |
| | positions 285 to | |
| | 414) | |
| 24 | Ig-like V-type | VRINGDGQEVLYLAEGDNVRLGCPYVLDPEDYGPNGLDIEWMQVNSDPAHHRENVFLSYQDKRI |
| | domain 1 human | NHGSLPHLQQRVRFAASDPSQYDASINLMNLQVSDTATYECRVKKTTMATRKVIVT |
| | VSIG8 (PODPA2-1, | |
| | v1 positions 22 to | |
| | 141) | |
| 25 | Ig-like V-type | PAVPMCWTEGHMTYGNDVVLKCYASGGSQPLSYKWAKISGHHYPYRAGSYTSQHSYHSELSYQ |
| | domain 2 human | ESFHSSINQGLNNGDLVLKDISRADDGLYQCTVANNVGYSVCVVEVKVS |
| | VSIG8 (PODPA2-1, | |
| | v1 positions 146 to | |
| | 257) | |
| 26 | Epitope recognised | SRGEVQTCSERRPI |
| | by anti-VISTA | |
| | antibody clones | |
| | 4M2-C12, 4M2-B4, | |
| | 4M2-C9, 4M2-D9, | |
| | 4M2-D5, 4M2-A8, | |
| | V4H1, V4H2, V4-C1, | |
| | V4-C9, V4-C24, | |
| | V4-C26, V4-C27, | |
| | V4-C28, V4-C30 | |
| | and V4-C31 | |
| 27 | Epitope recognised | FQDLHLHHGGHQAA |
| | by anti-VISTA | |
| | antibody clones | |
| | 2M1-B12 and 2M1- | |
| | D2 | |
| 28 | Epitope recognised | CLVVEIRHHHSEHR |
| | by anti-VISTA | |
| | antibody clones | |
| | 1M2-D2, 13D5p, | |
| | 13D5-1 and 13D5- | |
| | 13 | |
| 29 | Epitope recognised | DKGHDVTFYKT |
| | by anti-VISTA | |
| | antibody clone 5M1- | |
| | A11 | |
| 30 | Epitope recognised | RHHHSEHRVHG |
| | by anti-VISTA | |
| | antibody clone 9M2- | |
| | C12 | |
| 31 | Positions 61 to 162 | DKGHDVTFYKTWYRSSRGEVQTCSERRPIRNLTFQDLHLHHGGHQAANTSHDLAQRHGLESAS |
| | of human VISTA | DHHGNFSITMRNLTLLDSGLYCCLVVEIRHHHSEHRVHG |
| | (Q9H7M9-1, v3) | |
| 32 | 4M2-C12 heavy | EVKLVESGPGLVKPSQSLSLTCTVTGYSITSDYAWNWIRQFPGNKLEWMGYITYSGNISYNPSLR |
| | chain variable | SRISITRDTSKNQFFLQLNSVTPEDTATYSCARSLYYPWYFDVWGAGTTVTVSS |
| | region | |
| 33 | 4M2-C12, 4M2-B4, | GYSITSDYA |
| | V4H2, 4M2-D9, V4- | |
| | C1, V4-C9 heavy | |
| | chain CDR1 | |
| 34 | 4M2-C12, 4M2-B4, | ITYSGNI |
| | V4H1, V4H2 heavy | |
| | chain CDR2 | |
| 35 | 4M2-C12, 4M2-B4, | ARSLYYPWYFDV |
| | V4H1, V4H2 heavy | |
| | chain CDR3 | |
| 36 | 4M2-C12 heavy | EVKLVESGPGLVKPSQSLSLTCTVT |
| | chain FR1 | |
| 37 | 4M2-C12, 4M2-B4 | WNWIRQFPGNKLEWMGY |
| | heavy chain FR2 | |
| 38 | 4M2-C12, 4M2-B4 | SYNPSLRSRISITRDTSKNQFFLQLNSVTPEDTATYSC |
| | heavy chain FR3 | |
| 39 | 4M2-C12, 4M2-B4, | WGAGTTVTVSS |
| | V4H1, V4H2, | |
| | 13D5p, 13D5-1, | |
| | 13D5-13 heavy | |
| | chain FR4 | |
| 40 | 4M2-C12 light chain | DIVITQTPAILSTSPGEKVTMTCRASSSVGYIHWYQQKPGSSPKPWIYATSNLASGVPARFSGSGS |
| | variable region | GTSNSLTITRVEAEDAATYYCQQWSSYPPITFGGGTKLEVK |
| 41 | 4M2-C12, 4M2-B4, | SSVGY |
| | V4H1, V4H2, V4- | |
| | C1, V4-C9, V4-C24, | |
| | V4-C26, V4-C27, | |
| | V4-C28, V4-C30, | |
| | V4-C31 light chain | |
| | CDR1 | |
| 42 | 4M2-C12, 4M2-B4, | ATS |
| | V4-C1, V4-C9, V4- | |
| | C28 light chain | |
| | CDR2 | |
| 43 | 4M2-C12, 4M2-B4, | QQWSSYPPIT |
| | V4H1, V4H2, V4-C1 | |
| | V4-C9, V4-C24, V4- | |
| | C26, V4-C27, V4- | |
| | C28, V4-C30, V4- | |
| | C31 light chain | |
| | CDR3 | |
| 44 | 4M2-C12 light chain | DIVITQTPAILSTSPGEKVTMTCRAS |
| | FR1 | |
| 45 | 4M2-C12, 4M2-B4 | IHWYQQKPGSSPKPWIY |
| | light chain FR2 | |
| 46 | 4M2-C12, 4M2-B4 | NLASGVPARFSGSGSGTSNSLTITRVEAEDAATYYC |
| | light chain FR3 | |
| 47 | 4M2-C12, 4M2-B4, | FGGGTKLEVK |
| | V4H1, V4H2, V4- | |
| | C1, V4-C9, V4-C24, | |
| | V4-C26, V4-C27, | |
| | V4-C28, V4-C30, | |
| | V4-C31 light chain | |
| | FR4 | |
| 48 | 4M2-B4 heavy | EVMLVESGPGLVKPSQSLSLTCTVTGYSITSDYAWNWIRQFPGNKLEWMGYITYSGNISYNPSLR |
| | chain variable | SRISITRDTSKNQFFLQLNSVTPEDTATYSCARSLYYPWYFDVWGAGTTVTVSS |
| | region | |
| 49 | 4M2-B4 heavy | EVMLVESGPGLVKPSQSLSLTCTVT |
| | chain FR1 | |
| 50 | 4M2-B4 light chain | DIVLTQTTAILSTSPGEKVTMTCRASSSVGYIHWYQQKPGSSPKPWIYATSNLASGVPARFSGSG |
| | variable region | SGTSNSLTITRVEAEDAATYYCQQWSSYPPITFGGGTKLEVK |
| 51 | 4M2-B4 light chain | DIVLTQTTAILSTSPGEKVTMTCRA |
| | FR1 | |
| 52 | V4H1 heavy chain | EVQLLESGGGLVQPGGSLRLSCAASGYTITSDYAMSWVRQAPGKGLEWVSVITYSGNISYADSV |
| | variable region | KGRFTISRDNSKNTLYLQMNSLRAEDTAVYYCARSLYYPWYFDVWGAGTTVTVSS |
| 53 | V4H1 heavy chain | GYTITSDYA |
| | CDR1 | |
| 54 | V4H1 heavy chain | EVQLLESGGGLVQPGGSLRLSCAAS |
| | FR1 | |
| 55 | V4H1 heavy chain | MSWVRQAPGKGLEWVSV |
| | FR2 | |
| 56 | V4H1 heavy chain | SYADSVKGRFTISRDNSKNTLYLQMNSLRAEDTAVYYC |
| | FR3 | |
| 57 | V4H1 light chain | EIVITQSPATLSLSPGERATLSCRASSSVGYLAWYQQKPGQAPRPLIYDTSNRATGIPARFSGSGS |
| | variable region | GTDNTLTISSLEPEDFAVYYCQQWSSYPPITFGGGTKLEVK |
| 58 | V4H1 light chain | DTS |
| | CDR2 | |
| 59 | V4H1, V4-C1 light | EIVITQSPATLSLSPGERATLSCRAS |
| | chain FR1 | |
| 60 | V4H1 light chain | LAWYQQKPGQAPRPLIY |
| | FR2 | |
| 61 | V4H1 light chain | NRATGIPARFSGSGSGTDNTLTISSLEPEDFAVYYC |
| | FR3 | |
| 62 | V4H2 heavy chain | QVQLQESGPGLVKPSDTLSLTCTVSGYSITSDYAWSWIRQPPGKGLEWIGYITYSGNISYNPSLKS |
| | variable region | RVTISVDTSKNQFSLKLSSVTAADTAVYDCARSLYYPWYFDVWGAGTTVTVSS |
| 63 | V4H2, V4-C1, V4- | QVQLQESGPGLVKPSDTLSLTCTVS |
| | C9, V4-C24, V4- | |
| | C26, V4-C27, V4- | |
| | C28, V4-C30, V4- | |
| | C31 heavy chain | |
| | FR1 | |
| 64 | V4H2 heavy chain | WSWIRQPPGKGLEWIGY |
| | FR2 | |
| 65 | V4H2 heavy chain | SYNPSLKSRVTISVDTSKNQFSLKLSSVTAADTAVYDC |
| | FR3 | |
| 66 | V4H2 light chain | DIQMTQSPSSLSASVGDRVTITCRASSSVGYLNWYQQKPGKAPKPLIYAASSLQSGVPSRFSGSG |
| | variable region | SGTDNTLTISSLQPEDFATYYCQQWSSYPPITFGGGTKLEVK |
| 67 | V4H2, 4M2-D9 light | AAS |
| | chain CDR2 | |
| 68 | V4H2 light chain | DIQMTQSPSSLSASVGDRVTITCRA |
| | FR1 | |
| 69 | V4H2 light chain | LNWYQQKPGKAPKPLIY |
| | FR2 | |
| 70 | V4H2 light chain | SLQSGVPSRFSGSGSGTDNTLTISSLQPEDFATYYC |
| | FR3 | |
| 71 | 2M1-B12 heavy | EVQLQQSGAELVRPGTSVKTSCKASGYTFTNYWLGWVKERAGHGLEWIGEIFPGGGHTNYKEKF |
| | chain variable | KGKATLTADTSSSTAYMKLSSLTSEDSAVYFCAQIPLYYGHYRSAYWGQGTLVTVSA |
| | region | |
| 72 | 2M1-B12, 2M1-D2, | GYTFTNYW |
| | 13D5p, 13D5-1, | |
| | 13D5-13 heavy | |
| | chain CDR1 | |
| 73 | 2M1-B12, 2M1-D2 | IFPGGGHT |
| | heavy chain CDR2 | |
| 74 | 2M1-B12, 2M1-D2 | AQIPLYYGHYRSAY |
| | heavy chain CDR3 | |
| 75 | 2M1-B12 heavy | EVQLQQSGAELVRPGTSVKTSCKAS |
| | chain FR1 | |
| 76 | 2M1-B12, 2M1-D2 | LGWVKERAGHGLEWIGE |
| | heavy chain FR2 | |
| 77 | 2M1-B12, 2M1-D2 | NYKEKFKGKATLTADTSSSTAYMKLSSLTSEDSAVYFC |
| | heavy chain FR3 | |
| 78 | 2M1-B12, 2M1-D2, | WGQGTLVTVSA |
| | 1M2-D2 heavy | |
| | chain FR4 | |
| 79 | 2M1-B12 light chain | DIQMMQSPASLSASVGETVAITCGASENIYGALNWYQRKQGKSPQLLIYGATNLADGMSSRFSGS |
| | variable region | GSGRQYSLKISSLHPDDVATYYCQNVLSTPYTFGGGTKLEIK |
| 80 | 2M1-B12, 2M1-D2 | ENIYGA |
| | light chain CDR1 | |
| 81 | 2M1-B12, 2M1-D2 | GAT |
| | light chain CDR2 | |
| 82 | 2M1-B12, 2M1-D2 | QNVLSTPYT |
| | light chain CDR3 | |
| 83 | 2M1-B12 light chain | DIQMMQSPASLSASVGETVAITCGAS |
| | FR1 | |
| 84 | 2M1-B12, 2M1-D2 | LNWYQRKQGKSPQLLIY |
| | light chain FR2 | |
| 85 | 2M1-B12, 2M1-D2 | NLADGMSSRFSGSGSGRQYSLKISSLHPDDVATYYC |
| | light chain FR3 | |
| 86 | 2M1-B12, 4M2-C9, | FGGGTKLEIK |
| | 2M1-D2, 4M2-D9, | |
| | 1M2-D2, 5M1-A11, | |
| | 4M2-D5, 4M2-A8, | |
| | 9M2-C12, 13D5p, | |
| | 13D5-1, 13d5-13 | |
| | light chain FR4 | |
| 87 | 4M2-C9 heavy | QVTLKECGPGILQPSQTLSLTCSFSGFSLSTSGMGVSWIRQPSGKGLEWLAHIYWDDDKRYNPSL |
| | chain variable | KSRLTISKDSSSNQVFLKITSVDTADTATYYCARRLDGYNDPYYFDYWGQGTTLTVSS |
| | region | |
| 88 | 4M2-C9, 5M1-A11 | GFSLSTSGMG |
| | heavy chain CDR1 | |
| 89 | 4M2-C9, 5M1-A11 | IYWDDDK |
| | heavy chain CDR2 | |
| 90 | 4M2-C9, 5M1-A11 | ARRLDGYNDPYYFDY |
| | heavy chain CDR3 | |
| 91 | 4M2-C9 heavy | QVTLKECGPGILQPSQTLSLTCSFS |
| | chain FR1 | |
| 92 | 4M2-C9, 5M1-A11 | VSWIRQPSGKGLEWLAH |
| | heavy chain FR2 | |
| 93 | 4M2-C9, 5M1-A11 | RYNPSLKSRLTISKDSSSNQVFLKITSVDTADTATYYC |
| | heavy chain FR3 | |
| 94 | 4M2-C9 heavy | WGQGTTLTVSS |
| | chain FR4 | |
| 95 | 4M2-C9 light chain | DIVMTQSPSSLSASLGDTVTITCHASQNVNVWLSWYQQKPGNIPKLLIYKASNLHAGVPSRFSGS |
| | variable region | GSGTGFTLTISSLQPEDIATYYCQQGQSYPLTFGGGTKLEIK |
| 96 | 4M2-C9 light chain | QNVNVW |
| | CDR1 | |
| 97 | 4M2-C9 light chain | KAS |
| | CDR2 | |
| 98 | 4M2-C9 light chain | QQGQSYPLT |
| | CDR3 | |
| 99 | 4M2-C9 light chain | DIVMTQSPSSLSASLGDTVTITCHAS |
| | FR1 | |
| 100 | 4M2-C9 light chain | LSWYQQKPGNIPKLLIY |
| | FR2 | |
| 101 | 4M2-C9 light chain | NLHAGVPSRFSGSGSGTGFTLTISSLQPEDIATYYC |
| | FR3 | |
| 102 | 2M1-D2 heavy | QVQLQQSGAELVRPGTSVKISCKASGYTFTNYWLGWVKERAGHGLEWIGEIFPGGGHTNYKEKF |
| | chain variable | KGKATLTADTSSSTAYMKLSSLTSEDSAVYFCAQIPLYYGHYRSAYWGQGTLVTVSA |
| | region | |
| 103 | 2M1-D2, 13D5p, | QVQLQQSGAELVRPGTSVKISCKAS |
| | 13D5-1, 13D5-13 | |
| | heavy chain FR1 | |
| 104 | 2M1-D2 light chain | DIVMTQSPASLSASVGETVAITCGASENIYGALNWYQRKQGKSPQLLIYGATNLADGMSSRFSGS |
| | variable region | GSGRQYSLKISSLHPDDVATYYCQNVLSTPYTFGGGTKLEIK |
| 105 | 2M1-D2 light chain | DIVMTQSPASLSASVGETVAITCGAS |
| | FR1 | |
| 106 | 4M2-D9 heavy | EVQLQESGPGLVKPSQSLSLTCTVTGYSITSDYAWNWIRQFPGNRLEWMGFISYSGFTTYSPSLE |
| | chain variable | SRISITRDTSKNQFFLQLISVTTEDTATYYCARNHYGGSYWYFDVWGAGTSVTVSS |
| | region | |
| 107 | 4M2-D9 heavy | ISYSGFT |
| | chain CDR2 | |
| 108 | 4M2-D9 heavy | ARNHYGGSYWYFDV |
| | chain CDR3 | |
| 109 | 4M2-D9 heavy | EVQLQESGPGLVKPSQSLSLTCTVT |
| | chain FR1 | |
| 110 | 4M2-D9 heavy | WNWIRQFPGNRLEWMGF |
| | chain FR2 | |
| 111 | 4M2-D9 heavy | TYSPSLESRISITRDTSKNQFFLQLISVTTEDTATYYC |
| | chain FR3 | |
| 112 | 4M2-D9 heavy | WGAGTSVTVSS |
| | chain FR4 | |
| 113 | 4M2-D9 light chain | DIVMTQSPGSLAVSLGQRATISCRASESVEYYGTSLMQWYLQKPGQPPKLLIYAASNVESGVPDR |
| | variable region | FRGSGSGTDFSLNIHPVEEDDIAMYFCQQSRKVPWTFGGGTKLEIK |
| 114 | 4M2-D9 light chain | ESVEYYGTSL |
| | CDR1 | |
| 115 | 4M2-D9 light chain | QQSRKVPWT |
| | CDR3 | |
| 116 | 4M2-D9 light chain | DIVMTQSPGSLAVSLGQRATISCRAS |
| | FR1 | |
| 117 | 4M2-D9 light chain | MQWYLQKPGQPPKLLIY |
| | FR2 | |
| 118 | 4M2-D9 light chain | NVESGVPDRFRGSGSGTDFSLNIHPVEEDDIAMYFC |
| | FR3 | |
| 119 | 1M2-D2 heavy | EVKVEESGGGLVQPGGSMKLSCAASGFTFSDAWMDWVHQSPEKGLEWVAEIRSKANNHATYYV |
| | chain variable | ESVEGRFTISRDDSKSSVFLQVNSLRPEDTGIYYCTRRDGYYFAYWGQGTLVTVSA |
| | region | |
| 120 | 1M2-D2 heavy | GFTFSDAW |
| | chain CDR1 | |
| 121 | 1M2-D2 heavy | IRSKANNHAT |
| | chain CDR2 | |
| 122 | 1M2-D2 heavy | TRRDGYYFAY |
| | chain CDR3 | |
| 123 | 1M2-D2 heavy | EVKVEESGGGLVQPGGSMKLSCAAS |
| | chain FR1 | |
| 124 | 1M2-D2 heavy | MDWVHQSPEKGLEWVAE |
| | chain FR2 | |
| 125 | 1M2-D2 heavy | YYVESVEGRFTISRDDSKSSVFLQVNSLRPEDTGIYYC |
| | chain FR3 | |
| 126 | 1M2-D2 light chain | DIVLTQSPAIMSASLGEEITLTCSASSSVRDMHWYQQKSGTSPKVLIYNTFNLASGVPSRFSGSGS |
| | variable region | GTFYSLTISSVEAGDAAAYYCHQWSSYPTFGGGTKLEIK |
| 127 | 1M2-D2 light chain | SSSVRD |
| | CDR1 | |
| 128 | 1M2-D2 light chain | NTF |
| | CDR2 | |
| 129 | 1M2-D2 light chain | HQWSSYPT |
| | CDR3 | |
| 130 | 1M2-D2 light chain | DIVLTQSPAIMSASLGEEITLTCSA |
| | FR1 | |
| 131 | 1M2-D2 light chain | MHWYQQKSGTSPKVLIY |
| | FR2 | |
| 132 | 1M2-D2 light chain | NLASGVPSRFSGSGSGTFYSLTISSVEAGDAAAYYC |
| | FR3 | |
| 133 | 5M1-A11 heavy | QVTLKVSGPGILQPSQTLSLTCSFSGFSLSTSGMGVSWIRQPSGKGLEWLAHIYWDDDKRYNPSL |
| | chain variable | KSRLTISKDSSSNQVFLKITSVDTADTATYYCARRLDGYNDPYYFDYWGQGTTLTVSS |
| | region | |
| 134 | 5M1-A11 heavy | QVTLKVSGPGILQPSQTLSLTCSFS |
| | chain FR1 | |
| 135 | 5M1-A11, 4M2-D5, | WGQGTTLTVSS |
| | 4M2-A8 heavy | |
| | chain FR4 | |
| 136 | 5M1-A11 light chain | DVVMTQTPALMSASPGEKVTMTCSASSSVSYMYWYQQKPRSSPKPWIYLTSNLASGVPARFSG |
| | variable region | SGSGTSYSLTISSMEAEDAATYYCQQWNSNPYTFGGGTKLEIK |
| 137 | 5M1-A11 light chain | SSVSY |
| | CDR1 | |
| 138 | 5M1-A11 light chain | LTS |
| | CDR2 | |
| 139 | 5M1-A11 light chain | QQWNSNPYT |
| | CDR3 | |
| 140 | 5M1-A11 light chain | DVVMTQTPALMSASPGEKVTMTCSAS |
| | FR1 | |
| 141 | 5M1-A11 light chain | MYWYQQKPRSSPKPWIY |
| | FR2 | |
| 142 | 5M1-A11 light chain | NLASGVPARFSGSGSGTSYSLTISSMEAEDAATYYC |
| | FR3 | |
| 143 | 4M2-D5 heavy | QVQLQQSGAELARPGASVRMSCKASGYTFTSYTMNWVKQRPGQGLEWIGFINPDSDYTTYDQK |
| | chain variable | FKDKATLTADSSSSTAYMQLSSLTYDDSAVYYCTRHSYGNYGDYWGQGTTLTVSS |
| | region | |
| 144 | 4M2-D5 heavy | GYTFTSYT |
| | chain CDR1 | |
| 145 | 4M2-D5 heavy | INPDSDYT |
| | chain CDR2 | |
| 146 | 4M2-D5 heavy | TRHSYGNYGDY |
| | chain CDR3 | |
| 147 | 4M2-D5 heavy | QVQLQQSGAELARPGASVRMSCKAS |
| | chain FR1 | |
| 148 | 4M2-D5 heavy | MNWVKQRPGQGLEWIGF |
| | chain FR2 | |
| 149 | 4M2-D5 heavy | TYDQKFKDKATLTADSSSSTAYMQLSSLTYDDSAVYYC |
| | chain FR3 | |
| 150 | 4M2-D5 light chain | DIVLTQSPKFLLVSAGDRVTITCKASQSVTNDVAWYQQKPGQSPKLLIYYASSRYTGVPDRFTGS |
| | variable region | GFGTDFTFTINTVQAEDLAVYFCQQDYSSPYTFGGGTKLEIK |
| 151 | 4M2-D5 light chain | QSVTND |
| | CDR1 | |
| 152 | 4M2-D5, 4M2-A8 | YAS |
| | light chain CDR2 | |
| 153 | 4M2-D5, 4M2-A8 | QQDYSSPYT |
| | light chain CDR3 | |
| 154 | 4M2-D5 light chain | DIVLTQSPKFLLVSAGDRVTITCKAS |
| | FR1 | |
| 155 | 4M2-D5, 4M2-A8 | VAWYQQKPGQSPKLLIY |
| | light chain FR2 | |
| 156 | 4M2-D5 light chain | SRYTGVPDRFTGSGFGTDFTFTINTVQAEDLAVYFC |
| | FR3 | |
| 157 | 4M2-A8 heavy | QVQLQQSGADLARPGASVKMSCKASGYTFIDYTVHWVKQRPGQGLEWIGFINPSNDYTSYNQKF |
| | chain variable | KDKASLTADTSSTTAYMQLSSLTSDDSAVYYCARHSYGNYGDYWGQGTTLTVSS |
| | region | |
| 158 | 4M2-A8 heavy | GYTFIDYT |
| | chain CDR1 | |
| 159 | 4M2-A8 heavy | INPSNDYT |
| | chain CDR2 | |
| 160 | 4M2-A8 heavy | ARHSYGNYGDY |
| | chain CDR3 | |
| 161 | 4M2-A8 heavy | QVQLQQSGADLARPGASVKMSCKAS |
| | chain FR1 | |
| 162 | 4M2-A8 heavy | VHWVKQRPGQGLEWIGF |
| | chain FR2 | |
| 163 | 4M2-A8 heavy | SYNQKFKDKASLTADTSSTTAYMQLSSLTSDDSAVYYC |
| | chain FR3 | |
| 164 | 4M2-A8 light chain | DIVMTQAPKFLLVSAGDRVTITCKASQSVTNGVAWYQQKPGQSPKLLIYYASNRYTGVPDRFTGS |
| | variable region | GFGTDFTFTISTVQAEDLAVYFCQQDYSSPYTFGGGTKLEIK |
| 165 | 4M2-A8 light chain | QSVTNG |
| | CDR1 | |
| 166 | 4M2-A8 light chain | DIVMTQAPKFLLVSAGDRVTITCKAS |
| | FR1 | |
| 167 | 4M2-A8 light chain | NRYTGVPDRFTGSGFGTDFTFTISTVQAEDLAVYFC |
| | FR3 | |
| 168 | 9M2-C12 heavy | QVQLQQSGAELVKPGASVRLSCKASGYTFTSYWMHWVRQRPGLGLEWIGEIDPSDSYTNCNQR |
| | chain variable | FKGKATLTVDKSSSTAYTQLSSLTSEDSAVYYCARWAYGPYAMDYWGQGTSVTVSS |
| | region | |
| 169 | 9M2-C12 heavy | GYTFTSYW |
| | chain CDR1 | |
| 170 | 9M2-C12 heavy | IDPSDSYT |
| | chain CDR2 | |
| 171 | 9M2-C12 heavy | ARWAYGPYAMDY |
| | chain CDR3 | |
| 172 | 9M2-C12 heavy | QVQLQQSGAELVKPGASVRLSCKAS |
| | chain FR1 | |
| 173 | 9M2-C12 heavy | MHWVRQRPGLGLEWIGE |
| | chain FR2 | |
| 174 | 9M2-C12 heavy | NCNQRFKGKATLTVDKSSSTAYTQLSSLTSEDSAVYYC |
| | chain FR3 | |
| 175 | 9M2-C12 heavy | WGQGTSVTVSS |
| | chain FR4 | |
| 176 | 9M2-C12 light chain | DIVMTQTPLSLPVSLGDQASISCRSSQSLVHSNGNTYLHWYLQKPGQSPKLLIYKVSNRFSGVPD |
| | variable region | RFSGSGSGTDFTLKISRVEAEDLGVYFCSQSTHVPWTFGGGTKLEIK |
| 177 | 9M2-C12 light chain | QSLVHSNGNTY |
| | CDR1 | |
| 178 | 9M2-C12, 13D5p, | KVS |
| | 13D5-1, 13D5-13 | |
| | light chain CDR2 | |
| 179 | 9M2-C12 light chain | SQSTHVPWT |
| | CDR3 | |
| 180 | 9M2-C12 light chain | DIVMTQTPLSLPVSLGDQASISCRSS |
| | FR1 | |
| 181 | 9M2-C12 light chain | LHWYLQKPGQSPKLLIY |
| | FR2 | |
| 182 | 9M2-C12 light chain | NRFSGVPDRFSGSGSGTDFTLKISRVEAEDLGVYFC |
| | FR3 | |
| 183 | 13D5p heavy chain | QVQLQQSGAELVRPGTSVKISCKASGYTFTNYWLGWVKQRPGHGLEWIGDIYPGGGYTNYNEKF |
| | variable region | KGKATLTADTSSSTAYMQLSSLTSEDSAVYFCARGGYYYGSSWYFDVWGAGTTVTVSS |
| 184 | 13D5p, 13D5-1, | IYPGGGYT |
| | 13D5-13 heavy | |
| | chain CDR2 | |
| 185 | 13D5p heavy chain | ARGGYYYGSSWYFDV |
| | CDR3 | |
| 186 | 13D5p, 13D5-1, | LGWVKQRPGHGLEWIGD |
| | 13D5-13 heavy | |
| | chain FR2 | |
| 187 | 13D5p, 13D5-1 | NYNEKFKGKATLTADTSSSTAYMQLSSLTSEDSAVYFC |
| | heavy chain FR3 | |
| 188 | 13D5p light chain | DVLMTQTPLSLPVSLGDQASISCRSSQSIVHSNGNTYLEWYLQKPGQSPKLLIYKVSNRFSGVPD |
| | variable region | RFSGSGSGTDFTLKISRVEAEDLGVYYCFQGSHVPPTFGGGTKLEIK |
| 189 | 13D5p light chain | QSIVHSNGNTY |
| | CDR1 | |
| 190 | 13D5p, 13D5-1, | FQGSHVPPT |
| | 13D5-13 light chain | |
| | CDR3 | |
| 191 | 13D5p, 13D5-1, | DVLMTQTPLSLPVSLGDQASISCRSS |
| | 13D5-13 light chain | |
| | FR1 | |
| 192 | 13D5p, 13D5-13 | LEWYLQKPGQSPKLLIY |
| | light chain FR2 | |
| 193 | 13D5p, 13D5-1 light | NRFSGVPDRFSGSGSGTDFTLKISRVEAEDLGVYYC |
| | chain FR3 | |
| 194 | 13D5-1 heavy chain | QVQLQQSGAELVRPGTSVKISCKASGYTFTNYWLGWVKQRPGHGLEWIGDIYPGGGYTNYNEKF |
| | variable region | KGKATLTADTSSSTAYMQLSSLTSEDSAVYFCVRSGYYYGSSWYFDVWGAGTTVTVSS |
| 195 | 13D5-1 heavy chain | VRSGYYYGSSWYFDV |
| | CDR3 | |
| 196 | 13D5-1 light chain | DVLMTQTPLSLPVSLGDQASISCRSSQSIVHSSGNTYLEWYLQKPDQSPKLLIYKVSNRFSGVPDR |
| | variable region | FSGSGSGTDFTLKISRVEAEDLGVYYCFQGSHVPPTFGGGTKLEIK |
| 197 | 13D5-1 light chain | QSIVHSSGNTY |
| | CDR1 | |
| 198 | 13D5-1 light chain | LEWYLQKPDQSPKLLIY |
| | FR2 | |
| 199 | 13D5-13 heavy | QVQLQQSGAELVRPGTSVKISCKASGYTFTNYWLGWVKQRPGHGLEWIGDIYPGGGYTNYNEKF |
| | chain variable | KGKATLTADISSSTAYMQLSSLTSEDSADYFCVRGGYYYGSSWYFDVWGAGTTVTVSS |
| | region | |
| 200 | 13D5-13 heavy | VRGGYYYGSSWYFDV |
| | chain CDR3 | |
| 201 | 13D5-13 heavy | NYNEKFKGKATLTADISSSTAYMQLSSLTSEDSADYFC |
| | chain FR3 | |
| 202 | 13D5-13 light chain | DVLMTQTPLSLPVSLGDQASISCRSSQSTVHSIGNTYLEWYLQKPGQSPKLLIYKVSNRFSGVPDR |
| | variable region | FSGSESGTDFTLKISRVEAEDLGVYYCFQGSHVPPTFGGGTKLEIK |
| 203 | 13D5-13 light chain | QSTVHSIGNTY |
| | CDR1 | |
| 204 | 13D5-13 light chain | NRFSGVPDRFSGSESGTDFTLKISRVEAEDLGVYYC |
| | FR3 | |
| 205 | Human lgG1 | ASTKGPSVFPLAPSSKSTSGGTAALGCLVKDYFPEPVTVSWNSGALTSGVHTFPAVLQSSGLYSL |
| | constant region | SSVVTVPSSSLGTQTYICNVNHKPSNTKVDKKVEPKSCDKTHTCPPCPAPELLGGPSVFLFPPKP |
| | (IGHG1; | KDTLMISRTPEVTCVVVDVSHEDPEVKFNWYVDGVEVHNAKTKPREEQYNSTYRVVSVLTVLHQ |
| | UniProt: P01857-1, | DWLNGKEYKCKVSNKALPAPIEKTISKAKGQPREPQVYTLPPSRDELTKNQVSLTCLVKGFYPSDI |
| | v1) | AVEWESNGQPENNYKTTPPVLDSDGSFFLYSKLTVDKSRWQQGNVFSCSVMHEALHNHYTQKS |
| | | LSLSPGK |
| 206 | CH1 lgG1 (positions | ASTKGPSVFPLAPSSKSTSGGTAALGCLVKDYFPEPVTVSWNSGALTSGVHTFPAVLQSSGLYSL |
| | 1-98 of P01857-1, | SSVVTVPSSSLGTQTYICNVNHKPSNTKVDKKV |
| | v1) | |
| 207 | Hinge IgG1 | EPKSCDKTHTCP |
| | (positions 99-110 of | |
| | P01857-1, v1) | |
| 208 | CH2 lgG1 (positions | PCPAPELLGGPSVFLFPPKPKDTLMISRTPEVTCVVVDVSHEDPEVKFNWYVDGVEVHNAKTKPR |
| | 111-223 of P01857- | EEQYNSTYRVVSVLTVLHQDWLNGKEYKCKVSNKALPAPIEKTISKAK |
| | 1, v1) | |
| 209 | CH3 lgG1 (positions | GQPREPQVYTLPPSRDELTKNQVSLTCLVKGFYPSDIAVEWESNGQPENNYKTTPPVLDSDGSF |
| | 224-330 of P01857- | FLYSKLTVDKSRWQQGNVFSCSVMHEALHNHYTQKSLSLSPGK |
| | 1, v1) | |
| 210 | CH3 (D356E, | GQPREPQVYTLPPSREEMTKNQVSLTCLVKGFYPSDIAVEWESNGQPENNYKTTPPVLDSDGSF |
| | L358M; positions | FLYSKLTVDKSRWQQGNVFSCSVMHEALHNHYTQKSLSLSPGK |
| | numbered | |
| | according to EU | |
| | numbering) | |
| 211 | Cκ CL (IGCK; | RTVAAPSVFIFPPSDEQLKSGTASVVCLLNNFYPREAKVQWKVDNALQSGNSQESVTEQDSKDS |
| | UniProt: P01834-1, | TYSLSSTLTLSKADYEKHKVYACEVTHQGLSSPVTKSFNRGEC |
| | v2) | |
| 212 | 4M2-C12 VH-CH1- | EVKLVESGPGLVKPSQSLSLTCTVTGYSITSDYAWNWIRQFPGNKLEWMGYITYSGNISYNPSLR |
| | CH2-CH3 | SRISITRDTSKNQFFLQLNSVTPEDTATYSCARSLYYPWYFDVWGAGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 213 | 4M2-C12 VL-Cκ | DIVITQTPAILSTSPGEKVTMTCRASSSVGYIHWYQQKPGSSPKPWIYATSNLASGVPARFSGSGS |
| | | GTSNSLTITRVEAEDAATYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 214 | 4M2-B4 VH-CH1- | EVMLVESGPGLVKPSQSLSLTCTVTGYSITSDYAWNWIRQFPGNKLEWMGYITYSGNISYNPSLR |
| | CH2-CH3 | SRISITRDTSKNQFFLQLNSVTPEDTATYSCARSLYYPWYFDVWGAGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 215 | 4M2-B4 VL-Cκ | DIVLTQTTAILSTSPGEKVTMTCRASSSVGYIHWYQQKPGSSPKPWIYATSNLASGVPARFSGSG |
| | | SGTSNSLTITRVEAEDAATYYCQQWSSYPPITFGGGTKLEVKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 216 | V4H1 VH-CH1- | EVQLLESGGGLVQPGGSLRLSCAASGYTITSDYAMSWVRQAPGKGLEWVSVITYSGNISYADSV |
| | CH2-CH3 | KGRFTISRDNSKNTLYLQMNSLRAEDTAVYYCARSLYYPWYFDVWGAGTTVTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 217 | V4H1 VL-Cκ | EIVITQSPATLSLSPGERATLSCRASSSVGYLAWYQQKPGQAPRPLIYDTSNRATGIPARFSGSGS |
| | | GTDNTLTISSLEPEDFAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 218 | V4H2 VH-CH1- | QVQLQESGPGLVKPSDTLSLTCTVSGYSITSDYAWSWIRQPPGKGLEWIGYITYSGNISYNPSLKS |
| | CH2-CH3 | RVTISVDTSKNQFSLKLSSVTAADTAVYDCARSLYYPWYFDVWGAGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 219 | V4H2 VL-Cκ | DIQMTQSPSSLSASVGDRVTITCRASSSVGYLNWYQQKPGKAPKPLIYAASSLQSGVPSRFSGSG |
| | | SGTDNTLTISSLQPEDFATYYCQQWSSYPPITFGGGTKLEVKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 220 | 2M1-B12 VH-CH1- | EVQLQQSGAELVRPGTSVKTSCKASGYTFTNYWLGWVKERAGHGLEWIGEIFPGGGHTNYKEKF |
| | CH2-CH3 | KGKATLTADTSSSTAYMKLSSLTSEDSAVYFCAQIPLYYGHYRSAYWGQGTLVTVSAASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 221 | 2M1-B12 VL-Cκ | DIQMMQSPASLSASVGETVAITCGASENIYGALNWYQRKQGKSPQLLIYGATNLADGMSSRFSGS |
| | | GSGRQYSLKISSLHPDDVATYYCQNVLSTPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 222 | 4M2-C9 VH-CH1- | QVTLKECGPGILQPSQTLSLTCSFSGFSLSTSGMGVSWIRQPSGKGLEWLAHIYWDDDKRYNPSL |
| | CH2-CH3 | KSRLTISKDSSSNQVFLKITSVDTADTATYYCARRLDGYNDPYYFDYWGQGTTLTVSSASTKGPS |
| | | VFPLAPSSKSTSGGTAALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEALHNHYTQKSLSLSPGK |
| 223 | 4M2-C9 VL-Cκ | DIVMTQSPSSLSASLGDTVTITCHASQNVNVWLSWYQQKPGNIPKLLIYKASNLHAGVPSRFSGS |
| | | GSGTGFTLTISSLQPEDIATYYCQQGQSYPLTFGGGTKLEIKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 224 | 2M1-D2 VH-CH1- | QVQLQQSGAELVRPGTSVKISCKASGYTFTNYWLGWVKERAGHGLEWIGEIFPGGGHTNYKEKF |
| | CH2-CH3 | KGKATLTADTSSSTAYMKLSSLTSEDSAVYFCAQIPLYYGHYRSAYWGQGTLVTVSAASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 225 | 2M1-D2 VL-Cκ | DIVMTQSPASLSASVGETVAITCGASENIYGALNWYQRKQGKSPQLLIYGATNLADGMSSRFSGS |
| | | GSGRQYSLKISSLHPDDVATYYCQNVLSTPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 226 | 4M2-D9 VH-CH1- | EVQLQESGPGLVKPSQSLSLTCTVTGYSITSDYAWNWIRQFPGNRLEWMGFISYSGFTTYSPSLE |
| | CH2-CH3 | SRISITRDTSKNQFFLQLISVTTEDTATYYCARNHYGGSYWYFDVWGAGTSVTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 227 | 4M2-D9 VL-Cκ | DIVMTQSPGSLAVSLGQRATISCRASESVEYYGTSLMQWYLQKPGQPPKLLIYAASNVESGVPDR |
| | | FRGSGSGTDFSLNIHPVEEDDIAMYFCQQSRKVPWTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 228 | 1M2-D2 VH-CH1- | EVKVEESGGGLVQPGGSMKLSCAASGFTFSDAWMDWVHQSPEKGLEWVAEIRSKANNHATYYV |
| | CH2-CH3 | ESVEGRFTISRDDSKSSVFLQVNSLRPEDTGIYYCTRRDGYYFAYWGQGTLVTVSAASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 229 | 1M2-D2 VL-Cκ | DIVLTQSPAIMSASLGEEITLTCSASSSVRDMHWYQQKSGTSPKVLIYNTFNLASGVPSRFSGSGS |
| | | GTFYSLTISSVEAGDAAAYYCHQWSSYPTFGGGTKLEIKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTH |
| | | QGLSSPVTKSFNRGEC |
| 230 | 5M1-A11 VH-CH1- | QVTLKVSGPGILQPSQTLSLTCSFSGFSLSTSGMGVSWIRQPSGKGLEWLAHIYWDDDKRYNPSL |
| | CH2-CH3 | KSRLTISKDSSSNQVFLKITSVDTADTATYYCARRLDGYNDPYYFDYWGQGTTLTVSSASTKGPS |
| | | VFPLAPSSKSTSGGTAALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEALHNHYTQKSLSLSPGK |
| 231 | 5M1-A11 VL-Cκ | DVVMTQTPALMSASPGEKVTMTCSASSSVSYMYWYQQKPRSSPKPWIYLTSNLASGVPARFSG |
| | | SGSGTSYSLTISSMEAEDAATYYCQQWNSNPYTFGGGTKLEIKRTVAAPSVFIFPPSDEQLKSGT |
| | | ASVVCLLNNFYPREAKVQWKVDNALQSGNSQESVTEQDSKDSTYSLSSTLTLSKADYEKHKVYA |
| | | CEVTHQGLSSPVTKSFNRGEC |
| 232 | 4M2-D5 VH-CH1- | QVQLQQSGAELARPGASVRMSCKASGYTFTSYTMNWVKQRPGQGLEWIGFINPDSDYTTYDQK |
| | CH2-CH3 | FKDKATLTADSSSSTAYMQLSSLTYDDSAVYYCTRHSYGNYGDYWGQGTTLTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 233 | 4M2-D5 VL-Cκ | DIVLTQSPKFLLVSAGDRVTITCKASQSVTNDVAWYQQKPGQSPKLLIYYASSRYTGVPDRFTGS |
| | | GFGTDFTFTINTVQAEDLAVYFCQQDYSSPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 234 | 4M2-A8 VH-CH1- | QVQLQQSGADLARPGASVKMSCKASGYTFIDYTVHWVKQRPGQGLEWIGFINPSNDYTSYNQKF |
| | CH2-CH3 | KDKASLTADTSSTTAYMQLSSLTSDDSAVYYCARHSYGNYGDYWGQGTTLTVSSASTKGPSVFP |
| | | LAPSSKSTSGGTAALGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQKSLSLSPGK |
| 235 | 4M2-A8 VL-Cκ | DIVMTQAPKFLLVSAGDRVTITCKASQSVTNGVAWYQQKPGQSPKLLIYYASNRYTGVPDRFTGS |
| | | GFGTDFTFTISTVQAEDLAVYFCQQDYSSPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 236 | 9M2-C12 VH-CH1- | QVQLQQSGAELVKPGASVRLSCKASGYTFTSYWMHWVRQRPGLGLEWIGEIDPSDSYTNCNQR |
| | CH2-CH3 | FKGKATLTVDKSSSTAYTQLSSLTSEDSAVYYCARWAYGPYAMDYWGQGTSVTVSSASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 237 | 9M2-C12 VL-Cκ | DIVMTQTPLSLPVSLGDQASISCRSSQSLVHSNGNTYLHWYLQKPGQSPKLLIYKVSNRFSGVPD |
| | | RFSGSGSGTDFTLKISRVEAEDLGVYFCSQSTHVPWTFGGGTKLEIKRTVAAPSVFIFPPSDEQLK |
| | | SGTASVVCLLNNFYPREAKVQWKVDNALQSGNSQESVTEQDSKDSTYSLSSTLTLSKADYEKHK |
| | | VYACEVTHQGLSSPVTKSFNRGEC |
| 238 | 13D5p VH-CH1- | QVQLQQSGAELVRPGTSVKISCKASGYTFTNYWLGWVKQRPGHGLEWIGDIYPGGGYTNYNEKF |
| | CH2-CH3 | KGKATLTADTSSSTAYMQLSSLTSEDSAVYFCARGGYYYGSSWYFDVWGAGTTVTVSSASTKGP |
| | | SVFPLAPSSKSTSGGTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNHYTQKSLSLSPGK |
| 239 | 13D5p VL-Cκ | DVLMTQTPLSLPVSLGDQASISCRSSQSIVHSNGNTYLEWYLQKPGQSPKLLIYKVSNRFSGVPD |
| | | RFSGSGSGTDFTLKISRVEAEDLGVYYCFQGSHVPPTFGGGTKLEIKRTVAAPSVFIFPPSDEQLK |
| | | SGTASVVCLLNNFYPREAKVQWKVDNALQSGNSQESVTEQDSKDSTYSLSSTLTLSKADYEKHK |
| | | VYACEVTHQGLSSPVTKSFNRGEC |
| 240 | 13D5-1 VH-CH1- | QVQLQQSGAELVRPGTSVKISCKASGYTFTNYWLGWVKQRPGHGLEWIGDIYPGGGYTNYNEKF |
| | CH2-CH3 | KGKATLTADTSSSTAYMQLSSLTSEDSAVYFCVRSGYYYGSSWYFDVWGAGTTVTVSSASTKGP |
| | | SVFPLAPSSKSTSGGTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNHYTQKSLSLSPGK |
| 241 | 13D5-1 VL-Cκ | DVLMTQTPLSLPVSLGDQASISCRSSQSIVHSSGNTYLEWYLQKPDQSPKLLIYKVSNRFSGVPDR |
| | | FSGSGSGTDFTLKISRVEAEDLGVYYCFQGSHVPPTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 242 | 13D5-13 VH-CH1- | QVQLQQSGAELVRPGTSVKISCKASGYTFTNYWLGWVKQRPGHGLEWIGDIYPGGGYTNYNEKF |
| | CH2-CH3 | KGKATLTADISSSTAYMQLSSLTSEDSADYFCVRGGYYYGSSWYFDVWGAGTTVTVSSASTKGP |
| | | SVFPLAPSSKSTSGGTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNHYTQKSLSLSPGK |
| 243 | 13D5-13 VL-Cκ | DVLMTQTPLSLPVSLGDQASISCRSSQSTVHSIGNTYLEWYLQKPGQSPKLLIYKVSNRFSGVPDR |
| | | FSGSESGTDFTLKISRVEAEDLGVYYCFQGSHVPPTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 244 | 4M2- | GYX₁ITSDYA |
| | C12/V4H1/V4H2 | wherein X₁ = S or T |
| | heavy chain CDR1 | |
| | consensus | |
| 245 | 4M2- | X₂X₃S |
| | C12/V4H1/V4H2 | wherein X₂ = A or D; X₃ = T or A |
| | light chain CDR2 | |
| | consensus | |
| 246 | 13D5p derived | X₄RX₅GYYYGSSWYFDV |
| | heavy chain CDR3 | wherein X₄ = V or A; X₅ = G or S |
| | consensus | |
| 247 | 13D5p derived light | QSX₆VHSX₇GNTY |
| | chain CDR1 | wherein X₆ = I or T; X₇ = N, S or I |
| | consensus | |
| 248 | 4M2-C12 mIgG2a | EVKLVESGPGLVKPSQSLSLTCTVTGYSITSDYAWNWIRQFPGNKLEWMGYITYSGNISYNPSLR |
| | HC | SRISITRDTSKNQFFLQLNSVTPEDTATYSCARSLYYPWYFDVWGAGTTVTVSSAKTTAPSVYPLA |
| | | PVCGDTTGSSVTLGCLVKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNLLGGPSVFIFPPKIKDVLMISLSPIVTCV |
| | | VVDVSEDDPDVQISWFVNNVEVHTAQTQTHREDYNSTLRVVSALPIQHQDWMSGKEFKCKVNNK |
| | | DLPAPIERTISKPKGSVRAPQVYVLPPPEEEMTKKQVTLTCMVTDFMPEDIYVEWTNNGKTELNYK |
| | | NTEPVLDSDGSYFMYSKLRVEKKNWVERNSYSCSVVHEGLHNHHTTKSFSRTPGK |
| 249 | 4M2-C12 mIgG2a | EVKLVESGPGLVKPSQSLSLTCTVTGYSITSDYAWNWIRQFPGNKLEWMGYITYSGNISYNPSLR |
| | LALA PG HC | SRISITRDTSKNQFFLQLNSVTPEDTATYSCARSLYYPWYFDVWGAGTTVTVSSAKTTAPSVYPLA |
| | | PVCGDTTGSSVTLGCLVKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNAAGGPSVFIFPPKIKDVLMISLSPIVTCV |
| | | VVDVSEDDPDVQISWFVNNVEVHTAQTQTHREDYNSTLRVVSALPIQHQDWMSGKEFKCKVNNK |
| | | DLGAPIERTISKPKGSVRAPQVYVLPPPEEEMTKKQVTLTCMVTDFMPEDIYVEWTNNGKTELNY |
| | | KNTEPVLDSDGSYFMYSKLRVEKKNWVERNSYSCSVVHEGLHNHHTTKSFSRTPGK |
| 250 | 4M2-C12 LC | DIVITQTPAILSTSPGEKVTMTCRASSSVGYIHWYQQKPGSSPKPWIYATSNLASGVPARFSGSGS |
| | | GTSNSLTITRVEAEDAATYYCQQWSSYPPITFGGGTKLEVKRADAAPTVSIFPPSSEQLTSGGASV |
| | | VCFLNNFYPKDINVKWKIDGSERQNGVLNSWTDQDSKDSTYSMSSTLTLTKDEYERHNSYTCEA |
| | | THKTSTSPIVKSFNRNEC |
| 251 | mIgG2a CH1 | AKTTAPSVYPLAPVCGDTTGSSVTLGCLVKGYFPEPVTLTWNSGSLSSGVHTFPAVLQSDLYTLS |
| | | SSVTVTSSTWPSQSITCNVAHPASSTKVDKKI |
| 252 | mIgG2a Hinge | EPRGPTIKPCPPCKCP |
| 253 | mIgG2a CH2 | APNLLGGPSVFIFPPKIKDVLMISLSPIVTCVVVDVSEDDPDVQISWFVNNVEVHTAQTQTHREDYN |
| | | STLRVVSALPIQHQDWMSGKEFKCKVNNKDLPAPIERTISKPK |
| 254 | mIgG2a CH2 LALA | APNAAGGPSVFIFPPKIKDVLMISLSPIVTCVVVDVSEDDPDVQISWFVNNVEVHTAQTQTHREDY |
| | PG | NSTLRVVSALPIQHQDWMSGKEFKCKVNNKDLGAPIERTISKPK |
| 255 | mIgG2a CH3 | GSVRAPQVYVLPPPEEEMTKKQVTLTCMVTDFMPEDIYVEWTNNGKTELNYKNTEPVLDSDGSY |
| | | FMYSKLRVEKKNWVERNSYSCSVVHEGLHNHHTTKSFSRTPGK |
| 256 | Mouse Ig gamma- | AKTTAPSVYPLAPVCGDTTGSSVTLGCLVKGYFPEPVTLTWNSGSLSSGVHTFPAVLQSDLYTLS |
| | 2A chain C region, | SSVTVTSSTWPSQSITCNVAHPASSTKVDKKIEPRGPTIKPCPPCKCPAPNLLGGPSVFIFPPKIKD |
| | A allele | VLMISLSPIVTCVVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTCMVTDFMPEDIYVE |
| | | WTNNGKTELNYKNTEPVLDSDGSYFMYSKLRVEKKNWVERNSYSCSVVHEGLHNHHTTKSFSR |
| | | TPGK |
| 257 | mIgG2a CH2, CH3 | APNAAGGPSVFIFPPKIKDVLMISLSPIVTCVVVDVSEDDPDVQISWFVNNVEVHTAQTQTHREDY |
| | LALA PG | NSTLRVVSALPIQHQDWMSGKEFKCKVNNKDLGAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNSYSC |
| | | SVVHEGLHNHHTTKSFSRTPGK |
| 258 | 4M2-C12 mIgG2a | EVKLVESGPGLVKPSQSLSLTCTVTGYSITSDYAWNWIRQFPGNKLEWMGYITYSGNISYNPSLR |
| | NQ HC | SRISITRDTSKNQFFLQLNSVTPEDTATYSCARSLYYPWYFDVWGAGTTVTVSSAKTTAPSVYPLA |
| | | PVCGDTTGSSVTLGCLVKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNLLGGPSVFIFPPKIKDVLMISLSPIVTCV |
| | | VVDVSEDDPDVQISWFVNNVEVHTAQTQTHREDYQSTLRVVSALPIQHQDWMSGKEFKCKVNN |
| | | KDLPAPIERTISKPKGSVRAPQVYVLPPPEEEMTKKQVTLTCMVTDFMPEDIYVEWTNNGKTELNY |
| | | KNTEPVLDSDGSYFMYSKLRVEKKNWVERNSYSCSVVHEGLHNHHTTKSFSRTPGK |
| 259 | mIgG2a CH2 NQ | APNLLGGPSVFIFPPKIKDVLMISLSPIVTCVVVDVSEDDPDVQISWFVNNVEVHTAQTQTHREDY |
| | | QSTLRVVSALPIQHQDWMSGKEFKCKVNNKDLPAPIERTISKPK |
| 260 | mIgG2a CH2, CH3 | APNLLGGPSVFIFPPKIKDVLMISLSPIVTCVVVDVSEDDPDVQISWFVNNVEVHTAQTQTHREDY |
| | NQ | QSTLRVVSALPIQHQDWMSGKEFKCKVNNKDLPAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNSYSC |
| | | SVVHEGLHNHHTTKSFSRTPGK |
| 261 | Mouse IGHG1 | AKTTPPSVYPLAPGSAAQTNSMVTLGCLVKGYFPEPVTVTWNSGSLSSGVHTFPAVLQSDLYTLS |
| | | SSVTVPSSTWPSETVTCNVAHPASSTKVDKKIVPRDCGCKPCICTVPEVSSVFIFPPKPKDVLTITL |
| | | TPKVTCVVVDISKDDPEVQFSWFVDDVEVHTAQTQPREEQFNSTFRSVSELPIMHQDWLNGKEF |
| | | KCRVNSAAFPAPIEKTISKTKGRPKAPQVYTIPPPKEQMAKDKVSLTCMITDFFPEDITVEWQWNG |
| | | QPAENYKNTQPIMNTNGSYFVYSKLNVQKSNWEAGNTFTCSVLHEGLHNHHTEKSLSHSPGK |
| 262 | mIgG1 CH1 | AKTTPPSVYPLAPGSAAQTNSMVTLGCLVKGYFPEPVTVTWNSGSLSSGVHTFPAVLQSDLYTLS |
| | | SSVTVPSSTWPSETVTCNVAHPASSTKVDKKI |
| 263 | mIgG1 Hinge | VPRDCGCKPCICT |
| 264 | mIgG1 CH2 | VPEVSSVFIFPPKPKDVLTITLTPKVTCVVVDISKDDPEVQFSWFVDDVEVHTAQTQPREEQFNST |
| | | FRSVSELPIMHQDWLNGKEFKCRVNSAAFPAPIEKTISKTK |
| 265 | mIgG1 CH3 | GRPKAPQVYTIPPPKEQMAKDKVSLTCMITDFFPEDITVEWQWNGQPAENYKNTQPIMNTNGSYF |
| | | VYSKLNVQKSNWEAGNTFTCSVLHEGLHNHHTEKSLSHSPGK |
| 266 | 4M2-C12 mIgG1 | EVKLVESGPGLVKPSQSLSLTCTVTGYSITSDYAWNWIRQFPGNKLEWMGYITYSGNISYNPSLR |
| | HC | SRISITRDTSKNQFFLQLNSVTPEDTATYSCARSLYYPWYFDVWGAGTTVTVSSAKTTPPSVYPLA |
| | | PGSAAQTNSMVTLGCLVKGYFPEPVTVTWNSGSLSSGVHTFPAVLQSDLYTLSSSVTVPSSTWP |
| | | SETVTCNVAHPASSTKVDKKIVPRDCGCKPCICTVPEVSSVFIFPPKPKDVLTITLTPKVTCVVVDIS |
| | | KDDPEVQFSWFVDDVEVHTAQTQPREEQFNSTFRSVSELPIMHQDWLNGKEFKCRVNSAAFPA |
| | | PIEKTISKTKGRPKAPQVYTIPPPKEQMAKDKVSLTCMITDFFPEDITVEWQWNGQPAENYKNTQP |
| | | IMNTNGSYFVYSKLNVQKSNWEAGNTFTCSVLHEGLHNHHTEKSLSHSPGK |
| 267 | IGN175A HC | QVQLQQSGAELMKPGASVKISCKATGYTFSTHWIEWVKQRPGHGLEWIGEILPGSGSTSYNEKF |
| | | KGKATFTADTSSNTAYMQLSSLTSEDSAVYYCARWLLYYYAMDYWGQGTSVTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 268 | IGN175A LC | DVLMTQTPLSLPVSLGDQASISCRSSQSIVHSNGNTYLEWYLQKPGQSPKLLIYKLSNRFSGVPDR |
| | | FSGSGSGTDFTLKISRVEAEDLGVYYCFQGSHFPYTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 269 | VSTB112 HC | QVQLVQSGAEVKKPGSSVKVSCKASGGTFSSYAISWVRQAPGQGLEWMGGIIPIFGTANYAQKF |
| | | QGRVTITADESTSTAYMELSSLRSEDTAVYYCARSSYGWSYEFDYWGQGTLVTVSSASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 270 | VSTB112 LC | DIQMTQSPSSLSASVGDRVTITCRASQSIDTRLNWYQQKPGKAPKLLIYSASSLQSGVPSRFSGS |
| | | GSGTDFTLTISSLQPEDFATYYCQQSAYNPITFGQGTKVEIKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 271 | VSTB112 major | PVDKGHDVTF |
| | epitope 1 | |
| 272 | VSTB112 major | RRPIRNLTFQDL |
| | epitope 2 | |
| 273 | VSTB112 minor | TWYRSSRGEVQTCS |
| | epitope 1 | |
| 274 | VSTB112 minor | EIRHHHSEHRVHGAMEL |
| | epitope 2 | |
| 275 | IGN175A epitope | FKVATPYSLYVCPEGQNVTLTCRLLGPVDKGH |
| 276 | V4-C1 heavy chain | QVQLQESGPGLVKPSDTLSLTCTVSGYSITSDYAWNWIRQTPGKGLEWIGYITYSGYISYNPSLRS |
| | variable region | RVTISRDTSKNQFSLKLSSVTAADTAVYSCARALYYPWYFDVWGTGTTVTVSS |
| 277 | V4-C1 heavy chain | ITYSGYI |
| | CDR2 | |
| 278 | V4-C1, V4-C9, V4- | ARALYYPWYFDV |
| | C24, V4-C26, V4- | |
| | C27, V4-C28, V4- | |
| | C30, V4-C31 heavy | |
| | chain CDR3 | |
| 279 | V4-C1, V4-C9 | WNWIRQTPGKGLEWIGY |
| | heavy chain FR2 | |
| 280 | V4-C1, V4-C9 | SYNPSLRSRVTISRDTSKNQFSLKLSSVTAADTAVYSC |
| | heavy chain FR3 | |
| 281 | V4-C1, V4-C9, V4- | WGTGTTVTVSS |
| | C24, V4-C26, V4- | |
| | C27, V4-C28, V4- | |
| | C30, V4-C31 heavy | |
| | chain FR4 | |
| 282 | V4-C1 light chain | EIVITQSPATLSLSPGERATLSCRASSSVGYIHWYQQKPGQAPRPLIYATSNRATGIPARFSGSGS |
| | variable region | GTDNTLTISSLEPEDSAVYYCQQWSSYPPITFGGGTKLEVK |
| 283 | V4-C1, V4-C9, V4- | IHWYQQKPGQAPRPLIY |
| | C24, V4-C27, V4- | |
| | C28, V4-C30, V4- | |
| | C31 light chain FR2 | |
| 284 | V4-C1, V4-C9, V4- | NRATGIPARFSGSGSGTDNTLTISSLEPEDSAVYYC |
| | C26, V4-C27 light | |
| | chain FR3 | |
| 285 | V4-C9 heavy chain | QVQLQESGPGLVKPSDTLSLTCTVSGYSITSDYAWNWIRQTPGKGLEWIGYITYSGYVSYNPSLR |
| | variable region | SRVTISRDTSKNQFSLKLSSVTAADTAVYSCARALYYPWYFDVWGTGTTVTVSS |
| 286 | V4-C9 heavy chain | ITYSGYV |
| | CDR2 | |
| 287 | V4-C9 light chain | EIVLTQSPATLSLSPGERATLSCRASSSVGYIHWYQQKPGQAPRPLIYATSNRATGIPARFSGSGS |
| | variable region | GTDNTLTISSLEPEDSAVYYCQQWSSYPPITFGGGTKLEVK |
| 288 | V4-C9, V4-C24, V4- | EIVLTQSPATLSLSPGERATLSCRAS |
| | C26, V4-C27, V4- | |
| | C28, V4-C30, V4- | |
| | C31 light chain FR1 | |
| 289 | V4-C24, V4-C26, | QVQLQESGPGLVKPSDTLSLTCTVSGYSITSDYTWNWIRQTPGKGLEWIGHITYSGSVSYNPSLR |
| | V4-C27, V4-C28, | SRVTISRDTSKNQFSLKLSSVTAADTATYSCARALYYPWYFDVWGTGTTVTVSS |
| | V4-C30, V4-C31 | |
| | heavy chain | |
| | variable region | |
| 290 | V4-C24, V4-C26, | GYSITSDYT |
| | V4-C27, V4-C28, | |
| | V4-C30, V4-C31 | |
| | heavy chain CDR1 | |
| 291 | V4-C24, V4-C26, | ITYSGSV |
| | V4-C27, V4-C28, | |
| | V4-C30, V4-C31 | |
| | heavy chain CDR2 | |
| 292 | V4-C24, V4-C26, | WNWIRQTPGKGLEWIGH |
| | V4-C27, V4-C28, | |
| | V4-C30, V4-C31 | |
| | heavy chain FR2 | |
| 293 | V4-C24, V4-C26, | SYNPSLRSRVTISRDTSKNQFSLKLSSVTAADTATYSC |
| | V4-C27, V4-C28, | |
| | V4-C30, V4-C31 | |
| | heavy chain FR3 | |
| 294 | V4-C24 light chain | EIVLTQSPATLSLSPGERATLSCRASSSVGYIHWYQQKPGQAPRPLIYTTSYRATGIPARFSGSGS |
| | variable region | GTDNTLTISSLEPEDSAVYYCQQWSSYPPITFGGGTKLEVK |
| 295 | V4-C24, V4-C26 | TTS |
| | light chain CDR2 | |
| 296 | V4-C24, V4-C28, | YRATGIPARFSGSGSGTDNTLTISSLEPEDSAVYYC |
| | V4-C30 light chain | |
| | FR3 | |
| 297 | V4-C26 light chain | EIVLTQSPATLSLSPGERATLSCRASSSVGYIHWYQQKPGQAPRPIIYTTSNRATGIPARFSGSGS |
| | variable region | GTDNTLTISSLEPEDSAVYYCQQWSSYPPITFGGGTKLEVK |
| 298 | V4-C26 light chain | IHWYQQKPGQAPRPIIY |
| | FR2 | |
| 299 | V4-C27 light chain | EIVLTQSPATLSLSPGERATLSCRASSSVGYIHWYQQKPGQAPRPLIYATYNRATGIPARFSGSGS |
| | variable region | GTDNTLTISSLEPEDSAVYYCQQWSSYPPITFGGGTKLEVK |
| 300 | V4-C27, V4-C30, | ATY |
| | V4-C31 light chain | |
| | CDR2 | |
| 301 | V4-C28 light chain | EIVLTQSPATLSLSPGERATLSCRASSSVGYIHWYQQKPGQAPRPLIYATSYRATGIPARFSGSGS |
| | variable region | GTDNTLTISSLEPEDSAVYYCQQWSSYPPITFGGGTKLEVK |
| 302 | V4-C30 light chain | EIVLTQSPATLSLSPGERATLSCRASSSVGYIHWYQQKPGQAPRPLIYATYYRATGIPARFSGSGS |
| | variable region | GTDNTLTISSLEPEDSAVYYCQQWSSYPPITFGGGTKLEVK |
| 303 | V4-C31 light chain | EIVLTQSPATLSLSPGERATLSCRASSSVGYIHWYQQKPGQAPRPLIYATYYRTTGIPARFSGSGS |
| | variable region | GTDNTLTISSLEPEDSAVYYCQQWSSYPPITFGGGTKLEVK |
| 304 | V4-C31 light chain | YRTTGIPARFSGSGSGTDNTLTISSLEPEDSAVYYC |
| | FR3 | |
| 305 | 4M2-C12 derived | GYX₈ITSDYX₉ |
| | heavy chain CDR1 | wherein X₈ = S or T; X₉ = T or A |
| | consensus | |
| 306 | 4M2-C12 derived | ITYSGX₁₀X₁₁ |
| | heavy chain CDR2 | wherein X₁₀ = S, N or Y; X₁₁ = Vor I |
| | consensus | |
| 307 | 4M2-C12 derived | ARX₁₂LYYPWYFDV |
| | heavy chain CDR3 | wherein X₁₂ = A or S |
| | consensus | |
| 308 | 4M2-C12 derived | X₁₃X₁₄X₁₅ |
| | light chain CDR2 | wherein X₁₃ = A, T or D; X₁₄ = T or A; X₁₅ = S or Y |
| | consensus | |
| 309 | C24/C26/C27 light | X₁₆TX₁₇ |
| | chain CDR2 | wherein X₁₆ = T or A; X₁₇ = S or Y |
| | consensus | |
| 310 | C24/C26/C27 light | EIVLTQSPATLSLSPGERATLSCRASSSVGYIHWYQQKPGQAPRPX₁₇LYX₁₈TX₁₉X₂₀YNRATGIPAR |
| | chain variable | FSGSGSGTDNTLTISSLEPEDSAVYYCQQWSSYPPITFGGGTKLEVK |
| | region consensus | wherein X₁₇ = L or 1; X₁₈ = T or A; X₁₉ = S or Y; X₂₀ = N or Y |
| 311 | V4-C1 VH-CH1- | QVQLQESGPGLVKPSDTLSLTCTVSGYSITSDYAWNWIRQTPGKGLEWIGYITYSGYISYNPSLRS |
| | CH2-CH3 | RVTISRDTSKNQFSLKLSSVTAADTAVYSCARALYYPWYFDVWGTGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 312 | V4-C1 VL-Cκ | EIVITQSPATLSLSPGERATLSCRASSSVGYIHWYQQKPGQAPRPLIYATSNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 313 | V4-C9 VH-CH1- | QVQLQESGPGLVKPSDTLSLTCTVSGYSITSDYAWNWIRQTPGKGLEWIGYITYSGYVSYNPSLR |
| | CH2-CH3 | SRVTISRDTSKNQFSLKLSSVTAADTAVYSCARALYYPWYFDVWGTGTTVTVSSASTKGPSVFPL |
| | | APSSKSTSGGTAALGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLSPGK |
| 314 | V4-C9 VL-Cκ | EIVLTQSPATLSLSPGERATLSCRASSSVGYIHWYQQKPGQAPRPLIYATSNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 315 | V4- | QVQLQESGPGLVKPSDTLSLTCTVSGYSITSDYTWNWIRQTPGKGLEWIGHITYSGSVSYNPSLR |
| | C24/C26/C27/C28/ | SRVTISRDTSKNQFSLKLSSVTAADTATYSCARALYYPWYFDVWGTGTTVTVSSASTKGPSVFPL |
| | C30/C31 VH-CH1- | APSSKSTSGGTAALGCLVKDYFPEPVTVSWNSGALTSGVHTFPAVLQSSGLYSLSSVVTVPSSSL |
| | CH2-CH3 | GTQTYICNVNHKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLSPGK |
| 316 | V4-C24 VL-Cκ | EIVLTQSPATLSLSPGERATLSCRASSSVGYIHWYQQKPGQAPRPLIYTTSYRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 317 | V4-C26 VL-Cκ | EIVLTQSPATLSLSPGERATLSCRASSSVGYIHWYQQKPGQAPRPIIYTTSNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 318 | V4-C27 VL-Cκ | EIVLTQSPATLSLSPGERATLSCRASSSVGYIHWYQQKPGQAPRPLIYATYNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 319 | V4-C28 VL-Cκ | EIVLTQSPATLSLSPGERATLSCRASSSVGYIHWYQQKPGQAPRPLIYATSYRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 320 | V4-C30 VL-Cκ | EIVLTQSPATLSLSPGERATLSCRASSSVGYIHWYQQKPGQAPRPLIYATYYRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 321 | V4-C31 VL-Cκ | EIVLTQSPATLSLSPGERATLSCRASSSVGYIHWYQQKPGQAPRPLIYATYYRTTGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 322 | VISTA sequence to | SRGEVQ |
| | which 4M2-C12 and | |
| | derivatives bind | |
| 323 | Human PSGL-1 | MPLQLLLLLILLGPGNSLQLWDTWADEAEKALGPLLARDRRQATEYEYLDYDFLPETEPPEMLRN |
| | isoform 1 (UniProt: | STDTTPLTGPGTPESTTVEPAARRSTGLDAGGAVTELTTELANMGNLSTDSAAMEIQTTQPAATE |
| | Q14242-1, v1) | AQTTQPVPTEAQTTPLAATEAQTTRLTATEAQTTPLAATEAQTTPPAATEAQTTQPTGLEAQTTAP |
| | | AAMEAQTTAPAAMEAQTTPPAAMEAQTTQTTAMEAQTTAPEATEAQTTQPTATEAQTTPLAAME |
| | | ALSTEPSATEALSMEPTTKRGLFIPFSVSSVTHKGIPMAASNLSVNYPVGAPDHISVKQCLLAILILA |
| | | LVATIFFVCTVVLAVRLSRKGHMYPVRNYSPTEMVCISSLLPDGGEGPSATANGGLSKAKSPGLTP |
| | | EPREDREGDDLTLHSFLP |
| 324 | Human PSGL-1 | MAVGASGLEGDKMAGAMPLQLLLLLILLGPGNSLQLWDTWADEAEKALGPLLARDRRQATEYEY |
| | isoform 2 (UniProt: | LDYDFLPETEPPEMLRNSTDTTPLTGPGTPESTTVEPAARRSTGLDAGGAVTELTTELANMGNLS |
| | Q14242-2) | TDSAAMEIQTTQPAATEAQTTQPVPTEAQTTPLAATEAQTTRLTATEAQTTPLAATEAQTTPPAAT |
| | | EAQTTQPTGLEAQTTAPAAMEAQTTAPAAMEAQTTPPAAMEAQTTQTTAMEAQTTAPEATEAQT |
| | | TQPTATEAQTTPLAAMEALSTEPSATEALSMEPTTKRGLFIPFSVSSVTHKGIPMAASNLSVNYPV |
| | | GAPDHISVKQCLLAILILALVATIFFVCTVVLAVRLSRKGHMYPVRNYSPTEMVCISSLLPDGGEGP |
| | | SATANGGLSKAKSPGLTPEPREDREGDDLTLHSFLP |
| 325 | Mature human | LQLWDTWADEAEKALGPLLARDRRQATEYEYLDYDFLPETEPPEMLRNSTDTTPLTGPGTPESTT |
| | PSGL-1 isoform 1 | VEPAARRSTGLDAGGAVTELTTELANMGNLSTDSAAMEIQTTQPAATEAQTTQPVPTEAQTTPLA |
| | (Q14242-1, v1 | ATEAQTTRLTATEAQTTPLAATEAQTTPPAATEAQTTQPTGLEAQTTAPAAMEAQTTAPAAMEAQ |
| | positions 18 to 412) | TTPPAAMEAQTTQTTAMEAQTTAPEATEAQTTQPTATEAQTTPLAAMEALSTEPSATEALSMEPT |
| | | TKRGLFIPFSVSSVTHKGIPMAASNLSVNYPVGAPDHISVKQCLLAILILALVATIFFVCTVVLAVRLS |
| | | RKGHMYPVRNYSPTEMVCISSLLPDGGEGPSATANGGLSKAKSPGLTPEPREDREGDDLTLHSF |
| | | LP |
| 326 | PSGL-1 | LQLWDTWADEAEKALGPLLARDRRQATEYEYLDYDFLPETEPPEMLRNSTDTTPLTGPGTPESTT |
| | extracellular domain | VEPAARRSTGLDAGGAVTELTTELANMGNLSTDSAAMEIQTTQPAATEAQTTQPVPTEAQTTPLA |
| | (Q14242-1, v1 | ATEAQTTRLTATEAQTTPLAATEAQTTPPAATEAQTTQPTGLEAQTTAPAAMEAQTTAPAAMEAQ |
| | positions 18 to 320) | TTPPAAMEAQTTQTTAMEAQTTAPEATEAQTTQPTATEAQTTPLAAMEALSTEPSATEALSMEPT |
| | | TKRGLFIPFSVSSVTHKGIPMAASNLSVNYPVGAPDHISVKQC |
| 327 | PSGL-1 | LLAILILALVATIFFVCTVVL |
| | transmembrane | |
| | domain (Q14242-1, | |
| | v1 positions 321 to | |
| | 341) | |
| 328 | PSGL-1 | AVRLSRKGHMYPVRNYSPTEMVCISSLLPDGGEGPSATANGGLSKAKSPGLTPEPREDREGDDL |
| | cytoplasmic domain | TLHSFLP |
| | (Q14242-1, v1 | |
| | positions 342 to | |
| | 412) | |
| 329 | PSGL-1 | QTTQPAATEAQTTQPVPTEAQTTPLAATEAQTTRLTATEAQTTPLAATEAQTTPPAATEAQTTQPT |
| | extracellular domain | GLEAQTTAPAAMEAQTTAPAAMEAQTTPPAAMEAQTTQTTAMEAQTTAPEATEAQTTQPTATEA |
| | repeat region | QTTPLAAMEA |
| | (Q14242-1, v1 | |
| | positions 122 to | |
| | 261) | |
| 330 | 4M2-C12 VH-CH1- | EVKLVESGPGLVKPSQSLSLTCTVTGYSITSDYAWNWIRQFPGNKLEWMGYITYSGNISYNPSLR |
| | CH2-CH3 IgG4 | SRISITRDTSKNQFFLQLNSVTPEDTATYSCARSLYYPWYFDVWGAGTTVTVSSASTKGPSVFPLA |
| | | PCSRSTSESTAALGCLVKDYFPEPVTVSWNSGALTSGVHTFPAVLQSSGLYSLSSVVTVPSSSLG |
| | | TKTYTCNVDHKPSNTKVDKRVESKYGPPCPSCPAPEFLGGPSVFLFPPKPKDTLMISRTPEVTCV |
| | | VVDVSQEDPEVQFNWYVDGVEVHNAKTKPREEQFNSTYRVVSVLTVLHQDWLNGKEYKCKVSN |
| | | KGLPSSIEKTISKAKGQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSLSLSLGK |
| 331 | V4-C26 | QVQLQESGPGLVKPSDTLSLTCTVSGYSITSDYTWNWIRQTPGKGLEWIGHITYSGSVSYNPSLR |
| | hIgG4(S228P) HC | SRVTISRDTSKNQFSLKLSSVTAADTATYSCARALYYPWYFDVWGTGTTVTVSSASTKGPSVFPL |
| | | APCSRSTSESTAALGCLVKDYFPEPVTVSWNSGALTSGVHTFPAVLQSSGLYSLSSVVTVPSSSL |
| | | GTKTYTCNVDHKPSNTKVDKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTVLHQDWLNGKEYKCKVS |
| | | NKGLPSSIEKTISKAKGQPREPQVYTLPPSQEEMTKNQVSLTCLVKGFYPSDIAVEWESNGQPEN |
| | | NYKTTPPVLDSDGSFFLYSRLTVDKSRWQEGNVFSCSVMHEALHNHYTQKSLSLSLGK |
| 332 | Human LRIG1 | MARPVRGGLGAPRRSPCLLLLWLLLLRLEPVTAAAGPRAPCAAACTCAGDSLDCGGRGLAALPG |
| | isoform 1 (Q96JA1- | DLPSWTRSLNLSYNKLSEIDPAGFEDLPNLQEVYLNNNELTAVPSLGAASSHVVSLFLQHNKIRSV |
| | 1, v2) | EGSQLKAYLSLEVLDLSLNNITEVRNTCFPHGPPIKELNLAGNRIGTLELGAFDGLSRSLLTLRLSK |
| | | NRITQLPVRAFKLPRLTQLDLNRNRIRLIEGLTFQGLNSLEVLKLQRNNISKLTDGAFWGLSKMHVL |
| | | HLEYNSLVEVNSGSLYGLTALHQLHLSNNSIARIHRKGWSFCQKLHELVLSFNNLTRLDEESLAEL |
| | | SSLSVLRLSHNSISHIAEGAFKGLRSLRVLDLDHNEISGTIEDTSGAFSGLDSLSKLTLFGNKIKSVA |
| | | KRAFSGLEGLEHLNLGGNAIRSVQFDAFVKMKNLKELHISSDSFLCDCQLKWLPPWLIGRMLQAF |
| | | VTATCAHPESLKGQSIFSVPPESFVCDDFLKPQIITQPETTMAMVGKDIRFTCSAASSSSSPMTFA |
| | | WKKDNEVLTNADMENFVHVHAQDGEVMEYTTILHLRQVTFGHEGRYQCVITNHFGSTYSHKARL |
| | | TVNVLPSFTKTPHDITIRTTTMARLECAATGHPNPQIAWQKDGGTDFPAARERRMHVMPDDDVFF |
| | | ITDVKIDDAGVYSCTAQNSAGSISANATLTVLETPSLVVPLEDRVVSVGETVALQCKATGNPPPRIT |
| | | WFKGDRPLSLTERHHLTPDNQLLVVQNVVAEDAGRYTCEMSNTLGTERAHSQLSVLPAAGCRKD |
| | | GTTVGIFTIAVVSSIVLTSLVWVCIIYQTRKKSEEYSVTNTDETVVPPDVPSYLSSQGTLSDRQETVV |
| | | RTEGGPQANGHIESNGVCPRDASHFPEPDTHSVACRQPKLCAGSAYHKEPWKAMEKAEGTPGP |
| | | HKMEHGGRVVCSDCNTEVDCYSRGQAFHPQPVSRDSAQPSAPNGPEPGGSDQEHSPHHQCS |
| | | RTAAGSCPECQGSLYPSNHDRMLTAVKKKPMASLDGKGDSSWTLARLYHPDSTELQPASSLTSG |
| | | SPERAEAQYLLVSNGHLPKACDASPESTPLTGQLPGKQRVPLLLAPKS |
| 333 | Mature human | AGPRAPCAAACTCAGDSLDCGGRGLAALPGDLPSWTRSLNLSYNKLSEIDPAGFEDLPNLQEVYL |
| | LRIG1 isoform 1 | NNNELTAVPSLGAASSHVVSLFLQHNKIRSVEGSQLKAYLSLEVLDLSLNNITEVRNTCFPHGPPIK |
| | | ELNLAGNRIGTLELGAFDGLSRSLLTLRLSKNRITQLPVRAFKLPRLTQLDLNRNRIRLIEGLTFQGL |
| | | NSLEVLKLQRNNISKLTDGAFWGLSKMHVLHLEYNSLVEVNSGSLYGLTALHQLHLSNNSIARIHR |
| | | KGWSFCQKLHELVLSFNNLTRLDEESLAELSSLSVLRLSHNSISHIAEGAFKGLRSLRVLDLDHNEI |
| | | SGTIEDTSGAFSGLDSLSKLTLFGNKIKSVAKRAFSGLEGLEHLNLGGNAIRSVQFDAFVKMKNLK |
| | | ELHISSDSFLCDCQLKWLPPWLIGRMLQAFVTATCAHPESLKGQSIFSVPPESFVCDDFLKPQIITQ |
| | | PETTMAMVGKDIRFTCSAASSSSSPMTFAWKKDNEVLTNADMENFVHVHAQDGEVMEYTTILHL |
| | | RQVTFGHEGRYQCVITNHFGSTYSHKARLTVNVLPSFTKTPHDITIRTTTMARLECAATGHPNPQI |
| | | AWQKDGGTDFPAARERRMHVMPDDDVFFITDVKIDDAGVYSCTAQNSAGSISANATLTVLETPSL |
| | | VVPLEDRVVSVGETVALQCKATGNPPPRITWFKGDRPLSLTERHHLTPDNQLLVVQNVVAEDAG |
| | | RYTCEMSNTLGTERAHSQLSVLPAAGCRKDGTTVGIFTIAVVSSIVLTSLVWVCIIYQTRKKSEEYS |
| | | VTNTDETVVPPDVPSYLSSQGTLSDRQETVVRTEGGPQANGHIESNGVCPRDASHFPEPDTHSV |
| | | ACRQPKLCAGSAYHKEPWKAMEKAEGTPGPHKMEHGGRVVCSDCNTEVDCYSRGQAFHPQPV |
| | | SRDSAQPSAPNGPEPGGSDQEHSPHHQCSRTAAGSCPECQGSLYPSNHDRMLTAVKKKPMAS |
| | | LDGKGDSSWTLARLYHPDSTELQPASSLTSGSPERAEAQYLLVSNGHLPKACDASPESTPLTGQL |
| | | PGKQRVPLLLAPKS |
| 334 | Human LRIG1 | MARPVRGGLGAPRRSPCLLLLWLLLLRLEPVTAAAGPRAPCAAACTCAGDSLDCGGRGLAALPG |
| | isoform 2 (Q96JA1- | DLPSWTRSLNLSYNKLSEIDPAGFEDLPNLQEVYLNNNELTAVPSLGAASSHVVSLFLQHNKIRSV |
| | 2) | EGSQLKAYLSLEVLDLSLNNITEVRNTCFPHGPPIKELNLAGNRIGTLELGAFDGLSRSLLTLRLSK |
| | | NRITQLPVRAFKLPRLTQLDLNRNRIRLIEGLTFQGLNSLEVLKLQRNNISKLTDGAFWGLSKMHVL |
| | | HLEYNSLVEVNSGSLYGLTALHQLHLSNNSIARIHRKGWSFCQKLHELVLSFNNLTRLDEESLAEL |
| | | SSLSVLRLSHNSISHIAEGAFKGLRSLRVLDLDHNEISGTIEDTSGAFSGLDSLSKLLLLEPSQSAGC |
| | | SSPSQPHMSAGGRTLFGNKIKSVAKRAFSGLEGLEHLNLGGNAIRSVQFDAFVKMKNLKELHISS |
| | | DSFLCDCQLKWLPPWLIGRMLQAFVTATCAHPESLKGQSIFSVPPESFVCDDFLKPQIITQPETTM |
| | | AMVGKDIRFTCSAASSSSSPMTFAWKKDNEVLTNADMENFVHVHAQDGEVMEYTTILHLRQVTF |
| | | GHEGRYQCVITNHFGSTYSHKARLTVNVLPSFTKTPHDITIRTTTMARLECAATGHPNPQIAWQKD |
| | | GGTDFPAAQTPSLVVPLEDRVVSVGETVALQCKATGNPPPRITWFKGDRPLSLTERHHLTPDNQL |
| | | LVVQNVVAEDAGRYTCEMSNTLGTERAHSQLSVLPAAGCRKDGTTVGIFTIAVVSSIVLTSLVWVC |
| | | IIYQTRKKSEEYSVTNTDETVVPPDVPSYLSSQGTLSDRQETVVRTEGGPQANGHIESNGVCPRD |
| | | ASHFPEPDTHSVACRQPKLCAGSAYHKEPWKAMEKAEGTPGPHKMEHGGRVVCSDCNTEVDC |
| | | YSRGQAFHPQPVSRDSAQPSAPNGPEPGGSDQEHSPHHQCSRTAAGSCPECQGSLYPSNHDR |
| | | MLTAVKKKPMASLDGKGDSSWTLARLYHPDSTELQPASSLTSGSPERAEAQYLLVSNGHLPKAC |
| | | DASPESTPLTGQLPGKQRVPLLLAPKS |
| 335 | Mature human | AGPRAPCAAACTCAGDSLDCGGRGLAALPGDLPSWTRSLNLSYNKLSEIDPAGFEDLPNLQEVYL |
| | LRIG1 isoform 2 | NNNELTAVPSLGAASSHVVSLFLQHNKIRSVEGSQLKAYLSLEVLDLSLNNITEVRNTCFPHGPPIK |
| | | ELNLAGNRIGTLELGAFDGLSRSLLTLRLSKNRITQLPVRAFKLPRLTQLDLNRNRIRLIEGLTFQGL |
| | | NSLEVLKLQRNNISKLTDGAFWGLSKMHVLHLEYNSLVEVNSGSLYGLTALHQLHLSNNSIARIHR |
| | | KGWSFCQKLHELVLSFNNLTRLDEESLAELSSLSVLRLSHNSISHIAEGAFKGLRSLRVLDLDHNEI |
| | | SGTIEDTSGAFSGLDSLSKLLLLEPSQSAGCSSPSQPHMSAGGRTLFGNKIKSVAKRAFSGLEGLE |
| | | HLNLGGNAIRSVQFDAFVKMKNLKELHISSDSFLCDCQLKWLPPWLIGRMLQAFVTATCAHPESL |
| | | KGQSIFSVPPESFVCDDFLKPQIITQPETTMAMVGKDIRFTCSAASSSSSPMTFAWKKDNEVLTNA |
| | | DMENFVHVHAQDGEVMEYTTILHLRQVTFGHEGRYQCVITNHFGSTYSHKARLTVNVLPSFTKTP |
| | | HDITIRTTTMARLECAATGHPNPQIAWQKDGGTDFPAAQTPSLVVPLEDRVVSVGETVALQCKAT |
| | | GNPPPRITWFKGDRPLSLTERHHLTPDNQLLVVQNVVAEDAGRYTCEMSNTLGTERAHSQLSVL |
| | | PAAGCRKDGTTVGIFTIAVVSSIVLTSLVWVCIIYQTRKKSEEYSVTNTDETVVPPDVPSYLSSQGT |
| | | LSDRQETVVRTEGGPQANGHIESNGVCPRDASHFPEPDTHSVACRQPKLCAGSAYHKEPWKAM |
| | | EKAEGTPGPHKMEHGGRVVCSDCNTEVDCYSRGQAFHPQPVSRDSAQPSAPNGPEPGGSDQE |
| | | HSPHHQCSRTAAGSCPECQGSLYPSNHDRMLTAVKKKPMASLDGKGDSSWTLARLYHPDSTEL |
| | | QPASSLTSGSPERAEAQYLLVSNGHLPKACDASPESTPLTGQLPGKQRVPLLLAPKS |
| 336 | Extracellular | AGPRAPCAAACTCAGDSLDCGGRGLAALPGDLPSWTRSLNLSYNKLSEIDPAGFEDLPNLQEVYL |
| | domain human | NNNELTAVPSLGAASSHVVSLFLQHNKIRSVEGSQLKAYLSLEVLDLSLNNITEVRNTCFPHGPPIK |
| | LRIG1 isoform 1 | ELNLAGNRIGTLELGAFDGLSRSLLTLRLSKNRITQLPVRAFKLPRLTQLDLNRNRIRLIEGLTFQGL |
| | | NSLEVLKLQRNNISKLTDGAFWGLSKMHVLHLEYNSLVEVNSGSLYGLTALHQLHLSNNSIARIHR |
| | | KGWSFCQKLHELVLSFNNLTRLDEESLAELSSLSVLRLSHNSISHIAEGAFKGLRSLRVLDLDHNEI |
| | | SGTIEDTSGAFSGLDSLSKLTLFGNKIKSVAKRAFSGLEGLEHLNLGGNAIRSVQFDAFVKMKNLK |
| | | ELHISSDSFLCDCQLKWLPPWLIGRMLQAFVTATCAHPESLKGQSIFSVPPESFVCDDFLKPQIITQ |
| | | PETTMAMVGKDIRFTCSAASSSSSPMTFAWKKDNEVLTNADMENFVHVHAQDGEVMEYTTILHL |
| | | RQVTFGHEGRYQCVITNHFGSTYSHKARLTVNVLPSFTKTPHDITIRTTTMARLECAATGHPNPQI |
| | | AWQKDGGTDFPAARERRMHVMPDDDVFFITDVKIDDAGVYSCTAQNSAGSISANATLTVLETPSL |
| | | VVPLEDRVVSVGETVALQCKATGNPPPRITWFKGDRPLSLTERHHLTPDNQLLVVQNVVAEDAG |
| | | RYTCEMSNTLGTERAHSQLSVLPAAGCRKDGTTVG |
| 337 | Extracellular | AGPRAPCAAACTCAGDSLDCGGRGLAALPGDLPSWTRSLNLSYNKLSEIDPAGFEDLPNLQEVYL |
| | domain human | NNNELTAVPSLGAASSHVVSLFLQHNKIRSVEGSQLKAYLSLEVLDLSLNNITEVRNTCFPHGPPIK |
| | LRIG1 isoform 2 | ELNLAGNRIGTLELGAFDGLSRSLLTLRLSKNRITQLPVRAFKLPRLTQLDLNRNRIRLIEGLTFQGL |
| | | NSLEVLKLQRNNISKLTDGAFWGLSKMHVLHLEYNSLVEVNSGSLYGLTALHQLHLSNNSIARIHR |
| | | KGWSFCQKLHELVLSFNNLTRLDEESLAELSSLSVLRLSHNSISHIAEGAFKGLRSLRVLDLDHNEI |
| | | SGTIEDTSGAFSGLDSLSKLLLLEPSQSAGCSSPSQPHMSAGGRTLFGNKIKSVAKRAFSGLEGLE |
| | | HLNLGGNAIRSVQFDAFVKMKNLKELHISSDSFLCDCQLKWLPPWLIGRMLQAFVTATCAHPESL |
| | | KGQSIFSVPPESFVCDDFLKPQIITQPETTMAMVGKDIRFTCSAASSSSSPMTFAWKKDNEVLTNA |
| | | DMENFVHVHAQDGEVMEYTTILHLRQVTFGHEGRYQCVITNHFGSTYSHKARLTVNVLPSFTKTP |
| | | HDITIRTTTMARLECAATGHPNPQIAWQKDGGTDFPAAQTPSLVVPLEDRVVSVGETVALQCKAT |
| | | GNPPPRITWFKGDRPLSLTERHHLTPDNQLLVVQNVVAEDAGRYTCEMSNTLGTERAHSQLSVL |
| | | PAAGCRKDGTTVG |
| 338 | Transmembrane | IFTIAVVSSIVLTSLVWVCII |
| | domain human | |
| | LRIG1 | |
| 339 | Cytoplasmic domain | YQTRKKSEEYSVTNTDETVVPPDVPSYLSSQGTLSDRQETVVRTEGGPQANGHIESNGVCPRDA |
| | human LRIG1 | SHFPEPDTHSVACRQPKLCAGSAYHKEPWKAMEKAEGTPGPHKMEHGGRVVCSDCNTEVDCY |
| | | SRGQAFHPQPVSRDSAQPSAPNGPEPGGSDQEHSPHHQCSRTAAGSCPECQGSLYPSNHDRM |
| | | LTAVKKKPMASLDGKGDSSWTLARLYHPDSTELQPASSLTSGSPERAEAQYLLVSNGHLPKACD |
| | | ASPESTPLTGQLPGKQRVPLLLAPKS |
| 340 | Region of VISTA | YKTWYRSSRGEVQTCSERRPIRNLTFQDL |
| | bound by 4M2-C12 | |
| | and derivative | |
| | clones | |
| 341 | Primary epitope of | ERRPIRNLTFQDL |
| | VISTA bound by | |
| | 4M2-C12 and | |
| | derivative clones | |
| 342 | Secondary epitope | YKTWYRSSRGEVQTCSE |
| | of VISTA bound by | |
| | 4M2-C12 and | |
| | derivative clones | |
| 343 | Region of VISTA to | FYKTWYRSSRGEVQTCSERRPIRNL |
| | which LRIG1 binds | |
| 344 | Human VISTA C-C' | TFYKTWYRSSRGEVQTCSERRPIRNLTFQDLHLHHGGH |
| | region | |
| 345 | Human IgG4 | ASTKGPSVFPLAPCSRSTSESTAALGCLVKDYFPEPVTVSWNSGALTSGVHTFPAVLQSSGLYSL |
| | constant region | SSVVTVPSSSLGTKTYTCNVDHKPSNTKVDKRVESKYGPPCPSCPAPEFLGGPSVFLFPPKPKDT |
| | (IGHG4; | LMISRTPEVTCVVVDVSQEDPEVQFNWYVDGVEVHNAKTKPREEQFNSTYRVVSVLTVLHQDWL |
| | UniProt:P01861-1, | NGKEYKCKVSNKGLPSSIEKTISKAKGQPREPQVYTLPPSQEEMTKNQVSLTCLVKGFYPSDIAVE |
| | v1) | WESNGQPENNYKTTPPVLDSDGSFFLYSRLTVDKSRWQEGNVFSCSVMHEALHNHYTQKSLSL |
| | | SLGK |
| 346 | Human IgG4 | ASTKGPSVFPLAPCSRSTSESTAALGCLVKDYFPEPVTVSWNSGALTSGVHTFPAVLQSSGLYSL |
| | (S228P) constant | SSVVTVPSSSLGTKTYTCNVDHKPSNTKVDKRVESKYGPPCPPCPAPEFLGGPSVFLFPPKPKDT |
| | region | LMISRTPEVTCVVVDVSQEDPEVQFNWYVDGVEVHNAKTKPREEQFNSTYRVVSVLTVLHQDWL |
| | | NGKEYKCKVSNKGLPSSIEKTISKAKGQPREPQVYTLPPSQEEMTKNQVSLTCLVKGFYPSDIAVE |
| | | WESNGQPENNYKTTPPVLDSDGSFFLYSRLTVDKSRWQEGNVFSCSVMHEALHNHYTQKSLSL |
| | | SLGK |
| 347 | Human IgG4 CH2- | APEFLGGPSVFLFPPKPKDTLMISRTPEVTCVVVDVSQEDPEVQFNWYVDGVEVHNAKTKPREE |
| | CH3 region | QFNSTYRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQEGNVF |
| | | SCSVMHEALHNHYTQKSLSLSLGK |
| 348 | Cynomolgus VISTA | MGVPTAPEAGCWRWGSLLFALFLAASLGPVAAFKVATLYSLYVCPEGQNVTLTCRVFGPVDKGH |
| | | DVTFYKTWYRSSRGEVQTCSERRPIRNLTFQDLHLHHGGHQAANTSHDLAQRHGLESASDHHG |
| | | NFSITMRNLTLLDSGLYCCLVVEIRHHHSEHRVHGAMELQVQTGKDAPSSCVAYPSSSQESENIT |
| | | AAALATGACIVGILCLPLILLLVYKQRQAASNRRAQELVRMDSNTQGIENPGFEASSPAQGILEAKV |
| | | RHPLSYVAQRQPSESGRHLLSEPGTPLSPPGPGDVFFPSLDPVPDSPNFEVI |
| 349 | Rat VISA (G3V9P3, | MGVPTVPEASSLRWGTLLLTIFLAASRGLVAAIKVTTPYSLYVCPEGQNVTLTCRILDSVSKGHDA |
| | v2) | NFLKTWFLSSRGEVQVCKEHRPIRNFISHHQHHRSHPAVNASHDQPQKHGLEIAYDNHGNFSITL |
| | | HNVTLSDSGLYCCLVIEVKHHHPERRLYGYMELQVQTGKGSASTCTAYPPNEQDSDSITAAALAT |
| | | GACIVGILCLPLILLLVYKQRQAASHRRAQELVRMDSNTQGIENPGYETTPPFQGMPEAKTRPPLS |
| | | YVAQRQPSESGRHLLSDPSTPLSPPGPGDVFFPSLDPVPDSPNSEAI |
| 350 | Mouse VISTA | MGVPAVPEASSPRWGTLLLAIFLAASRGLVAAFKVTTPYSLYVCPEGQNATLTCRILGPVSKGHDV |
| | (Q9D659, v2) | TIYKTWYLSSRGEVQMCKEHRPIRNFTLQHLQHHGSHLKANASHDQPQKHGLELASDHHGNFSIT |
| | | LRNVTPRDSGLYCCLVIELKNHHPEQRFYGSMELQVQAGKGSGSTCMASNEQDSDSITAAALAT |
| | | GACIVGILCLPLILLLVYKQRQVASHRRAQELVRMDSNTQGIENPGFETTPPFQGMPEAKTRPPLS |
| | | YVAQRQPSESGRYLLSDPSTPLSPPGPGDVFFPSLDPVPDSPNSEAI |

### Numbered Paragraphs

The following numbered paragraphs (paras) provide further statements of features and combinations of features which are contemplated in connection with the present disclosure:
1. An antigen-binding molecule, optionally isolated, which is capable of binding to VISTA and inhibiting VISTA-mediated signalling, independently of Fc-mediated function.
2. The antigen-binding molecule according to para 1, which is capable of binding to VISTA in the Ig-like V-type domain.
3. The antigen-binding molecule according to para 1 or para 2, wherein the antigen-binding molecule is capable of binding to a polypeptide comprising or consisting of the amino acid sequence of SEQ ID NO:6.
4. The antigen-binding molecule according to any one of paras 1 to 3, wherein the antigen-binding molecule is capable of binding to a polypeptide comprising or consisting of the amino acid sequence of SEQ ID NO:31.
5. The antigen-binding molecule according to any one of paras 1 to 4, wherein the antigen-binding molecule does not compete with IGN175A for binding to VISTA.
6. The antigen-binding molecule according to any one of paras 1 to 5, wherein the antigen-binding molecule is not capable of binding to a peptide consisting of the amino acid sequence of SEQ ID NO:275.
7. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:305
      HC-CDR2 having the amino acid sequence of SEQ ID NO:306
      HC-CDR3 having the amino acid sequence of SEQ ID NO:307; and
   (ii) a light chain variable (VL) region incorporating the following CDRs:
      LC-CDR1 having the amino acid sequence of SEQ ID NO:41
      LC-CDR2 having the amino acid sequence of SEQ ID NO:308
      LC-CDR3 having the amino acid sequence of SEQ ID NO:43.
8. The antigen-binding molecule according to any one of paras 1 to 7, wherein the antigen-binding molecule comprises:
   (i) a heavy chain variable (VH) region incorporating the following CDRs:
      HC-CDR1 having the amino acid sequence of SEQ ID NO:290
      HC-CDR2 having the amino acid sequence of SEQ ID NO:291
      HC-CDR3 having the amino acid sequence of SEQ ID NO:278; and
   (ii) a light chain variable (VL) region incorporating the following CDRs:
      LC-CDR1 having the amino acid sequence of SEQ ID NO:41
      LC-CDR2 having the amino acid sequence of SEQ ID NO:309
      LC-CDR3 having the amino acid sequence of SEQ ID NO:43.
9. The antigen-binding molecule according to any one of paras 1 to 8, wherein the antigen-binding molecule comprises:
   (i) a heavy chain variable (VH) region incorporating the following CDRs:
      HC-CDR1 having the amino acid sequence of SEQ ID NO:290
      HC-CDR2 having the amino acid sequence of SEQ ID NO:291
      HC-CDR3 having the amino acid sequence of SEQ ID NO:278; and
   (ii) a light chain variable (VL) region incorporating the following CDRs:
      LC-CDR1 having the amino acid sequence of SEQ ID NO:41
      LC-CDR2 having the amino acid sequence of SEQ ID NO:295
      LC-CDR3 having the amino acid sequence of SEQ ID NO:43.
10. The antigen-binding molecule according to any one of paras 1 to 8, wherein the antigen-binding molecule comprises:
   (i) a heavy chain variable (VH) region incorporating the following CDRs:
      HC-CDR1 having the amino acid sequence of SEQ ID NO:290
      HC-CDR2 having the amino acid sequence of SEQ ID NO:291
      HC-CDR3 having the amino acid sequence of SEQ ID NO:278; and
   (ii) a light chain variable (VL) region incorporating the following CDRs:
      LC-CDR1 having the amino acid sequence of SEQ ID NO:41
      LC-CDR2 having the amino acid sequence of SEQ ID NO:300
      LC-CDR3 having the amino acid sequence of SEQ ID NO:43.
11. The antigen-binding molecule according to any one of paras 1 to 8, wherein the antigen-binding molecule comprises:
   (i) a heavy chain variable (VH) region incorporating the following CDRs:
      HC-CDR1 having the amino acid sequence of SEQ ID NO:33
      HC-CDR2 having the amino acid sequence of SEQ ID NO:277
      HC-CDR3 having the amino acid sequence of SEQ ID NO:278; and
   (ii) a light chain variable (VL) region incorporating the following CDRs:
      LC-CDR1 having the amino acid sequence of SEQ ID NO:41
      LC-CDR2 having the amino acid sequence of SEQ ID NO:42
      LC-CDR3 having the amino acid sequence of SEQ ID NO:43.
12. The antigen-binding molecule according to any one of paras 1 to 8, wherein the antigen-binding molecule comprises:
   (i) a heavy chain variable (VH) region incorporating the following CDRs:
      HC-CDR1 having the amino acid sequence of SEQ ID NO:33
      HC-CDR2 having the amino acid sequence of SEQ ID NO:286
      HC-CDR3 having the amino acid sequence of SEQ ID NO:278; and
   (ii) a light chain variable (VL) region incorporating the following CDRs:
      LC-CDR1 having the amino acid sequence of SEQ ID NO:41
      LC-CDR2 having the amino acid sequence of SEQ ID NO:42
      LC-CDR3 having the amino acid sequence of SEQ ID NO:43.
13. The antigen-binding molecule according to any one of paras 1 to 8, wherein the antigen-binding molecule comprises:
   (i) a heavy chain variable (VH) region incorporating the following CDRs:
      HC-CDR1 having the amino acid sequence of SEQ ID NO:290
      HC-CDR2 having the amino acid sequence of SEQ ID NO:291
      HC-CDR3 having the amino acid sequence of SEQ ID NO:278; and
   (ii) a light chain variable (VL) region incorporating the following CDRs:
      LC-CDR1 having the amino acid sequence of SEQ ID NO:41
      LC-CDR2 having the amino acid sequence of SEQ ID NO:42
      LC-CDR3 having the amino acid sequence of SEQ ID NO:43.
14. The antigen-binding molecule according to any one of paras 1 to 8, wherein the antigen-binding molecule comprises:
   (i) a heavy chain variable (VH) region incorporating the following CDRs:
      HC-CDR1 having the amino acid sequence of SEQ ID NO:290
      HC-CDR2 having the amino acid sequence of SEQ ID NO:291
      HC-CDR3 having the amino acid sequence of SEQ ID NO:278; and
   (ii) a light chain variable (VL) region incorporating the following CDRs:
      LC-CDR1 having the amino acid sequence of SEQ ID NO:41
      LC-CDR2 having the amino acid sequence of SEQ ID NO:300
      LC-CDR3 having the amino acid sequence of SEQ ID NO:43.
15. The antigen-binding molecule according to any one of paras 1 to 7, wherein the antigen-binding molecule comprises:
   (i) a heavy chain variable (VH) region incorporating the following CDRs:
      HC-CDR1 having the amino acid sequence of SEQ ID NO:33
      HC-CDR2 having the amino acid sequence of SEQ ID NO:34
      HC-CDR3 having the amino acid sequence of SEQ ID NO:35; and
   (ii) a light chain variable (VL) region incorporating the following CDRs:
      LC-CDR1 having the amino acid sequence of SEQ ID NO:41
      LC-CDR2 having the amino acid sequence of SEQ ID NO:42
      LC-CDR3 having the amino acid sequence of SEQ ID NO:43.
16. The antigen-binding molecule according to any one of paras 1 to 7, wherein the antigen-binding molecule comprises:
   (i) a heavy chain variable (VH) region incorporating the following CDRs:
      HC-CDR1 having the amino acid sequence of SEQ ID NO:33
      HC-CDR2 having the amino acid sequence of SEQ ID NO:34
      HC-CDR3 having the amino acid sequence of SEQ ID NO:35; and
   (ii) a light chain variable (VL) region incorporating the following CDRs:
      LC-CDR1 having the amino acid sequence of SEQ ID NO:41
      LC-CDR2 having the amino acid sequence of SEQ ID NO:67
      LC-CDR3 having the amino acid sequence of SEQ ID NO:43.
17. The antigen-binding molecule according to any one of paras 1 to 7, wherein the antigen-binding molecule comprises:
   (i) a heavy chain variable (VH) region incorporating the following CDRs:
      HC-CDR1 having the amino acid sequence of SEQ ID NO:53
      HC-CDR2 having the amino acid sequence of SEQ ID NO:34
      HC-CDR3 having the amino acid sequence of SEQ ID NO:35; and
   (ii) a light chain variable (VL) region incorporating the following CDRs:
      LC-CDR1 having the amino acid sequence of SEQ ID NO:41
      LC-CDR2 having the amino acid sequence of SEQ ID NO:58
      LC-CDR3 having the amino acid sequence of SEQ ID NO:43.
18. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:72
      HC-CDR2 having the amino acid sequence of SEQ ID NO:73
      HC-CDR3 having the amino acid sequence of SEQ ID NO:74; and
   (ii) a light chain variable (VL) region incorporating the following CDRs:
      LC-CDR1 having the amino acid sequence of SEQ ID NO:80
      LC-CDR2 having the amino acid sequence of SEQ ID NO:81
      LC-CDR3 having the amino acid sequence of SEQ ID NO:82.
19. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:88
      HC-CDR2 having the amino acid sequence of SEQ ID NO:89
      HC-CDR3 having the amino acid sequence of SEQ ID NO:90; and
   (ii) a light chain variable (VL) region incorporating the following CDRs:
      LC-CDR1 having the amino acid sequence of SEQ ID NO:96
      LC-CDR2 having the amino acid sequence of SEQ ID NO:97
      LC-CDR3 having the amino acid sequence of SEQ ID NO:98.
20. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:88
      HC-CDR2 having the amino acid sequence of SEQ ID NO:89
      HC-CDR3 having the amino acid sequence of SEQ ID NO:90; and
   (ii) a light chain variable (VL) region incorporating the following CDRs:
      LC-CDR1 having the amino acid sequence of SEQ ID NO:137
      LC-CDR2 having the amino acid sequence of SEQ ID NO:138
      LC-CDR3 having the amino acid sequence of SEQ ID NO:139.
21. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:33
      HC-CDR2 having the amino acid sequence of SEQ ID NO:107
      HC-CDR3 having the amino acid sequence of SEQ ID NO:108,
   or a variant thereof in which one or two or three amino acids in one or more of HC-CDR1, HC-CDR2, or HC-CDR3 are substituted with another amino acid; and
   (ii) a light chain variable (VL) region incorporating the following CDRs:
      LC-CDR1 having the amino acid sequence of SEQ ID NO:114
      LC-CDR2 having the amino acid sequence of SEQ ID NO:67
      LC-CDR3 having the amino acid sequence of SEQ ID NO:115.
22. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:120
      HC-CDR2 having the amino acid sequence of SEQ ID NO:121
      HC-CDR3 having the amino acid sequence of SEQ ID NO:122; and
   (ii) a light chain variable (VL) region incorporating the following CDRs:
      LC-CDR1 having the amino acid sequence of SEQ ID NO:127
      LC-CDR2 having the amino acid sequence of SEQ ID NO:128
      LC-CDR3 having the amino acid sequence of SEQ ID NO:129.
23. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:144
      HC-CDR2 having the amino acid sequence of SEQ ID NO:145
      HC-CDR3 having the amino acid sequence of SEQ ID NO:146; and
   (ii) a light chain variable (VL) region incorporating the following CDRs:
      LC-CDR1 having the amino acid sequence of SEQ ID NO:151
      LC-CDR2 having the amino acid sequence of SEQ ID NO:152
      LC-CDR3 having the amino acid sequence of SEQ ID NO:153.
24. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:158
      HC-CDR2 having the amino acid sequence of SEQ ID NO:159
      HC-CDR3 having the amino acid sequence of SEQ ID NO:160; and
   (ii) a light chain variable (VL) region incorporating the following CDRs:
      LC-CDR1 having the amino acid sequence of SEQ ID NO:165
      LC-CDR2 having the amino acid sequence of SEQ ID NO:152
      LC-CDR3 having the amino acid sequence of SEQ ID NO:153.
25. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:169
      HC-CDR2 having the amino acid sequence of SEQ ID NO:170
      HC-CDR3 having the amino acid sequence of SEQ ID NO:171; and
   (ii) a light chain variable (VL) region incorporating the following CDRs:
      LC-CDR1 having the amino acid sequence of SEQ ID NO:177
      LC-CDR2 having the amino acid sequence of SEQ ID NO:178
      LC-CDR3 having the amino acid sequence of SEQ ID NO:179.
26. The antigen-binding molecule according to any one of paras 1 to 6, wherein the antigen-binding molecule comprises:
   (i) a heavy chain variable (VH) region incorporating the following CDRs:
      HC-CDR1 having the amino acid sequence of SEQ ID NO:72
      HC-CDR2 having the amino acid sequence of SEQ ID NO:184
      HC-CDR3 having the amino acid sequence of SEQ ID NO:246; and
   (ii) a light chain variable (VL) region incorporating the following CDRs:
      LC-CDR1 having the amino acid sequence of SEQ ID NO:247
      LC-CDR2 having the amino acid sequence of SEQ ID NO:178
      LC-CDR3 having the amino acid sequence of SEQ ID NO:190.
27. The antigen-binding molecule according to any one of paras 1 to 6 or para 26, wherein the antigen-binding molecule comprises:
   (i) a heavy chain variable (VH) region incorporating the following CDRs:
      HC-CDR1 having the amino acid sequence of SEQ ID NO:72
      HC-CDR2 having the amino acid sequence of SEQ ID NO:184
      HC-CDR3 having the amino acid sequence of SEQ ID NO:185; and
   (ii) a light chain variable (VL) region incorporating the following CDRs:
      LC-CDR1 having the amino acid sequence of SEQ ID NO:189
      LC-CDR2 having the amino acid sequence of SEQ ID NO:178
      LC-CDR3 having the amino acid sequence of SEQ ID NO:190.
28. The antigen-binding molecule according to any one of paras 1 to 6 or para 26, wherein the antigen-binding molecule comprises:
   (i) a heavy chain variable (VH) region incorporating the following CDRs:
      HC-CDR1 having the amino acid sequence of SEQ ID NO:72
      HC-CDR2 having the amino acid sequence of SEQ ID NO:184
      HC-CDR3 having the amino acid sequence of SEQ ID NO:195; and
   (ii) a light chain variable (VL) region incorporating the following CDRs:
      LC-CDR1 having the amino acid sequence of SEQ ID NO:197
      LC-CDR2 having the amino acid sequence of SEQ ID NO:178
      LC-CDR3 having the amino acid sequence of SEQ ID NO:190.
29. The antigen-binding molecule according to any one of paras 1 to 6 or para 26, wherein the antigen-binding molecule comprises:
   (i) a heavy chain variable (VH) region incorporating the following CDRs:
      HC-CDR1 having the amino acid sequence of SEQ ID NO:72
      HC-CDR2 having the amino acid sequence of SEQ ID NO:184
      HC-CDR3 having the amino acid sequence of SEQ ID NO:200; and
   (ii) a light chain variable (VL) region incorporating the following CDRs:
      LC-CDR1 having the amino acid sequence of SEQ ID NO:203
      LC-CDR2 having the amino acid sequence of SEQ ID NO:178
      LC-CDR3 having the amino acid sequence of SEQ ID NO:190.
30. The antigen-binding molecule according to any one of paras 1 to 6, wherein the antigen-binding molecule comprises:
a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:310;
or
   a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:294;
or
   a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:297;
or
   a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:299;
or
   a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:301;
or
   a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:302;
or
   a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:303;
or
   a VH region comprising an amino acid sequence having at least 70% sequence identity to the amino acid sequence of SEQ ID NO:276; and
   a VL region comprising an amino acid sequence having at least 70% sequence identity to the amino acid sequence of SEQ ID NO:282;
or
   a VH region comprising an amino acid sequence having at least 70% sequence identity to the amino acid sequence of SEQ ID NO:285; and
   a VL region comprising an amino acid sequence having at least 70% sequence identity to the amino acid sequence of SEQ ID NO:287;
a VH region comprising an amino acid sequence having at least 70% sequence identity to the amino acid sequence of SEQ ID NO:32; and
   a VL region comprising an amino acid sequence having at least 70% sequence identity to the amino acid sequence of SEQ ID NO:40;
or
   a VH region comprising an amino acid sequence having at least 70% sequence identity to the amino acid sequence of SEQ ID NO:52; and
   a VL region comprising an amino acid sequence having at least 70% sequence identity to the amino acid sequence of SEQ ID NO:57;
or
   a VH region comprising an amino acid sequence having at least 70% sequence identity to the amino acid sequence of SEQ ID NO:62; and
   a VL region comprising an amino acid sequence having at least 70% sequence identity to the amino acid sequence of SEQ ID NO:66;
or
   a VH region comprising an amino acid sequence having at least 70% sequence identity to the amino acid sequence of SEQ ID NO:48; and
   a VL region comprising an amino acid sequence having at least 70% sequence identity to the amino acid sequence of SEQ ID NO:50;
or.
   a VH region comprising an amino acid sequence having at least 70% sequence identity to the amino acid sequence of SEQ ID NO:87; and
   a VL region comprising an amino acid sequence having at least 70% sequence identity to the amino acid sequence of SEQ ID NO:95;
or
   a VH region comprising an amino acid sequence having at least 70% sequence identity to the amino acid sequence of SEQ ID NO:106; and
   a VL region comprising an amino acid sequence having at least 70% sequence identity to the amino acid sequence of SEQ ID NO:113;
or
   a VH region comprising an amino acid sequence having at least 70% sequence identity to the amino acid sequence of SEQ ID NO:143; and
   a VL region comprising an amino acid sequence having at least 70% sequence identity to the amino acid sequence of SEQ ID NO:150;
or
   a VH region comprising an amino acid sequence having at least 70% sequence identity to the amino acid sequence of SEQ ID NO:157; and
   a VL region comprising an amino acid sequence having at least 70% sequence identity to the amino acid sequence of SEQ ID NO:164;
or
   a VH region comprising an amino acid sequence having at least 70% sequence identity to the amino acid sequence of SEQ ID NO:71; and
   a VL region comprising an amino acid sequence having at least 70% sequence identity to the amino acid sequence of SEQ ID NO:79;
or
   a VH region comprising an amino acid sequence having at least 70% sequence identity to the amino acid sequence of SEQ ID NO:102; and
   a VL region comprising an amino acid sequence having at least 70% sequence identity to the amino acid sequence of SEQ ID NO:104;
or
   a VH region comprising an amino acid sequence having at least 70% sequence identity to the amino acid sequence of SEQ ID NO:119; and
   a VL region comprising an amino acid sequence having at least 70% sequence identity to the amino acid sequence of SEQ ID NO:126;
or
   a VH region comprising an amino acid sequence having at least 70% sequence identity to the amino acid sequence of SEQ ID NO:183; and
   a VL region comprising an amino acid sequence having at least 70% sequence identity to the amino acid sequence of SEQ ID NO:188;
or
   a VH region comprising an amino acid sequence having at least 70% sequence identity to the amino acid sequence of SEQ ID NO:194; and
   a VL region comprising an amino acid sequence having at least 70% sequence identity to the amino acid sequence of SEQ ID NO:196;
or
   a VH region comprising an amino acid sequence having at least 70% sequence identity to the amino acid sequence of SEQ ID NO:199; and
   a VL region comprising an amino acid sequence having at least 70% sequence identity to the amino acid sequence of SEQ ID NO:202;
or
   a VH region comprising an amino acid sequence having at least 70% sequence identity to the amino acid sequence of SEQ ID NO:133; and
   a VL region comprising an amino acid sequence having at least 70% sequence identity to the amino acid sequence of SEQ ID NO:136;
or
   a VH region comprising an amino acid sequence having at least 70% sequence identity to the amino acid sequence of SEQ ID NO:168; and
   a VL region comprising an amino acid sequence having at least 70% sequence identity to the amino acid sequence of SEQ ID NO:176.
31. The antigen-binding molecule according to any one of paras 1 to 30, wherein the antigen-binding molecule is capable of binding to human VISTA and one or more of: mouse VISTA and cynomolgus macaque VISTA.
32. An antigen-binding molecule, optionally isolated, comprising (i) an antigen-binding molecule according to any one of paras 1 to 31, and (ii) an antigen-binding molecule capable of binding to an antigen other than VISTA.
33. The antigen-binding molecule according to any one of paras 1 to 32, wherein the antigen-binding molecule is capable of binding to cells expressing VISTA at the cell surface.
34. The antigen-binding molecule according to any one of paras 1 to 33, wherein the antigen-binding molecule is capable of inhibiting interaction between VISTA and an interaction partner for VISTA.
35. The antigen-binding molecule according to any one of paras 1 to 34, wherein the antigen-binding molecule is capable of inhibiting VISTA-mediated signalling.
36. The antigen-binding molecule according to any one of paras 1 to 35, wherein the antigen-binding molecule is capable of increasing proliferation and/or cytokine production by effector immune cells.
37. A chimeric antigen receptor (CAR) comprising an antigen-binding molecule according to any one of paras 1 to 36.
38. A nucleic acid, or a plurality of nucleic acids, optionally isolated, encoding an antigen-binding molecule according to any one of paras 1 to 36 or a CAR according to para 37.
39. An expression vector, or a plurality of expression vectors, comprising a nucleic acid or a plurality of nucleic acids according to para 38.
40. A cell comprising an antigen-binding molecule according to any one of paras 1 to 36, a CAR according to para 37, a nucleic acid or a plurality of nucleic acids according to para 38, or an expression vector or a plurality of expression vectors according to para 39.
41. A method comprising culturing a cell comprising a nucleic acid or a plurality of nucleic acids according to para 38, or an expression vector or a plurality of expression vectors according to para 39, under conditions suitable for expression of the antigen-binding molecule or CAR from the nucleic acid(s) or expression vector(s).
42. A composition comprising an antigen-binding molecule according to any one of paras 1 to 36, a CAR according to para 37, a nucleic acid or a plurality of nucleic acids according to para 38, an expression vector or a plurality of expression vectors according to para 39, or a cell according to para 40.
43. The composition according to para 42, additionally comprising an agent capable of inhibiting signalling mediated by an immune checkpoint molecule other than VISTA, optionally wherein the immune checkpoint molecule other than VISTA is selected from PD-1, CTLA-4, LAG-3, TIM-3, TIGIT and BTLA.
44. An antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43 for use in a method of medical treatment or prophylaxis.
45. An antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, for use in a method of treatment or prevention of a cancer or an infectious disease.
46. Use of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, in the manufacture of a medicament for use in a method of treatment or prevention of a cancer or an infectious disease.
47. A method of treating or preventing a cancer or an infectious disease, comprising administering to a subject a therapeutically or prophylactically effective amount of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43.
48. The antigen-binding molecule, CAR, nucleic acid or plurality of nucleic acids, expression vector or plurality of expression vectors, cell or composition for use according to para 45, the use according to para 46 or the method according to para 47, wherein the cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.
49. The antigen-binding molecule, CAR, nucleic acid or plurality of nucleic acids, expression vector or plurality of expression vectors, cell or composition for use according to any one of paras 45 to 48, wherein the cancer is selected from: colorectal cancer, pancreatic cancer, breast cancer (e.g. triple-negative breast cancer), liver cancer, prostate cancer, ovarian cancer, head and neck cancer, leukemia, lymphoma, melanoma, thymoma, lung cancer, non-small cell lung cancer (NSCLC) and a solid tumor.
50. An antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, for use in a method of treatment or prevention of a disease in which myeloid-derived suppressor cells (MDSCs) are pathologically implicated.
51. Use of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, in the manufacture of a medicament for use in a method of treatment or prevention of a disease in which myeloid-derived suppressor cells (MDSCs) are pathologically implicated.
52. A method of treating or preventing a disease in which myeloid-derived suppressor cells (MDSCs) are pathologically implicated, comprising administering to a subject a therapeutically or prophylactically effective amount of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43.
53. The antigen-binding molecule, CAR, nucleic acid or plurality of nucleic acids, expression vector or plurality of expression vectors, cell or composition for use, the use, or the method according to any one of paras 45 to 52, wherein the method additionally comprises administration of an agent capable of inhibiting signalling mediated by an immune checkpoint molecule other than VISTA, optionally wherein the immune checkpoint molecule other than VISTA is selected from PD-1, CTLA-4, LAG-3, TIM-3, TIGIT or BTLA.
54. A method of inhibiting VISTA-mediated signalling, comprising contacting VISTA-expressing cells with an antigen-binding molecule according to any one of paras 1 to 36.
55. A method for inhibiting the activity of myeloid-derived suppressor cells (MDSCs), the method comprising contacting MDSCs with an antigen-binding molecule according to any one of paras 1 to 36.
56. A method for increasing the number or activity of effector immune cells, the method comprising inhibiting the activity of VISTA-expressing cells with an antigen-binding molecule according to any one of paras 1 to 36.
57. An in vitro complex, optionally isolated, comprising an antigen-binding molecule according to any one of paras 1 to 36 bound to VISTA.
58. A method comprising contacting a sample containing, or suspected to contain, VISTA with an antigen-binding molecule according to any one of paras 1 to 36, and detecting the formation of a complex of the antigen-binding molecule with VISTA.
59. A method of selecting or stratifying a subject for treatment with a VISTA-targeted agent, the method comprising contacting, in vitro, a sample from the subject with an antigen-binding molecule according to any one of paras 1 to 36 and detecting the formation of a complex of the antigen-binding molecule with VISTA.
60. Use of an antigen-binding molecule according to any one of paras 1 to 36 as an in vitro or in vivo diagnostic or prognostic agent.
61. Use of an antigen-binding molecule according to any one of paras 1 to 36 in a method for detecting, localizing or imaging a cancer, optionally wherein the cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.
62. The use according to para 61, wherein the cancer is selected from: colorectal cancer, pancreatic cancer, breast cancer (e.g. triple-negative breast cancer), liver cancer, prostate cancer, ovarian cancer, head and neck cancer, leukemia, lymphoma, melanoma, thymoma, lung cancer, non-small cell lung cancer (NSCLC) and a solid tumor.

The following further numbered paragraphs (paras) provide further statements of features and combinations of features which are contemplated in connection with the present disclosure:
1b. An antigen-binding molecule that binds to VISTA for use in a method of treating or preventing a cancer in a subject, wherein the cancer is characterised by the presence of: (i) cells expressing VISTA, and (ii) cells expressing an interaction partner for VISTA that binds to the C-C′ region of VISTA; and wherein the antigen-binding molecule inhibits interaction between VISTA and the interaction partner for VISTA.
2b. Use of an antigen-binding molecule that binds to VISTA in the manufacture of a medicament for treating or preventing a cancer in a subject, wherein the cancer is characterised by the presence of: (i) cells expressing VISTA, and (ii) cells expressing an interaction partner for VISTA that binds to the C-C′ region of VISTA; and wherein the antigen-binding molecule inhibits interaction between VISTA and the interaction partner for VISTA.
3b. A method of treating or preventing a cancer in a subject, wherein the method comprises administering to a subject a therapeutically or prophylactically effective amount of an antigen-binding molecule that binds to VISTA, wherein the cancer is characterised by the presence of: (i) cells expressing VISTA, and (ii) cells expressing an interaction partner for VISTA that binds to the C-C′ region of VISTA; and wherein the antigen-binding molecule inhibits interaction between VISTA and the interaction partner for VISTA.
4b. The antigen-binding molecule for use according to para 1 b, the use according to para 2b, or the method according to para 3, wherein the cancer comprises a tumor comprising: (i) cells expressing VISTA, and (ii) cells expressing an interaction partner for VISTA.
5b. A method of selecting a subject for treatment with an antigen-binding molecule that inhibits interaction between VISTA and an interaction partner for VISTA that binds to the C-C′ region of VISTA, comprising:
   (a) analysing a subject's cancer in order to determine whether the cancer is characterised by the presence of: (i) cells expressing VISTA, and (ii) cells expressing an interaction partner for VISTA that binds to the C-C′ region of VISTA; and
   (b) selecting a subject for treatment with an antigen-binding molecule that inhibits interaction between VISTA and an interaction partner for VISTA that binds to the C-C′ region of VISTA where the subject's cancer is determined in step (a) to comprise cells expressing VISTA, and cells expressing an interaction partner for VISTA that binds to the C-C′ region of VISTA.
6b. The method according to para 5b, wherein the method further comprises:
   (c) administering an antigen-binding molecule that inhibits interaction between VISTA and an interaction partner for VISTA that binds to the C-C′ region of VISTA to a subject selected for treatment in step (b).
7b. The antigen-binding molecule for use, the use or the method according to any one of paras 1b to 6b, wherein the interaction partner for VISTA that binds to the C-C′ region of VISTA is selected from LRIG1 and VSIG3.
8b. The antigen-binding molecule for use, the use or the method according to any one of paras 1b to 7b, wherein the antigen-binding molecule binds to VISTA via contact with one or more amino acids of the region shown in SEQ ID NO:340.
9b. The antigen-binding molecule for use, the use or the method according to any one of paras 1b to 8b, wherein the antigen-binding molecule comprises:
   (i) a heavy chain variable (VH) region incorporating the following CDRs:
      HC-CDR1 having the amino acid sequence of SEQ ID NO:305
      HC-CDR2 having the amino acid sequence of SEQ ID NO:306
      HC-CDR3 having the amino acid sequence of SEQ ID NO:307; and
   (ii) a light chain variable (VL) region incorporating the following CDRs:
      LC-CDR1 having the amino acid sequence of SEQ ID NO:41
      LC-CDR2 having the amino acid sequence of SEQ ID NO:308
      LC-CDR3 having the amino acid sequence of SEQ ID NO:43.
10b. The antigen-binding molecule for use, the use, or the method according to any one of paras 1b to 9b, wherein the antigen-binding molecule comprises:
   (i) a VH region incorporating the following CDRs:
      HC-CDR1 having the amino acid sequence of SEQ ID NO:290
      HC-CDR2 having the amino acid sequence of SEQ ID NO:291
      HC-CDR3 having the amino acid sequence of SEQ ID NO:278; and
   (ii) a VL region incorporating the following CDRs:
      LC-CDR1 having the amino acid sequence of SEQ ID NO:41
      LC-CDR2 having the amino acid sequence of SEQ ID NO:295
      LC-CDR3 having the amino acid sequence of SEQ ID NO:43.
11 b. The antigen-binding molecule for use, the use, or the method according to any one of paras 1b to 10b, wherein the antigen-binding molecule comprises:
   a VH region comprising an amino acid sequence having at least 70% sequence identity to the amino acid sequence of SEQ ID NO:289; and
   a VL region comprising an amino acid sequence having at least 70% sequence identity to the amino acid sequence of SEQ ID NO:297.
12b. The antigen-binding molecule for use, the use, or the method according to any one of paras 1b to 11 b, wherein the antigen-binding molecule comprises:
   a VH region incorporating the following framework regions (FRs):
   HC-FR1 having the amino acid sequence of SEQ ID NO:63
   HC-FR2 having the amino acid sequence of SEQ ID NO:292
   HC-FR3 having the amino acid sequence of SEQ ID NO:293
   HC-FR4 having the amino acid sequence of SEQ ID NO:281.
13b. The antigen-binding molecule for use, the use, or the method according to any one of paras 1b to 12b, wherein the antigen-binding molecule comprises:
   a VL region incorporating the following framework regions (FRs):
   LC-FR1 having the amino acid sequence of SEQ ID NO:288
   LC-FR2 having the amino acid sequence of SEQ ID NO:298
   LC-FR3 having the amino acid sequence of SEQ ID NO:284
   LC-FR4 having the amino acid sequence of SEQ ID NO:47.
14b. The antigen-binding molecule for use, the use, or the method according to any one of paras 1b to 13b, wherein the antigen-binding molecule comprises a heavy chain comprising the amino acid sequence of SEQ ID NO:331.
15b. The antigen-binding molecule for use, the use, or the method according to any one of paras 1b to
14b, wherein the antigen-binding molecule comprises a light chain comprising the amino acid sequence of SEQ ID NO:317.
16b. The antigen-binding molecule for use, the use or the method according to any one of paras 1b to
15b, wherein the cancer is selected from: a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.
17b. The antigen-binding molecule for use, the use or the method according to para 16, wherein the cancer is selected from: colorectal cancer, pancreatic cancer, breast cancer, triple-negative breast cancer, liver cancer, prostate cancer, ovarian cancer, head and neck cancer, leukemia, lymphoma, melanoma, thymoma, lung cancer, non-small cell lung cancer (NSCLC) and a solid tumor.

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may preferably performed in vitro. The term “in vitro” is intended to encompass procedures performed with cells in culture whereas the term “in vivo” is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIG. 1. Sensorgrams showing the background-subtracted binding signal for anti-VISTA antibodies V4-C26, 13F3 and VSTB1112 to human VISTA and mouse VISTA as determined by Bio-Layer Interferometry.

FIGS. 2A and 2B. Graphs showing the effects of administration of V4-C26.hIgG4 on tumor volume and survival of mice in a CT26 cell-derived mouse model of colon carcinoma. (2A) shows tumor volume over time, and (2B) shows percent survival over time for mice administered with anti-VISTA antibody V4-C26 hIgG4 or vehicle control.

FIGS. 3A to 3H. Scatterplots and images showing VISTA expression by cells in healthy tissues and in certain cancers, and VSIG3 expression in certain cancers. (3A) Immune cell cluster identification by RNA single-cell analysis of 10× Genomics data comprising 68,000 healthy PBMCs. (3B, 3C) UMAP and summary plot of single-cell RNA-seq data for VISTA expression in different cell clusters, where cDC refers to Classical Dendritic Cells, pDC to Plasmacytoid Dendritic Cells and HSPC to Hematopoietic Stem and Progenitor Cells. (3D) Representative immunohistochemical staining for healthy spleen, bone marrow, breast and lung TMAs (n=99) stained with 4M2-C12-mIgG2a at 0.02 mg/ml; magnification, 200×. (3E) Representative staining for VISTA in triple negative breast cancer (TNBC; n=126) and non-small cell lung cancer (NSCLC; n=140) TMAs stained with 4M2-C12-mIgG2a at 0.02 mg/ml; magnification, 200×, and (3F) % of TNBC, NSCLC, hepato-cellular carcinoma (HCC) and mesothelioma patients with negative (0), low (1), moderate (2) or high (3) staining intensity for VISTA. (3G) Representative staining for VSIG3 in TNBC (n=126) and NSCLC (n=140) TMAs stained with anti-VSIG3 antibody at 0.003 mg/ml; magnification, 200×, and (3H) % of TNBC, NSCLC, HCC and mesothelioma patients with negative (0), low (1), moderate (2) or high (3) staining intensity for VSIG3.

FIGS. 4A to 4G. VISTA protein structures showing binding regions for interaction partners and antibody V4-C26 hIgG4. (4A) VISTA homology model superimposed with the complex of PD-L1 bound to its ligand PD-1 from PDB:5IUS. (4B) Front C-C′ s sheets and loop of VISTA protein constituting the predicted binding interface for physiologically relevant binding partners. (4C) 3D overlay model of VISTA protein from mouse, rats, cynomolgus monkey and humans showing sequence conservation of the C-C′ s sheets and loop. (4D) 3D model of the computationally predicted target region for antibody blockade of VISTA. (4E) Predicted VISTA homology model with residues implicated in VSIG3 binding. (4F) Epitope mapping analysis of V4-C26 hIgG4 bound to VISTA using hydrogen-deuterium exchange mass spectrometry (HDXMS).

FIGS. 5A to 5D. Graphs showing binding specificity of V4-C26 hIgG4. (5A) Binding of V4-C26 hIgG4 to human B7 family antigens, PD-1 and CTLA-4 as indicated, as determined by ELISA (data shown are mean n=2 measurements and error bars are SEM). (5B) Binding of V4-C26 hIgG4 to human, non-human primate (NHP), rat or mouse VISTA orthologs, as determined by ELISA (data shown are mean of n=3 measurements and error bars are SEM). (5C) Binding of V4-C26 hIgG4 to HEK293T cells overexpressing human, non-human primate (NHP), rat or mouse VISTA orthologs, as determined by flow cytometry (data shown are mean of n=3 measurements and error bars are SEM). (5D) Binding of V4-C26 hIgG4 to CD11 b+ myeloid cells isolated from human, NHP, rat or mouse PBMCs (data shown are mean of n=3 measurements and error bars are SEM).

FIGS. 6A to 6E. Graphs and bar charts showing the results of analysis of the ability of V4-C26 hIgG4 to inhibit interaction between VISTA and key binding partners, and inhibit downstream activity. (6A) Inhibition of VISTA-VSIG3 interaction, as determined by competition ELISA (data shown are mean of n=3 measurements and error bars are SEM). (6B) Analysis of VSIG3 suppression of IFN-γ levels, measured by ELISA at 24 hr, from PBMC cultured in plates coated with αCD3 monoclonal antibody (OKT3) and VSIG3-Fc at ratios of 0:1 or 1:2 (data shown are mean of n=3 measurements; error bars are SEM and P-value was obtained by paired t-test, *p<0.05). (6C) Analysis of VSIG3 suppression of IFN-γ levels, measured by ELISA at 24 hr, from PBMC cultured in plates coated with αCD3 monoclonal antibody (OKT3) and VSIG3-Fc at at a ratio of 1:2, in presence of V4-C26 hIgG4 (HMBD-002), VSTB112 or IgG4 isotype control (data shown are mean of n=3 measurements; error bars are SEM and P-value was obtained by one-way ANOVA (Tukey's multiple comparison test), **p<0.01). (6D) Anti-CD3 antibody-induced IFN-γ secretion from MDSCs co-cultured with autologous PBMCs after 96 hrs, in the presence or absence of V4-C26 hIgG4 (HMBD-002), VSTB112 or IgG4 isotype control, as measured by ELISA (data shown are mean of n=6 measurements and error bars are SEM and P-value was obtained by two-way ANOVA (Tukey's multiple comparison test), ****p<0.0001). (6E) Inhibition of neutrophil migration to the bottom chambers of transwell coated with C5a and measured using CellTiter-Glo (data shown are mean of n=3 measurements and error bars are SEM).

FIGS. 7A and 7B. Graphs and bar chart showing the results of analysis of effect of VISTA blockade by V4-C26 hIgG4 on immune activation. (7A) Levels of the indicated cytokines measured at 96 hrs by Luminex, from the supernatant of an allogenic mixed lymphocyte reaction. Concentrations of V4-C26 hIgG4 and anti PD-1 antibody, Pembrolizumab (annotated as Pembro) are shown in pg/ml. Data were normalized to isotype control. Data shown are mean of n=10 and error bars are SEM. P-values were obtained by one-way ANOVA (Tukey's multiple comparison test), *p<0.05, ***p<0.001, ****p<0.0001. (7B) Speed2 pathway analysis from bulk RNA-sequencing of MLR samples (n=10). Data was normalized to isotype control, represented as Mean+SD and the p-adjusted values were obtained from bulk RNA seq analysis, **p<0.01, ***p<0.001.

FIGS. 8A to 8G. Graphs and bar charts showing V4-C26 hIgG4 anti-tumour responses in cell-derived xenograft (CDX) models. Mice were randomized and dosed with concentrations of test articles at indicated time-points. Tumour volumes were measured twice a week. Each data point represents the mean tumour volume+/−SEM from n=10 mice. (8A) Results in a syngeneic CDX model in which female BALB/c mice were subcutaneously implanted with CT26 cells. (8B) Results in a syngeneic CDX model in which female BALB/c mice were subcutaneously implanted with CT26 cells, and administered with different doses of V4-C26 hIgG4, as indicated. (8C) Results in a syngeneic CDX model in which female BALB/c mice were orthotopically implanted with VISTA overexpressing 4T-1 cells. (8D) Results in a CDX model in which CD34-engrafted humanized female HiMice were subcutaneously implanted with HCT15 cells. (8E) Results in a CDX model in which CD34-engrafted humanized female HiMice were subcutaneously implanted with A549 cells. (8F) Results of cellular profiling by flow cytometry, showing tumour-infiltrating leukocyte (TIL) populations in the tumor microenvironment in the CT26 CDX model (data shown are mean of n=3 and error bars are SEM). (8G) Results of a CT26 antigen recall assay measured by ex vivo culture of TILs (CD45+) with CT26 cells to measure lysis, or culture of tumour-enriched T cells (CD4+/CD8+) with CT26 cells to measure T cell activation via IFN-γ secretion using ELISA (data shown are mean of n=3 and error bars are SEM. All p-values were obtained by unpaired t-tests, *p<0.05, **p<0.01; each data point represents a mouse).

FIGS. 9A and 9B. Alignment and VISTA protein structures showing (9A) multiple sequence alignment of human VISTA (SEQ ID NO:1) and cynomolgous monkey (SEQ ID NO:348), rat (SEQ ID NO:349) and mouse (SEQ ID NO:350) homologs, and (9B) VISTA protein structure highlighting residues involved in binding with VSIG3 (Arg86, Phe94, Gln95), PSGL1 (His98, His100, His153, His154, His155) and LRIG1 (Thr82, Arg87).

FIGS. 10A to 10C. Graphs showing binding specificity and anti-tumor efficacy of 4M2-C12-mIgG2a. (10A) Binding of 4M2-C12-mIgG2a to human B7 family antigens, PD-1 and CTLA-4 as indicated, as determined by ELISA. (10B) Results associated with treatment of mice with 4M2-C12-mIgG2a (V4P mIgG2A), 4M2-C12-mIgG2a comprising Fc-silencing LALAPG substitutions in the Fc region (V4P mIgG2A-LALAPG), or vehicle on (Vehicle Control) in a syngeneic CDX model in which mice were subcutaneously implanted with EL4 cells. (10C) Results associated with treatment of mice with 4M2-C12-mIgG2a (V4P mIgG2A), 4M2-C12-mIgG2a comprising Fc-silencing LALAPG substitutions in the Fc region (V4P mIgG2A-LALAPG), or vehicle on (Vehicle Control) in a syngeneic CDX model in which mice were subcutaneously implanted with CT26 cells.

FIGS. 11A and 11B. Graphs showing binding affinity of V4-C26 hIgG4 (HMBD-002) to (11A) FcγRIII and (11B) C1 q proteins as assessed by ELISA. Data shown are mean of n=3 measurements and error bars are SEM.

FIGS. 12A to 12G. Sensorgrams and tables relating to characterisation of binding of V4-C26 hIgG4 to VISTA and homologs. (12A) Results of epitope binning analysis by biolayer interferometry, investigating binding to human VISTA by V4-C26 hIgG4, VSTB112 and IGN175A. Signals were aligned to baseline. (12B) Results of epitope binning analysis by biolayer interferometry, investigating binding to mouse VISTA by V4-C26 hIgG4, 13F3 and MH5A. Signals were aligned to baseline. (12C to 12F) Sensorgrams and calculated Kon, Kou and K_{D} for binding of V4-C26 hIgG4 to (12C) human VISTA, (12D) Non-human primate (NHP) VISTA, (12E) Rat VISTA, and (12F) Mouse VISTA. (12G) Graph and table showing of V4-C26 hIgG4 to human VISTA across pH range 5.5-7.5, as determined by ELISA (data shown are mean of n=3 measurements and error bars are SEM).

FIGS. 13A to 13C. Graphs showing (13A) interaction between VSIG3 and VISTA, and (13B) interaction between LRIG1 and VISTA, and (13C) inhibition of interaction between LRIG1 and VISTA by V4-C26 hIgG4 (HMBD-002), as determined by ELISA.

FIGS. 14A and 14B. Bar chart and graphs showing the effects of treatment with V4-C26 hIgG4 (HMBD-002) on expression in PBMCs of genes involved in immune responses. (14A) Results of KEGG pathway analysis of bule RNA-seq data from PBMCs cultured for 96 hours in the presence of V4-C26 hIgG4 or IgG4 isotype control, showing enrichment of transcript levels in genes associated with the TLR, TNFα, JAK-STAT and IL-17 signalling pathways. (14B) Enrichment of transcript levels in genes associated with type-I and Type-II interferon genes from bulk RNA-sequencing of MLR samples, for reactions performed in the presence of V4-C26 hIgG4 (HMBD-002), IgG4 isotype control or pembrolizumab (n=10; data represented as mean+SD and the p-adjusted values were obtained from bulk RNA seq analysis as *p<0.05, ***p<0.001).

FIGS. 15A to 15G. Graphs relating to pharmacokinetics and toxicity of V4-C26 hIgG4. (15A to 15C) Serum concentration of V4-C26 hIgG4 in tumor-bearing and non-tumor bearing (15A) Balb/c mice, (15B) Sprague Dawley rats, and (15C) Cynomolgus monkeys. V4-C26 hIgG4 was administered in a single dose at the indicated concentration, via intraperitoneal injection. (15D to 15G) Results from ex vivo cytokine release assays performed with (15D and 15E) human whole blood, or (15F and 15G) isolated PBMCs obtained from healthy donors (n=10), showing the level of (15D, 15F) IL-2 and (15E, 15G) IL-6 after stimulation for 24 hours with the indicated amount of V4-C26 hIgG4, IgG4 isotype control, (15D and 15E) staphylococcal enterotoxin B (SEB) or (15F and 15G) anti-CD3 antibody.

FIGS. 16A and 16B. Bar charts showing effects of V4-C26 hIgG4 (HMBD-002) on tumour-infiltrating CD8+ T cell populations in the tumour microenvironment in a CT26 cell-derived xenograft (CDX) model. (16A) Results of cellular profiling by multicolour flow cytometry showing levels of CD8+ T cells (left) and tumour antigen-specific CD8+ T cells (right). Frequency of gp70 antigen-specific CD8 T cells in tumor was determined via the MuLV env gp70 423-431 pentamer (Prolmmune Ltd.) (16B) Results of cellular profiling by multicolour flow cytometry showing cytotoxic response and activation of CD8+ T cells.

FIGS. 17A and 17B. Bar charts showing effects of V4-C26 hIgG4 (HMBD-002) on tumour-associated macrophage (TAMs) populations in the tumour microenvironment in a CT26 cell-derived xenograft (CDX) model. (17A) Results of cellular profiling by multicolour flow cytometry showing levels of MHCII− macrophages. (17B) Results of cellular profiling by multicolour flow cytometry showing activation of M1 macrophages.

FIG. 18. Graphs showing effects of V4-C26 hIgG4 (HMBD-002) in transcription of genes associated with pro-inflammatory macrophage activation, positive regulation of T cell cytotoxicity and T cell cytolytic activity in CT26 tumours. Genes are ordered from highest fold increase on the left, through to highest fold decrease on the right.

FIGS. 19A and 19B. Graphs and bar charts showing effects of V4-C26 hIgG4 (HMBD-002) in combination with aPD-1. (19A) Results in a syngeneic CDX model in which female BALB/c mice were subcutaneously implanted with CT26 cells, and administered with different doses of V4-C26 hIgG4, aPD-1 or V4-C26 hIgG4 in combination with aPD-1, as indicated. (19B) Results of cellular profiling by flow cytometry, showing tumour antigen-specific CD8+ T cells and tumour antigen-specific exhausted CD8+ T cells in the tumor microenvironment in the CT26 CDX model.

FIG. 20. Graph showing V4-C26 hIgG4 anti-tumour response in a patient-derived xenograft (PDX) malignant pleural epithelioid mesothelioma model.

### EXAMPLES

### Examples and Figures of WO 2019/185879 A1

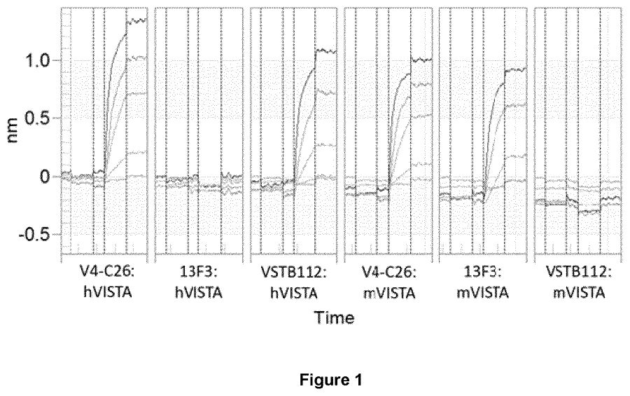

The entire disclosure of WO 2019/185879 A1 is incorporated by reference in its entirety herein. In particular, Examples 1 to 16 and corresponding FIGS. 1 to 65C of WO 2019/185879 A1, which illustrate structural and functional properties of the VISTA-binding antigen-binding molecules disclosed in WO 2019/185879 A1, are hereby incorporated by reference.

### Example 1: V4-C26

### 1.1 Characterisation of V4-C26 in WO 2019/185879 A1

The VISTA-binding antibody clone designated V4-C26 is described in WO 2019/185879 A1, which is incorporated by reference hereinabove.

V4-C26 comprises the heavy chain variable region shown in SEQ ID NO:289, and the light chain variable region shown in SEQ ID NO:297. Example 13 of WO 2019/185879 A1 describes a molecule (molecule [24]) comprising the VH and VL regions of V4-C26 in human IgG1/VK format, formed of SEQ ID NO:315 and SEQ ID NO:317.

Example 13 and FIG. 53 of WO 2019/185879 A1 show that analysis of V4-C26 using IMGT DomainGapAlign (Ehrenmann et al., Nucleic Acids Res., 38, D301-307 (2010)) and IEDB deimmunization (Dhanda et al., Immunology. (2018) 153(1):118-132) tools revealed that V4-C26 has sufficient homology to human germline heavy and light chains to be considered humanised (i.e. >85%), and numbers of potentially immunogenic peptides few enough to be considered safe and not to present developability issues.

Example 13.1 and FIGS. 45, 46 and 47 of WO 2019/185879 A1 show the results of the analysis of binding of V4-C26 to various different proteins by Biolayer Interferometry. FIGS. 45D, 46B and 47C show that V4-C26 binds to both human VISTA and mouse VISTA with high affinity, and FIG. 45C shows that V4-C26 displays no cross-reactivity with human PD-L1.

Example 13.2 and FIGS. 49, 50, 51, 56 and 57 of WO 2019/185879 A1 show the results of the analysis of binding of V4-C26 to various different proteins by ELISA. FIGS. 49C, 50C and 51C show that V4-C26 has a very low EC₅₀ for binding to human VISTA and mouse VISTA. FIGS. 56C, 57C and 57I show that V4-C26 displays binding to human, mouse, rat and cynomolgus monkey VISTA, yet displays no cross-reactivity for other human B7 protein family members (B7H3, B7H4, B7H6, B7H7, PD-1, CTLA-4).

Example 13.3 and FIGS. 58B and 58C of WO 2019/185879 A1 show that V4-C26 binds to cells expressing human VISTA or mouse VISTA, as determined by flow cytometry.

Example 13.4 and FIG. 52J of WO 2019/185879 A1 show that V4-C26 is thermostable, having a melting temperature of 72.9° C. as determined by Differential Scanning Fluorimetry.

1.2 Comparison of binding to human and mouse VISTA for V4-C26, VSTB112 and mAb 13F3 Binding to human VISTA and mouse VISTA was analysed for anti-VISTA antibodies V4-C26 (i.e. molecule [24] of Example 13 of WO 2019/185879 A1), VSTB112 IgG1 (comprising VSTB112 HC (SEQ ID NO: 269)+VSTB112 LC (SEQ ID NO: 270) and mAb 13F3 (BioXCell Cat. No. BE0310).

Bio-Layer Interferometry (BLI) experiments were performed using the Octet QK384 system (ForteBio). All measurements were performed at 25° C.

Briefly, Anti-Penta-HIS (HIS1K) coated biosensor tips (Pall ForteBio, USA) were incubated for 60 sec in PBS buffer (pH 7.2) to obtain a first baseline, and tips were subsequently loaded for 120 sec with 270 nM HIS-tagged human VISTA or mouse VISTA, in PBS (pH 7.2).

After loading, biosensors were incubated for 60 sec in PBS buffer pH 7.2 to obtain a second baseline, and were then incubated for 120 sec with a 4 point, 2 fold dilution series of the test antibodies (concentrations: 250 nM, 125 nM, 62.5 nM and 31.3 nM) in PBS pH 7.2 to obtain association curves.

Finally, the biosensors were incubated for 120 sec in PBS pH 7.2 to obtain dissociation curves.

The results are shown in FIG. 1. V4-C26 was found to display binding to both human VISTA and mouse VISTA. By contrast, mAb 13F3 displayed binding to mouse VISTA but not human VISTA, and VSTB112 displayed binding to human VISTA but not mouse VISTA.

1.3 Tumor Growth Inhibition by V4-C26 hIgG4

The antigen-binding molecule V4-C26 hIgG4 comprising the heavy chain of SEQ ID NO:331 and the light chain of SEQ ID NO:317 was evaluated in a syngeneic cell line-derived mouse model of colon carcinoma for its ability to inhibit tumor growth in vivo.

CT26 cells were obtained from ATCC, and cultured in RPMI-1640 supplemented with 10% fetal bovine serum and 1% Pen/Strep, at 37° C. in a 5% CO₂ incubator.

CT26 cell-derived tumors were established by injecting 1×10⁵ CT26 cells subcutaneously into the right flanks of ˜6-8 week-old female BALB/c mice.

3 days post-implantation, and biweekly thereafter, mice were administered by intraperitoneal injection with 25 mg/kg V4-C26 hIgG4, or an equal volume of vehicle as a control condition (8 mice per treatment group).

Tumor volume was measured 3 times a week using a digital caliper, and calculated using the formula [L×W2/2]. The study end point was considered to have been reached once the tumors of the control arm measured >1.5 cm in length.

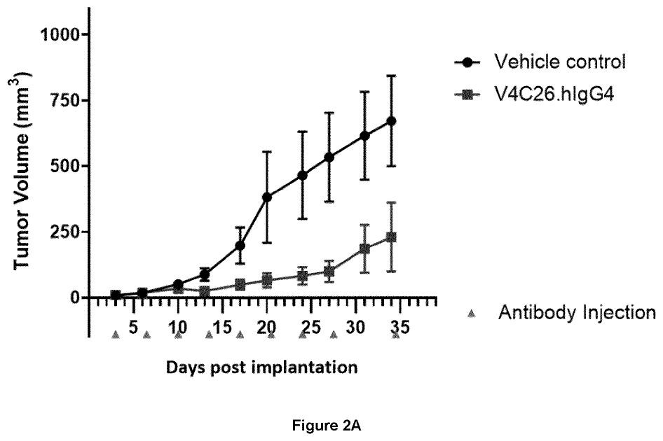

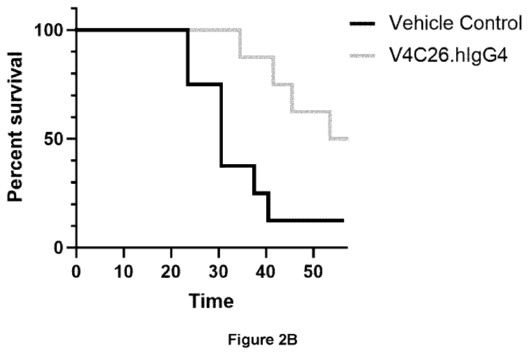

The results are shown in FIGS. 2A and 2B. Mice administered with V4-C26 hIgG4 displayed significant inhibition of tumor growth and improved survival relative to mice administered with vehicle.

### Example 2: Materials and Methods for Examples 3 to 7

### ELISA Binding Assay

384-well plates were coated with 1 μg/ml of target antigen diluted in PBS for 16 hrs at 4° C. After blocking for 1 hr with 1% BSA in Tris-buffered saline (TBS) at room temperature, V4-C26 hIgG4 or human IgG4 isotype control (Biolegend #403702) were serially diluted using 1% BSA made with 1×PBS at neutral pH 7 and added to the plate. For testing the binding of test article at different pH, 1% BSA was made using 1×PBS at pH of 7.5, 6.5, 6, 5.5 or 5. Post 1 hr incubation at room temperature, plates were washed three times with TBS containing 0.05% Tween 20 (TBS-T) and incubated with 1:7000 of goat anti-human IgG Fc-HRP (Abcam #ab97225) for 1 hr at room temperature. After washing, plates were developed with colorimetric detection substrate 3,3′,5,5′-tetramethylbenzidine (Turbo-TMB; Pierce) for 10 min. The reaction was stopped with 2M H2SO4, and OD was measured at 450 nm on a BioTek Synergy HT.

### Flow Cytometry and Analysis

V4-C26 hIgG4 binding to cell surface expressed VISTA on either PBMC, or HEK293T cells engineered to express VISTA, was measured by flow cytometry. Wild type HEK293T cells were transiently transfected with VISTA cDNA expression plasmids encoding human, NHP, rat, and mouse VISTA (Sinobiological) using lipofectamine 2000 (ThermoFisher Scientific #11668019) following the manufacturer's protocol. PBMCs from human, NHP, rat and mouse were procured from commercial vendor (Accegen) and blocked with Fc block (Human TruStain FcX, Biolegend #422302, Mouse TruStain Fcx, Biolegend #101320, Anti-Rat CD32, BD Pharmingen #550270 and Rhesus FcR Binding Inhibitor, ThermoFisher #14-9165-42) prior to staining. V4-C26 hIgG4 or isotype control antibodies were conjugated with APC as per manufacturers protocol.

For FACS, cells were incubated with different concentration of APC tagged V4-C26 hIgG4 or isotype control as indicated in the figure for 40 mins at 4° C. To identify myeloid cells, PBMCs were further incubated with CD45 FITC and CD11 b PE. Cells were washed again and resuspended in 200pL of FACS flow buffer (PBS with 5 mM EDTA) for flow cytometric analysis using MACSQuant 10 (Miltenyi). After acquisition, all raw data were analyzed using Flowlogic software. Cells were gated using forward and side scatter, and the percentage of positive cells was determined.

### Immunohistochemistry

Tissue microarrays (TMA) of TNBC, NSCLC, Mesothelioma and liver cancer patients (USBiomax, catalog #BR1301, #LC1401, #MS481d, #LV8013a) comprising formalin fixed paraffin embedded (FFPE) tissues were stained for VISTA (4M2-C12-mIgG2a; dilution 1:800) and VSIG3 (LS Biosciences #LS-C338858; dilution 1:300) and normal TMA (USBiomax, catalog #FDA999q) was also stained for VISTA (4M2-C12-mIgG2a). Slides were dried in a desiccator for 15 mins⁻¹ hr and placed in EnVision™ FLEX Target Retrieval Solution, low pH (Dako, K8005/DM829) for 20 mins at 97° C. for antigen retrieval. Slides were placed in Envision Flex buffer (1×) for 10 minutes prior to transferring to the Omnis instrument for staining. Slides were stained and counterstained on the Dako Link Omnis with the Envision Flex+ detection system (kit K800) using the kit-based protocol, followed by rinsing, dehydrating and coverslipping. The TMAs were semi-quantitatively scored by light microscopy to determine the relative intensity of staining (0-3+ intensity scale), distribution and localization of VISTA and VSIG3 protein expression in normal and tumor tissues.

### VISTA-VSIG3 Inhibition Assay

VISTA-VSIG3 binding was confirmed using recombinant human VISTA-Fc protein (R&D #7126-B7) or irrelevant antigen (Human recombinant CD47 protein, Sinobiological #112283-HCCH) with standard ELISA method. For the inhibition assay, 384-well plates were coated with 2 μg/ml of human VISTA-Fc recombinant protein (R&D #7126-B7) diluted in PBS for 24 hrs at 4° C. After blocking for 2 hrs with 1% BSA at room temperature, plates were incubated with either V4-C26 hIgG4 or isotype control for 30 mins at room temperature. After 30 mins, biotinylated recombinant human VSIG3-Fc (Recombinant Human VSIG3. Fc was biotinylated using Invitrogen kit #21455) at 24 nM (EC₅₀ of VISTA-VSIG3 binding) was added for 2 hrs. Post incubation, plates were washed three times with TBST and incubated with streptavidin HRP (R&D #DY998) antibody for 1 hr at room temperature followed by three further washes. Colorimetric reactions were developed using standard protocol as described for ELISA above.

Cytokine Release Post V4-C26 hIgG4 Blocking of VISTA-VSIG3 PBMCs were cultured in plates coated with αCD3 monoclonal antibody (eBioscience #16-0037) and VSIG3-Fc (R&D #9229-VS) at ratios of either 1:0 (2 pg/ml αCD3 alone) and 1:2 (2 pg/ml αCD3: 4 μg/ml of VSIG3-Fc). Cells were then treated with either V4-C26 hIgG4, VSTB112 or IgG4 isotype control (Biolegend #403702) at the indicated concentration and plate was incubated at 37° C. Supernatant was harvested after 24 hrs and IFN-γ levels was measured using Human IFN-γ Uncoated ELISA kit (Invitrogen #88-7316).

### MDSC-T Cell Co-Culture

Monocytes were isolated from fresh human PBMCs via negative enrichment using Classical Monocyte Isolation Kit (Miltenyi, Germany, #130-117-337). Subsequently, monocytes were differentiated to MDSCs for 7 days in the presence of GM-CSF (10 ng/ml) (PeproTech, USA, #300-03) and IL-6 (10 ng/ml) (PeproTech, USA, #200-06). MDSCs were harvested and cultured with freshly isolated autologous PBMCs at 3:1 ratio in the presence of human anti-CD3 antibody (OKT3, 1 μg/ml) (BioLegend, USA, #317326) and in the presence or absence of test articles as indicated. Supernatant was harvested after 96 hrs and IFN-γ levels were determined via ELISA (ThermoFisher, USA, #88-7316-88).

### Neutrophil Chemotaxis Assay

Neutrophils were isolated from whole blood using MACSxpress® Whole Blood Neutrophil Isolation Kit (Miltenyi #130-104-434) and incubated with either V4-C26 hIgG4, VSTB112 or isotype control, at the indicated concentration, for 60 mins at 37° C. Post incubation, neutrophils were seeded in the upper chamber (300 μl/well) of 24 well transwell plate (Thermo Fisher #1406287) and media, with or without C5a at 50 ng/ml (Acro Biosystem #C5A-H5116), was added to the lower chamber (600 μl). Cells were incubated in the transwell for 1 hr at 37° C., after which ATP levels of the migrated neutrophils in the lower chamber was measured using CellTiter-Glo (Promega #G7571) and luminescence was measured by Victor Nova (Perkin Elmer).

### Human Allogeneic Mixed Lymphocytic Reaction (MLR)

Fresh PBMCs were isolated from human whole blood (5 donors in total) using Lymphoprep (Stemcell Technologies, #07861), following manufacture's protocol and re-suspended with CellGenix GMP DC Medium at 5×106 cells/ml. 50 μl of DC medium was added into each well of 96-well round bottom plate followed by 50 μl of PBMCs from 2 donors at 1:1 ratio in the presence of either 50 μl of V4-C26 hIgG4 or isotype control hIgG4 (InvivoGen #bgal-mab114) at 30, 10 and 1 μg/mL. A total of 10 donor pairs were used. Cells were incubated at 37° C. for 96 hrs. Supernatants were collected at 96 hrs and the supernatant cytokine levels were detected by Luminex (R&D #LXSAHM-04/07). Data was normalized to hIg4 isotype control by subtracting isotype control values from test samples.

### Animal Experiments

Balb/c mice were purchased from InVivos or Jackson Labs and CD34 engrafted humanized HiMice mice were purchased from Invivocue. All animals were housed under specific pathogen-free conditions in an AALAC accredited facility and treated in strict compliance with the Institutional Animal Care and Use Committee guidelines.

### In Vivo Tumor Growth Assays

Mice were subcutaneously implanted with tumor cells (105 for CT26, 10⁶ for HCT15 and 5×10⁶ for A549) in the right flank or for orthotopic breast model, tumor cells were implanted into the mammary fat pad (2×10⁴ 4T1 cells). 3-6 days post implantation, mice were treated twice a week with the indicated dose and interval of the test articles. Treatment was administered intraperitoneally for all subcutaneous models, and intratumorally for the orthotopic model. Tumor volume was measured using calipers as described in Thakkar et al., Mol Cancer Ther (2020) 19: 490-501).

### Statistical Analysis

Statistical analysis was performed using GraphPad Prism. Data acquired with two variables (dose titrations) was analyzed with 2-way ANOVAs followed by Tukey's multiple comparisons test. For comparisons between two groups, an unpaired t-test was performed. Values of *p≤0.05, **p≤0.01, ***p≤0.001, ****p≤0.0001, were considered significant.

### Antibody Isolation

Six- to eight-week-old female BALB/c mice were repeatedly immunized with custom immunogens and adjuvants. Twenty-four hours after the final immunization, total splenocytes were isolated and fused with the myeloma cell line P3X63.Ag8.653 (ATCC) using ClonaCell-HY Hybridoma Cloning Kit, in accordance with the manufacturer's instructions (Stemcell Technologies). After 7 to 10 days, single hybridoma clones were isolated and antibody-producing hybridomas were selected by screening supernatants for antigen binding using ELISA and flow cytometry. Variable regions of selected clones were amplified (SMARTer RACE 5/3′ Kit, Clontech), sequenced and cloned into expression vector and expressed in mammalian cells for in vitro functional testing.

### Hybridoma Production

Approximately 6-week-old female BALB/c mice were obtained from InVivos (Singapore). Animals were housed under specific pathogen-free conditions and were treated in compliance with the Institutional Animal Care and Use Committee (IACUC) guidelines. Using our proprietary immunization protocol (mAbHits), mice were immunized with proprietary mixtures of custom immunogens. Total splenocytes were isolated and fused with the myeloma cell line P3X63.Ag8.653 (ATCC, USA) using polyethylene glycol (PEG) and ClonaCell-HY Hybridoma Cloning Kit (Stemcell Technologies, Canada), according to manufacturer's instructions. Monoclonal hybridomas were selected and supernatants from the resulting clones were screened by enzyme linked immunosorbent assay (ELISA) and fluorescent activated cell sorting (FACS).

### Antibody Variable Region Cloning and Sequencing

Total RNA was extracted from hybridoma cells using TRIzol reagent (Life Technologies, Inc., USA) according to the manufacturer's instructions. Double-stranded cDNA was synthesized using SMARTer RACE 5/3′ Kit (Clontech™, USA). The race-ready cDNAs were amplified using SeqAmp DNA Polymerase (Clontech™, USA) and primer mixes. The resulting variable heavy (VH) and light (VL) amplicons were cloned into pJET1.2/blunt vector using CloneJET PCR Cloning Kit (Thermo Scientific, USA) and purified PCR amplicons were sequenced through AITBiotech Pte. Ltd. (Singapore). The sequencing data was analysed using the international IMGT (ImMunoGeneTics) information system (Lefranc et al., Nucleic Acids Res (2015) 43: D413-D422) to characterize the individual complementarity-determining regions (CDRs) and framework sequences.

### Humanization and Affinity Maturation of Mouse Antibody

Humanization of the variable regions of the mouse sequence was performed by CDR grafting into human framework sequences and backmutation of residues in canonical positions to preserve antigen binding. The humanised antibody was further affinity matured against mouse VISTA to increase the cross-species affinity by random mutagenesis using yeast scFv surface display. Briefly, ScFv bearing the parental VH and VL in a single chain was amplified by mutagenesis PCR (Agilent Technologies GeneMorph II Random Mutagenesis Kit) for library creation, the resulting library was further amplified and cloned into yeast expression vector pCTcon2 (Addgene) for yeast surface expression by electroporation. The transfected cells were screened by mouse VISTA binding using FACS and high binder cell was sorted by BD FACS Aria II cell sorter. Selected high binder clones were enriched by 2 additional rounds of sorting. Cells were plated in SDCAA to isolate single clones followed by DNA extraction and sequencing.

### Cell Lines

All cell lines were purchased from ATCC and cultured as recommended. Cells were maintained in culture medium supplemented with 10% FBS and 1% Pen/Strep (ThermoFisher) and cultured at 37° C., in 5% CO₂ incubators. Prior to use, fresh vials of cells were thawed and passaged 2-5 times. And Mycoplasma testing was performed by PCR (DreamTaq Green PCR master mix, K1081, ThermoFisher) and using the Agilent Mycosensor assay kit, according to manufacturer's instructions.

### Stable Cell Line Generation

CHO-K1 cells at an optimal density of 1×10⁶ cell/ml were electroporated with 5 μg of linearized IgG expression plasmid using 4D-Nucleofector kit (Lonza) according to manufacturer's protocol. Electroporated cells were cultured in static cell incubator in a 6-well plate containing 2 ml growth medium for 24 hr. Subsequently, medium was exchanged to selection medium containing 250 nM Methotrexate (Sigma) and 200 μg/ml Zeocin (InvivoGen). Cells were spun down and re-suspended in fresh selection medium and re-seeded to a density of 5×10⁵ cell/ml once per week. Selection was completed when 95% viability was restored. Cells were transferred to shaker-incubator.

### Antibody Production and Purification

Antibodies were produced by cultivation of stable cells in Fed-Batch mode in a shaker-incubator and subsequently purified from culture supernatants by affinity, size exclusion or mixed modal anion exchange followed by a final anion exchange chromatography. Antibody purity was assessed by size exclusion chromatography and SDS-PAGE.

### VISTA Sequence Analysis and Visualization

Multiple sequence alignments were performed with MultAlign (Corpet, Nucleic Acids Res (1988) 16: 10881-10890) and visualized with EPScript (Robert and Gouet, Nucleic Acids Res (2014) 42: W320-W324). 3D models were generated using UCSF Chimera (Pettersen et al., J Comput Chem (2004) 25: 1605-1612).

### VISTA-LRIG1 Inhibition Assay

VISTA-LRIG1 binding was confirmed using recombinant human VISTA-Fc protein (R&D #7126-B7) or irrelevant antigen (Human recombinant CD47 protein, Sinobiological #112283-HCCH) with a standard ELISA method. For inhibition assay, 384-well plates were coated with 5 μg/ml of human VISTA-Fc recombinant protein (R&D #7126-B7) diluted in PBS for 24 hrs at 4° C. After blocking for 2 hrs with 1% BSA at room temperature, plates were incubated with either V4-C26 hIgG4 or isotype control (Biolegend #403702) for 60 mins at room temperature. After 60 mins, recombinant human LRIG1.HIS at 1 μg/ml (EC₅₀ of VISTA-LRIG1 binding) was added for 2 hrs. Post incubation, plates were washed three times with TBST and incubated with anti-HIS HRP (Abcam #ab1187) antibody for 1 hr at room temperature followed by three further washes. Colorimetric reactions were developed using standard protocol as described for ELISA above.

### Epitope Mapping by Hydrogen-Deuterium Exchange Mass Spectrometry (HDXMS)

Epitope mapping by HDXMS was conducted using HIS-tagged human VISTA (residues 33-194, Sino Biological #13482-H08H) and V4-C26 hIgG4 following procedures described in Wales et al., Anal Chem (2008) 80: 6815-6820. Briefly, free VISTA was diluted in deuterated PBS with final deuterium oxide (D20) concentration at 90%. For VISTA/V4-C26 hIgG4 complex, VISTA and V4-C26 hIgG4 were mixed in 2:1 ratio and incubated for 15 mins at 25° C. prior to deuterium labelling. Deuterium labelling reactions were carried out at 25° C. for 1-, 10-, 30- and 100-min time points. Samples were then subjected to pepsin proteolytic cleavage followed by separation on an ACQUITY C18 column (1.0×100 mm) by nanoACQUITY UPLC (Waters, Manchester, UK) and detection by Synapt G2-Si mass spectrometer (Waters, Manchester, UK), operated in HDMSE mode. Peptide identification and deuterium uptake monitoring were respectively performed using Protein Lynx Global Server 3.0.1 and DynamX 3.0 (Waters). Deuterium uptake for the peptides were calculated as differences in masses of the centroids of deuterated and undeuterated samples (Wales et al., Methods Mol Biology (2013) 1007: 263-288) and reported as an average of triplicate measurements (Masson et al., Nat Methods (2019) 16: 595-602).

### Epitope Binning

For epitope binning, human VISTA-HIS, or mouse VISTA-HIS recombinant protein (Sino Biological Inc.) in PBS was immobilized to Anti-Penta HIS sensor (HIS1K, Molecular Device) on an Octet QK384 (Molecular Device) instrument, for 5 mins. Sensors were briefly washed in PBS for 30 s before loading 400 nM saturating antibody in PBS for 10 mins at a shake speed of 1,000 rpm. Subsequently, biosensors were washed for 2 min before immersing in 400 nM competing antibody in PBS for 7.5 mins at a shake speed of 1,000 rpm. Binding events, correlated to the change in wavelength (nm shift) reported from the sensorgram, are monitored at the detector in real time.

### Cross-Species Antibody Binding Affinity Measurement

The affinity of V4-C26 hIgG4 was determined by Surface Plasmon Resonance (SPR) using a Biacore. The assay was performed using a CM5 sensor chip (Cytiva #29104988). A Biacore HIS capture kit (GE Healthcare #28-9950-56) was employed to immobilize human, NHP, rat and mouse VISTA-HIS on-chip surface or left alone for background signal correction. Briefly, VISTA-HIS was captured for a contact time of 60 s at a flow rate of 5 μl/min. V4-C26 hIgG4 was flowed in two-fold serial dilutions from 50E⁻⁹ M to 390E⁻¹² M for human, NHP and rat VISTA and from 12.5^{E-9} M to 390E⁻¹² M for mouse VISTA, at a flow rate of 30 μl/min for 90 sec association and 3000 sec dissociation at RT. The obtained sensograms were analyzed using Biacore T200 software and K_{D} was calculated by fitting to a 1:1 binding kinetics model.

### Antibody-Dependent Cellular Cytotoxicity and Complement-Dependent Cytotoxicity

96 well plates were coated with either 1 μg/well of human C1q protein or 0.5 ug/well of human CD16a in PBS at 4° C. Post overnight incubation, plates were washed thrice and blocked for 1 hr with blocking buffer at room temperature. Plates were incubated with V4-C26 hIgG4 or isotype control for 1 hr at room temperature and incubated with 1:7000 dilution of HRP-conjugated anti-human Fc antibody for 1 hr at room temperature. Colorimetric reactions were developed using standard protocol as described for ELISA above.

### Sc-RNA-Seq

To determine the cell types in which VISTA transcripts were most highly expressed, a publicly available 68k PBMC dataset was retrieved from the 10× Genomics website (Stuart et al., Biorxiv (2018), 460147) and processed using Seurat v3.2 (Stuart et al., Cell (2019) 177: 1888-1902.e21). Cells with more than 6% mitochondrial transcripts, or less than 200 distinct transcripts were excluded. Data was normalized using default parameters and variable features identified using the VST method with 20,000 features. Data was subsequently scaled and then a PCA decomposition run with 15 principal components. A UMAP projection was carried out using default parameters and the FindClusters function run with a resolution of 0.8. To label the clusters the PBMC3k dataset (Hafemeister and Satija, Biorxiv (2019), 576827) from the SeuratData package was used as a reference to map cell identities.

### Bulk RNA-Seq

10 samples per condition (V4-C26, V9, anti-PD1, and IgG4) were taken from the MLR assay 96 hours after treatment. These were prepared for RNA-seq using the NEBNext Ultra II RNA library preparation kit (NEB #E7770S) and run on an Illumina NovaSeq S4 flowcell with v1.5 chemistry. Adaptor-trimmed and filtered data was evaluated using FASTQC and MultiQC (Ewels et al., Bioinformatics (2016) 32: 3047-3048) and reads aligned to the GRC37 human reference transcriptome using Kallisto v0.46 (Bray et al., Nat Biotechnol (2016) 34: 525-527). Data was imported to R using Txlmport (Soneson et al., F1000research (2015) 4: 1521). Normalized gene counts and gene-level differential expression was obtained using DESeq2 (Love et al., Genome Biol (2014) 15: 550). Pathway activity was estimated using the SPEED2 (Rydenfelt et al., Nucleic Acids Res (2020) 48: W307-W312) package using default parameters.

### CT26 Tumor Profiling

Single cell suspensions were generated by digestion of finely cut tumor tissue with 0.1 mg/ml of DNase I (Sigma, USA, #11284932001) and 1 mg/ml Collagenase (Sigma, USA, #1108866001) for 45 minutes at 37° C. Fc receptors were blocked with Human TruStain FcX (BioLegend, USA, #422302). Cells were stained with fluorophore conjugated antibodies for markers of immune cells (Table S4), washed and resuspended in buffer (1×PBS+0.5% BSA+1 mM EDTA). Frequency of immune cell populations was determined via flow cytometry. Data was acquired on MACSQuant 10 (Milteny Biotec, Germany, #130-096-343) and analyzed using FlowLogic software V7.

### CT26 Antigen Recall Assay

Single cell tumor suspensions were generated as described earlier. TILs (CD45+) or T cells (CD4+/CD8+) were enriched from the cell suspension using CD45 MicroBeads (Milteny Biotec, Germany, #130-110-618) or CD4/CD8 MicroBeads (Milteny Biotec, Germany, #130-116-480) as per manufacturer's protocol. CT26 cells (T: target cells), seeded 24 hrs prior, were co-cultured with enriched TILs or T cells (E: effector cells) from tumors for 72 hours at effector to target (E:T) cell ratios as indicated. All conditions were in triplicates. Cell viability was determined by CellTiter Glo (Promega, USA, #G7571). IFN-γ levels in supernatants were determined by ELISA (Invitrogen, USA, #BMS606). Lysis was calculated as: % Lysis=((T−E:T)/T)*100.

### Pharmacokinetics

A single dose pharmacokinetic profile of V4-C26 hIgG4 was evaluated in male and female Balb/c mice, Sprague Dawley rats and cynomolgus monkeys. V4-C26 hIgG4 was administered in a single dose at the indicated concentration via intraperitoneal injection in mice, IV bolus infusion into the tail vein in rats, and IV bolus infusion into the peripheral vein in monkeys. Blood was drawn at different timepoints post dosing and antibody concentration in the serum was quantified by ELISA. The parameters for the pharmacokinetic analysis were derived from a non-compartmental model: maximum concentration (Cmax), AUC (0-336 hr), AUC (0-infinity), half-life (t½), clearance (CL) and volume of distribution at steady state (Vss).

### Example 3: Distribution of VISTA Expression

### 3.1 VISTA is Predominantly Expressed on Myeloid-Derived Cells in Healthy Tissues

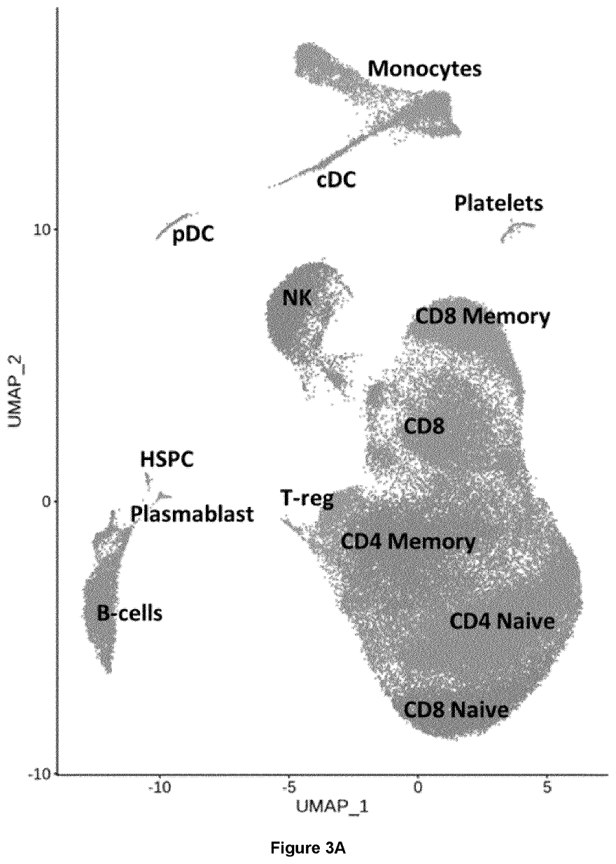

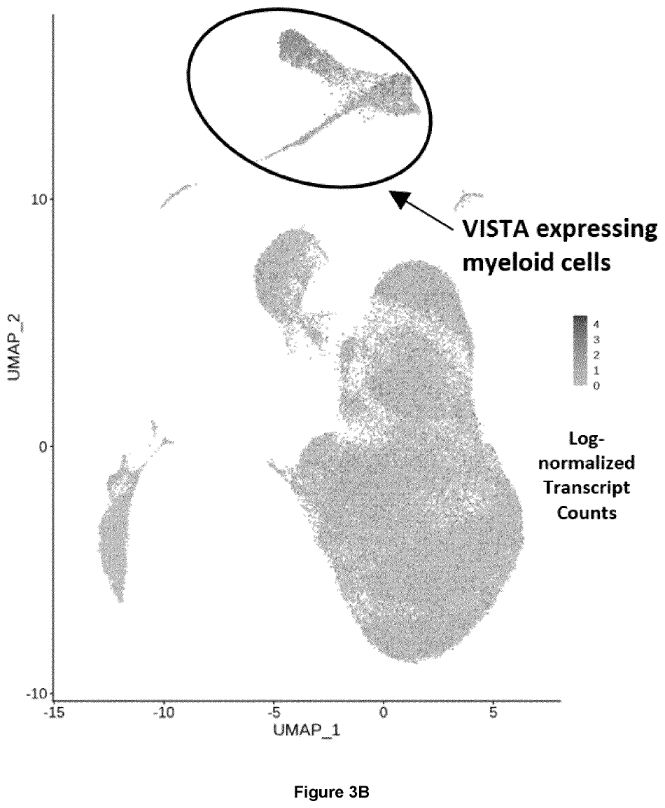

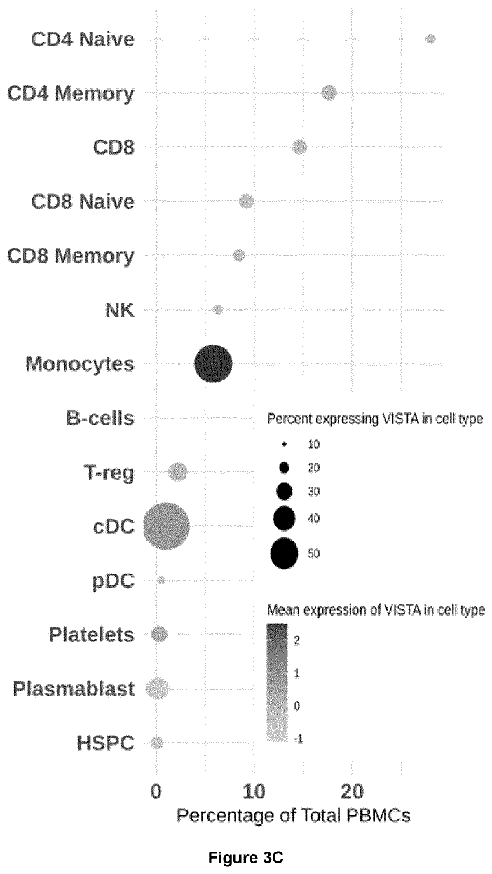

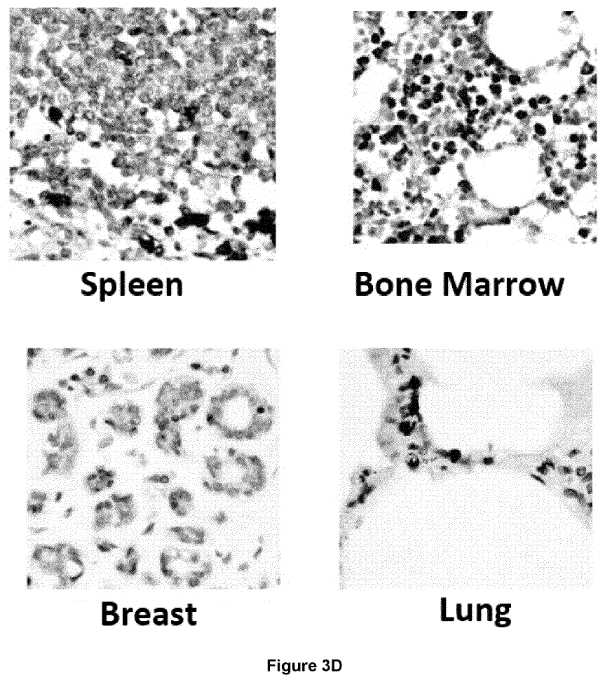

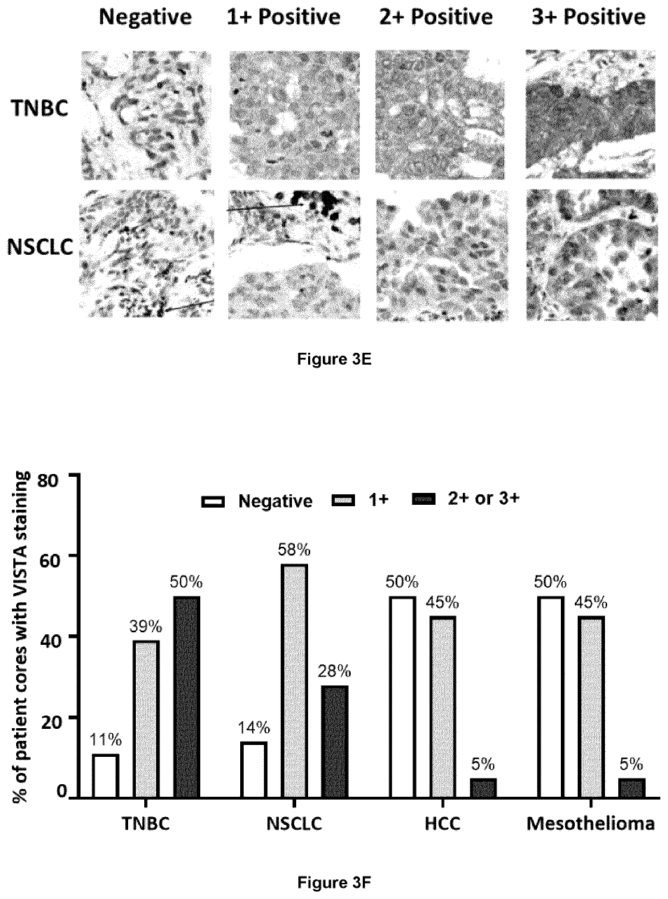

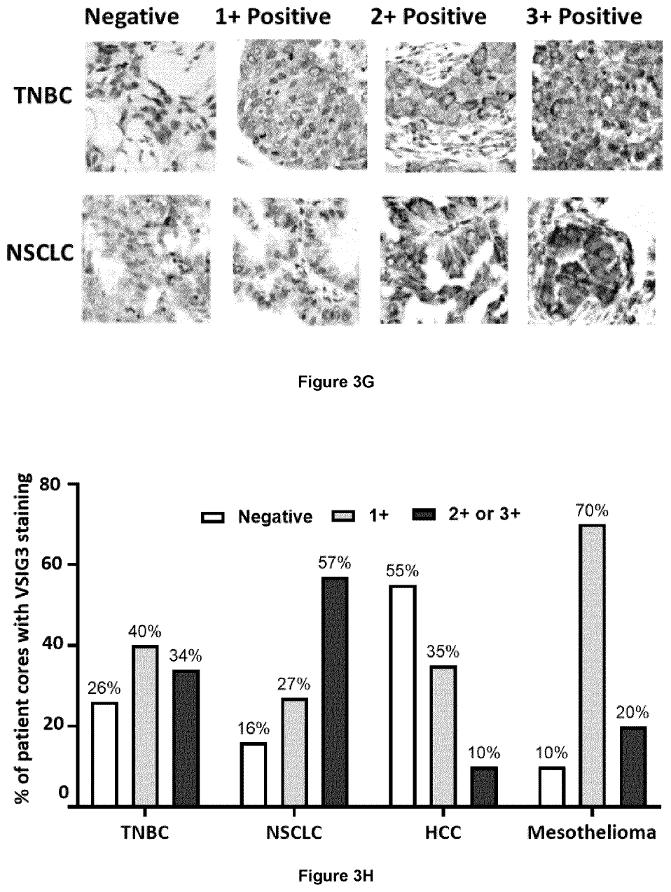

The distribution of VISTA expression was studied in healthy tissues to evaluate the optimal strategy for a VISTA antagonist. A single cell (sc) RNA-seq dataset from 10× Genomics comprising 68,000 PBMCs [www.1 Oxgenomics.com/single-cell-gene-expression/datasets] was analysed. This revealed the presence of 14 major cell clusters (FIG. 3A). Although low levels of VISTA transcripts were identified in many cell populations, high expression was confined to myeloid-derived monocytes and dendritic cells in healthy human donors, with limited expression in T cells (FIGS. 3B, 3C).

VISTA protein expression in healthy human tissues was further characterized using immunohistochemistry (IHC) on formalin fixed paraffin embedded (FFPE) tissue microarray (TMA) sections (FIG. 3D). The highest VISTA levels were detected in lymphoid organs (e.g. spleen and bone marrow) and tissues with significant infiltration by leukocytes (e.g. breast and lung). The distribution of VISTA across healthy tissues and diverse immune cells strongly supports the need to antagonize VISTA without depleting VISTA expressing cells for optimal therapeutic benefit and tolerable safety.

### 3.2 Solid Tumours Including TNBC and NSCLC Show Significant Expression of VISTA and VSIG3

The expression of VISTA and VSIG3 was assessed via immunohistochemistry in formalin fixed paraffin embedded (FFPE) tissue microarray (TMA) sections of four solid cancers, including triple negative breast cancer (TNBC), non-small cell lung cancer (NSCLC), hepato-cellular carcinoma (HCC) and mesothelioma. Immunohistochemistry used 4M2-C12-mIgG2a (which is formed of the polypeptides of SEQ ID NOs:248 and 250) or an anti-VSIG3 antibody.

TNBC and NSCLC patients showed the highest VISTA expression, with 89% and 85%, of the cores showing moderate-high staining intensity, respectively (FIG. 3F). The expression of VSIG3 was evaluated further. VSIG3 showed moderate to high staining in mesothelioma (90%), NSCLC (84%), and TNBC (74%) (FIG. 3H). The observed high expression of both VISTA and VSIG3 in TNBC and NSCLC, is consistent with reports that the co-inhibitory function of VISTA may be mediated through VISTA-VSIG3 interactions suppressing T cell function (Wang et al., Immunology (2019) 156: 74-85) and suggests that these could be priority indications to investigate the benefits of VISTA antagonism.

### Example 4: Analysis of VISTA Binding by Interaction Partners and V4-C26 Antibody

Given the expression pattern of VISTA on healthy cells, V4-C26 was developed as an IgG4 isotype anti-VISTA antibody to inhibit VISTA function by blocking interactions with its binding partners without depleting VISTA expressing cells through Fc-mediated effector functions. Antibody V4-C26 hIgG4 comprises the heavy chain of SEQ ID NO:331 and the light chain of SEQ ID NO:317.

### 4.1 VISTA Binding by Interaction Partners

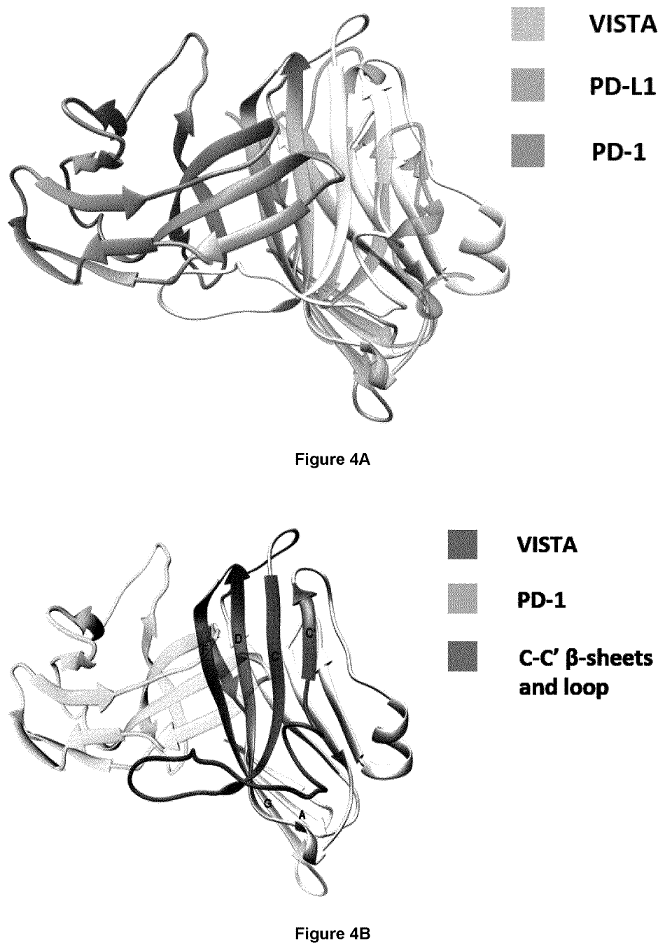

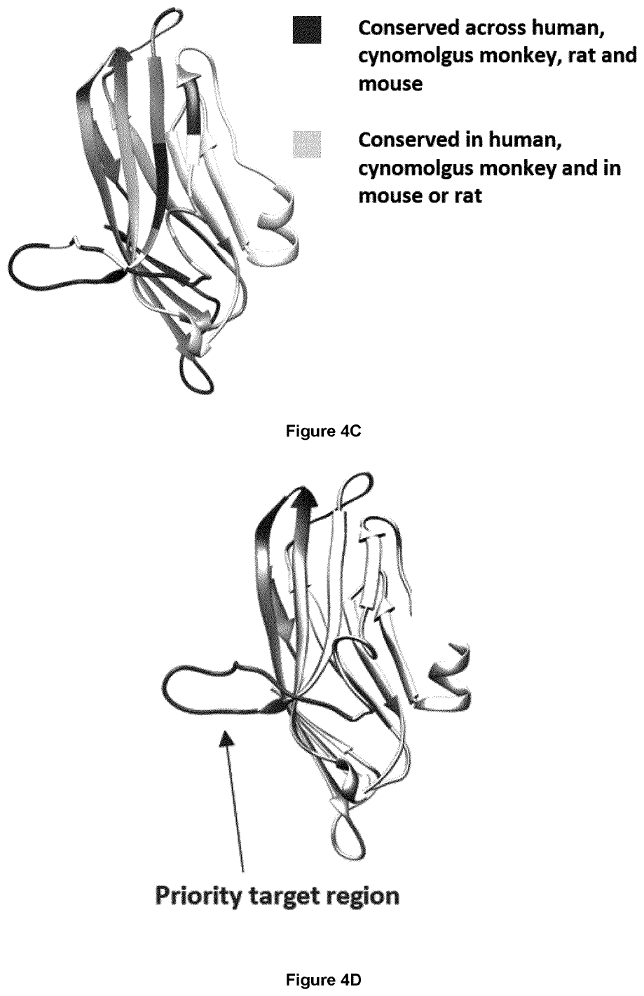

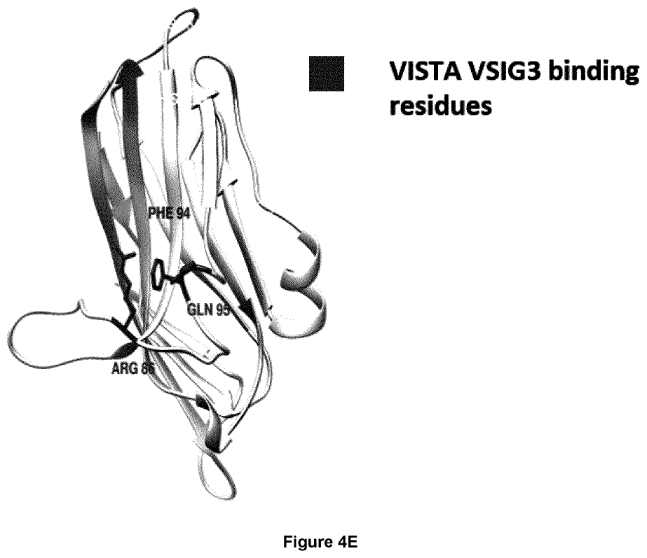

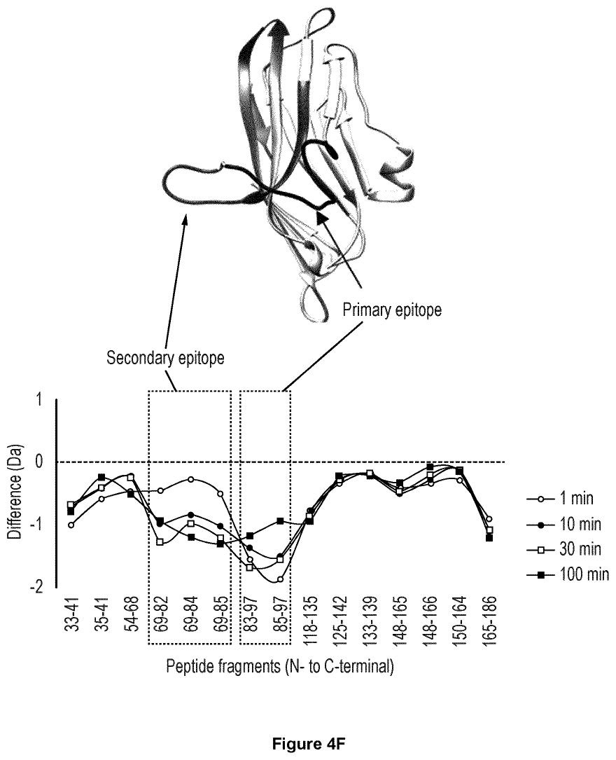

VISTA is a B7 family receptor so it was hypothesised that the interaction surface for VISTA and its binding partners would be conserved across other B7 receptors, including PD1/PD-L1 (FIG. 4A). To confirm, the PD1/PD-L1 complex (PDB: 5IUS) was used as an input structure for the prediction algorithm, which then superimposed the VISTA structure (601L) onto the complex.

Integrating this input structural data with the available sequence data for VISTA in humans and model species, the algorithm highlighted antibody-available regions along the F, D, C, C′ β-sheets and the C-C′ loop likely to be involved in mediating the interaction with VISTA's cognate receptor/ligand (FIG. 4B), and prioritized target regions in the C-C′ loop that are unique to VISTA within the human proteome and highly conserved across model species (FIGS. 4C, 4D).

The functional nature of this region is supported by known interaction residues for VSIG3 that are located within this C-C′ loop region, i.e. resides R86, F94 and Q95 (FIG. 4E).

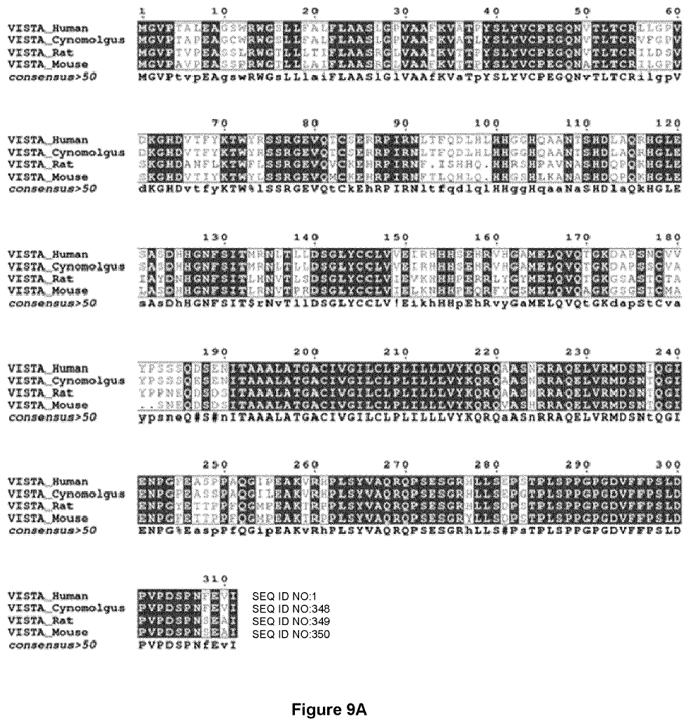

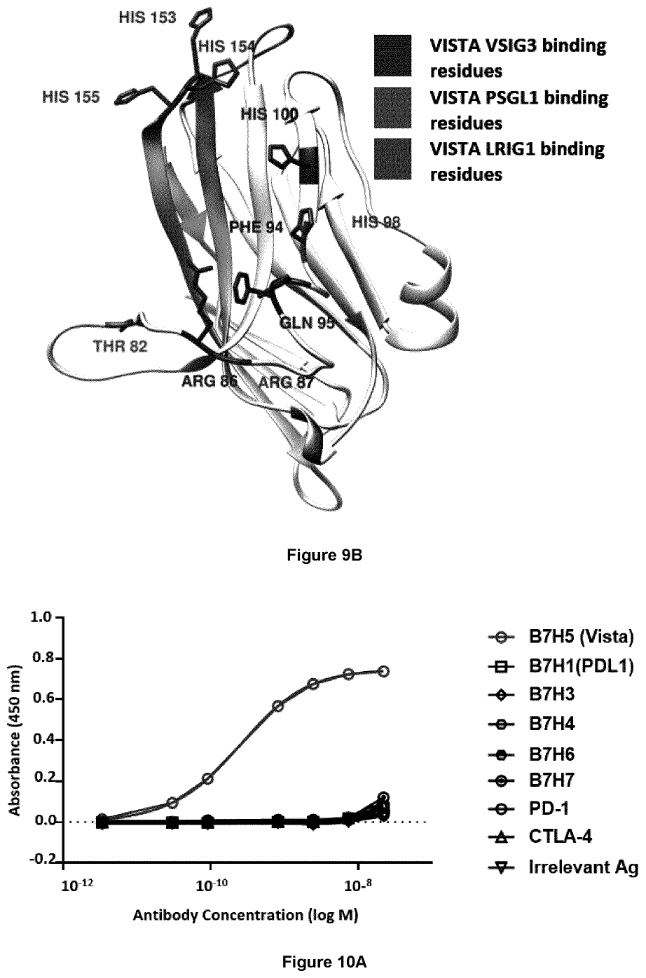

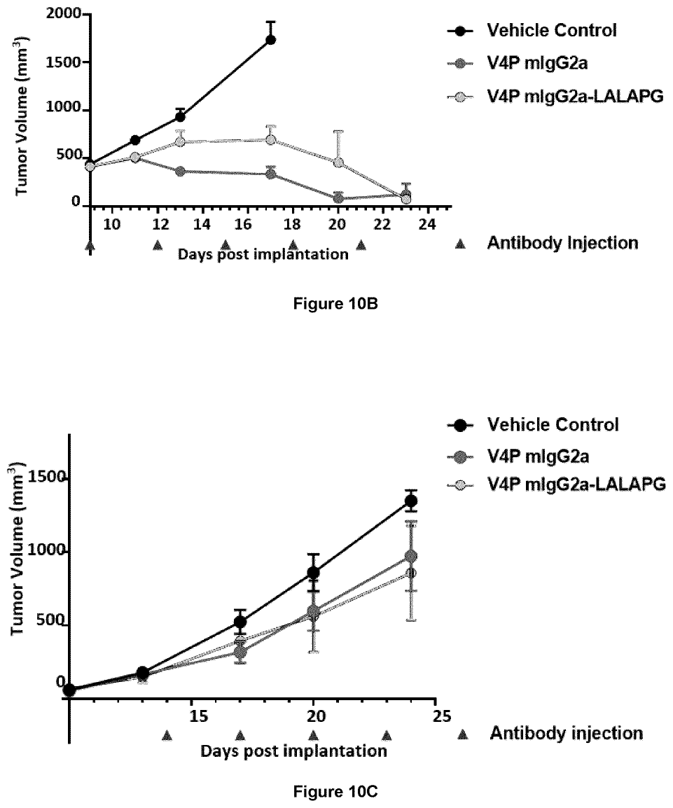

Further, the proposed residues for VISTA-LRIG1 interaction also fall within this region. WO/2019/165233 A1 discloses that LRIG1 binds to VISTA in the vicinity of amino acid positions 68-92 of VISTA (SEQ ID NO:343), and homology modelling predicts VISTA residues T82 and R87 to be key positions for interaction with LRIG1 (FIG. 9B).

The residues of VISTA involved in interaction with PSGL1 are more distant; Johnston et al., Nature (2019) 574: 565-570 discloses VISTA residues H98, H100, H153, H154 and H155 to be the key positions for interaction between VISTA and PSGL1 (FIG. 9B).

4.2 V4-C26 hIgG4 Epitope Mapping Analysis

Epitope mapping analysis by hydrogen-deuterium exchange mass spectrometry (HDXMS) was conducted for the VISTA-V4-C26 hIgG4 complex to confirm the V4-C26 hIgG4 binding region. Comparison of the deuterium exchange between V4-C26 hIgG4-bound VISTA and free VISTA showed that significant decrease in deuterium uptake was observed at 2 loci spanning residues 69-85 (SEQ ID NO:342) and 85-97 (SEQ ID NO:341)—see FIG. 4F, indicating that these regions were protected against deuterium exchange via protein-protein interaction. Furthermore, the reduced deuterium exchange at early timepoints observed for peptide 85-97 suggested that this is the primary epitope of V4-C26 hIgG4. Reduction of deuterium exchange at later timepoints observed for peptide 69-85 may represent a secondary binding event subsequent to binding of the primary epitope.

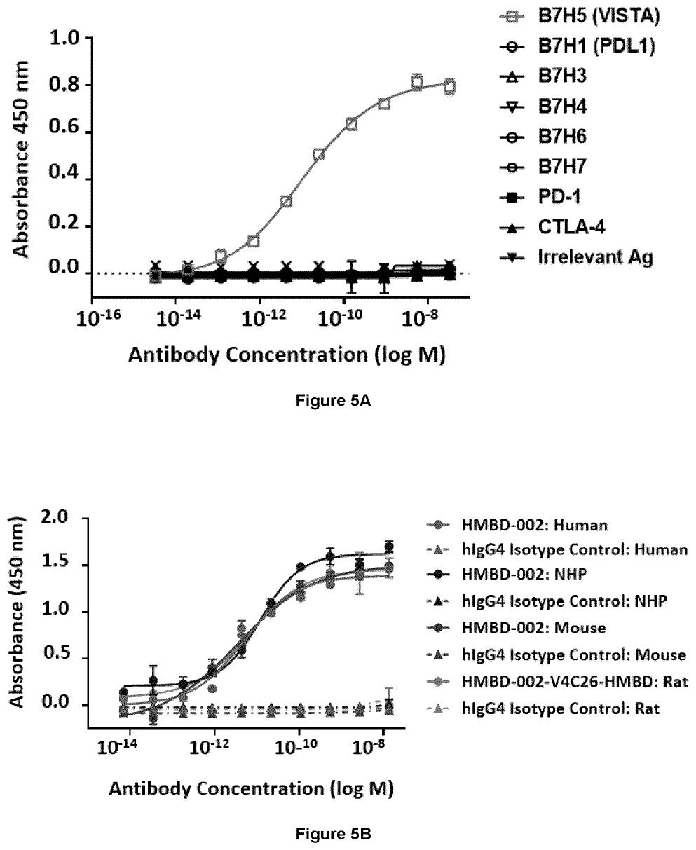

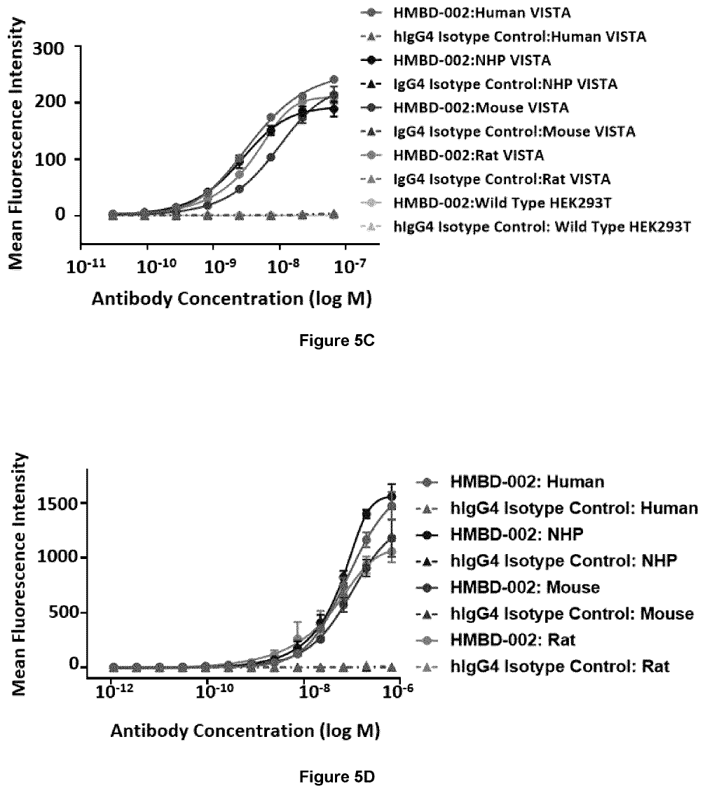

Mapping of these identified peptides to the VISTA structure confirmed that these sites overlapped with the predicted target region (see FIG. 5D) and include three critical amino acid residues for VSIG3 binding (R86, F94 and Q95, FIG. 9B).

Thus, V4-C26 hIgG4 binds with picomolar affinity to a species-conserved epitope within the predicted functional C-C′ region of VISTA (SEQ ID NO:344) with an epitope that overlaps with key residues for both VSIG3 and LRIG1 binding, and is able to block their interactions with VISTA.

The binding site of V4-C26 hIgG4 was then compared to other published VISTA antibodies by epitope binning with the human VISTA-specific VSTB112 and IGN175A antibodies (described e.g. in WO 2015/097536 A2 and WO 2014/197849 A2, respectively), and mouse VISTA ortholog specific 13F3 and MH5A antibodies.

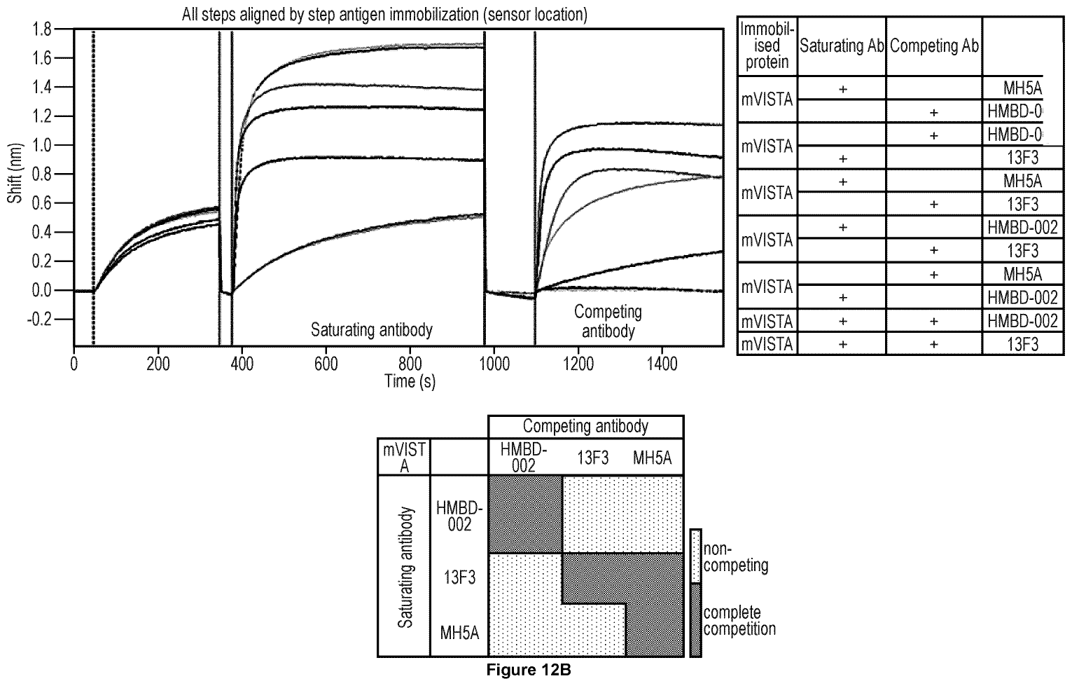

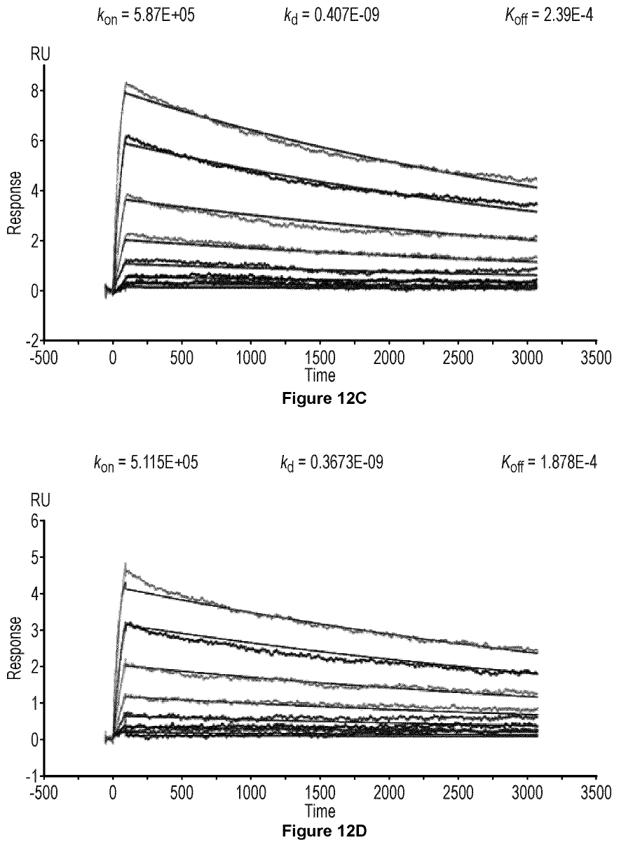

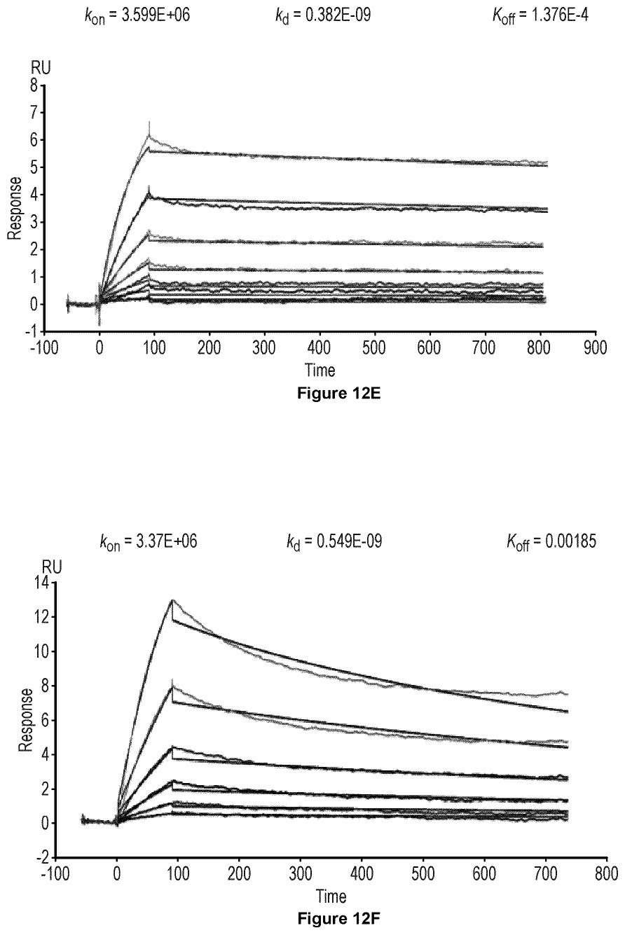

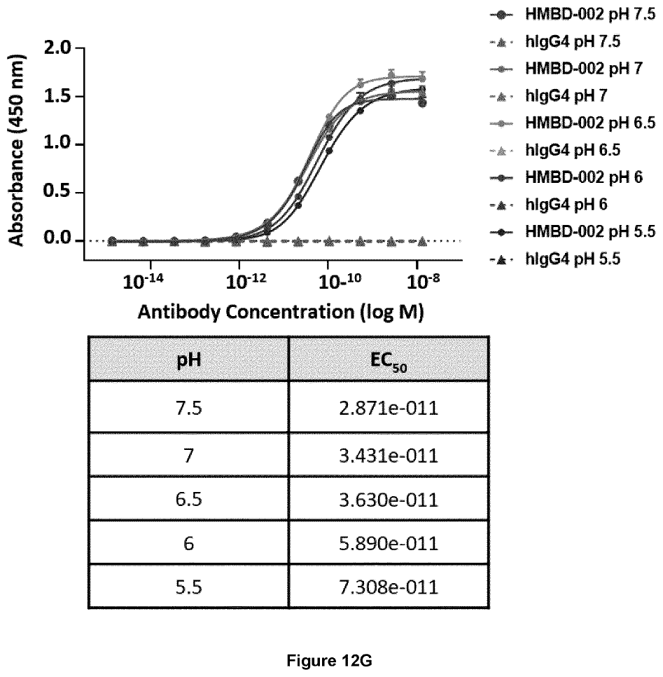

Lack of interference in binding indicated that V4-C26 hIgG4 and IGN175A bind to topologically distant epitopes on human VISTA (FIG. 12A). Similarly, 13F3, MH5A and V4-C26 hIgG4 are largely non-competing for binding to mouse VISTA (FIG. 12B). The partial interference of binding observed between VSTB112 and V4-C26 hIgG4 suggests that the epitope of VSTB112 is distinct but closer than IGN175A to the epitope of V4-C26 hIgG4. FIG. 1 demonstrates that the epitope bound by VSTB112 is distinct to that of V4-C26, as VSTB112 is unable to bind to mouse VISTA.

The only other murine cross-reactive anti-VISTA antibody, SG7, currently in early development, binds to a different region and shows much lower affinity to murine VISTA compared to human VISTA (N. Mehta et al., Sci Rep-uk 10, 15171 (2020)).

In conclusion, V4-C26 hIgG4 binds to a unique epitope on human and mouse VISTA orthologs.

### 4.3 Analysis of ADCC and CDC Functionality

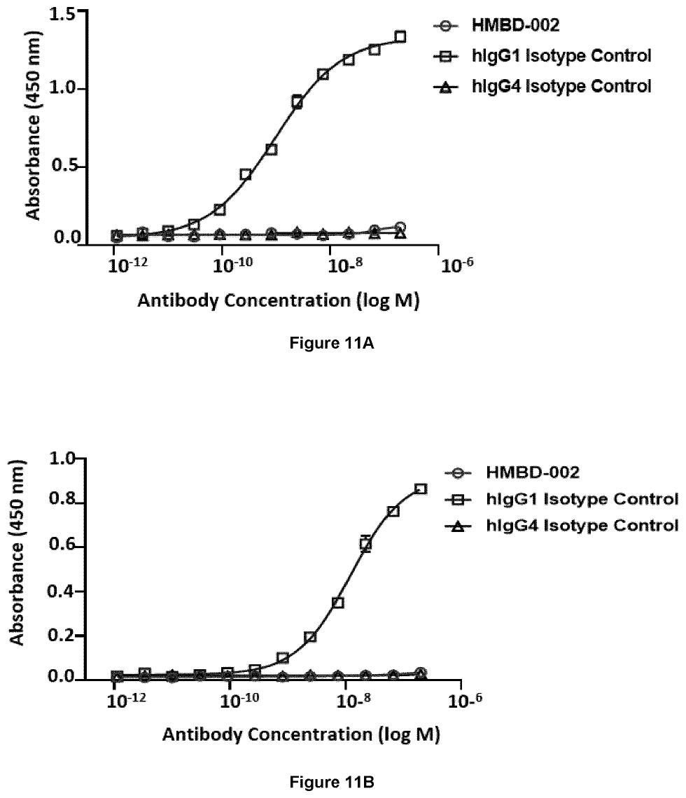

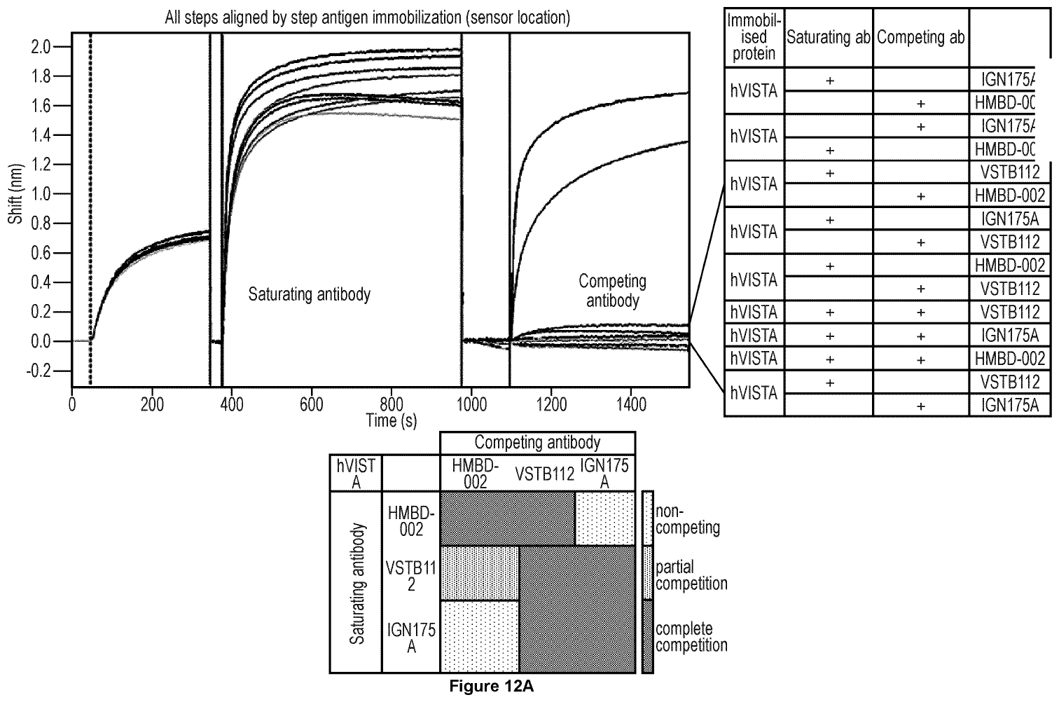

To rule out the potential for V4-C26 hIgG4 to cause ADCC and CDC reactions, its binding affinity to the respective FcγRIII and C1q proteins was evaluated. No binding was observed between FcγRIII and C1q proteins by ELISA (FIGS. 11A and 11B), which would preclude ADCC or CDC mechanisms depleting VISTA expressing cells.

This feature makes V4-C26 hIgG4 distinct from previously developed VISTA targeting antibodies that have used an IgG1 Fc isotype, known to trigger cell depletion by ADCC and CDC.

### Example 5: Analysis of Biophysical Properties of V4-C26 hIqG4

The biophysical properties of V4-C26 hIgG4 were evaluated.

Binding specificity of V4-C26 hIgG4 for VISTA was measured by ELISA.

FIG. 5A shows that V4-C26 hIgG4 displays highly specific binding to VISTA among the other related members of the B7 family (B7H1/PDL-1, B7H3, B7H4, B7H6, B7H7), as well as PD-1 and CTLA-4.

To support the use of rodent and non-human primate (NHP) species for efficacy, safety and PK models, the ability of V4-C26 hIgG4 to bind to VISTA orthologs was assessed using ELISA and SPR (Biacore).

FIG. 5B shows that V4-C26 hIgG4 demonstrates dose-dependent binding to human, NHP, rat and mouse VISTA-HIS protein by ELISA with an EC₅₀ of 5.117 pM, 12.15 pM, 6.689 pM, and 3.549 pM, respectively. V4-C26 hIgG4 was also observed to bind to human, NHP, rat and mouse VISTA orthologs with similar picomolar affinities (Kd) of 407 pM, 367 pM, 382 pM, and 549 pM respectively using SPR (Biacore)—see FIGS. 12C to 12F.

Cell surface binding of V4-C26 hIgG4 to HEK293T cells expressing recombinant VISTA orthologs, as well as to myeloid cells within PBMC of relevant pre-clinical species, was further confirmed by FACS.

FIG. 5C shows that V4-C26 hIgG4 showed dose-dependent binding to VISTA orthologs of all species tested, with comparable EC₅₀ values of 3.738 nM, 2.571 nM, 4.133 nM and 8.94 nM for human, NHP, rat, and mouse VISTA expressing HEK293T cells, respectively. There was no non-specific binding observed to wild type HEK293T cells, which do not express VISTA.

FIG. 5D shows that V4-C26 hIgG4 also showed comparable dose-dependent binding to endogenous VISTA expressed on the myeloid cells of all pre-clinical species tested, with EC₅₀ of 108 nM, 67.6 nM, 48.6 nM and 111 nM for human, NHP, rat, and mouse VISTA, respectively.

Some tumour environments have been reported to be hypoxic, characterized by relatively low pH. As VISTA contains many exposed histidine residues that are susceptible to protonation and may affect antibody binding, we evaluated the effect of pH on V4-C26 hIgG4 binding. FIG. 12G shows that V4-C26 hIgG4 was observed to bind VISTA with comparable affinity at pH 5.5-7.5, as assessed by ELISA, confirming that V4-C26 hIgG4 binds to VISTA across the range of physiologically relevant pH, and that the binding site is distinct from the histidine-rich regions.

Thus, the binding of V4-C26 hIgG4 is highly selective and maintained even in low pH conditions that may represent those in the tumour microenvironment.

### Example 6: Ability of V4-C26 hIqG4 to Block VISTA Functionality

6.1 V4-C26 hIgG4 Inhibits Binding Between VISTA and Key Interaction Partners

V4-C26 hIgG4 binds to the C-C′ region of VISTA (see Example 3), which is hypothesized to be a common functional region for VISTA to bind to multiple partners. Thus, V4-C26 hIgG4 was assessed for its ability to inhibit these interactions. ELISA-based recombinant protein binding assays were developed that modelled the binding between VISTA and its binding partners VSIG3 and LRIG1.

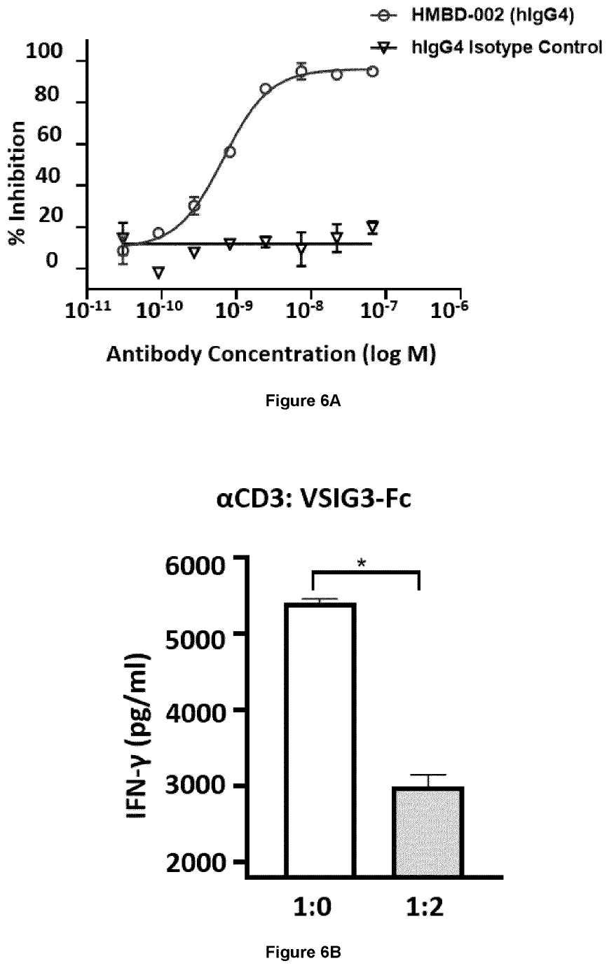

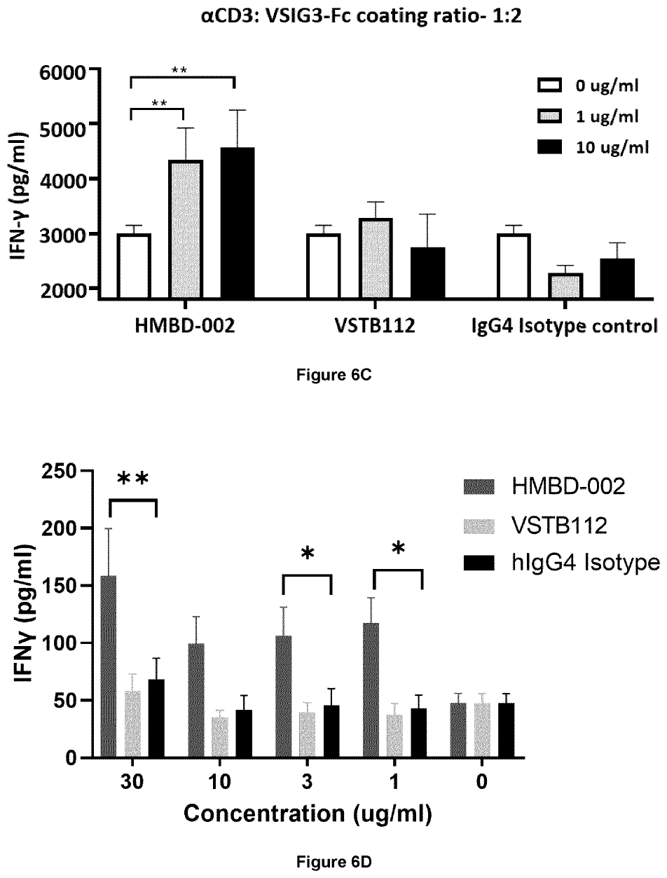

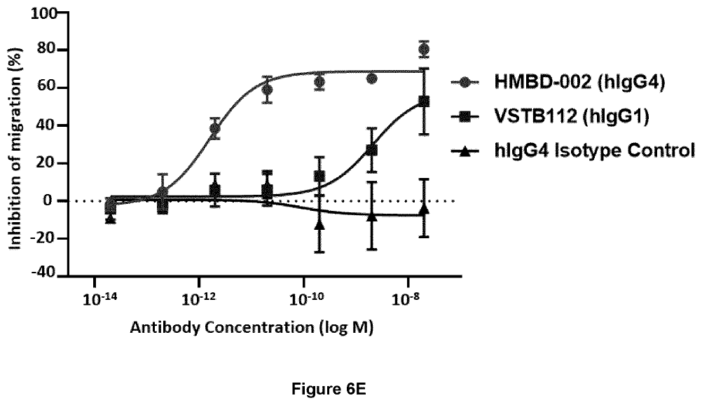

FIG. 6A shows that V4-C26 hIgG4 can antagonize VISTA-VSIG3 interaction in a dose-dependent manner, with an IC₅₀ of 673 pM.

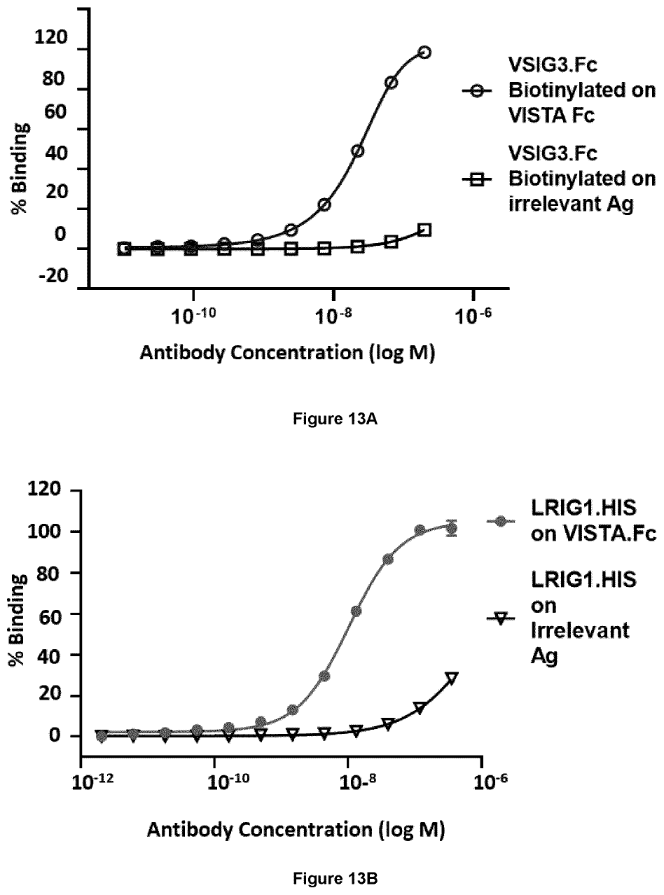

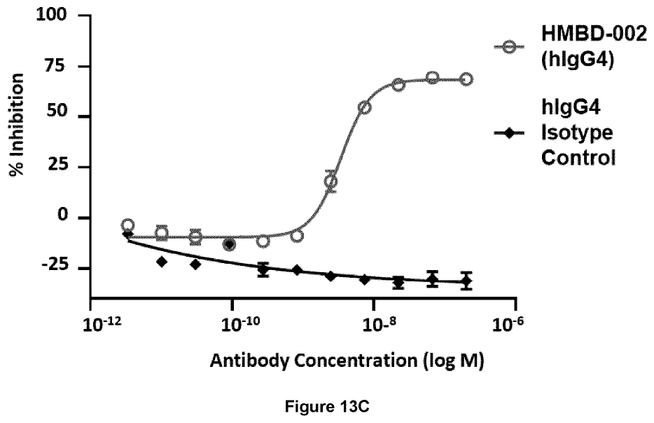

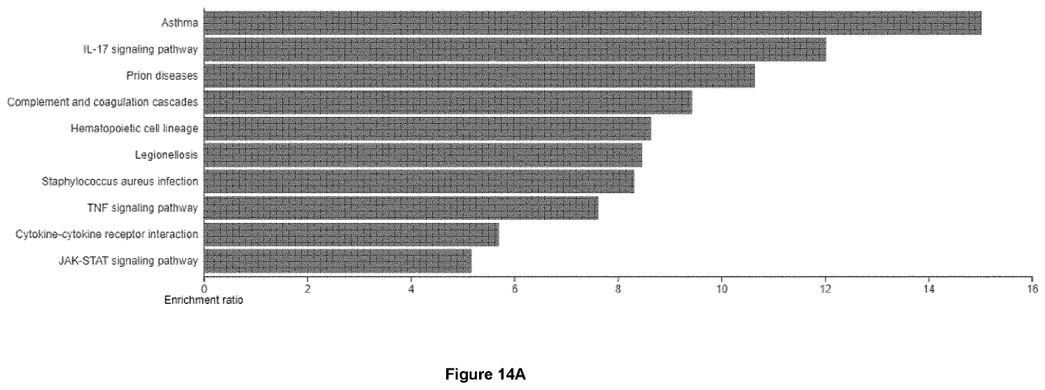

Similarly, FIG. 13C shows that V4-C26 hIgG4 demonstrates a dose dependent inhibition of VISTA-LRIG1 binding, with a IC₅₀ value of 3.4 nM.

To evaluate the functional effect of inhibiting VISTA-VSIG3 interaction with V4-C26 hIgG4, a surrogate assay for tumoricidal T cell activity was performed (e.g. as described in J. Terhune, et al., Nato Adv Sci Inst Se 1, 527-549 (2013)). In this assay, an anti-CD3 antibody mimics T cell receptor ligation, resulting in IFN-γ secretion that can be quantified in the supernatant by ELISA. As expected, incubating human PBMCs with VSIG3 significantly suppressed the release of IFN-γ (FIG. 6B). Notably, this suppression could be effectively released in a dose-dependent manner with the addition of V4-C26 hIgG4, but not VSTB112, to the media (FIG. 6C).

These data support the hypothesis that V4-C26 hIgG4 functionally blocks interactions of VISTA with key protein partners, such as VSIG3 and LRIG1, and further suggest a potential mechanism of action of V4-C26 hIgG4—blockade of VISTA-VSIG3 mediated suppression of pro-inflammatory IFN-γ secretion from activated T cells.

6.2 V4-C26 hIgG4 Modulates Myeloid Function

To investigate the effect of VISTA blockade on myeloid cells that express the highest levels of VISTA, V4-C26 hIgG4 treatment was evaluated in several in vitro models of myeloid function.

First, VISTA blockade by V4-C26 hIgG4 was assessed for its effect on the function of human monocytic MDSCs. Previous studies have reported that MDSCs contribute significantly to suppression of T cell function in the TME (L. Wang, et al., Oncoimmunology 7, e1469594 (2018)). Monocytes differentiated to MDSCs for 7 days with GM-CSF and IL-6 were co-cultured with autologous PBMCs. T cells were then stimulated with an anti-CD3 antibody and IFN-γ levels in the culture supernatant were measured by ELISA.

FIG. 6D shows that addition of V4-C26 hIgG4, but not VSTB112, successfully reversed MDSC-mediated T cell suppression in response to anti-CD3 stimulation as indicated by the enhanced levels of IFN-γ.

Granulocytes such as neutrophils are an integral part of the innate immune response but can adversely affect cancer progression. Although it is challenging to model the function of granulocytic (g)-MDSCs in vitro, it is known that g-MDSCs can arise from neutrophils that have infiltrated the tumour microenvironment (G. E. Kaiko, et al., Immunology 123, 326-338 (2008)). The effect of V4-C26 hIgG4-mediated VISTA blockade on neutrophil chemotaxis was explored using a transwell assay. In this assay, neutrophils migrate between chambers towards a physiologically relevant chemoattractant, C5a, and the percent of cells that have migrated to the lower chamber is then quantified via a luminescence readout.

FIG. 6E shows that V4-C26 hIgG4 potently inhibited neutrophil migration in a dose-dependent manner. VSTB112, in contrast, could only suppress neutrophil migration at much higher concentrations. Collectively these results demonstrate that V4-C26 hIgG4 potently neutralizes VISTA on myeloid cells leading to the increased secretion of pro-inflammatory cytokines and decreased migration of neutrophils.

Thus, V4-C26 hIgG4 neutralizes VISTA activity by inhibiting VISTA-LRIG1 and VISTA-VSIG3 binding, attenuating VSIG3-mediated suppression of IFN-γ release from activated T cells, reducing MDSC-mediated T cell suppression and inhibiting neutrophil chemotaxis.

6.3 V4-C26 hIgG4 Polarises the Immune Cell Milieu Towards a T_{H}1/T_{H}17 Immune Response

To investigate the functional effect of VISTA blockade by V4-C26 hIgG4 in more complex in vitro models of immune activation, an allogenic Mixed Lymphocyte Reaction (MLR) assay was used. Briefly, PBMCs from 5 independent healthy human donors were mixed pairwise to model a self/anti-self immune response and were then cultured for up to 96 hours in the presence or absence of V4-C26 hIgG4 before supernatants were analyzed for cytokine levels. The transcriptome of the cells was also analyzed using bulk RNA-seq. Cytokine levels in the supernatant were quantified by Luminex assay.

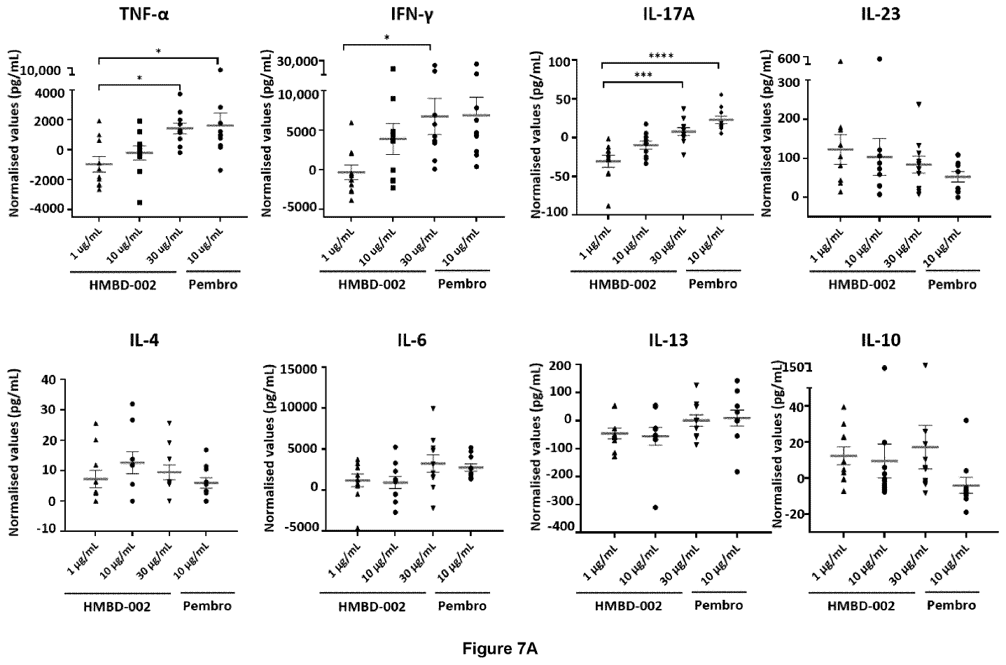

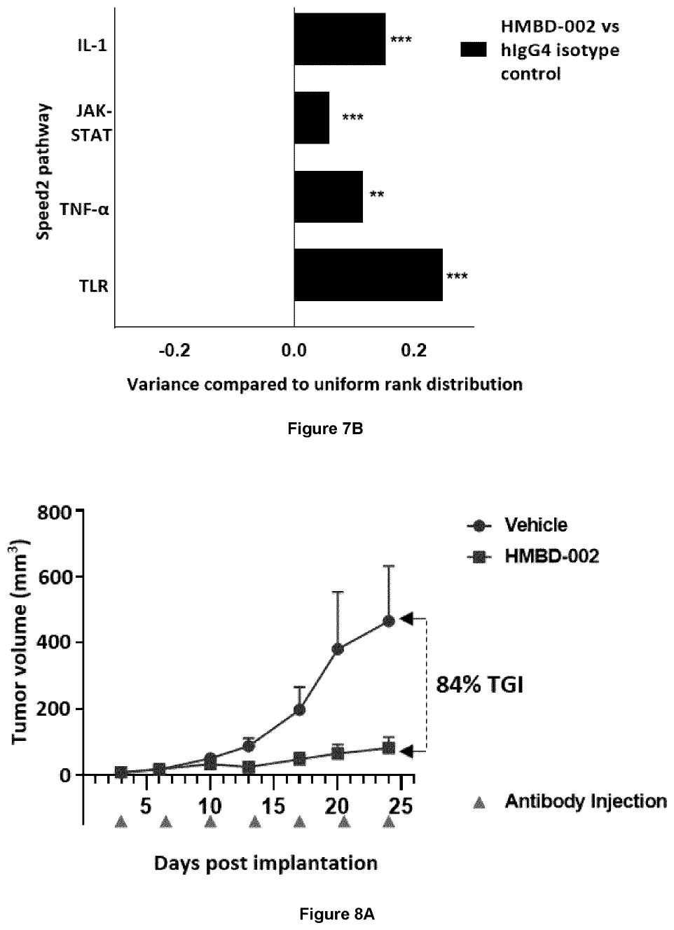

FIG. 7A shows that V4-C26 hIgG4 induced a significant dose dependent increase in the levels of IFN-γ, TNF-α and IL-17A at 96 hours, comparable to the effect seen with PD-1/PD-L1 blockade by an anti-PD-1 antibody, Pembrolizumab, but no significant changes in the levels of IL-4, IL-10 and IL-13 (Th2 cytokines) or IL-6. These changes in cytokine levels are indicative of a shift to a Th1/Th17 response.

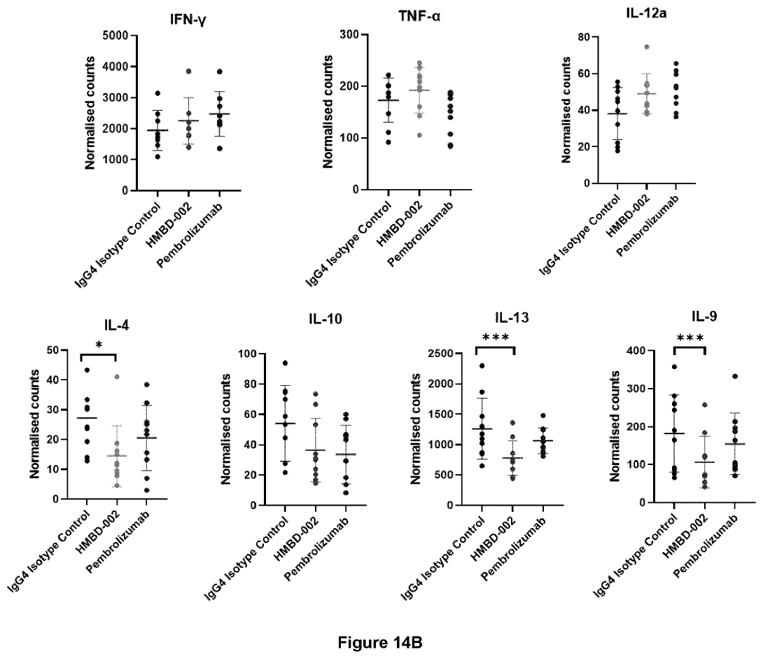

This conclusion was further supported by bulk RNA-seq that highlighted an enrichment of transcript levels in genes associated with TLR, TNF-α, IL-1, JAK-STAT and IL-17 signalling pathways (FIGS. 7B, 14A) along with an increase in transcript levels of Th1 associated genes such as IFN-γ, TNF-α and IL-12A and a decrease in transcript levels of Th2 associated genes IL-4, IL-10, IL-13 and IL-9 (FIG. 14B).

Collectively, these results show that V4-C26 hIgG4 blockade of VISTA can polarize the immune cell milieu toward an enhanced Th1/Th17 immune response. This is consistent with murine VISTA knockout models that lead to psoriasis and experimental autoimmune encephalomyelitis (EAE) that have previously been reported to be characterized by Th1/Th17 responses (N. Li et al., Sci Rep-uk 7, 1485 (2017)).

6.4 V4-C26 hIgG4 Treatment Induces Strong Anti-Tumour Responses in Multiple Immune Competent Murine CDX Models of Solid Tumours

To explore the pro-inflammatory and anti-tumorigenic effects of V4-C26 hIgG4 in vivo, tumour growth inhibition studies were conducted in multiple solid tumour models that included a syngeneic murine cell-derived xenograft (CDX) subcutaneous model of colon cancer (CT26), a checkpoint inhibitor-resistant orthotopic CDX model of VISTA expressing breast cancer (4T1) and CD34 engrafted humanized mouse models of human lung cancer (A549) and colorectal cancer (HCT15).

First, Balb/c mice were subcutaneously implanted (right flank) with CT26 tumours and treated biweekly with 500 μg (˜25 mg/kg) of V4-C26 hIgG4 (intraperitoneal) from 3 days post implantation.

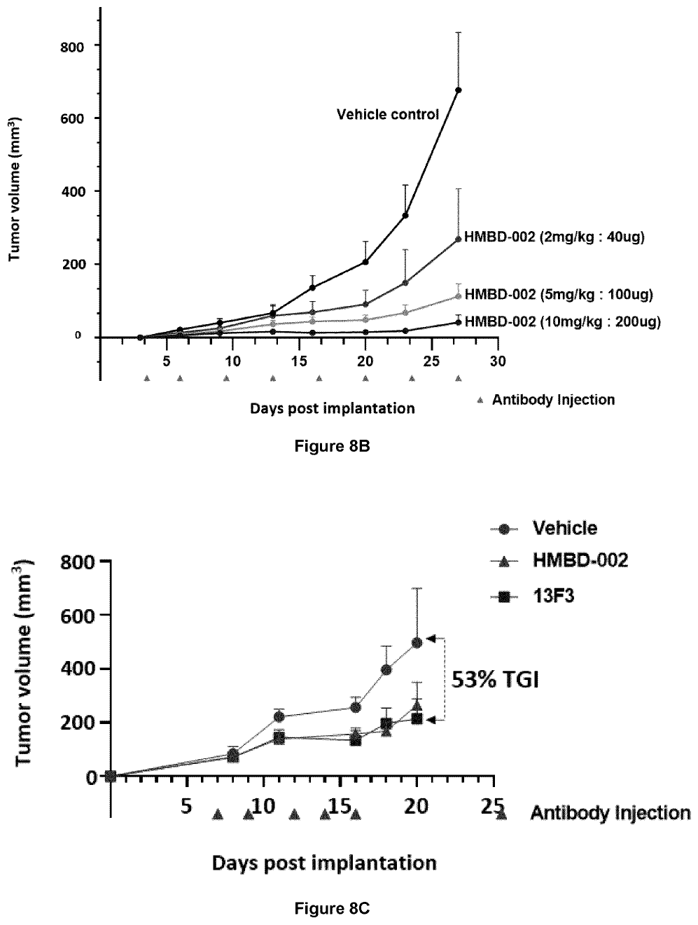

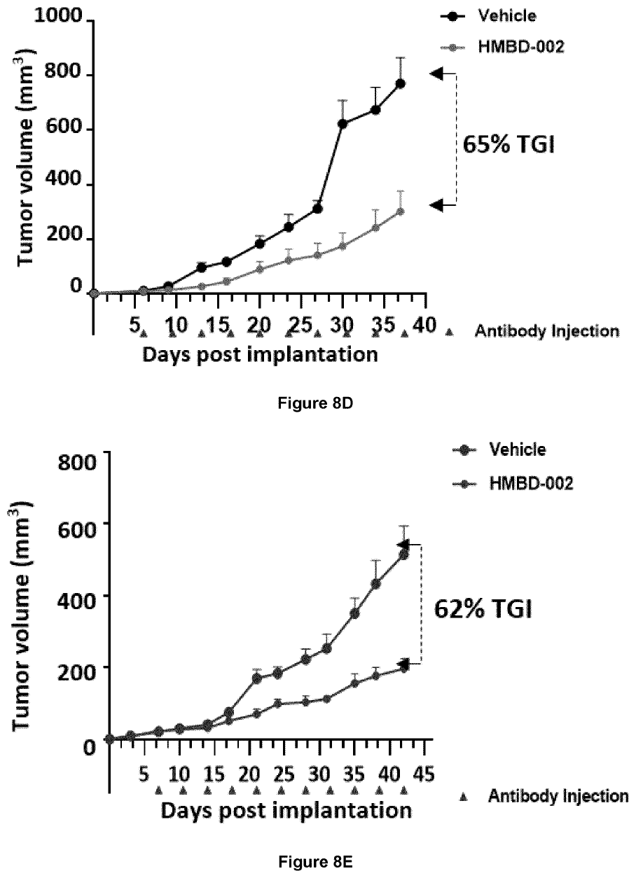

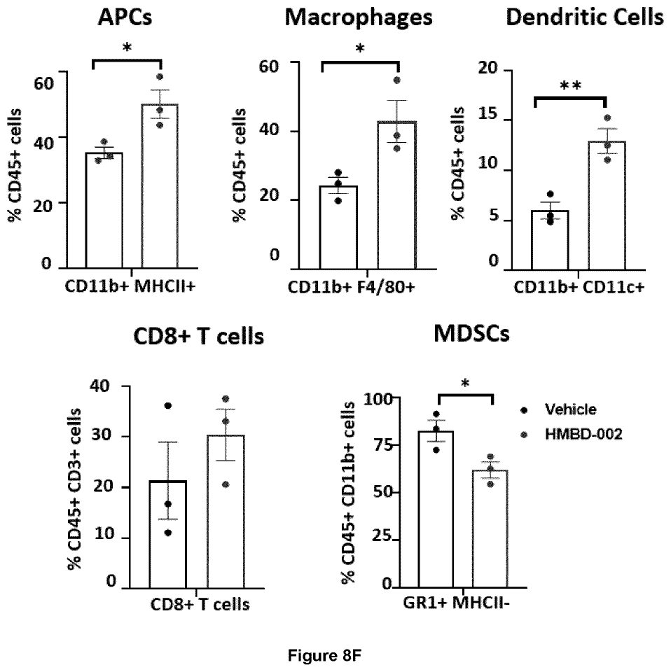

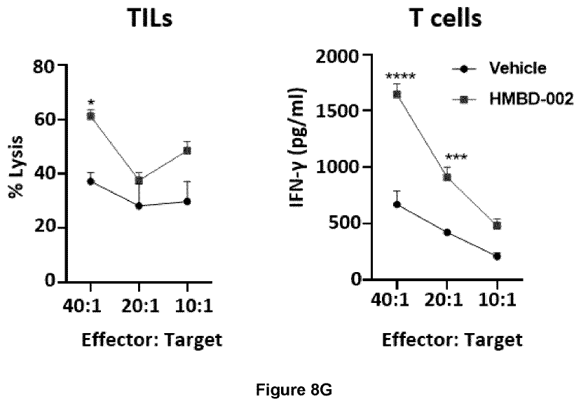

FIG. 8A shows that V4-C26 hIgG4 demonstrated significant single agent efficacy with 84% inhibition of tumour growth (TGI) compared to vehicle.

In separate experiments in the CT26 CDX model, mice were treated with lower doses of V4-C26 hIgG4 (200 μg, 100 μg or 40 μg) biweekly from 3 days post-implantation, in order to determine the minimum dose at which V4-C26 hIgG4 is effective in vivo. FIG. 8B shows that V4-C26 hIgG4 was found to be effective at all of the doses tested, with robust tumor growth inhibition observed at doses of 100 μg (˜5 mg/kg) and above.

Secondly, a murine orthotopic model of breast cancer was established by implanting 4T1 cells overexpressing VISTA in the mammary fat pads of Balb/c mice. Mice were treated intratumorally with 50 μg of either V4-C26 hIgG4 or the anti-mouse VISTA antibody 13F3 on days 7, 9, 12, 14, 16 post-implantation. 13F3 is frequently used as a mouse surrogate for studying the in vivo efficacy of anti-VISTA antibodies.

FIG. 8C shows again that V4-C26 hIgG4 showed significant TGI (53%), which was comparable to that of 13F3 suggesting the two antibodies may share a common mechanism of action.

Finally, the efficacy of V4-C26 hIgG4 was tested in humanized mice engrafted with CD34+ cord blood hematopoietic stem cells. These humanized mice received a GM-CSF/IL3 boost that stably reconstituted multiple human cell lineages such as T, B and myeloid cells in organs and blood, two days prior to tumor implantation (Table S1). After reconstitution for 3-4 months, mice were subcutaneously implanted with either human HCT15 colorectal cancer cells or human A549 lung cancer cells and treated biweekly (intraperitoneal with 500 μg (˜25 mg/kg) of either V4-C26 hIgG4 or vehicle control starting at 5 days post implantation.

FIGS. 8D and 8E show that, as observed in the syngeneic models, V4-C26 hIgG4 showed significant single agent anti-tumour efficacy of 65% and 62% TGI in these HCT15 colorectal and A549 lung humanized CDX models, respectively.

Thus, V4-C26 hIgG4 demonstrates strong anti-tumour responses as a single agent in multiple CDX models of solid tumours. The similar tumour inhibition observed after V4-C26 hIgG4 treatment of both fully immune-competent murine tumour models and murine tumour models recapitulating the human immune compartment, suggests a conserved, and potentially translatable, mechanism of action shared between mouse and human anti-tumour immune responses.

6.5 V4-C26 hIgG4 Treatment Remodels the Tumour Microenvironment of Murine CDX Models, Increasing Activated Effector Immune Cells and Decreasing Suppressive Cells

To understand the mechanism of the anti-tumour efficacy observed for V4-C26 hIgG4 in murine models, tumours from the syngeneic CT26 colon cancer models were further profiled using FACS.

FIG. 8F shows that V4-C26 hIgG4 treatment was observed to significantly increase the percentage of CD11 b+MHCII+(antigen presenting cells), CD11 b+F4/80+(macrophages) and CD11c+(DCs) in the tumour microenvironment. An increase in CD8+ T cells was also observed, although this was not statistically significant. In contrast, the frequency of broadly-suppressive MDSCs (CD11 b+GR1+MHCII−) was significantly lower in the tumours of treated mice compared to vehicle control.

To further assess the mechanism by which these changes in immune cell numbers were associated with the functional state of tumour infiltrating lymphocytes (TILs), we performed an antigen recall assay by co-culturing CT26 cells and isolated TILs from the CDX models in vitro.

FIG. 8G shows that, 72 hours after coculture, TILs isolated from tumours treated with V4-C26 hIgG4 showed significantly higher lysis of CT26 cells. This increased activity was further confirmed with an ex vivo co-culture assay of infiltrating T cells with CT26 cells, where T cells from treated mice showed significantly higher IFN-γ levels, as measured by ELISA, compared to untreated mice.

These results suggest that VISTA blockade by V4-C26 hIgG4 increases the levels of inflammatory effector cells with concomitant decrease of immunosuppressive MDSCs in the tumour microenvironment and enhances the antigen-specific cytotoxic activity of TILs, likely contributing to the anti-tumour efficacy of V4-C26 hIgG4. V4-C26 hIgG4 is able to remodel the tumour microenvironment to an anti-tumorigenic, pro-inflammatory phenotype, consistent with the primary mechanism of action being manipulation of the highly VISTA-positive myeloid compartment.

### Example 7: V4-C26 hIqG4 Exhibits Favourable Pharmacokinetic Profiles in Multiple Species

Therapeutic antibodies should possess half-lives in plasma compatible with appropriate dosing regimens. Previous anti-VISTA antibodies have demonstrated poor PK characterized by rapid serum clearance (R. J. Johnston et al., Nature 574, 565-570 (2019)), which may be explained by Fc-effector functions such as ADCC, especially of neutrophils, leading to rapid cell turnover of VISTA expressing cells and causing a significant antibody sink. The pharmacokinetic profile of V4-C26 hIgG4 was therefore evaluated in healthy and tumour-bearing mice, and healthy rats and NHP.

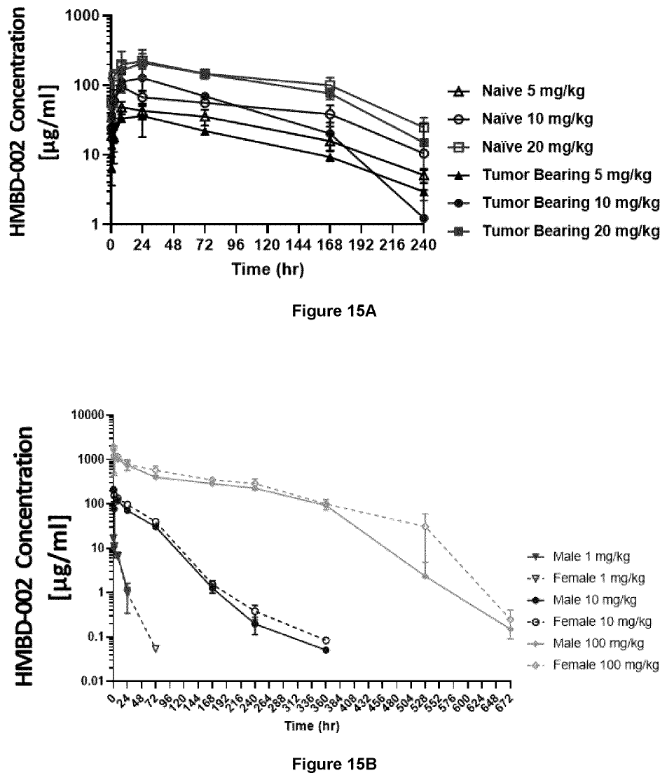

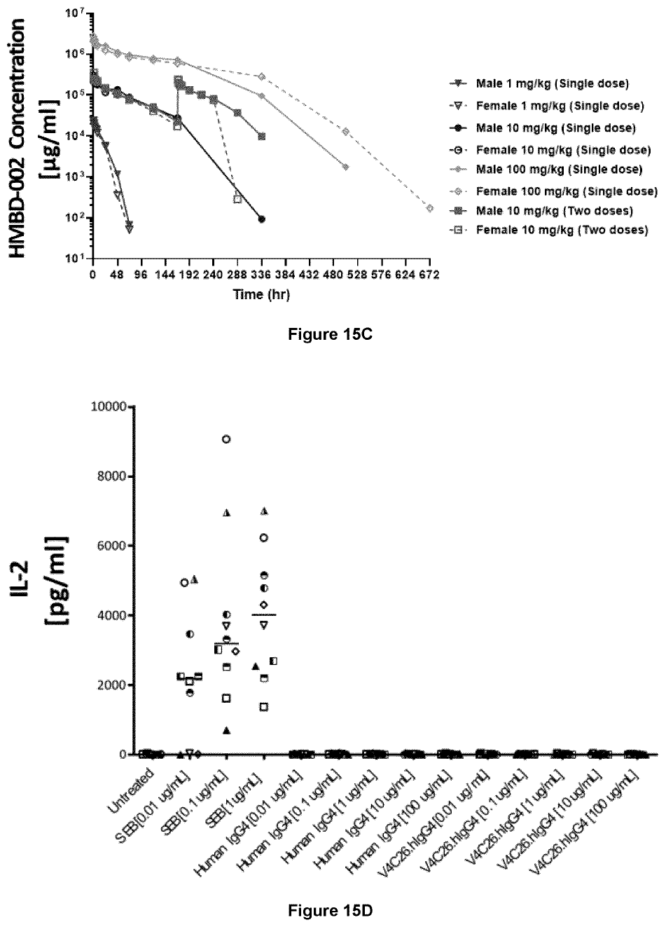

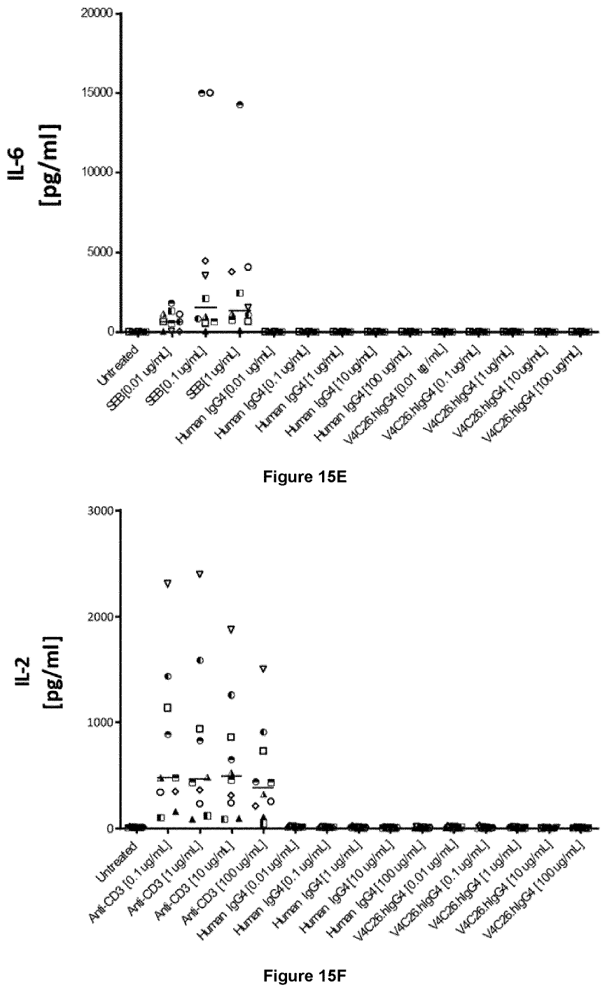

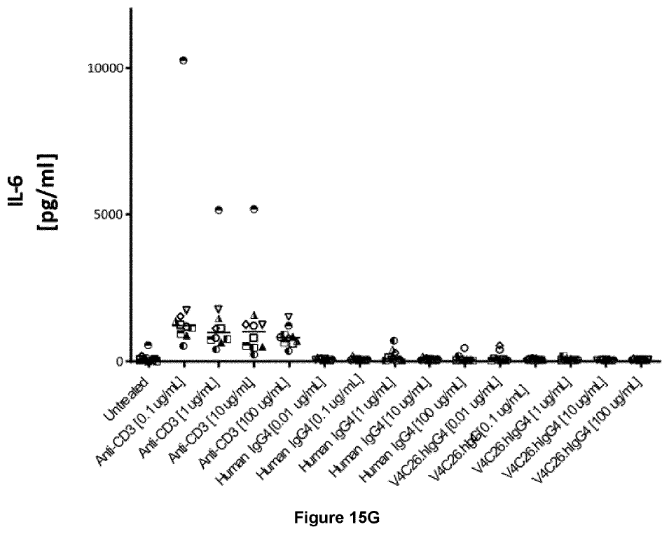

FIG. 15A shows representative pharmacokinetic profiles for tumour-bearing and non-tumour bearing Balb/c mice that had been dosed intraperitoneally with increasing doses of V4-C26 hIgG4 (5-20 mg/kg). Non-tumour bearing mice demonstrated linear PK, however, profiles in tumour bearing mice were non-linear, likely due to the higher levels of the target and increased target mediated drug disposition (TMDD). V4-C26 hIgG4 serum half-life in the tumour bearing mice was calculated as 26.9 to 57.2 hours across doses, while in the non-tumour bearing mice serum half-life was 61.1 to 80.8 hours.

In Sprague-Dawley rats and cynomolgus monkeys, the PK profile was determined from measurements in both male and female animals. V4-C26 hIgG4 was administered at single doses of 1 mg/kg, 10 mg/kg, and 100 mg/kg, as an intravenous (IV) bolus infusion into the tail vein in rats, and as an IV bolus infusion into the peripheral vein in monkeys. In both species, the PK profiles of V4-C26 hIgG4 did not differ between the genders and dose proportional increases in exposure were observed for Cmax values across all doses. Serum half-life for V4-C26 hIgG4 increased with each dosing group. The mean observed half-life across both genders in rat were 6.3, 25.5, and 67.5 hours for 1, 10, and 100 mg/kg, respectively, whereas the mean observed half-life across both genders in cynomolgus monkey were 8.6, 41.3, and 34.9 hours for 1, 10, and 100 mg/kg, respectively (FIGS. 151B, 15C).

Together, these results demonstrate that V4-C26 hIgG4 has a favourable PK profile in multiple species without the rapid clearance that has been noted for other anti-VISTA antibodies with a depleting IgG1 Fc domain.

Optimal therapeutic antibodies should demonstrate minimal toxicity to normal tissues and avoid detrimental side effects such as cytokine release syndrome (as reported for other IgG1 isotype anti-VISTA depleting antibodies at sub therapeutic doses (NCT02671955)). As V4-C26 hIgG4 shows species conserved binding of VISTA orthologs, animals from the above PK studies were also monitored for adverse effects of V4-C26 hIgG4 dosing, as part of concurrent tolerability studies. After a single IV injection in Sprague-Dawley rats and cynomolgus monkey, both species showed no treatment related morbidity/mortality or clinical signs, and no treatment related changes in body weight, food consumption, clinical chemistry, or hematology parameters that continued for the duration of the 28-day observation period.

Additionally, ex vivo cytokine release assays were conducted with human whole blood and isolated PBMCs from healthy donors, to assess the potential immunotoxicity of V4-C26 hIgG4 by evaluating the levels of IL-2, IL-4, IL-6, IL-10, TNF-α and IFN-γ in culture supernatants using a cytometric bead array approach. Cytokine levels were measured after stimulation for 24 hours with either V4-C26 hIgG4, a positive control (anti-CD3 or staphylococcal enterotoxin B), or a negative isotype control in soluble stimulation format. V4-C26 hIgG4 did not elicit any significant cytokine release in blood or in purified PBMCs (FIGS. 15D to 15G).

Together these results demonstrate that V4-C26 hIgG4 is well tolerated in rats and cynomolgus monkeys and further suggests a low risk for potential immunotoxicity caused by cytokine release.

### Example 8: V4-C26 hIG4 Enhances CD8+ T Cell Activation and Reprograms Tumour-Associated Macrophaqes

To further understand the mechanism of anti-tumour efficacy observed for V4-C26 hIgG4 in murine models, tumours from the syngeneic CT26 colon cancer models were profiled by flow cytometry to assess in more detail the ability of V4-C26 hIgG4 to remodel the tumour microenvironment. The murine colon cancer model was used as this exhibited robust infiltrations of multiple immune cells into tumours and allowed for simultaneous investigation of VISTA-mediated immune modulation in multiple cell subsets.

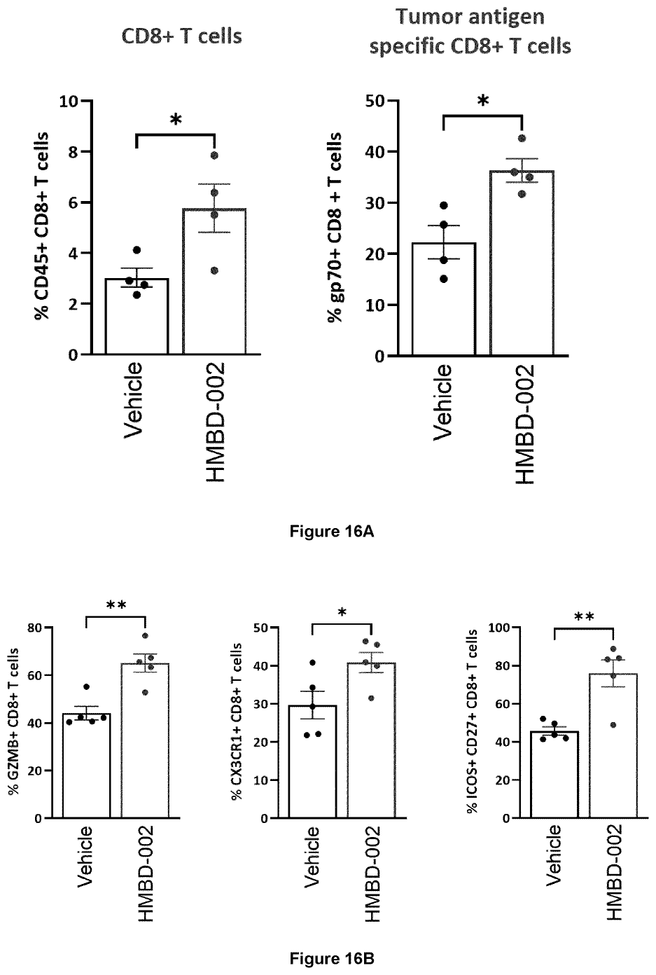

FIG. 16A shows that V4-C26 hIgG4 treatment was observed to significantly increase the percentage of CD45+CD8+ T cells and gp70+CD8+ T cells (tumour antigen-specific CD8+ T cells) in the tumour microenvironment.

FIG. 16B shows that V4-C26 hIgG4 treatment was observed to significantly increase the percentage of GZMB+CD8+ T cells, CX3CR1+CD8+ T cells and ICOS+CD27+CD8+ T cells in the tumour microenvironment.

These results demonstrate that VISTA blockade by V4-C26 hIgG4 increases levels of CD8+ T cells and of tumour antigen-specific CD8+ T cell subset. A concurrent increase in CD8+ T cell activation was seen with an upregulation of several cytotoxicity associated markers, including Granzyme B.

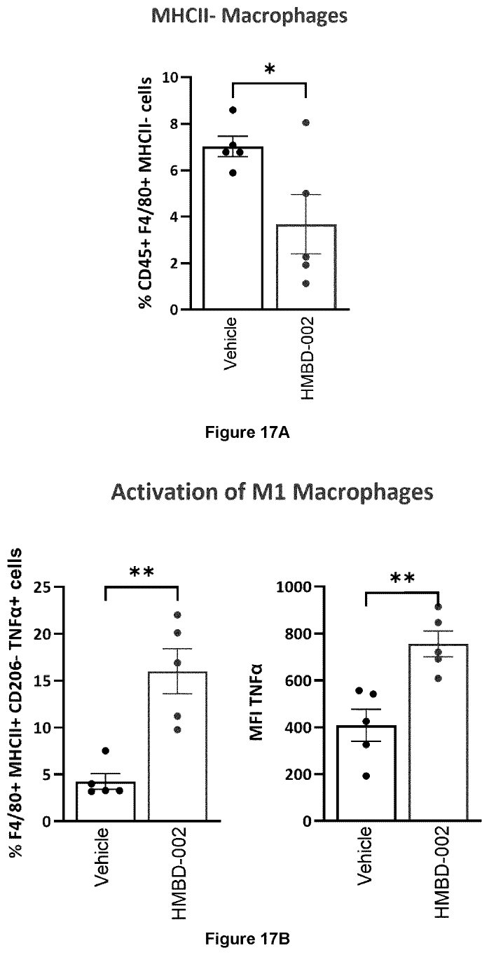

FIG. 17A shows that V4-C26 hIgG4 treatment was observed to significantly decrease the percentage of CD45+F4/80+MHCII− cells (MHCII− macrophages) in the tumour microenvironment.

FIG. 17B shows that V4-C26 hIgG4 treatment was observed to significantly increase the percentage of F4/80+MHCII+CD206-TNFα+ cells (M1 macrophages), and to significantly increase the mean fluorescence intensity of TNFα.

These results demonstrate that VISTA blockade by V4-C26 hIgG4 polarized macrophages to an activated pro-inflammatory anti-tumor phenotype with significant increases in TNFa and MHCII expressing macrophage subsets.

### Example 9: V4-C26 hIG4 Treatment Promotes Transcriptional Programs Associated with Anti-Tumor Immune Response

To determine molecular changes mediated by V4-C26 hIgG4 (HMBD-002), the transcriptome of CT26 tumors treated with vehicle control or V4-C26 hIgG4 were analysed via RNA seq.

Briefly, mouse tumours (150-200 mm²) were dissociated using gentleMACS™ tissue dissociator (Miltenyi), following the manufacturer's protocol. RNA was extracted using RNAeasy mini kit (Qiagen) following the manufacturer's protocol. Stranded RNA-seq libraries were prepared after rRNA depletion using Illumina Stranded total RNA prep kit with Ribo-zero plus. Paired-end sequencing was performed on Illumina's Novaseq platform to obtain 40M (12 Gb) reads per sample. The resulting reads were processed by the nf-core RNA-seq pipeline. The reads were aligned to the mouse reference genome downloaded from iGenomes (GRCm38 Ensembl release 81). Gene counts were obtained with Salmon, the matrix of counts were loaded into R and the differential expression analysis was performed using DESeq2.

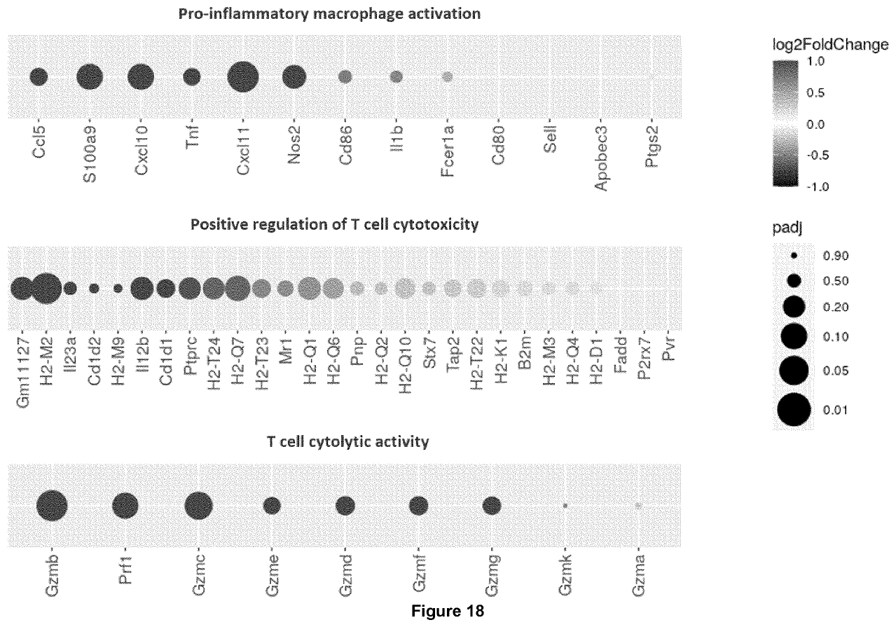

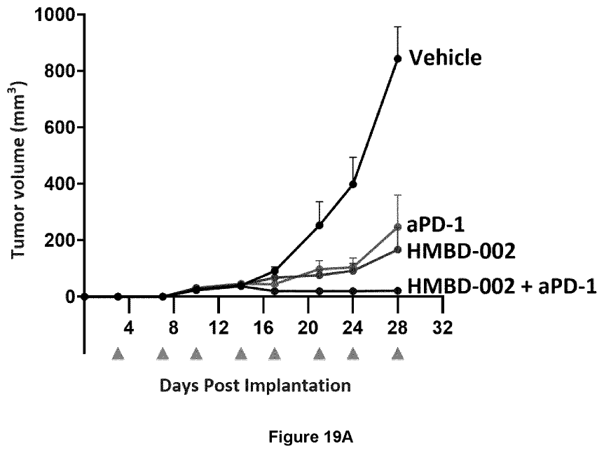

FIG. 18 shows that V4-C26 hIgG4 treatment was observed to significantly upregulate transcription of multiple genes associated with pro-inflammatory macrophage activation positive regulation of T cell cytotoxicity and T cell cytolytic activity in CT26 tumours. Each gene measured showed upregulation, apart from Apobec3, Ptgs2, P2rx7, and Pvr which showed no change or a slight downregulation.

These results demonstrate that VISTA blockade by V4-C26 hIgG4 led to upregulation of multiple genes involved in macrophage mediated pro-inflammatory immune response and cytotoxic activity of T cells. The results were in line with flow cytometry-based profiling of CT26 tumors (see Example 8) as a number of genes including TNFα and granzyme B were upregulated both at the protein and RNA level.

### Example 10: V4-C26 hIG4 Treatment in Combination with aPD-1 Induces Enhanced Anti-Tumour Response

To explore the pro-inflammatory and anti-tumorigenic effects of V4-C26 hIgG4 in combination with aPD-1, tumour growth inhibition studies were conducted in a syngeneic murine cell-derived xenograft (CDX) subcutaneous model of colon cancer (CT26). Tumours from the models were also profiled using FACS to understand the mechanism of the anti-tumour efficacy.

Balb/c mice were subcutaneously implanted with CT26 tumours and treated (intraperitoneal) biweekly from 3 days post implantation with:
PBS (Vehicle)
V4-C26 hIgG4 at 25 mg/kg
aPD-1 (Bioxcell, BE0033-2) at 10 mg/kg
V4-C26 hIgG4 at 25 mg/kg and aPD-1 (Bioxcell, BE0033-2) at 10 mg/kg.

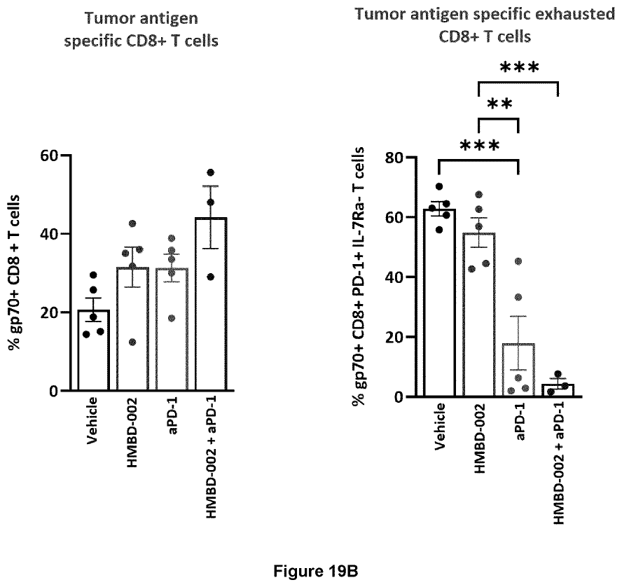

FIG. 19A shows that V4-C26 hIgG4 and aPD-1 as monotherapies demonstrated strong single agent efficacies. The combination of V4-C26 hIgG4 and aPD-1 enhances anti-tumour efficacy when compared to monotherapy V4-C26 hIgG4 or aPD-1.

FIG. 19B shows that V4-C26 hIgG4 and aPD-1 as monotherapies increase levels of gp70+CD8+ T cells (tumour antigen-specific T cells) and deceased levels of gp70+CD8+PD-1+IL-7Ra− T cells (tumour antigen-specific exhausted T cells). The combination of V4-C26 hIgG4 and aPD-1 further increases levels of gp70+CD8+ T cells (tumour antigen-specific T cells) and significantly decreases levels of gp70+CD8+PD-1+IL-7Ra− T cells (tumour antigen-specific exhausted T cells) when compared to monotherapy V4-C26 hIgG4 or aPD-1.

These results demonstrate that VISTA blockade by V4-C26 hIgG4 in combination with aPD-1 action has enhanced anti-tumour effect and increases levels of antigen-specific CD8+ T cells with reduced exhaustion.

### Example 11: V4-C26 hIqG4 Treatment in a Humanised Mesothelioma PDX Model

Mesothelioma is among the high VISTA expressing cancers and epithelioid mesothelioma has been reported to have highest VISTA expression among mesothelioma subtypes. To explore the anti-tumorigenic effects of V4-C26 hIgG4 in mesothelioma, tumor growth inhibition studies were conducted in a humanized VISTA positive patient-derived xenograft (PDX) model of malignant pleural epithelioid mesothelioma.

At day −60, humanized CD34+NCG mice (NOD-Prkdc^{em26cd52}Il2rg^{em26cd22}/NjuCrl) (Charles River Labs) were subcutaneously implanted with PNX0411 PDX tumor.

Treatment with PBS (Vehicle) or V4-C26 hIgG4 at 25 mg/kg commenced (Day 0), when the tumors reached a mean tumor size of approximately 90-100 mm³. Treatment was performed intraperitoneally biweekly.

At the end of the study (approximately 6-8 weeks after treatment initiation), blood and tumor samples are collected for cytokine and FACS analysis, respectively. Cytokine analysis is performed using the validated immunoassay-based MesoScale (MSD) kit for IL1b, IL2, IL4, IL6, IL8, IL10, IL12/p70, IL13, TNF-α, IFN-β levels. Flow cytometry analysis is performed for human markers such as Live/Dead, huCD45, huCD3, huCD8, huCD4, huCD56, HLA-DR, PD-1, huCD14, and huVISTA.

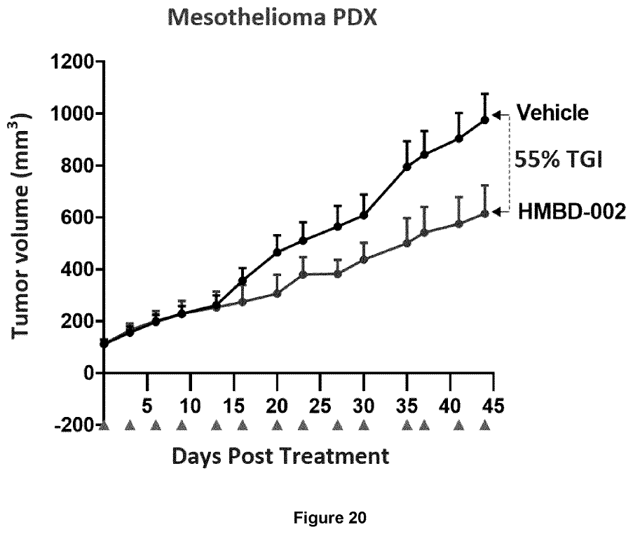

FIG. 20 shows that mice treated with V4-C26 hIgG4 showed a significant reduction of tumour growth, demonstrating that V4-C26 hIgG4 as a monotherapy has strong anti-tumor efficacy in malignant pleural epithelioid mesothelioma PDX.

1. An antigen-binding molecule that binds to VISTA for use in a method of treating or preventing a cancer in a subject, wherein the treatment or prophylaxis comprises:
(i) increasing the number and/or proportion of antigen-specific CD8+ T cells;
(ii) increasing CD8+ T cell activity;
(iii) reducing the level of T cell exhaustion;
(iv) reducing the number and/or proportion of tumour-associated macrophages (TAMs);
(v) increasing the number and/or proportion of M1-type macrophages; and/or
(vi) increasing M1-type macrophage activity.

2. Use of an antigen-binding molecule that binds to VISTA in the manufacture of a medicament for treating or preventing a cancer in a subject, wherein the treatment or prophylaxis comprises:
(i) increasing the number and/or proportion of antigen-specific CD8+ T cells;
(ii) increasing CD8+ T cell activity;
(iii) reducing the level of T cell exhaustion;
(iv) reducing the number and/or proportion of tumour-associated macrophages (TAMs);
(v) increasing the number and/or proportion of M1-type macrophages; and/or
(vi) increasing M1-type macrophage activity.

3. A method of treating or preventing a cancer in a subject, wherein the method comprises administering to a subject a therapeutically or prophylactically effective amount of an antigen-binding molecule that binds to VISTA, wherein the treatment or prophylaxis comprises:
(i) increasing the number and/or proportion of antigen-specific CD8+ T cells;
(ii) increasing CD8+ T cell activity;
(iii) reducing the level of T cell exhaustion;
(iv) reducing the number and/or proportion of tumour-associated macrophages (TAMs);
(v) increasing the number and/or proportion of M1-type macrophages; and/or
(vi) increasing M1-type macrophage activity.

4. The antigen-binding molecule for use according to claim 1, the use according to claim 2, or the method according to claim 3, wherein the cancer comprises a tumor comprising cells expressing VISTA.

5. A method of selecting a subject for treatment with an antigen-binding molecule that binds to VISTA, comprising:
(a) analysing a subject's cancer in order to determine whether the cancer is characterised by:
(i) low number and/or proportion of antigen-specific CD8+ T cells;
(ii) low CD8+ T cell activity;
(iii) presence and/or high levels of exhausted T cells;
(iv) presence and/or high number and/or proportion of TAMs;
(v) low number and/or proportion of M1-type macrophages; and/or
(vi) low M1-type macrophage activity; and
(b) selecting a subject for treatment with an antigen-binding molecule that binds to VISTA where the subject's cancer is determined in step (a) to have one or more of (i) to (vi).

6. A method of determining the response in a patient to treatment with an antigen-binding molecule that binds to VISTA, comprising:
(a) analysing a subject's cancer at a first timepoint in order to determine the:
(i) number and/or proportion of antigen-specific CD8+ T cells;
(ii) CD8+ T cell activity;
(iii) level of T cell exhaustion;
(iv) number and/or proportion of tumour-associated macrophages (TAMs);
(v) number and/or proportion of M1-type macrophages; and/or
(vi) M1-type macrophage activity;
(b) analysing a subject's cancer at a subsequent timepoint in order to determine one or more of (i) to (vi); and
(c) determining the difference between (a) and (b), wherein a(n):
(i) increased number and/or proportion of antigen-specific CD8+ T cells;
(ii) increased CD8+ T cells activity;
(iii) reduced level of T cell exhaustion;
(iv) reduced number and/or proportion of tumour-associated macrophages (TAMs);
(v) increased number and/or proportion of M1-type macrophages; and/or
(vi) increased M1-type macrophage activity,
in (b) relative to (a) signifies a positive response to treatment with an antigen-binding molecule that binds to VISTA.

7. The antigen-binding molecule for use, the use or the method according to any one of claims 1 to 6, wherein the antigen-binding molecule comprises:
(i) a heavy chain variable (VH) region incorporating the following CDRs:
HC-CDR1 having the amino acid sequence of SEQ ID NO:305
HC-CDR2 having the amino acid sequence of SEQ ID NO:306
HC-CDR3 having the amino acid sequence of SEQ ID NO:307; and
(ii) a light chain variable (VL) region incorporating the following CDRs:
LC-CDR1 having the amino acid sequence of SEQ ID NO:41
LC-CDR2 having the amino acid sequence of SEQ ID NO:308
LC-CDR3 having the amino acid sequence of SEQ ID NO:43.

8. The antigen-binding molecule for use, the use, or the method according to any one of claims 1 to 7, wherein the antigen-binding molecule comprises:
(i) a VH region incorporating the following CDRs:
HC-CDR1 having the amino acid sequence of SEQ ID NO:290
HC-CDR2 having the amino acid sequence of SEQ ID NO:291
HC-CDR3 having the amino acid sequence of SEQ ID NO:278; and
(ii) a VL region incorporating the following CDRs:
LC-CDR1 having the amino acid sequence of SEQ ID NO:41
LC-CDR2 having the amino acid sequence of SEQ ID NO:295
LC-CDR3 having the amino acid sequence of SEQ ID NO:43.

9. The antigen-binding molecule for use, the use, or the method according to any one of claims 1 to 8, wherein the antigen-binding molecule comprises:
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:297.

10. The antigen-binding molecule for use, the use, or the method according to any one of claims 1 to 9, wherein the antigen-binding molecule comprises:
a VH region incorporating the following framework regions (FRs):
HC-FR1 having the amino acid sequence of SEQ ID NO:63
HC-FR2 having the amino acid sequence of SEQ ID NO:292
HC-FR3 having the amino acid sequence of SEQ ID NO:293
HC-FR4 having the amino acid sequence of SEQ ID NO:281.

11. The antigen-binding molecule for use, the use, or the method according to any one of claims 1 to 10, wherein the antigen-binding molecule comprises:
a VL region incorporating the following framework regions (FRs):
LC-FR1 having the amino acid sequence of SEQ ID NO:288
LC-FR2 having the amino acid sequence of SEQ ID NO:298
LC-FR3 having the amino acid sequence of SEQ ID NO:284
LC-FR4 having the amino acid sequence of SEQ ID NO:47.

12. The antigen-binding molecule for use, the use, or the method according to any one of claims 1 to 11, wherein the antigen-binding molecule comprises a heavy chain comprising the amino acid sequence of SEQ ID NO:331.

13. The antigen-binding molecule for use, the use, or the method according to any one of claims 1 to 12, wherein the antigen-binding molecule comprises a light chain comprising the amino acid sequence of SEQ ID NO:317.

14. The antigen-binding molecule for use, the use or the method according to any one of claims 1 to 13, wherein the cancer is selected from: a hematological cancer, leukemia, acute myeloid leukemia, lymphoma, B cell lymphoma, T cell lymphoma, multiple myeloma, mesothelioma, epithelioid mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

15. The antigen-binding molecule for use, the use or the method according to claim 14, wherein the cancer is selected from: colorectal cancer, pancreatic cancer, breast cancer, triple-negative breast cancer, liver cancer, prostate cancer, ovarian cancer, head and neck cancer, leukemia, lymphoma, melanoma, thymoma, lung cancer, non-small cell lung cancer (NSCLC) and a solid tumor.

16. The antigen-binding molecule for use, the use or the method according to claim 14, wherein the cancer is epithelioid mesothelioma.
